(19) United States
(12) Patent Application Publication
(10) Pub. No.: US 2010/0029655 A1
(43) Pub. Date: Feb. 4, 2010

(54) Processes For The Preparation Of Anti-Viral Compounds And Compositions Containing Them

(76) Inventors: Martin Robert Leivers, Durham, NC (US); Ryan Lauchli, San Mateo, CA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/499,828

(22) Filed: Jul. 9, 2009

Related U.S. Application Data
(60) Provisional application No. 61/129,691, filed on Jul. 11, 2008.

(51) Int. Cl. A61K 31/5025; C07D 487/04; A61P 31/12

(52) U.S. Cl. 514/248; 544/236

(57) ABSTRACT

Disclosed are processes for the preparation of compounds of formula I and compositions that comprise said compounds of formula I.

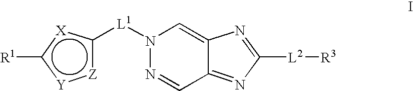

Also disclosed are processes for the preparation of compounds of formula III and compositions that comprise said compounds of formula III.

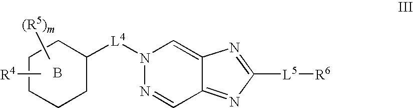

### CROSS-REFERENCE TO RELATED PATENTS AND PATENT APPLICATIONS

This application is related to and claims the priority benefit of U.S. Provisional Patent Application Ser. No. 61/129,691 filed Jul. 11, 2008, which is incorporated by reference herein in its entirety.

### BACKGROUND OF THE INVENTION

Processes for the preparation of antiviral compounds and compositions that contain said antiviral compounds are disclosed.

The following publications are cited in this application as superscript numbers:
1. Szabo, E. et al., Pathol. Oncol. Res. 2003, 9:215-221.
2. Hoofnagle J. H., Hepatology 1997, 26:15S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem 39, 223-237, 2004.
9. Watashi, K. et al., Molecular Cell, 19, 111-122, 2005
10. Horsmans, Y. et al, Hepatology, 42, 724-731, 2005

Chronic infection with HCV is a major health problem associated with liver cirrhosis, hepatocellular carcinoma, and liver failure. An estimated 170 million chronic carriers worldwide are at risk of developing liver disease. 2 In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of 3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory, and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. These include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

In view of the worldwide epidemic level of HCV and other members of the Flaviviridae family of viruses, and further in view of the limited treatment options, there is a strong need for new, effective drugs for treating infections caused by these viruses. Moreover, there is a strong need for processes to prepare these new, effective drugs and for compositions that comprise said drugs.

### DETAILED DESCRIPTION OF THE PREFERRED EMBODIMENTS

Provided are processes for the preparation of a compound of formula I:

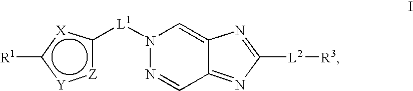

wherein

a) when X is CR² or N, one of Y or Z is O and the other of Y or Z is N; or one of Y or Z is N and the other of Y or Z is NR^{a};

b) when X is O, NR^{a}, or S(O)ₚ wherein p is 0 or 1, one of Y or Z is N and the other of Y or Z is N or CR²;

L¹ is L³;

L² is a bond or L³;

L³ is independently C₃₋₆ cycloalkylene or is C₁₋₅ alkylene where one or two —CH₂— groups of said C₂₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;

R^{a} and R^{b} are independently H, alkyl, or substituted alkyl;

R¹ and R³ are independently selected from aryl, substituted aryl, heteroaryl, substituted heteroaryl, heterocyclyl, substituted heterocyclyl, cycloalkyl, and substituted cycloallcyl;

and

R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano.

Also provided is a composition comprising:

(1) a compound of formula I or a salt or solvate thereof

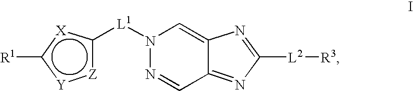

wherein

a) when X is CR² or N, one of Y or Z is O and the other of Y or Z is N; or one of Y or Z is N and the other of Y or Z is NR^{a};

b) when X is O, NR^{a}, or S(O)ₚ wherein p is 0 or 1, one of Y or Z is N and the other of Y or Z is N or CR²;

L¹ is L³;

L is a bond or L³;

L³ is independently C₃₋₆ cycloalkylene or is C₂₋₅ alkylene where one or two —CH₂— groups of said C₂₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;

R^{a} and R^{b} are independently H, alkyl, or substituted alkyl;

R¹ and R³ are independently selected from aryl, substituted aryl, heteroaryl, substituted heteroaryl, heterocyclyl, substituted heterocyclyl, cycloalkyl, and substituted cycloalkyl;

and

R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano; and

(2) a detectable amount of one or more compounds selected from:

a compound of formula 1.1

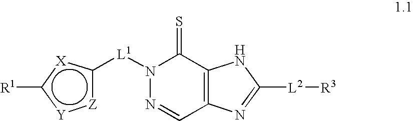

or a salt thereof;

P₂S₅;

a compound of formula 2.1

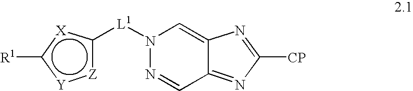

or a salt thereof, wherein CP is a substituent that can undergo a coupling reaction;

a compound of formula 14.1

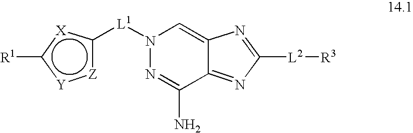

or a salt thereof, wherein L¹, L², R¹, R³, X, Y, and Z are as defined above;

a compound comprising tin, zinc, magnesium, silicon, or boron;

a compound comprising palladium, nickel, iron, or copper;

hydrazine;

and

Cs₂CO₃.

Provided are processes for the preparation of a compound of formula III:

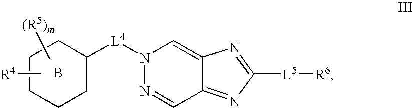

wherein

ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;

L⁴ is L⁶;

L⁵ is a bond or L⁶;

L⁶ is independently C₃₋₆ cycloalkylene or is C₂₋₅ alkylene where one or two —CH₂— groups of said C₂₋₅ alkylene are optionally replaced with —NR⁷—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵;

R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;

R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁷ is independently H, alkyl, or substituted alkyl;

m is 0, 1, 2, 3, or 4;

and

provided that the compound of formula III is not 4′-(2-butyl-imidazo[4,5-d]-pyridazin-5-ylmethyl)-biphenyl-2-carboxylic acid.

Also provided is a composition comprising:

(1) a compound of formula III or a salt or solvate thereof

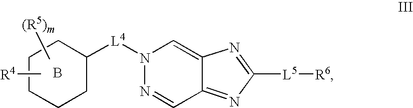

wherein

ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;

L⁴ is L⁶;

L⁵ is a bond or L⁶;

L⁶ is independently C₃₋₆ cycloalkylene or is C_{1 5} alkylene where one or two —CH₂— groups of said C₂₋₅ alkylene are optionally replaced with —NR⁷—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵;

R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;

R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁷ is independently H, alkyl, or substituted alkyl;

m is 0, 1, 2, 3, or 4;

provided that the compound of formula III is not 4′-(2-butyl-imidazo[4,5-d]-pyridazin-5-ylmethyl)-biphenyl-2-carboxylic acid.

and

(2) a detectable amount of one or more compounds selected from:

a compound of formula 6.1

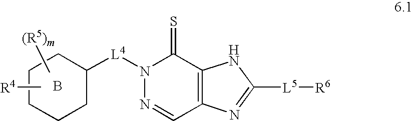

or a salt thereof;

P₂S₅;

a compound of formula 7.1

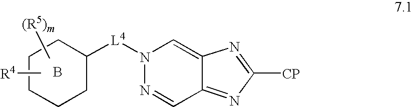

or a salt thereof, wherein CP is a substituent that can undergo a coupling reaction;

a compound of formula 15.1

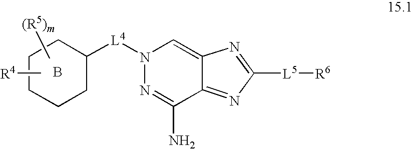

or a salt thereof, wherein L⁴, L⁵, R⁴, R⁵, R⁶, and m are as defined above;

a compound comprising tin, zinc, magnesium, silicon, or boron;

a compound comprising palladium, nickel, iron, or copper;

hydrazine;

and

Cs₂CO₃.

Those and other embodiments are further described in the text that follows.

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings:

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C<ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy” refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy” refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In some embodiments, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “substituted heterocycle” or “substituted heterocycloalkyl” or “substituted heterocyclyl” refers to heterocyclic groups, as defined herein, that are substituted with from 1 to 5 or in some embodiments I to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1 -dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

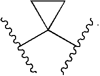

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alcenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. In some embodiments, solvents are volatile, non-toxic, and/or acceptable for administration to humans in trace amounts. Suitable solvents include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N—— moiety such as pyrazoles, imidazoles, benzimidazoles, triazoles, and tetrazoles.

“Leaving group” or “LG” refers to an atom or group of atoms that disconnect, either charged or uncharged, from a compound thereby leaving a portion of the compound that can be considered to be the main fragment. Common leaving groups are well known to those skilled in the art and include, for example, halogen, hydroxy, alkoxy, substituted alkoxy, sulfonyloxy, water, and dinitrogen.

“A substituent that can undergo a coupling reaction” or “CP” or “M” refers to an atom or group of atoms that can participate in a “coupling reaction.” Substituents that can undergo coupling reactions are well known to those skilled in the art and include, for example, hydrogen, halogen, alkynyl, substituted alkynyl, alkenyl, substituted alkenyl, organotin, organoboron, organosilyl, organomagnesium, and organotrifluoroborate.

“Organotin” refers to compounds that comprise tin and have at least one tin-carbon bond.

“Organoboron” refers to compounds that comprise boron and have at least one boron-carbon bond.

“Organosilyl” refers to compounds that comprise silicon and have at least one silicon-carbon bond.

“Organomagnesium” refers to compounds that comprise magnesium and have at least one magnesium-carbon bond.

“Organotrifluoroborate” refers to compounds that comprise BF₃ and have at least one boron-carbon bond.

“Coupling reaction” refers to a reaction that is catalyzed by at least one metal or at least one compound comprising a metal and that results in the formation of a carbon-carbon bond. The metals that can be used are well known to those skilled in the art and include, for example, palladium, nickel, iron, and copper.

“Sulfurizing” refers to a process wherein sulfur is incorporated into a reactant. A “sulfurizing reagent” refers to a compound that can incorporate sulfur into a reactant. Sulfurizing reagents are well known to those skilled in the art and include, for example, P₂S₅ and Lawesson's reagent.

“Desulfurizing” refers to a process wherein sulfur is removed from a reactant. A “desulfurizing reagent” refers to a compound that can remove sulfur atom from a reactant. Desulfurizing reagents are well known to those skilled in the art and include, for example, Raney nickel.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless otherwise indicated, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, provided are processes for the preparation of a compound of formula I:

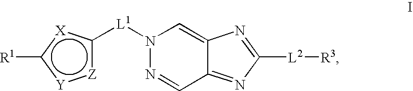

wherein

a) when X is CR² or N, one of Y or Z is O and the other of Y or Z is N; or one of Y or Z is N and the other of Y or Z is NR^{a};

b) when X is O, NR^{a}, or S(O)ₚ wherein p is 0 or 1, one of Y or Z is N and the other of Y or Z is N or CR²;

L¹ is L³;

L² is a bond or L³;

L³ is independently C₃₋₆ cycloalkylene or is C₂₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;

R^{a} and R^{b} are independently H, alkyl, or substituted alkyl;

R¹ and R³ are independently selected from aryl, substituted aryl, heteroaryl, substituted heteroaryl, heterocyclyl, substituted heterocyclyl, cycloalkyl, and substituted cycloalkyl;

and

R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano.

In some embodiments, provided is a process for the preparation of a compound formula I

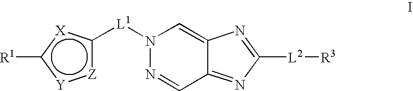

that comprises converting a compound of formula 1.1

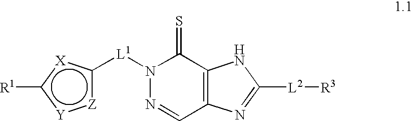

to the compound of formula I.

In some embodiments of formula I L¹ is C₁₋₃ alkylene. In some embodiments, L¹ is CH₂.

In some embodiments of formula I X is CR², Y is N, and Z is O. In some embodiments of formula I X is CH, Y is N, and Z is O.

In some embodiments of formula I R¹ is an optionally substituted aryl. In some embodiments of formula I R¹ is an optionally substituted phenyl. In some embodiments of formula I R¹ is a phenyl optionally substituted with at least one group selected from alkyl, haloalkyl, alkoxy, substituted alkoxy, and halogen. In some embodiments of formula I R¹ is a phenyl optionally substituted with at least one group selected from —CF₃, —OCH₃, substituted methoxy, Cl, and F.

In some embodiments of formula I L² is a bond.

In some embodiments of formula I R³ is an optionally substituted aryl. In some embodiments of formula I R³ is an optionally substituted phenyl. In some embodiments of formula I R³ is a phenyl optionally substituted with at least halogen. In some embodiments of formula I R³ is a phenyl optionally substituted with at least one F.

In some embodiments, the compound of formula I

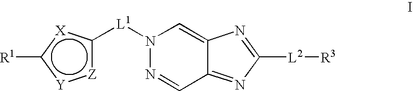

is prepared by a process comprising

reacting the compound of formula 1.1

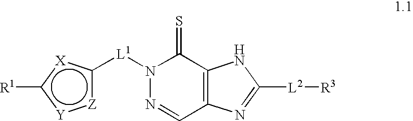

with a desulfurizing reagent.

In some embodiments, the desulfurizing reagent is Raney nickel.

In some embodiments, the compound of formula 1.1

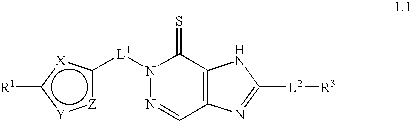

is prepared by a process comprising

reacting a compound of formula 1.2

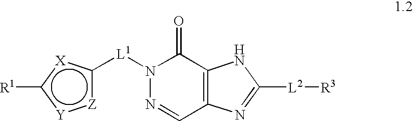

with a sulfurizing reagent.

In some embodiments, the sulfurizing reagent is P₂S₅. In some embodiments, the sulfurizing reagent is Lawesson's reagent.

In some embodiments, the compound of formula 1.2

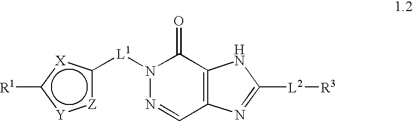

is prepared by a process comprising

cyclizing a compound of formula 1.3

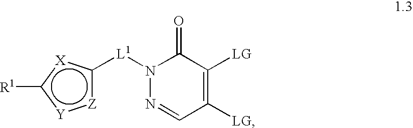

wherein

each LG is independently chosen and is a leaving group.

In some embodiments, at least one LG of the compound of formula 1.3 is a halogen. In some embodiments, at least one LG of the compound of formula 1.3 is Br.

In some embodiments, the cyclization of the compound of formula 1.3

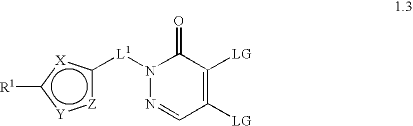

occurs with a compound of formula 1.4

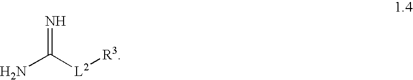

In some embodiments, the cyclization of the compound of formula 1.3 occurs with Cs₂CO₃. In some embodiments, the cyclization of the compound of formula 1.3 occurs with Cs₂CO₃ and CuI. In some embodiments, the cyclization of the compound of formula 1.3 occurs with microwave irradiation.

In some embodiments, the compound of formula 1.3

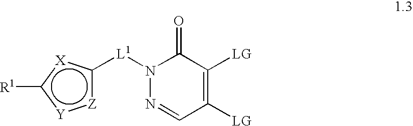

is prepared by a process comprising

cyclizing a compound of formula 1.5

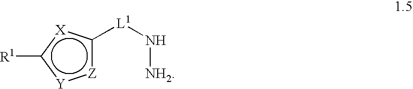

In some embodiments, the cyclization of the compound of formula 1.5 occurs with mucobromic acid.

In some embodiments, the compound of formula 1.5

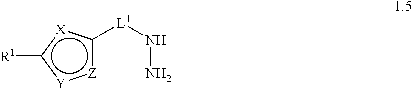

is prepared by a process comprising

reacting a compound of formula 1.6

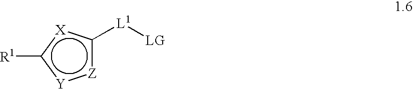

with hydrazine,

wherein

LG is a leaving group.

In some embodiments, the LG of the compound of formula 1.6 is a halogen, hydroxy, alkoxy, substituted alkoxy, or sulfonyloxy. In some embodiments, the LG of the compound of formula 1.6 is a halogen. In some embodiments, the LG of the compound of formula 1.6 is Cl. In some embodiments, the LG of the compound of formula 1.6 is hydroxy. In some embodiments, the LG of the compound of formula 1.6 is a sulfonyloxy. In some embodiments, the LG of the compound of formula 1.6 is —OSO₂C₆H₄-4-CH₃. In some embodiments, the LG of the compound of formula 1.6 is —OSO₂CH₃. In some embodiments, the LG of the compound of formula 1.6 is —OSO₂CF₃.

In some embodiments, a process for the preparation of a compound of formula I is as shown in Scheme 1

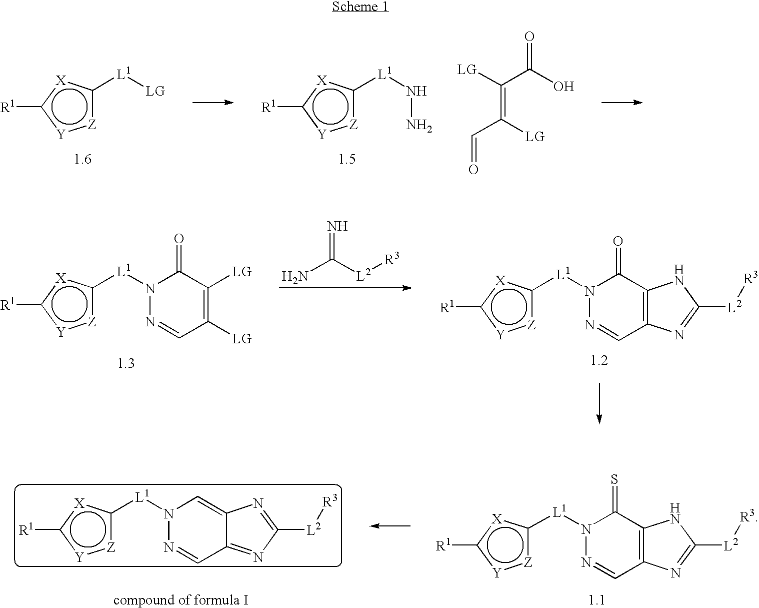

Scheme 1 shows the synthesis of compounds of formula I where R¹, R³, L¹, L², X, Y, Z, and LG are previously defined. The substituted hydrazine 1.5 is formed from displacement of the corresponding electrophiles such as chloroalkyl heterocycles 1.6 with hydrazine. The compounds 1.5 are then cyclized to form compounds 1.3, which are in turn cyclized with amidines giving 2,5-disubstituted-3,5-dihydro-imidazo[4,5-d]pyridazin-4-ones 1.2. These are then converted to the compound of formula I through treatment with reagents such as P₂S₅ followed by reduction with Raney nickel.

In some embodiments, provided is a process for the preparation of a compound formula I

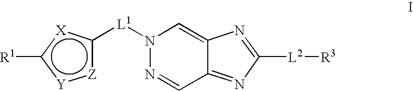

that comprises converting a compound of formula 2.1

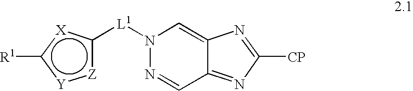

to the compound of formula I,
wherein

CP is a substituent that can undergo a coupling reaction.

In some embodiments of formula I L¹ is C₁₋₃ alkylene. In some embodiments, L¹ is CH₂.

In some embodiments of formula I X is CR², Y is N, and Z is O. In some embodiments of formula I X is CH, Y is N, and Z is O.

In some embodiments of formula I R¹ is an optionally substituted aryl. In some embodiments of formula I R¹ is an optionally substituted phenyl. In some embodiments of formula I R¹ is a phenyl optionally substituted with at least one group selected from alkyl, haloalkyl, and alkoxy. In some embodiments of formula I R¹ is a phenyl optionally substituted with at least one group selected from —CF₃, —OCH₂CH₂CH₃, and —OCH₂CH₂CH₂CH₃.

In some embodiments of formula I L² is a bond.

In some embodiments of formula I R³ is an optionally substituted aryl or heteroaryl. In some embodiments of formula I R³ is an optionally substituted phenyl. In some embodiments of formula I R³ is a phenyl optionally substituted with at least one group selected from acyl, alkyl, alkoxy, amino, aminocarbonyl, haloalkyl, halogen, and hydroxy. In some embodiments of formula I R³ is a heteroaryl optionally substituted with at least one alkyl. In some embodiments of formula I R³ is a heteroaryl optionally substituted with at least one —CH₃.

In some embodiments, the compound of formula I

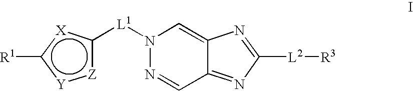

is prepared by a process comprising

coupling a compound of formula 2.1

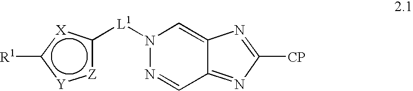

under conditions appropriate to form a new carbon-carbon bond.

In some embodiments, the CP of the compound of formula 2.1 is a halogen or sulfonlyoxy. In some embodiments, the CP of the compound of formula 2.1 is a halogen. In some embodiments, the CP of the compound of formula 2.1 is Br. In some embodiments, the CP of the compound of formula 2.1 is a sulfonyloxy. In some embodiments, the CP of the compound of formula 2.1 is —OSO₂CF₃.

In some embodiments the compound of formula 2.1 is coupled with a compound of formula 2.2

M-L²-R³ 2.2

wherein

M is a substituent that can undergo a coupling reaction.

In some embodiments, the compound of formula 2.2 comprises tin, zinc, magnesium, silicon, or boron. In some embodiments, the compound of formula 2.2 is an organotin, organozinc, organomagnesium, organosilyl, organoboron, or organotrifluoroborate compound.

In some embodiments, the organoboron of formula 2.2 is a boronic acid or boronic ester of formula 2.3

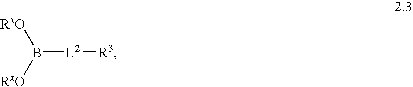

wherein

each R^{x} is independently selected from hydrogen, alkyl, or substituted alkyl

and,

wherein

the R^{x} groups, if alkyl or substituted alkyl, can optionally be connected.

In some embodiments, the organoboron of formula 2.2 is a boronic acid of formula 2.4

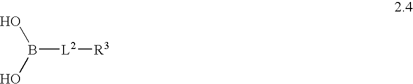

In some embodiments, the coupling reaction of the compound of formula 2.1 occurs in the presence of a metal catalyst. In some embodiments the metal catalyst comprises palladium, nickel, iron, or copper. In some embodiments, the metal catalyst is tetrakistriphenylphosphine palladium.

In some embodiments, the compound of formula 2.1

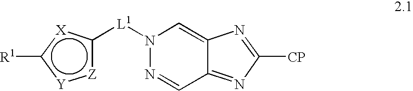

is prepared by a process comprising

reacting a compound of formula 2.5

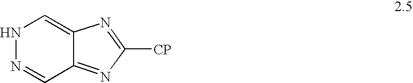

with the compound of formula 1.6

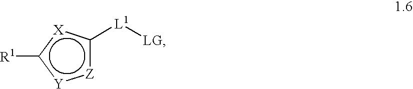

wherein

LG is a leaving group.

In some embodiments, the LG of the compound of formula 1.6 is a halogen, hydroxy, alkoxy, substituted alkoxy, or sulfonyloxy. In some embodiments, the LG of the compound of formula 1.6 is a halogen. In some embodiments, the LG of the compound of formula 1.6 is Cl. In some embodiments, the LG of the compound of formula 1.6 is hydroxy. In some embodiments, the LG of the compound of formula 1.6 is a sulfonyloxy. In some embodiments, the LG of the compound of formula 1.6 is —OSO₂C₆H₄-4-CH₃. In some embodiments, the LG of the compound of formula 1.6 is —OSO₂CH₃. In some embodiments, the LG of the compound of formula 1.6 is —OSO₂CF₃.

In some embodiments, the compound of formula 2.5

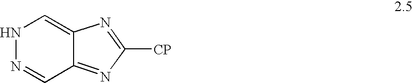

is prepared by a process comprising

a) reacting a compound of formula 2.6

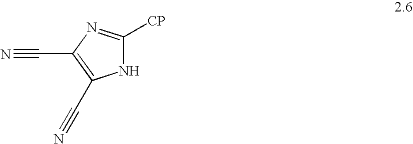

with a reducing reagent

and

b) cyclizing the resulting product to form the compound of formula 2.5.

In some embodiments, the reducing reagent in the reaction with the compound of formula 2.6 diisobutylaluminum hydride.

In some embodiments, the product from the reaction of the compound of formula 2.6 with the reducing reagent is cyclized with hydrazine.

In some embodiments, a process for the preparation of a compound of formula I is as shown in Scheme 2

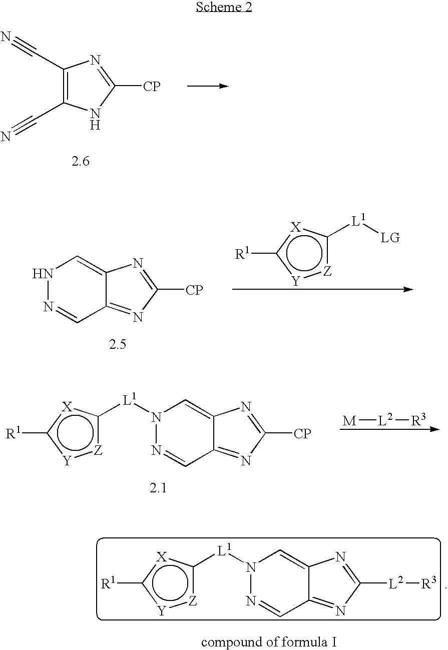

Scheme 2 shows the synthesis of compounds of formula I where R¹, R³, L¹, L², X, Y, Z, LG, CP, and M are previously defined. The dinitrile 2.6 (Heterocycles, 29, 1325, 1989) is reduced with reagents such as DIBAL-H in a solvent such as THF and subsequently cyclized with hydrazine or its derivatives to give 2-substituted-5H-imidazo[4,5-d]pyridazine 2.5. These are then alkylated with electrophiles such as chloroalkyl heterocycles giving the 2-substituted-5-substituted-imidazo[4,5-d]pyridazines 2.1. They can be converted to the compound of formula I through coupling reactions such as the Suzuki reaction.

In some embodiments, provided is a process for the preparation of a compound formula I

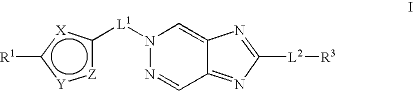

that comprises converting a compound of formula 3.1

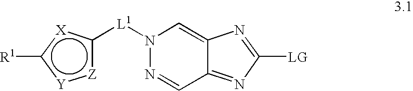

to the compound of formula I,
wherein

LG is a leaving group.

In some embodiments of formula I L¹ is C₁₋₃ alkylene. In some embodiments, L¹ is CH₂.

In some embodiments of formula I X is CR², Y is N, and Z is O. In some embodiments of formula I X is CH, Y is N, and Z is O.

In some embodiments of formula I R¹ is an optionally substituted aryl. In some embodiments of formula I R¹ is an optionally substituted phenyl. In some embodiments of formula I R¹ is a phenyl optionally substituted with at least one alkyl or haloalkyl. In some embodiments of formula I R¹ is a phenyl optionally substituted with at least one —CF₃.

In some embodiments of formula I L² is a bond.

In some embodiments of formula I R³ is a substituted amino or a heterocycle. In some embodiments of formula I R³ is a substituted amino optionally substituted with at least one group selected from hydrogen, aryl, alkyl, substituted alkyl, and heterocycle.

In some embodiments, the compound of formula I

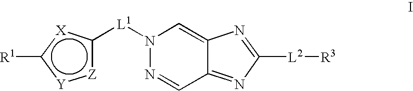

is prepared by a process comprising

reacting a compound of formula 3.1

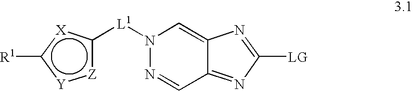

with a compound comprising nitrogen, oxygen or sulfur.

In some embodiments, the LG of the compound of formula 3.1 is a halogen, hydroxy, alkoxy, substituted alkoxy, or sulfonyloxy. In some embodiments, the LG of the compound of formula 3.1 is a halogen. In some embodiments, the LG of the compound of formula 3.1 is Br.

In some embodiments, the compound comprising nitrogen is chosen from aniline, morpholine, piperidine, phenylmethanamine, N-methyl(phenyl)methanamine, 1,2,3,4-tetrahydroquinoline, (2-fluorophenyl)methanamine, (2,3-diflurophenyl)methanamine, 2-phenylethanamine, 1-phenylethanamine, 1,2,3,4-tetrahydroisoquinoline, 2,3-dihydro-1H-inden-1-amine, 1,2,3,4-tetrahydronaphthalen-1-amine, and isoindoline.

In some embodiments, the reaction of the compound of formula 3.1 with a compound comprising nitrogen is heated.

In some embodiments, the reaction of the compound of formula 3.1 occurs with microwave irradiation.

In some embodiments, the compound of formula 3.1

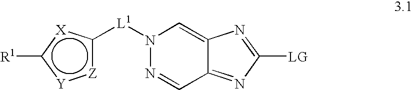

is prepared by a process comprising

reacting a compound of formula 3.2

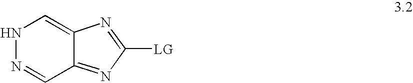

with the compound of formula 1.6

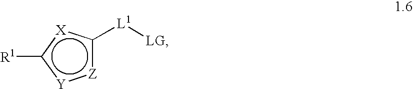

wherein

each LG is independently chosen and each is a leaving group.

In some embodiments, at least one LG in the compounds of formula 3.2 and 1.6 is halogen, hydroxy, alkoxy, substituted alkoxy, or sulfonyloxy. In some embodiments, at least one LG in the compounds of formula 3.2 and 1.6 is a halogen. In some embodiments, at least one LG is Cl. In some embodiments, the LG in the compound of formula 1.6 is Cl. In some embodiments, at least one LG is Br. In some embodiments, the LG in the compound of formula 3.2 is Br. In some embodiments, at least one LG in the compounds of formula 3.2 and 1.6 is hydroxy. In some embodiments, at least one LG in the compounds of formula 3.2 and 1.6 is sulfonyloxy. In some embodiments, at least one LG in the compounds of formula 3.2 and 1.6 is —OSO₂C₆H₄-4-CH₃. In some embodiments, at least one LG in the compounds of formula 3.2 and 1.6 is —OSO₂CH₃. In some embodiments, at least one LG in the compounds of formula 3.2 and 1.6 is —OSO₂CF₃.

In some embodiments, the compound of formula 3.2

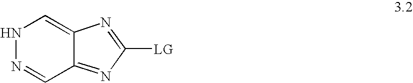

is prepared by a process comprising

a) reacting a compound of formula 3.3

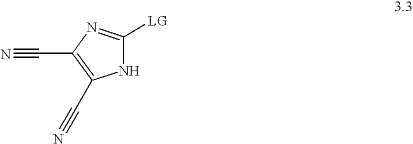

with a reducing reagent

and

b) cyclizing the resulting product to form the compound of formula 3.2.

In some embodiments, the reducing reagent in the reaction with the compound of formula 3.3 is diisobutylaluminum hydride.

In some embodiments, the product from the reaction of the compound of formula 3.3 with the reducing reagent is cyclized with hydrazine.

In some embodiments, a process for the preparation of compounds of formula I is as shown in Scheme 3

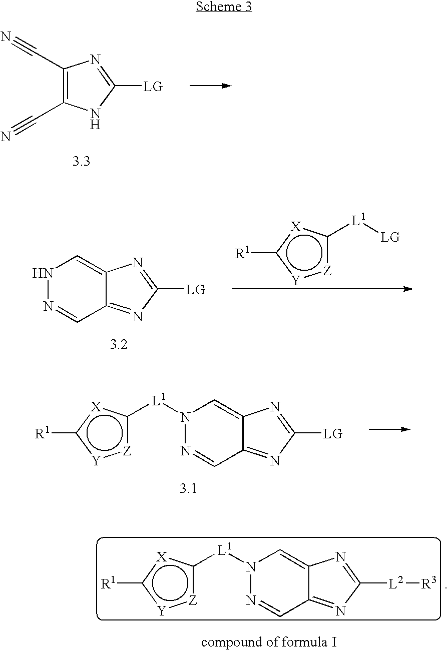

Scheme 3 shows the synthesis of compounds of formula I where R¹, R³, L¹, L², X, Y, Z, and LG are previously defined. The dinitrile 3.3 (Heterocycles, 29, 1325, 1989) is reduced with reagents such as DIBAL-H in a solvent such as THF and subsequently cyclized with hydrazine or its derivatives to give 2-substituted-5H-imidazo[4,5-d]pyridazine 3.2. These are then alkylated with electrophiles such as chloroalkyl heterocycles giving the 2-substituted-5-substituted-imidazo[4,5-d]pyridazines 3.1. They can be converted to the compound of formula I through reactions with a compound comprising nitrogen, oxygen or sulfur.

In some embodiments, provided is a process for the preparation of a compound formula I

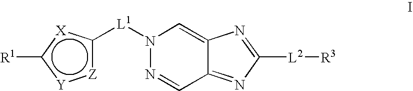

that comprises converting a compound of formula II

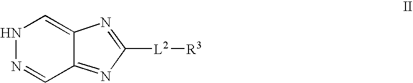

to the compound of formula I.

In some embodiments, the process of converting the compound of formula II

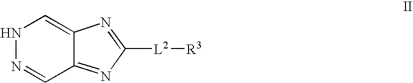

to the compound of formula I

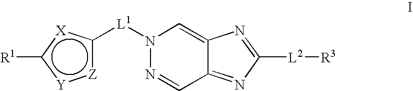

comprises reacting the compound of formula II with a compound of formula 1.6

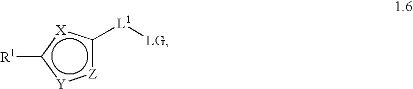

wherein

LG is a leaving group.

In some embodiments, L¹ is C₁₋₃ alkylene. In some embodiments, L¹ is C₁₋₃ alkylene optionally substituted with one to two alkyl groups. In some embodiments, L¹ is CH₂.

In some embodiments of formula I X is CR², Y is N, and Z is O. In some embodiments of formula I X is CR², Y is O, and Z is N. In some embodiments of formula I X is CH, Y is N, and Z is O. In some embodiments of formula I X is CH, Y is O, and Z is N. In some embodiments of formula I X is N, Y is N, and Z is O. In some embodiments of formula I X is N, Y is O, and Z is N. In some embodiments of formula I X is O, Y is N, and Z is N. In some embodiments of formula I X is O, Y is CR², and Z is N. In some embodiments of formula I X is O, Y is CH, and Z is N.

In some embodiments of formula I R¹ is an optionally substituted aryl or heteroaryl. In some embodiments of formula I R¹ is an optionally substituted phenyl. In some embodiments of formula I R¹ is a phenyl optionally substituted with an alkyl. In some embodiments of formula I R¹ is a heteroaryl optionally substituted with at least one group selected from alkyl, haloalkyl, or halogen. In some embodiments of formula I R¹ is a heteroaryl optionally substituted with at least one group selected from —CH₃, —CF₃, F, or Br.

In some embodiments of formula I L² is a bond.

In some embodiments of formula I R³ is an optionally substituted aryl. In some embodiments of formula I R³ is an optionally substituted phenyl. In some embodiments of formula I R³ is a phenyl optionally substituted with at least one halogen. In some embodiments of formula I R³ is a heteroaryl optionally substituted with at least one F.

In some embodiments, the LG of the compound of formula 1.6 is a halogen, hydroxy, alkoxy, substituted, or sulfonyloxy. In some embodiments, the LG of the compound of formula 1.6 is a halogen. In some embodiments, the LG of the compound of formula 1.6 is Cl. In some embodiments, the LG of the compound of formula 1.6 is hydroxy. In some embodiments, the LG of the compound of formula 1.6 is a sulfonyloxy. In some embodiments, the LG of the compound of formula 1.6 is —OSO₂C₆H₄-4-CH₃. In some embodiments, the LG of the compound of formula 1.6 is —OSO₂CH₃. In some embodiments, the LG of the compound of formula 1.6 is —OSO₂CF₃.

In some embodiments, a process for the preparation of a compound of formula I is as shown in Scheme 4

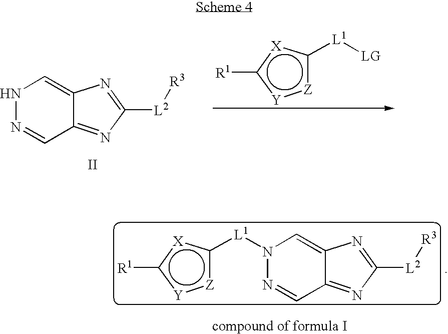

Scheme 4 shows the synthesis of compounds of formula I where R¹, R³, L¹, L², X, Y, Z, and LG are previously defined. The 2-substituted-5H-imidazo[4,5-d]pyridazine II are alkylated with electrophiles such as chloroalkyl heterocycles giving the compound of formula I.

In some embodiments, the compound of formula II

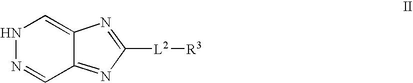

is prepared by a process comprising

a) reacting a compound of formula 4.1

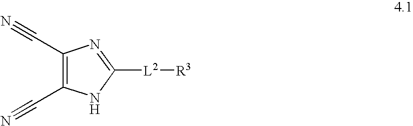

with a reducing reagent

and

b) cyclizing the resulting product to form the compound of formula II.

In some embodiments, the reducing reagent in the reaction with the compound of formula 4.1 is diisobutylaluminum hydride.

In some embodiments, the product from the reaction of the compound of formula 4.1 with the reducing agent is cyclized with hydrazine.

In some embodiments, the compound of formula 4.1

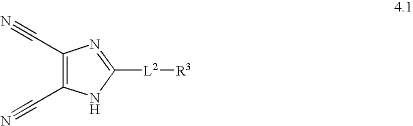

is prepared by a process comprising

cyclizing a compound of formula 4.2

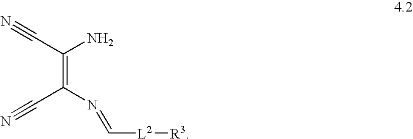

In some embodiments, the cyclization occurs with N-chlorosuccinimide and nicotinamide.

In some embodiments, the compound of formula 4.2

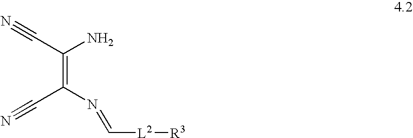

is prepared by a process comprising

reacting a compound of formula 4.3

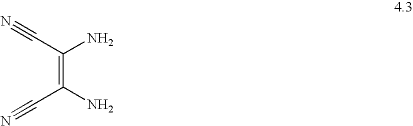

with a compound of formula 4.4

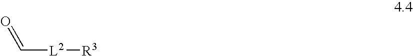

In some embodiments, a process for the preparation of a compound of formula II is as shown in Scheme 5

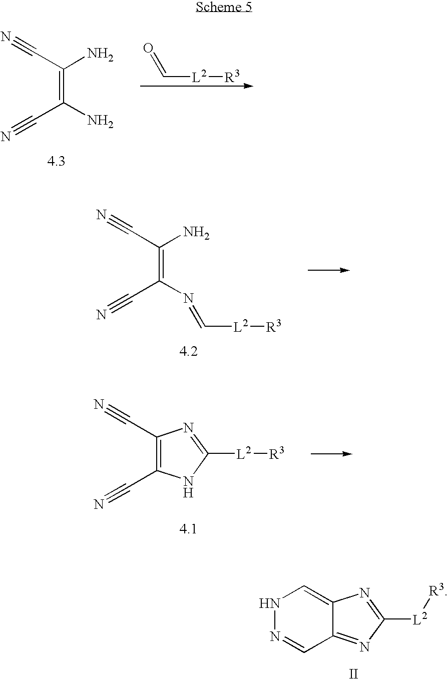

Scheme 5 shows the synthesis of compounds of formula II where R³ and L² are previously defined. The dinitrile 4.3 is condensed with aldehydes of formula H(O)C-L²R³ and oxidatively cyclized to the 2-substituted imidazole 4,5 dinitrile 4.1. This is then reduced with reagents such as DIBAL-H in a solvent such as THF and subsequently cyclized with hydrazine or its derivatives to give 2-substituted-5H-imidazo[4,5-d]pyridazine II.

In some embodiments, the compound of formula II

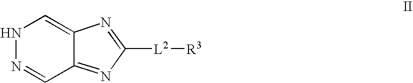

is prepared by a process comprising

a) saponifying a compound of formula 5.1

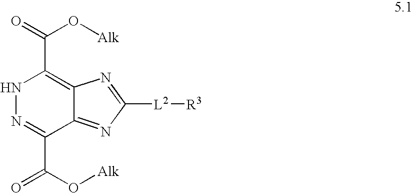

to form a compound of formula 5.2

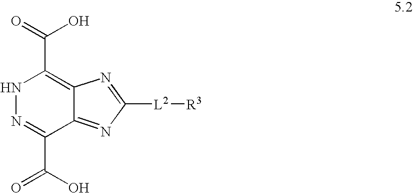

and

b) decarboxylating the compound of formula 5.2 to form the compound of formula II,

wherein

each Alk is independently chosen and each is an alkyl or substituted alkyl.

In some embodiments, the saponification and decarboxylation of the compound of formula 5.2 occur in the presence of hydrochloric acid and water.

In some embodiments, the Alk in the compound of formula 5.1 is CH₃.

In some embodiments, the compound of formula 5.1

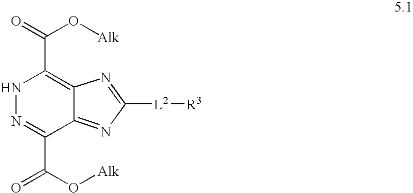

is prepared by a process comprising

reacting a compound of formula 5.3

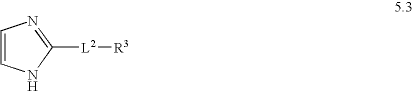

with a compound of formula 5.4

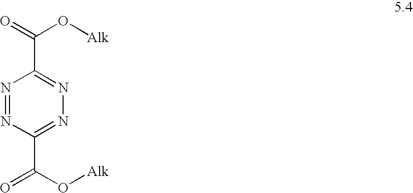

to form the compound of formula 5.1.

In some embodiments, the reaction is heated.

In some embodiments, the compound of formula 5.3

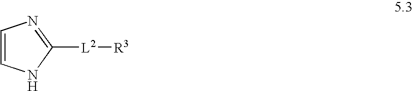

is prepared by a process comprising

reacting a compound of formula 4.4

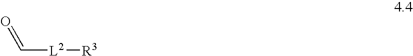

with glyoxal and ammonia.

In some embodiments, a process for the preparation of a compound of formula II is as shown in Scheme 6

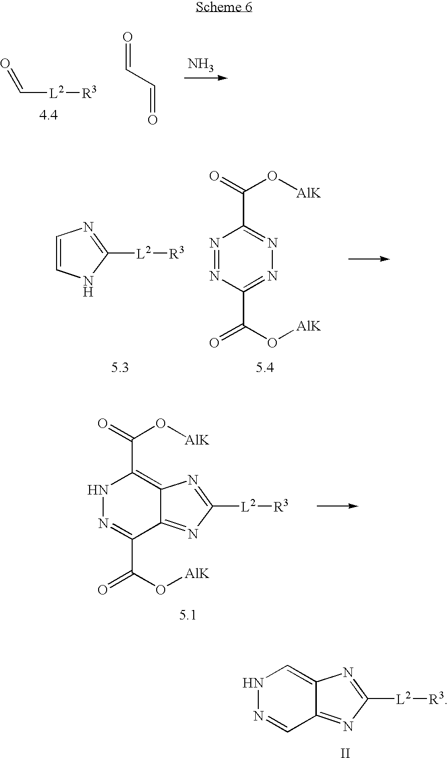

Scheme 6 shows the synthesis of compounds of formula II where R³, L², and Alk are previously defined. The imidazole 5.3 is formed in one step from the corresponding aldehyde 4.4 through condensation with glyoxal and ammonia. The 2-substituted imidazole 5.3 is condensed with reagents such as [1,2,4,5]Tetrazine-3,6-dicarboxylic acid dialkyl ester 5.4 (Org. Syn. Coll. Vol. 9, p 335, 1998). The intermediate 5.1 is then saponified and decarboxylated yielding the compound of formula II.

In some embodiments, the process of converting the compound of formula II

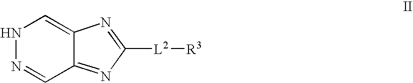

to the compound of formula I

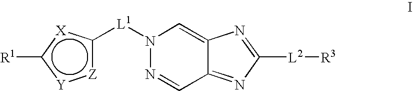

comprises

a) reacting the compound of formula II with a compound of formula 6.1

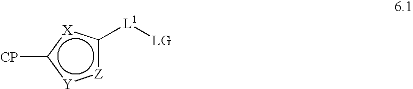

to form a compound of formula 6.2

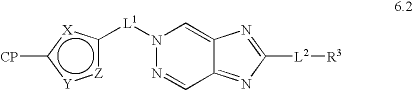

and

b) coupling the compound of formula 6.2 under conditions appropriate to form a carbon-carbon bond

wherein

LG is a leaving group

and

CP is a substituent that can undergo a coupling reaction.

In some embodiments the LG of the compound of formula 6.1 is a halogen, hydroxy, alkoxy, substituted alkoxy, or sulfonyloxy. In some embodiments, the LG of the compound of formula 6.1 is a halogen. In some embodiments, the LG of the compound of formula 6.1 is Cl. In some embodiments, the LG of the compound of formula 6.1 is hydroxy. In some embodiments, the LG of the compound of formula 6.1 is a sulfonyloxy. In some embodiments, the LG of the compound of formula 6.1 is —OSO₂C₆H₄-4-CH₃. In some embodiments, the LG of the compound of formula 6.1 is —OSO₂CH₃. In some embodiments, the LG of the compound of formula 6.1 is —OSO₂CF₃.

In some embodiments the compound of formula 6.1 is coupled with a compound of formula 6.3

M-R¹ 6.3

to form the compound of formula I,
wherein

M is a substituent that can undergo a coupling reaction.

In some embodiments, the compound of formula 6.3 comprises tin, zinc, magnesium, silicon, or boron. In some embodiments, the compound of formula 6.3 can be an organotin, organozinc, organomagnesium, organosilyl, organoboron, or organotrifluoroborate compound.

In some embodiments, the organoboron of formula 6.3 is a boronic acid or boronic ester of formula 6.4

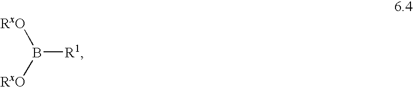

wherein

each R^{x} is independently selected from hydrogen, alkyl, or substituted alkyl

and,

wherein

the R^{x} groups, if alkyl or substituted alkyl, can optionally be connected.

In some embodiments, the organoboron of formula 6.3 is a boronic acid of formula 6.5

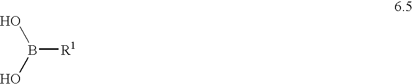

In some embodiments, the CP of the compound of formula 6.2 is a halogen or sulfonyloxy. In some embodiments, the CP of the compound of formula 6.2 is a halogen. In some embodiments, the CP of the compound of formula 6.2 is Br. In some embodiments, the CP of the compound of formula 6.2 is a sulfonyloxy. In some embodiments, the CP of the compound of formula 6.2 is —OSO₂CF₃.

In some embodiments, the coupling reaction of the compound of formula 6.2 occurs in the presence of at least one compound comprising palladium, nickel, iron, or copper. In some embodiments, the coupling reaction of the compound of formula 6.2 occurs in the presence of tetrakistriphenylphosphine palladium.

In some embodiments, a process for the preparation of a compound of formula II is as shown in Scheme 7

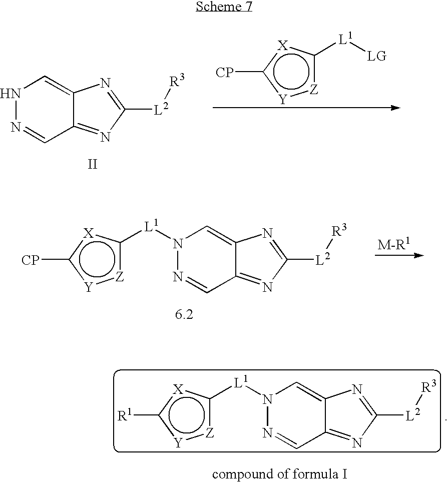

Scheme 7 shows the synthesis of compounds of formula I where R¹, R³, L¹, L², X, Y, Z, LG, CP, and M are previously defined. The 2-substituted-5H-imidazo[4,5-d]pyridazine II is alkylated with electrophiles such as chloroalkyl heterocycles yielding compounds of formula 6.2 which can then be converted to the compound of formula I.

In some embodiments, the process of converting the compound of formula II

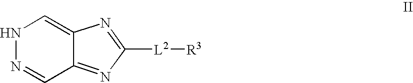

to the compound of formula I

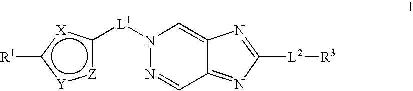

comprises

a) reacting the compound of formula II with a compound of formula 7.1

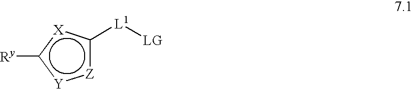

to form a compound of formula 7.2

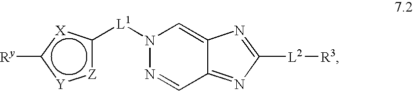

b) reacting the compound of formula 7.2 with a suitable reagent to form a compound of formula 7.3

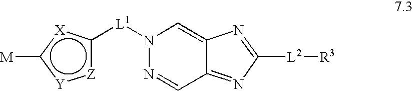

and

c) coupling the compound of formula 7.3 with a compound of formula 7.4

CP—R¹ 7.4

under conditions appropriate to form a carbon-carbon bond,
wherein

LG is a leaving group,

R^{y} is a halogen or sulfonyloxy,

and

M and CP are each substituents that can undergo a coupling reaction.

In some embodiments, the LG of the compound of formula 7.1 is a halogen, hydroxy, alkoxy, substituted alkoxy, or sulfonyloxy. In some embodiments, the LG of the compound of formula 7.1 is a halogen. In some embodiments, the LG of the compound of formula 7.1 is Cl. In some embodiments, the LG of the compound of formula 7.1 is hydroxy. In some embodiments, the LG of the compound of formula 7.1 is a sulfonyloxy. In some embodiments, the LG of the compound of formula 7.1 is —OSO₂C₆H₄-4-CH₃, —OSO₂CH₃, or —OSO₂CF₃.

In some embodiments, the R^{y} of the compound of formula 7.1 is Br. In some embodiments, the R^{y} of the compound of formula 7.1 is —OSO₂CF₃.

In some embodiments, the M of the compound of formula 7.3 comprises tin, zinc, magnesium, silicon, or boron. In some embodiments, the M of the compound of formula 7.3 comprises boron.

In some embodiments, the coupling reaction of step (c) occurs in the presence of at least one compound comprising palladium, nickel, iron, or copper. In some embodiments, the coupling reaction of step (c) occurs in the presence of tetrakistriphenylphosphine palladium.

In some embodiments, a process for the preparation of a compound of formula II is as shown in Scheme 8

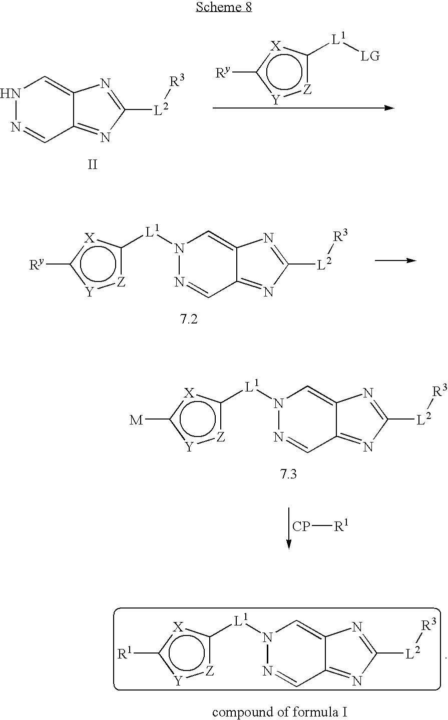

Scheme 8 shows the synthesis of compounds of formula I where R¹, R³, L¹, L², X, Y, Z, LG, CP, M, and R^{y} are previously defined. The 2-substituted-5H-imidazo[4,5-d]pyridazine II is alkylated with electrophiles such as chloroalkyl heterocycles yielding the products 7.2 which can then be converted to compounds of 7.3. These can then undergo coupling reactions to provide the compound of formula I.

Also provided is a composition comprising: (1) a compound of formula I or a salt or solvate thereof

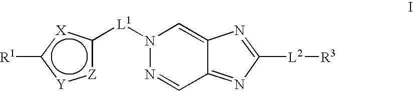

wherein

a) when X is CR² or N, one of Y or Z is O and the other of Y or Z is N; or one of Y or Z is N and the other of Y or Z is NR^{a};

b) when X is O, NR^{a}, or S(O)ₚ wherein p is 0 or 1, one of Y or Z is N and the other of Y or Z is N or CR²;

L¹ is L³;

L² is a bond or L³;

L³ is independently C₃₋₆ cycloalkylene or is C₁₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;

R^{a} and R^{b} are independently H, alkyl, or substituted alkyl;

R¹ and R³ are independently selected from aryl, substituted aryl, heteroaryl, substituted heteroaryl, heterocyclyl, substituted heterocyclyl, cycloalkyl, and substituted cycloalkyl;

and

R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano; and

(2) a detectable amount of one or more compounds selected from:

a compound of formula 1.1

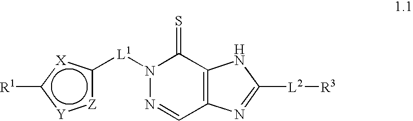

or a salt thereof;

P₂S₅;

a compound of formula 2.1

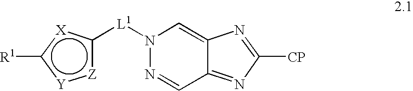

or a salt thereof, wherein CP is a group that can undergo a coupling reaction;

a compound of formula 14.1

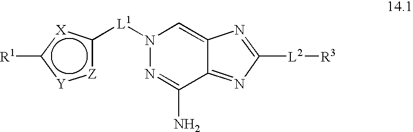

or a salt thereof, wherein L¹, L², R¹, R³, X, Y, and Z are as defined above;

a compound comprising tin, zinc, magnesium, silicon, or boron;

a compound comprising palladium, nickel, iron, or copper;

hydrazine;

and

Cs₂CO₃.

Provided are processes for the preparation of a compound of formula III:

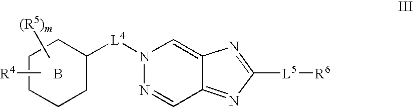

wherein

ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 1 0-membered bicyclic ring;

L⁴ is L⁶;

L⁵ is a bond or L⁶;

L⁶ is independently C₃₋₆ cycloalkylene or is C₁₋₅ alkylene where one or two —CH₂— groups of said C₁₋₅ alkylene are optionally replaced with —NRC—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵;

R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;

R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁷ is independently H, alkyl, or substituted alkyl;

m is 0, 1, 2, 3, or 4; and

provided that the compound of formula III is not 4′-(2-butyl-imidazo[4,5-d]-pyridazin-5-ylmethyl)-biphenyl-2-carboxylic acid.

In some embodiments, provided is a process for the preparation of a compound of formula III

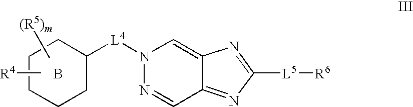

that comprises converting a compound formula 8.1

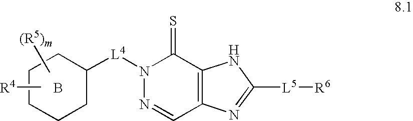

to the compound of formula III.

In some embodiments, the compound of formula III

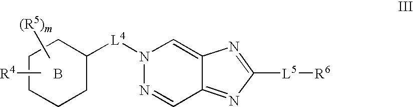

is prepared by a process comprising

reacting the compound of formula 8.1

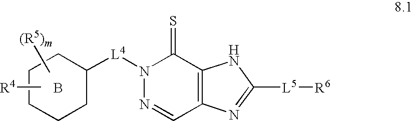

with a desulfurizing reagent.

In some embodiments, the desulfurizing reagent is Raney nickel.

In some embodiments, the compound of formula 8.1

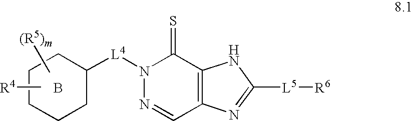

is prepared by a process comprising

reacting a compound of formula 8.2

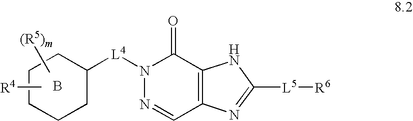

with a sulfurizing reagent.

In some embodiments, the sulfurizing reagent is P₂S₅. In some embodiments, the sulfurizing reagent is Lawesson's reagent.

In some embodiments, the compound of formula 8.2

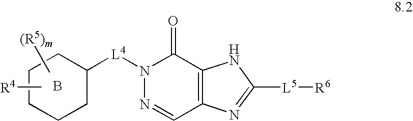

is prepared by a process comprising

cyclizing a compound of formula 8.3

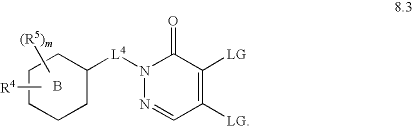

In some embodiments, the cyclization of the compound of formula 8.3

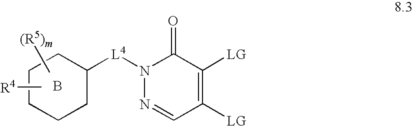

occurs with a compound of formula 8.4

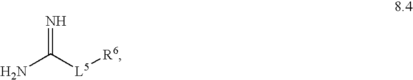

In some embodiments, at least one LG of the compound of formula 8.3 is a halogen. In some embodiments, at least one LG of the compound of formula 8.3 is Br.

In some embodiments, the cyclization of the compound of formula 8.3 occurs with Cs₂CO₃. In some embodiments, the cyclization of the compound of formula 8.3 occurs with Cs₂CO₃ and CuI. In some embodiments, the cyclization of the compound of formula 8.3 occurs with microwave irradiation.

In some embodiments, the compound of formula 8.3

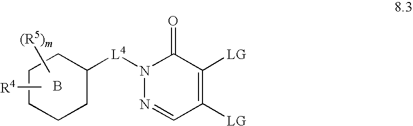

is prepared by a process comprising

cyclizing a compound of formula 8.5

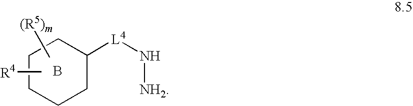

In some embodiments, the cyclization of the compound of formula 8.5 occurs with mucobromic acid.

In some embodiments, the compound of formula 8.5

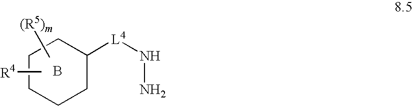

is prepared by a process comprising

reacting a compound of formula 8.6

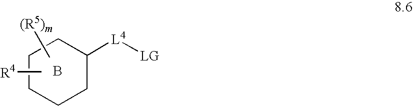

with hydrazine,

wherein

LG is a leaving group.

In some embodiments, the LG of the compound of formula 8.6 is a halogen, hydroxy, alkoxy, substituted alkoxy, or sulfonyloxy. In some embodiments, the LG of the compound of formula 8.6 is a halogen. In some embodiments, the LG of the compound of formula 8.6 is Cl. In some embodiments, the LG of the compound of formula 8.6 is hydroxy. In some embodiments, the LG of the compound of formula 8.6 is a sulfonyloxy. In some embodiments, the LG of the compound of formula 8.6 is —OSO₂C₆H₄-4-CH₃. In some embodiments, the LG of the compound of formula 8.6 is —OSO₂CH₃. In some embodiments, the LG of the compound of formula 8.6 is —OSO₂CF₃.

In some embodiments, a process for the preparation of a compound of formula II is as shown in Scheme 9

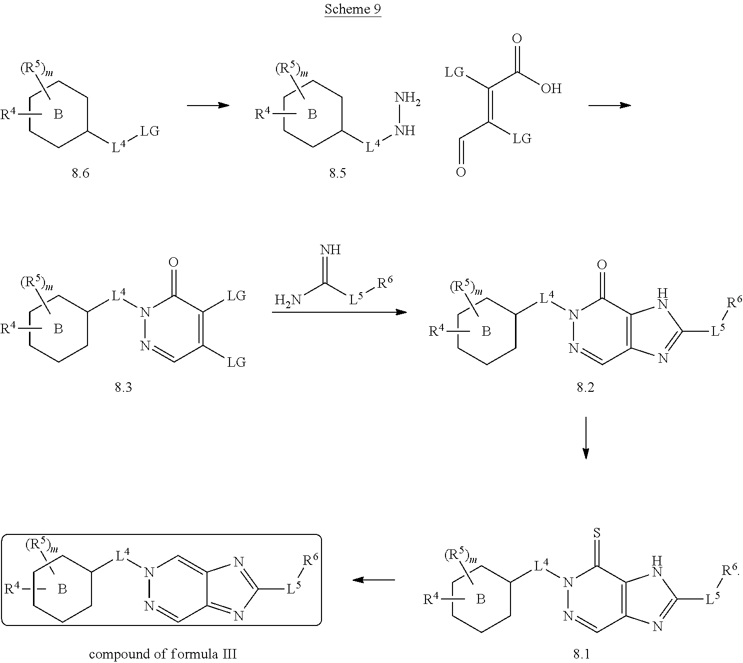

Scheme 9 shows the synthesis of compounds of formula III where R⁴, R⁵, R⁶, L⁴, L⁵, m, and LG are previously defined. The substituted hydrazine 8.5 is formed from displacement of the corresponding electrophiles such as chloroalkyl heterocycles 8.6 with hydrazine. The compounds 8.5 are then cyclized to form compounds of formula 8.3, which are in turn cyclized with amidines giving 2,5-disubstituted-3,5-dihydro-imidazo[4,5-d]pyridazin-4-ones 8.2. These are then converted to the compound of formula III through treatment with reagents such as P₂S₅ followed by reduction with Raney nickel.

In some embodiments, provided is a process for the preparation of a compound of formula III

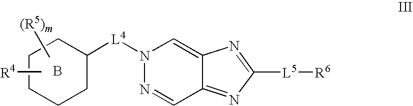

that comprises converting a compound of formula 9.1

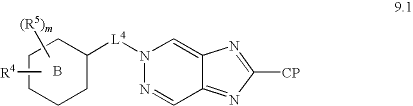

to the compound of formula III,

wherein

CP is a group that can undergo a coupling reaction.

In some embodiments, the compound of formula III

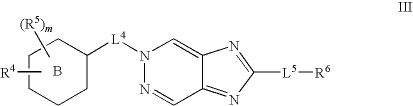

is prepared by a process comprising

coupling a compound of formula 9.1

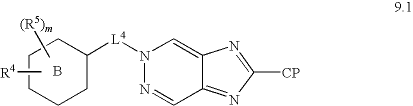

under conditions appropriate to form a carbon-carbon bond.

In some embodiments, the CP of formula 9.1 is a halogen or sulfonlyoxy. In some embodiments, the CP of formula 9.1 is a halogen. In some embodiments, the CP of formula 9.1 is Br. In some embodiments, the CP of formula 9.1 is a sulfonyloxy. In some embodiments, the CP of formula 9.1 is —OSO₂CF₃.

In some embodiments the compound of formula 9.1 is coupled with a compound of formula 9.2

M-L⁵-R⁶ 9.2

In some embodiments, the compound of formula 9.2 comprisies tin, zinc, magnesium, silicon, or boron. In some embodiments, the compound of formula 9.2 is an organotin, organozinc, organomagnesium, organosilyl, organoboron, or organotrifluoroborate compound.

In some embodiments, the organoboron of formula 9.2 is a boronic acid or boronic ester of formula 9.3

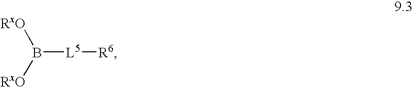

wherein

each R^{x} is independently selected from hydrogen, alkyl, or substituted alkyl and,

wherein

the R^{x} groups, if alkyl or substituted alky, can optionally be connected.

In some embodiments, the organoboron of formula 9.2 is a boronic acid of formula 9.4

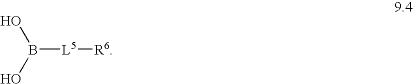

In some embodiments, the coupling reaction of the compound of formula 9.1 occurs in the presence of a metal catalyst. In some embodiments the metal catalyst comprises palladium, nickel, iron, or copper. In some embodiments, the metal catalyst is tetrakistriphenylphosphine palladium.

In some embodiments, the compound of formula 9.1

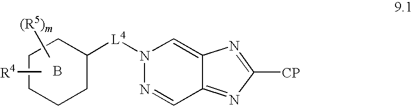

is prepared by a process comprising

reacting a compound of formula 2.5

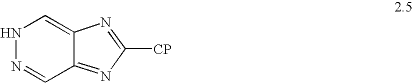

with a compound of formula 9.5

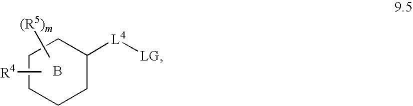

wherein

LG is a leaving group.

In some embodiments, the LG of the compound of formula 9.5 is a halogen, hydroxy, alkoxy, substituted alkoxy, or sulfonyloxy. In some embodiments, the LG of the compound of formula 9.5 is a halogen. In some embodiments, the LG of the compound of formula 9.5 is Cl. In some embodiments, the LG of the compound of formula 9.5 is hydroxy. In some embodiments, the LG of the compound of formula 9.5 is a sulfonyloxy. In some embodiments, the LG of the compound of formula 9.5 is —OSO₂C₆H₄-4-CH₃. In some embodiments, the LG of the compound of formula 9.5 is —OSO₂CH₃. In some embodiments, the LG of the compound of formula 9.5 is —OSO₂CF₃.

In some embodiments, a process for the preparation of a compound of formula I is as shown in Scheme 10

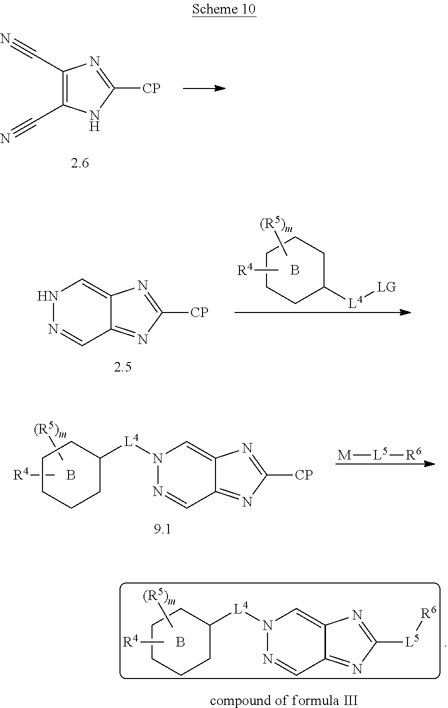

Scheme 10 shows the synthesis of compounds of formula III where R⁴, R⁵, R⁶, L⁴, L⁵, m, LG, CP, and M are previously defined. The dinitrile 2.6 (Heterocycles, 29, 1325, 1989) is reduced with reagents such as DIBAL-H in a solvent such as THF and subsequently cyclized with hydrazine or its derivatives to give 2-substituted-5H-imidazo[4,5-d]pyridazine 2.5. These are then alkylated with electrophiles such as chloroalkyl heterocycles giving the 2-substituted-5-substituted-imidazo[4,5-d]pyridazines 9.1. They can be converted to the compound of formula III through coupling reactions such as the Suzuki reaction.

In some embodiments, provided is a process for the preparation of a compound formula III

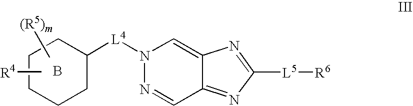

that comprises converting a compound of formula 10.1

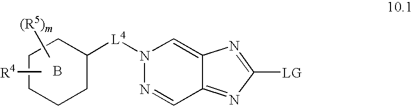

to the compound of formula III,

wherein

LG is a leaving group.

In some embodiments, the compound of formula III

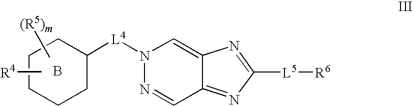

is prepared by a process comprising

reacting a compound of formula 10.1

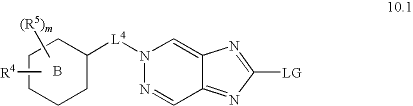

with a compound comprising nitrogen, oxygen or sulfur.

In some embodiments, the LG of the compound of formula 10.1 is a halogen, hydroxy, alkoxy, substituted alkoxy, or sulfonyloxy. In some embodiments, the LG of the compound of formula 10.1 is a halogen. In some embodiments, the LG of the compound of formula 10.1 is Br.

In some embodiments, the compound comprising nitrogen is chosen from aniline, morpholine, piperidine, phenylmethanamine, N-methyl(phenyl)methanamine, 1,2,3,4-tetrahydroquinoline, (2-fluorophenyl)methanamine, (2,3-diflurophenyl)methanamine, 2-phenylethanamine, 1 -phenylethanamine, 1,2,3,4-tetrahydroisoquinoline, 2,3-dihydro-1H-inden-1-amine, 1,2,3,4-tetrahydronaphthalen- 1 -amine, and isoindoline.

In some embodiments, the reaction of the compound of formula 10.1 with a compound comprising nitrogen is heated.

In some embodiments, the reaction of the compound of formula 10.1 occurs with microwave irradiation.

In some embodiments, the compound of formula 10.1

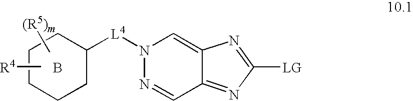

is prepared by a process comprising

reacting the compound of formula 3.2

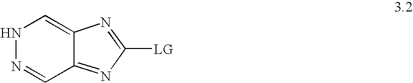

with the compound of formula 9.5

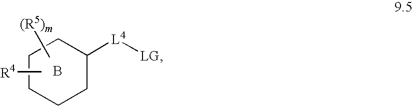

wherein

each LG is independently chosen and each is a leaving group.

In some embodiments, at least one LG in the compounds of formula 3.2 and 9.5 is halogen, hydroxy, alkoxy, substituted alkoxy, or sulfonyloxy. In some embodiments, at least one LG in the compounds of formula 3.2 and 9.5 is a halogen. In some embodiments, at least one LG is Cl. In some embodiments, the LG in the compound of formula 9.5 is Cl. In some embodiments, at least one LG in the compounds of formula 3.2 and 9.5 is Br. In some embodiments, the LG in the compound of formula 3.2 is Br. In some embodiments, at least one LG in the compounds of formula 3.2 and 9.5 is hydroxy. In some embodiments, at least one LG in the compounds of formula 3.2 and 9.5 is sulfonyloxy. In some embodiments, at least one LG in the compounds of formula 3.2 and 9.5 is —OSO₂C₆H₄-4-CH₃. In some embodiments, at least one LG in the compounds of formula 3.2 and 9.5 is —OSO₂CH₃. In some embodiments, at least one LG in the compounds of formula 3.2 and 9.5 is —OSO₂CF₃.

In some embodiments, a process for the preparation of a compound of formula I is as shown in Scheme 11

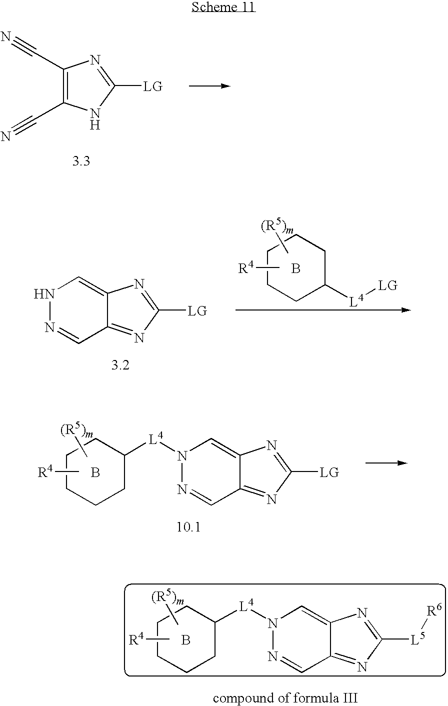

Scheme 11 shows the synthesis of compounds of formula III where R⁴, R⁵, R⁶, L⁴, L⁵, m, and LG are previously defined. The dinitrile 3.3 (Heterocycles, 29, 1325, 1989) is reduced with reagents such as DIBAL-H in a solvent such as THIF and subsequently cyclized with hydrazine or its derivatives to give 2-substituted-5H-imidazo[4,5-d]pyridazine 3.2. These are then alkylated with electrophiles such as chloroalkyl heterocycles giving the 2-substituted-5-substituted-imidazo[4,5-d]pyridazines 10.1. They can be converted to the compound of formula III through reaction with a compound comprising nitrogen, oxygen or sulfur.

In some embodiments, provided is a process for the preparation of a compound formula III

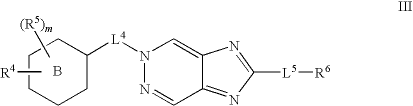

that comprises converting a compound of formula IV

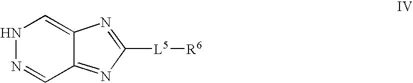

to the compound of formula III.

In some embodiments, the process of converting the compound of formula IV

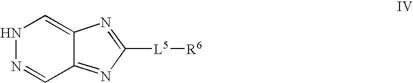

to the compound of formula III

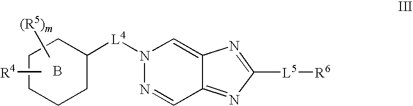

comprises reacting the compound of formula IV with a compound of formula 9.5

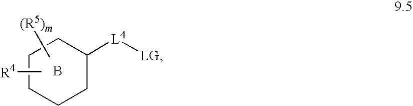

wherein

LG is a leaving group.

In some embodiments, the LG of the compound of formula 9.5 is a halogen, hydroxy, alkoxy, substituted alkoxy, or sulfonyloxy. In some embodiments, the LG of the compound of formula 9.5 is a halogen. In some embodiments, the LG of the compound of formula 9.5 is Cl. In some embodiments, the LG of the compound of formula 9.5 is hydroxy. In some embodiments, the LG of the compound of formula 9.5 is a sulfonyloxy. In some embodiments, the LG of the compound of formula 9.5 is —OSO₂C₆H₄-4-CH₃. In some embodiments, the LG of the compound of formula 9.5 is —OSO₂CH₃. In some embodiments, the LG of the compound of formula 9.5 is —OSO₂CF₃.

In some embodiments, a process for the preparation of a compound of formula I is as shown in Scheme 12

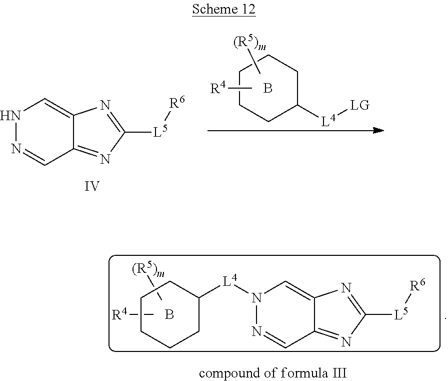

Scheme 12 shows the synthesis of compounds of formula III where R⁴, R⁵, R⁶, L⁴, L⁵, m, and LG are previously defined. The 2-substituted-5H-imidazo[4,5-d]pyridazine IV are alkylated with electrophiles such as chloroalkyl heterocycles giving the compound of formula m.

In some embodiments, the compound of formula IV

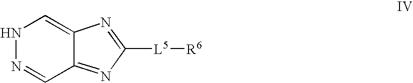

is prepared by a process comprising

a) reacting a compound of formula 10.1

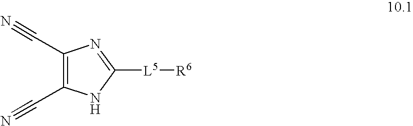

with a reducing reagent

and

b) cyclizing the resulting product to form the compound of formula IV.

In some embodiments, the reducing reagent in the reaction with the compound of formula 10.1 is diisobutylaluminum hydride.

In some embodiments, the product from the reaction of the compound of formula 10.1 with the reducing agent is cyclized with hydrazine.

In some embodiments, the compound of formula 10.1

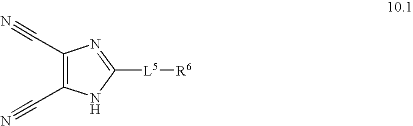

is prepared by a process comprising

cyclizing a compound of formula 10.2

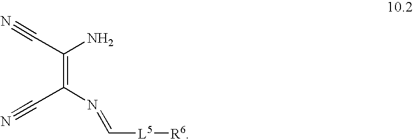

In some embodiments, the cyclization occurs with N-chlorosuccinimide and nicotinamide.

In some embodiments, the compound of formula 10.2

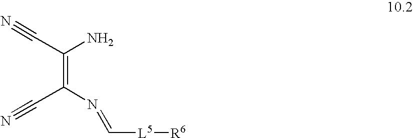

is prepared by a process comprising

reacting a compound of formula 4.3

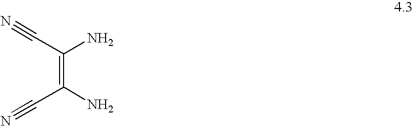

with a compound of formula 10.3

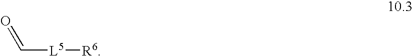

In some embodiments, a process for the preparation of a compound of formula IV is as shown in Scheme 13

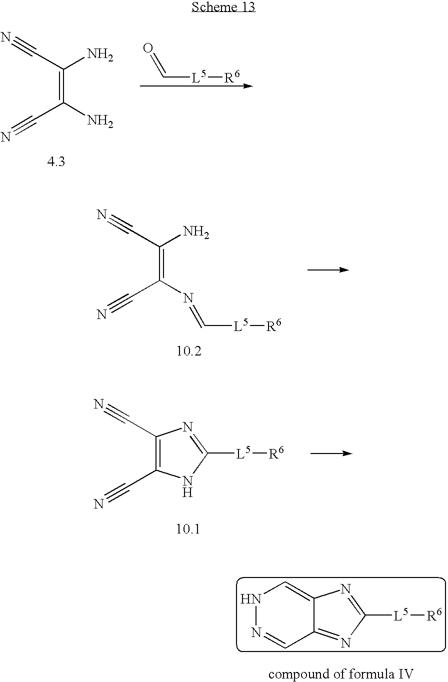

Scheme 13 shows the synthesis of compounds of formula IV where R⁶ and L⁵, are previously defined. The dinitrile 4.3 is condensed with aldehydes of formula H(O)C-L⁵R⁶ and oxidatively cyclized to the 2-substituted imidazole 4,5 dinitrile 10.1. This is then reduced with reagents such as DIBAL-H in a solvent such as THF and subsequently cyclized with hydrazine or its derivatives to yield 2-substituted-5H-imidazo[4,5-d]pyridazine IV.

In some embodiments, the compound of formula IV

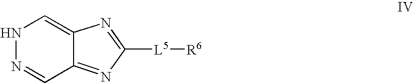

is prepared by a process comprising

a) saponifying a compound of formula 11.1

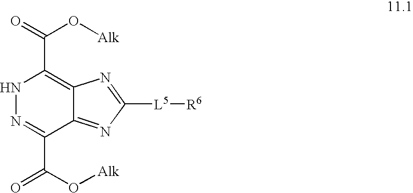

to form a compound of formula 11.2

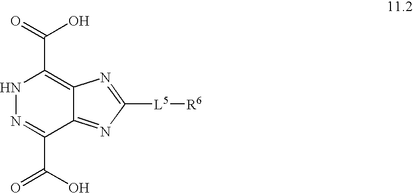

and

b) decarboxylating the compound of formula 11.2 to form the compound of formula IV,

wherein

each Alk is independently chosen and each is an alkyl or substituted alkyl.

In some embodiments, the saponification and decarboxylation of the compound of formula 11.2 occur in the presence of hydrochloric acid and water.

In some embodiments, the Alk in the compound of formula 11.1 is CH₃.

In some embodiments, the compound of formula 11.1

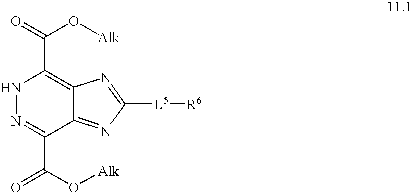

is prepared by a process comprising

reacting a compound of formula 11.3

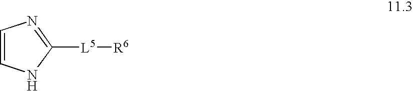

with a compound of formula 11.4

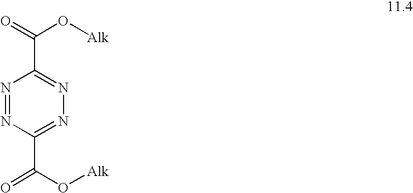

to form the compound of formula 11.1.

In some embodiments, the reaction is heated.

In some embodiments, the compound of formula 11.3

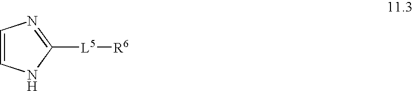

is prepared by a process comprising

reacting a compound of formula 10.3

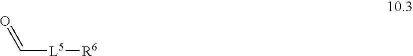

with glyoxal and ammonia.

In some embodiments, a process for the preparation of a compound of formula IV is as shown in Scheme 14

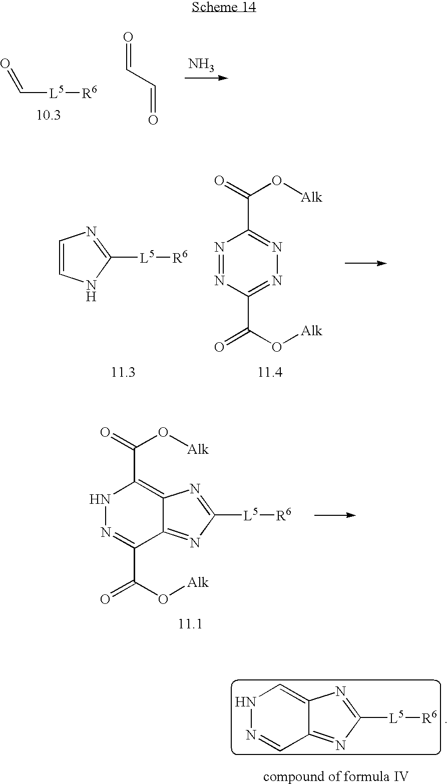

Scheme 14 shows the synthesis of compounds of formula IV where R⁶, L⁵, and Alk are previously defined. The imidazole 11.3 is formed in one step from the corresponding aldehyde 10.3 through condensation with glyoxal and ammonia. The 2-substituted imidazole 11.3 is condensed with reagents such as [1,2,4,5]Tetrazine-3,6-dicarboxylic acid dialkyl ester 11.4 (Org. Syn. Coll. Vol. 9, p 335, 1998). The intermediate 11.1 is then saponified and decarboxylated yielding the compound of formula IV.

In some embodiments, the process of converting the compound of formula IV

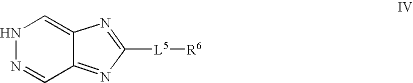

to the compound of formula III

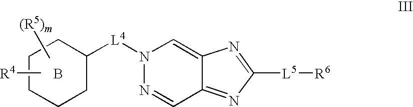

comprises

a) reacting the compound of formula IV with a compound of formula 12.1

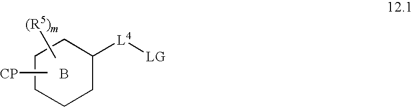

to form a compound of formula 12.2

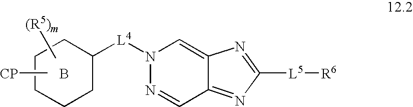

and

b) coupling the compound of formula 12.2 under conditions appropriate to form a carbon-carbon bond

wherein

LG is a leaving group

and

CP is a substituent that can undergo a coupling reaction.

In some embodiments the LG of the compound of formula 12.1 is a halogen, hydroxy, alkoxy, substituted alkoxy, or sulfonyloxy. In some embodiments, the LG of the compound of formula 12.1 is a halogen. In some embodiments, the LG of the compound of formula 12.1 is Cl. In some embodiments, the LG of the compound of formula 12.1 is hydroxy. In some embodiments, the LG of the compound of formula 12.1 is a sulfonyloxy. In some embodiments, the LG of the compound of formula 12.1 is —OSO₂C₆H₄-4-CH₃. In some embodiments, the LG of the compound of formula 12.1 is —OSO₂CH₃. In some embodiments, the LG of the compound of formula 12.1 is —OSO₂CF₃.

In some embodiments the compound of formula 12.2 is coupled with a compound of formula 12.3

M-R⁴ 12.3

to form the compound of formula IV,

wherein

M is a substituent that can undergo a coupling reaction.

In some embodiments, the compound of formula 12.3 comprises tin, zinc, magnesium, silicon, or boron. In some embodiments, the compound of formula 12.3 can be an organotin, organozinc, organomagnesium, organosilyl, organoboron, or organotrifluoroborate compound.

In some embodiments, the organoboron of formula 12.3 is a boronic acid or boronic ester of formula 12.4

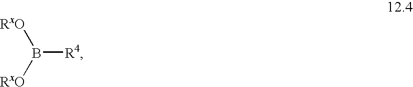

wherein

each R^{x} is independently selected from hydrogen, alkyl, or substituted alkyl

and,

wherein

the R^{x} groups, if alkyl or substituted alkyl, can optionally be connected.

In some embodiments, the organoboron of formula 12.3 is a boronic acid of formula 12.5

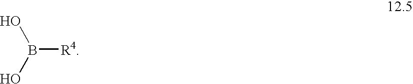

In some embodiments, the CP of formula 12.2 is a halogen or sulfonyloxy. In some embodiments, the CP of formula 12.2 is a halogen. In some embodiments, the CP of formula 12.2 is Br. In some embodiments, the CP of formula 12.2 is a sulfonyloxy. In some embodiments, the CP of formula 12.2 is —OSO₂CF₃.

In some embodiments, the coupling reaction of the compound of formula 12.2 occurs in the presence of at least one compound comprising palladium, nickel, iron, or copper. In some embodiments, the coupling reaction of the compound of formula 12.2 occurs in the presence of tetrakistriphenylphosphine palladium.

In some embodiments, a process for the preparation of a compound of formula III is as shown in Scheme 15

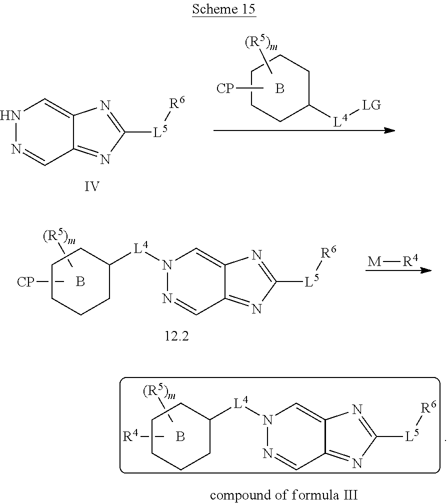

Scheme 15 shows the synthesis of compounds of formula III where R⁴, R⁵, R⁶, L⁴, L⁵, m, LG, CP, and M are previously defined. The 2-substituted-5H-imidazo[4,5-d]pyridazine IV is alkylated with electrophiles such as chloroalkyl heterocycles giving the products 12.2 which can then be converted to the compound of formula III.

In some embodiments, the the process of converting the compound of formula IV

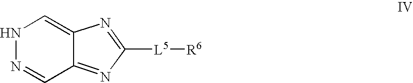

to the compound of formula III

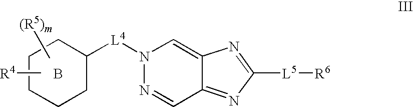

comprises

a) reacting the compound of formula IV with a compound of formula 13.1

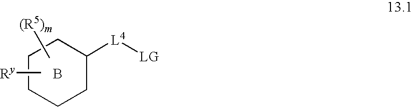

to form a compound of formula 13.2

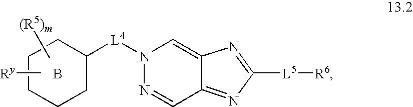

b) reacting the compound of formula 13.2 with a suitable reagent to form a compound of formula 13.3

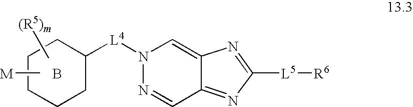

and

c) coupling the compound of formula 13.3 with a compound of formula 13.4

CP—R⁴ 13.4

under conditions appropriate to form a carbon-carbon bond,

wherein

LG is a leaving group,

R^{y} is a halogen or sulfonyloxy,

and

M and CP are substituents that can undergo a coupling reaction.

In some embodiments, the LG of the compound of formula 13.1 is a halogen, hydroxy, alkoxy, substituted alkoxy, or sulfonyloxy. In some embodiments, the LG of the compound of formula 13.1 is a halogen. In some embodiments, the LG of the compound of formula 13.1 is Cl. In some embodiments, the LG of the compound of formula 13.1 is hydroxy. In some embodiments, the LG of the compound of formula 13.1 is a sulfonyloxy. In some embodiments, the LG of the compound of formula 13.1 is —OSO₂C₆H₄-4-CH₃, —OSO₂CH₃, or —OSO₂CF₃.

In some embodiments, the R^{y} of the compound of formula 13.1 is Br. In some embodiments, the R^{y} of the compound of formula 13.1 is —OSO₂CF₃.

In some embodiments, the M of the compound of formula 13.3 comprises tin, zinc, magnesium, silicon, or boron. In some embodiments, the M of the compound of formula 13.3 comprises boron.

In some embodiments, the coupling reaction of step (c) occurs in the presence of at least one compound comprising palladium, nickel, iron, or copper. In some embodiments, the coupling reaction of step (c) occurs in the presence of tetrakistriphenylphosphine palladium.

In some embodiments, a process for the preparation of a compound of formula III is as shown in Scheme 16

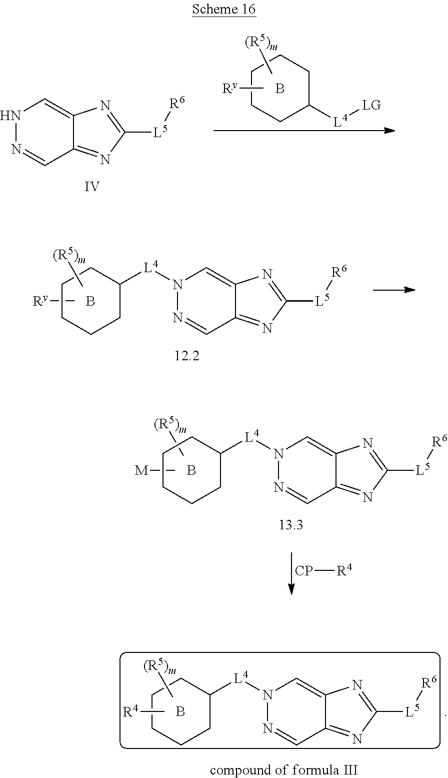

Scheme 16 shows the synthesis of compounds of formula I where R⁴, R⁵, R⁶, L⁴, L⁵, m, LG, CP, M, and R^{y} are previously defined. The 2-substituted-5H-imidazo[4,5-d]pyridazine IV is alkylated with electrophiles such as chloroalkyl heterocycles giving the products 13.2 which can then be converted to 13.3. These can then undergo coupling reactions to provide the compound of formula III.

In some embodiments, provided is a process for the preparation of a compound formula III

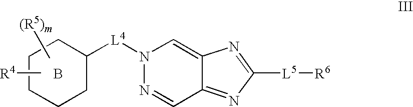

that comprises converting a compound of formula IV

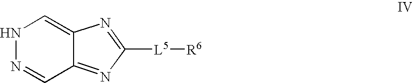

to the compound of formula III.

The compound of formula IV may be prepared by any of the processes described above.

In some embodiments, the process of converting the compound of formula IV

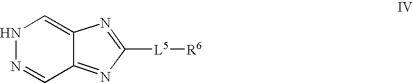

to the compound of formula III

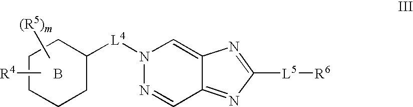

comprises reacting the compound of formula IV with a compound of formula 9.5

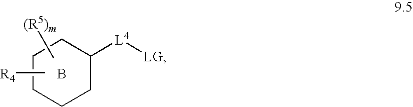

wherein LG is a leaving group.

In some embodiments, LG in the compound of formula 9.5 is a halogen, hydroxy, alkoxy, substituted alkoxy, or sulfonyloxy. In some embodiments, LG in the compound of formula 9.5 is a halogen. In some embodiments, LG in the compound of formula 9.5 is Cl. In some embodiments, LG in the compound of formula 9.5 is hydroxy. In some embodiments, LG in the compound of formula 9.5 is a sulfonyloxy. In some embodiments, LG in the compound of formula 9.5 is —OSO₂C₆H₄-4-CH₃. In some embodiments, LG in the compound of formula 9.5 is —OSO₂CH₃. In some embodiments, LG in the compound of formula 9.5 is —OSO₂CF₃.

In some embodiments, the compound of formula 9.5 is prepared by a process comprising reacting a compound of formula 16.1

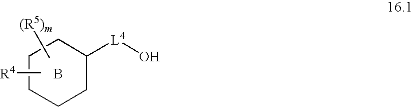

with a compound containing LG to form the compound of formula 9.5.

In some embodiments, the compound containing LG is a halogenated compound, such as a chlorinated compound (e.g., thionyl chloride or phosphorous tribromide).

In some embodiments, the compound of formula 16.1 is prepared by a process comprising reacting a compound of formula 16.2

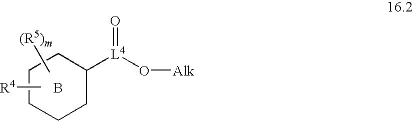

with a reducing reagent to form the compound of formula 16.1, wherein Alk is alkyl or substituted alkyl.

In some embodiments, Alk in the compound of formula 16.2 is CH₃.

In some embodiments, the reducing agent is selected from the group consisting of lithium aluminum hydride, sodium borohydride and diisobutylaluminum hydride.

In some embodiments, the compound of formula 16.2 is prepared by a process comprising reacting a compound of formula 16.3

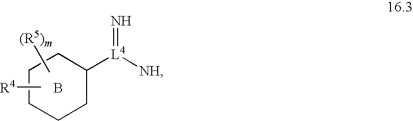

or a salt thereof, with an alkoxy ester to form the compound of formula 16.2.

In some embodiments, reaction of the compound of formula 16.3 (or salt thereof) with the alkoxy ester occurs in the presence of nitrogen. In some embodiments, the reaction is heated.

In some embodiments, the alkoxy ester is methyl-2-[bis(methyloxy)methyl]-3-hydroxy-2-propenoate or a salt thereof, such as a sodium salt.

In some embodiments, the compound of formula 16.3 (or salt thereof) is prepared by a process comprising reacting a compound of formula 16.4

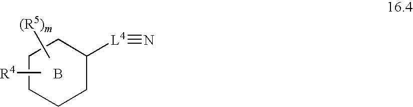

with a nucleophilic base to form the compound of formula 16.3 or a salt thereof.

In some embodiments, reaction of the compound of formula 16.4 with the nucleophilic base occurs in the presence of nitrogen. In some embodiments, the reaction is heated.

In some embodiments, the nucleophilic base is a salt of hexamethyldisilazide, such as a sodium salt.

In some embodiments, a process for the preparation of a compound of formula III is as shown in Scheme 17:

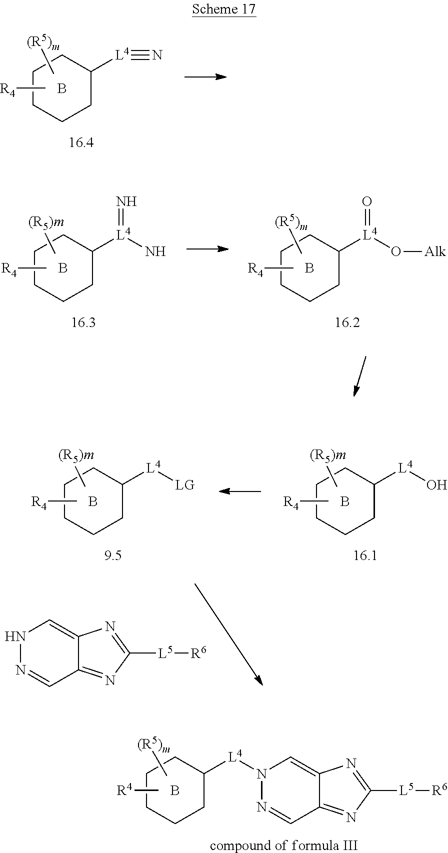

Scheme 17 shows the synthesis of compounds of formula III where R⁴, R⁵, R⁶, L⁴, L⁵, m, and LG are previously defined. Nitrile 16.4 is treated with a nucleophilic base (e.g., potassium hexamethyldisilazide) in the present of a solvent (e.g., tetrahydrofuran) and optionally an acid (e.g., HCl) to give carboximidamide 16.3 (or a salt thereof). Alternatively, carboximidamide 16.3 may be formed through other known reactions, such as the Pinner reaction, which involves the reaction of a nitrile with an alcohol under acid catalysis to form an alkyl imidate salt, which then reacts with ammonia or amine to form the amidine. Regardless, carboximidamide 16.3 (or a salt thereof) reacts with an alkoxy ester (e.g., methy-2-[bis(methyloxy)methyl]-3-hydroxy-propenoate sodium salt) under nitrogen and heat, and in the presence of a solvent (e.g., N,N-Dimethylformadide), to give alkyl ester 16.2. Alkyl ester 16.2 is reacted with a reducing agent (e.g., lithium aluminum hydride, sodium borohydride, or diisobutylaluminum hydride) in the presence of a solvent (e.g., tetrahydrofuran) to give alcohol 16.1. Nucleophilic substitution of the hydroxy group in alcohol 16.1 is accomplished through reagents such as thionyl chloride or phosphorous tribromide in the presence of a solvent (e.g., chloroform) to give 9.5 (e.g., chloroalkyl heterocycle). The 2-substituted-5H-imidazo[4,5-d]pyridazine IV is alkylated with 9.5 yielding the compound of formula III.

Also provided is a composition comprising:

(1) a compound of formula III or a salt or solvate thereof

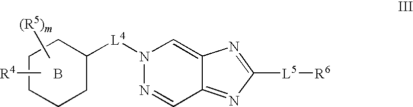

wherein

ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;

L⁴ is L⁶;

L⁵ is a bond or L⁶;

L⁶ is independently C₃₋₆ cycloalkylene or is C₁₋₅ alkylene where one or two —CH₂— groups of said C₁₋₅ alkylene are optionally replaced with —NR⁷—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵;

R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;

R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;

R⁷ is independently H, alkyl, or substituted alkyl;

m is 0, 1,2, 3, or4; and

provided that the compound of Formula I is not 4′-(2-butyl-imidazo[4,5-d]-pyridazin-5-ylmethyl)-biphenyl-2-carboxylic acid.

and

(2) a detectable amount of one or more compounds selected from:

a compound of formula 6.1

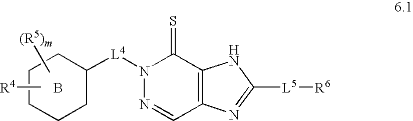

or a salt thereof; P₂S₅;

a compound of formula 7.1

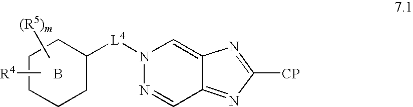

or a salt thereof, wherein CP is a group that can undergo a coupling reaction;

a compound of formula 15.1

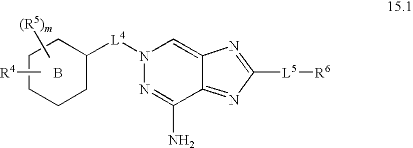

or a salt thereof, wherein L⁴, L⁵, R⁴, R⁵, R⁶, and m are as defined above;

a compound comprising tin, zinc, magnesium, silicon, or boron;

a compound comprising palladium, nickel, iron, or copper;

hydrazine;

and

Cs₂CO₃.

In some embodiments, the processes described herein are used to prepare the compounds included in Table 1 or pharmaceutically acceptable salts or solvates thereof.

TABLE 1

| Com- | | |
|---|---|---|
| pound | | |
| # | Structure | Name |
| 101 | 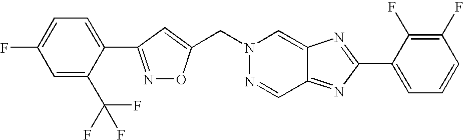 | 2-(2,3-Difluoro-phenyl)- 5-[3-(4-fluoro- 2-trifluoromethyl-phenyl)-isoxazol- 5-ylmethyl]-5H-imidazo[4,5- d]pyridazine |
| 102 | 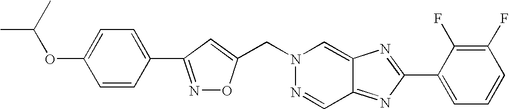 | 2-(2,3-Difluoro-phenyl)-5-[3-(4- isopropoxy-phenyl)-isoxazol-5- ylmethyl]-5H-imidazo[4,5- d]pyridazine |
| 103 | 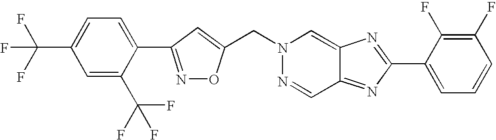 | 5-[3-(2,4-Bis- trifluoromethyl-phenyl)- isoxazol-5-ylmethyl]-2-(2,3-difluoro- phenyl)-5H-imidazo[4,5- d]pyridazine |
| 104 | 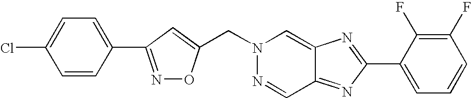 | 5-[3-(4-Chloro-phenyl)-isoxazol-5- ylmethyl]-2-(2,3-difluoro-phenyl)- 5H-imidazo[4,5-d]pyridazine |
| 105 | 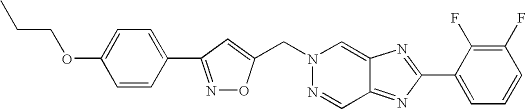 | 2-(2,3-Difluoro-phenyl)-5-[3-(4- propoxy-phenyl)-isoxazol-5- ylmethyl]- 5H-imidazo[4,5-d]pyridazine |
| 106 | 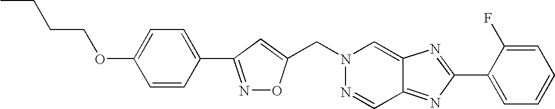 | 5-[3-(4-Butoxy-phenyl)-isoxazol-5- ylmethyl]-2-(2-fluoro-phenyl)-5H- imidazo[4,5-d]pyridazine |
| 107 | 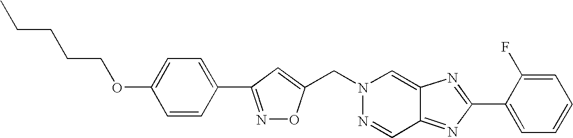 | 2-(2-Fluoro-phenyl)-5-[3- (4-pentyloxy- phenyl)-isoxazol-5-ylmethyl]-5H- imidazo[4,5-d]pyridazine |
| 108 | 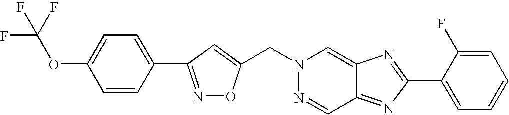 | 2-(2-Fluoro-phenyl)-5-[3-(4- trifluoromethoxy-phenyl)-isoxazol-5- ylmethyl]-5H-imidazo[4,5- d]pyridazine |
| 109 | 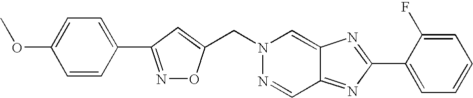 | 2-(2-Fluoro-phenyl)-5-[3- (4-methoxy- phenyl)-isoxazol-5-ylmethyl]-5H- imidazo[4,5-d]pyridazine |
| 110 | 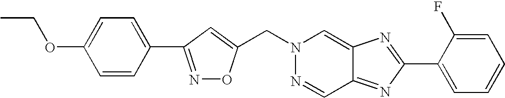 | 5-[3-(4-Ethoxy-phenyl)-isoxazol-5- ylmethyl]-2-(2-fluoro-phenyl)-5H- imidazo[4,5-d]pyridazine |
| 111 | 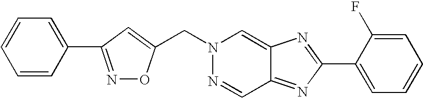 | 2-(2-Fluoro-phenyl)-5-(3-phenyl- isoxazol-5-ylmethyl)-5H- imidazo[4,5- d]pyridazine |
| 112 | 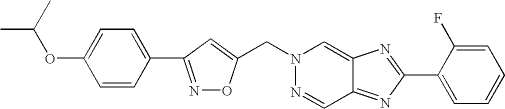 | 2-(2-Fluoro-phenyl)-5-[3-(4- isopropoxy-phenyl)-isoxazol-5- ylmethyl]-5H-imidazo[4,5- d]pyridazine |
| 113 | 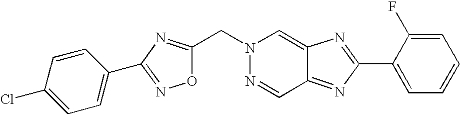 | 5-[3-(4-Chloro-phenyl)- [1,2,4]oxadiazol-5-ylmethyl]-2-(2- fluoro-phenyl)-5H-imidazo[4,5- d]pyridazine |
| 114 | 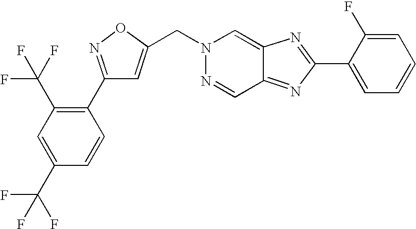 | 5-[3-(2,4-Bis-trifluoromethyl- phenyl)- isoxazo-5-ylmethyl]-2-(2-fluoro- phenyl)-5H- imidazo[4,5-dipyridazine |
| 115 | 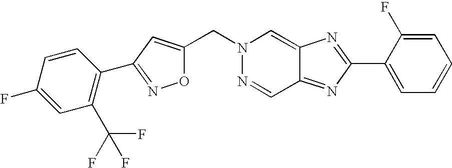 | 2-(2-Fluoro-phenyl)-5-[3- (4-fluoro-2- trifluoromethyl-phenyl)-isoxazol-5- ylmethyl]-5H-imidazo[4,5- d]pyridazine |
| 116 | 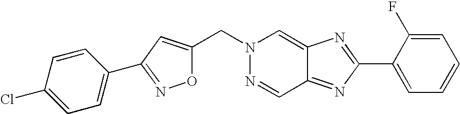 | 5-[3-(4-Chloro-phenyl)-isoxazol-5- ylmethyl]-2-(2-fluoro-phenyl)-5H- imidazo[4,5- d]pyridazine |
| 117 | 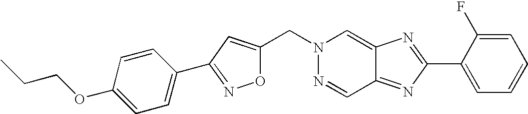 | 2-(2-Fluoro-phenyl)-5- [3-(4-propoxy- phenyl)-isoxazol-5-ylmethyl]-5H- imidazo[4,5- d]pyridazine |
| 119 | 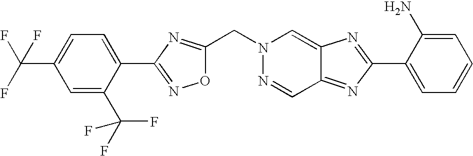 | 2-{5-[3-(2,4-Bis-trifluoromethyl- phenyl)-isoxazol-5-ylmethyl]-5H- imidazo[4,5- d]pyridazin-2-yl}- phenylamine |
| 120 | 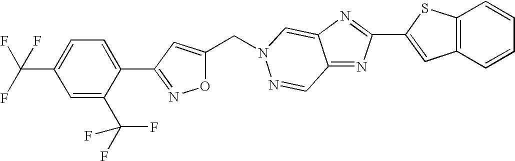 | 2-Benzo[b]-thiophen-2-yl- 5-[3-(2,4-bis- trifluoromethyl-phenyl)-isoxazol-5- ylmethyl]-5H-imidazo[4,5- d]pyridazine |
| 121 | 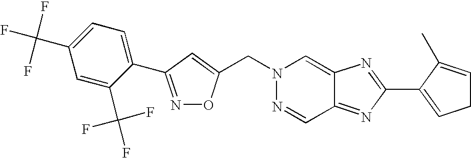 | 5-[3-(2,4-Bis- trifluoromethyl-phenyl)- isoxazol-5-ylmethyl]-2-(4-methyl- thiophen-3-yl)-5H-imidazo[4,5- d]pyridazine |
| 122 | 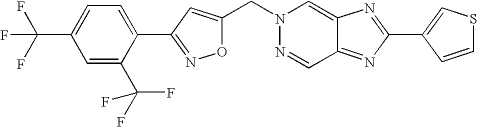 | 5-[3-(2,4-Bis- trifluoromethyl-phenyl)- isoxazol-5-ylmethyl]- 2-thiophen-3-yl- 5H-imidazo[4,5- d]pyridazine |
| 123 | 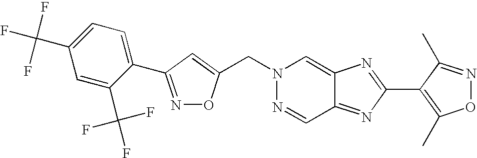 | 5-[3-(2,4-Bis- trifluoromethyl-phenyl)- isoxazol-5-ylmethyl]- 2-(3,5-dimethyl- isoxazol-4-yl)-5H-imidazo[4,5- d]pyridazine |
| 124 | 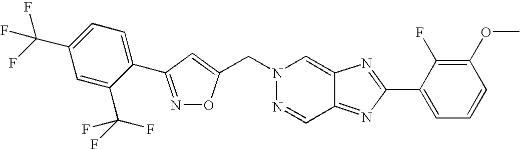 | 5-[3-(2,4-Bis- trifluoromethyl-phenyl)- isoxazol-5-ylmethyl]-2-(2-fluoro-3- methoxy-phenyl)-5H-imidazo[4,5- d]pyridazine |
| 125 | 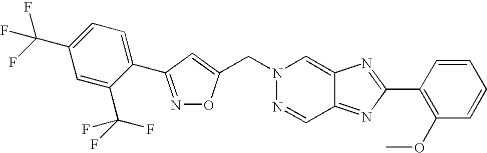 | 5-[3-(2,4-Bis- trifluoromethyl-phenyl)- isoxazol-5-ylmethyl]-2-(2-methoxy- phenyl)-5H-imidazo[4,5- d]pyridazine |
| 126 | 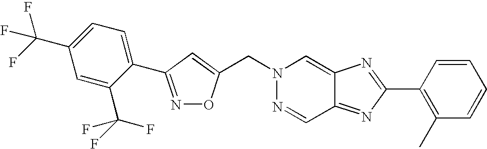 | 5-[3-(2,4-Bis- trifluoromethyl-phenyl)- isoxazol-5-ylmethyl]-2-o-tolyl-5H- imidazo[4,5- d]pyridazine |
| 127 | 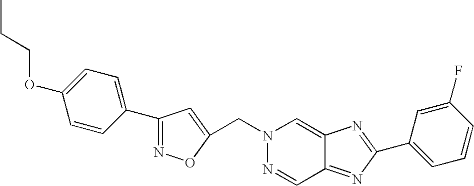 | 2-(3-Fluoro-phenyl)- 5-[3-(4-propoxy- phenyl)-isoxazol-5-ylmethyl]-5H- imidazo[4,5- d]pyridazine |
| 128 | 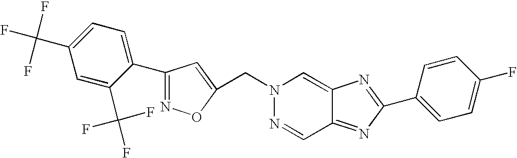 | 5-[3-(2,4-Bis- trifluoromethyl-phenyl)- isoxazol-5-ylmethyl]-2-(4-fluoro- phenyl)-5H-imidazo[4,5- d]pyridazine |
| 129 | 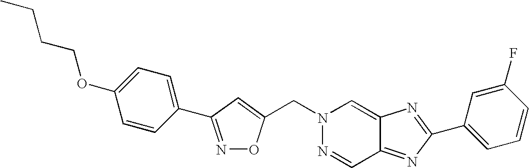 | 5-[3-(4-Butoxy-phenyl)-isoxazol-5- ylmethyl]-2-(3-fluoro-phenyl)-5H- imidazo[4,5- d]pyridazine |
| 130 | 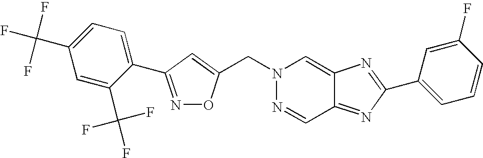 | 5-[3-(2,4-Bis- trifluoromethyl-phenyl)- isoxazol-5-ylmethyl]-2-(3-fluoro- phenyl)-5H-imidazo[4,5- d]pyridazine |
| 131 | 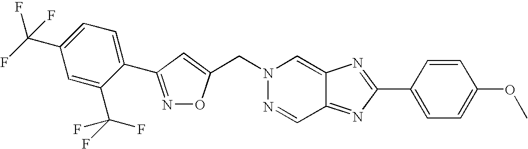 | 5-[3-(2,4-Bis- trifluoromethyl-phenyl)- isoxazol-5-ylmethyl]-2-(4-methoxy- phenyl)-5H-imidazo[4,5- d]pyridazine |
| 132 | 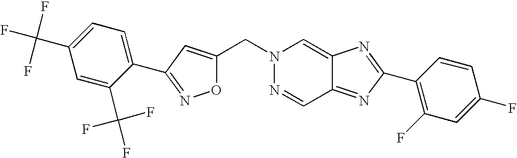 | 5-[3-(2,4-Bis- trifluoromethyl-phenyl)- isoxazol-5-ylmethyl]-2-(2,4-difluoro- phenyl)-5H-imidazo[4,5- d]pyridazine |
| 133 | 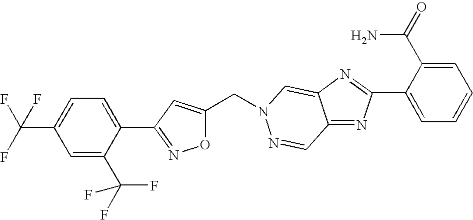 | 2-{5-[3-(2,4-Bis-trifluoromethyl- phenyl)-isoxazol-5-ylmethyl]-5H- imidazo[4,5- d]pyridazin-2-yl}- benzamide |
| 134 | 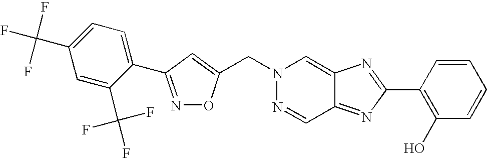 | 2-{5-[3-(2,4-Bis- trifluoromethyl- phenyl)-isoxazol-5-ylmethyl]-5H- imidazo[4,5- d]pyridazin-2-yl}-phenol |
| 135 | 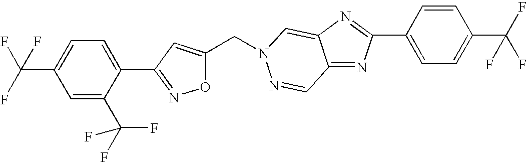 | 5-[3-(2,4-Bis- trifluoromethyl-phenyl)- isoxazol-5-ylmethyl]-2-(4- trifluoromethyl-phenyl)-5H- imidazo[4,5- d]pyridazine |
| 136 | 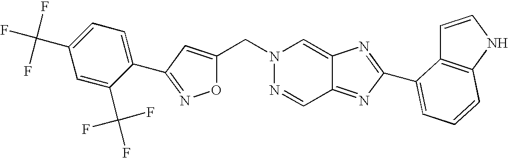 | 5-[3-(2,4-Bis- trifluoromethyl-phenyl)- isoxazol-5-ylmethyl]-2-(1H- indol-4-yl)- 5H-imidazo[4,5- d]pyridazine |
| 137 | 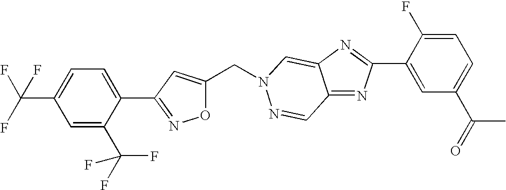 | 1-(3-{5-[3-(2,4-Bis- trifluoromethyl- phenyl)-isoxazol-5-ylmethyl]-5H- imidazo[4,5- d]pyridazin-2-yl}-4-fluoro- phenyl)-ethanone |
| 138 | 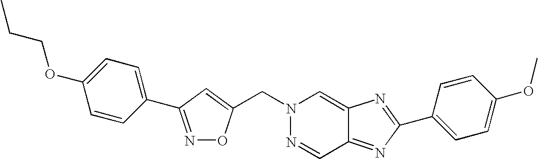 | 2-(4-Methoxy-phenyl)-5- [3-(4-propoxy- phenyl)-isoxazol-5-ylmethyl]-5H- imidazo[4,5- d]pyridazine |
| 139 | 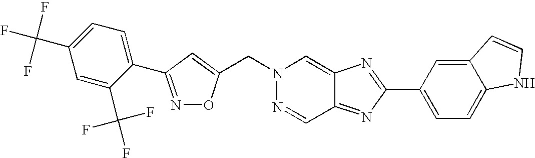 | 5-[3-(2,4-Bis- trifluoromethyl-phenyl)- isoxazol-5-ylmethyl]-2- (1H-indol-5-yl)- 5H-imidazo[4,5- d]pyridazine |
| 140 | 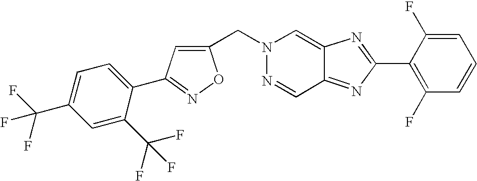 | 5-[3-(2,4-Bis- trifluoromethyl-phenyl)- isoxazol-5-ylmethyl]-2-(2,6-difluoro- phenyl)-5H-imidazo[4,5- d]pyridazine |
| 141 | 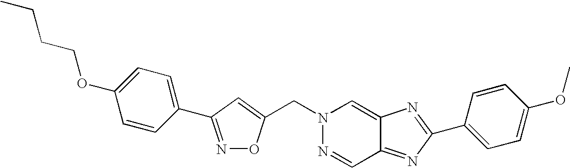 | 5-[3-(4-Butoxy-phenyl)-isoxazol-5- ylmethyl]-2-(4-methoxy-phenyl)-5H- imidazo[4,5- d]pyridazine |
| 142 | 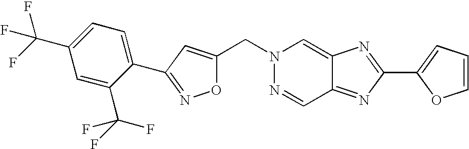 | 5-[3-(2,4-Bis- trifluoromethyl-phenyl)- isoxazol-5-ylmethyl]-2- furan-2-yl-5H- imidazo[4,5- d]pyridazine |
| 143 | 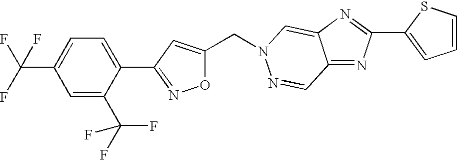 | 5-[3-(2,4-Bis- trifluoromethyl-phenyl)- isoxazol-5-ylmethyl]-2- thiophen-2-yl- 5H-imidazo[4,5- d]pyridazine |
| 144 | 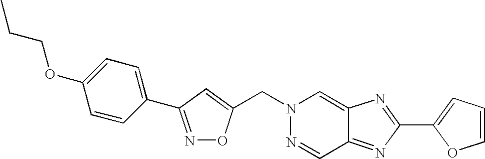 | 2-Furan-2-yl-5-[3- (4-propoxy-phenyl)- isoxazol-5-ylmethyl]- 5H-imidazo[4,5- d]pyridazine |
| 145 | 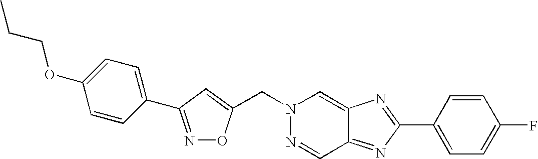 | 2-(4-Fluoro-phenyl)-5- [3-(4-propoxy- phenyl)-isoxazol-5-ylmethyl]-5H- imidazo[4,5- d]pyridazine |
| 146 | 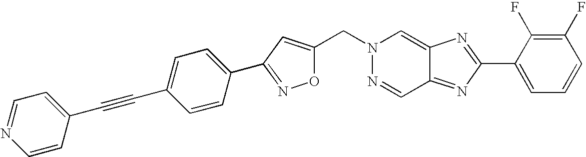 | 2-(2,3-Difluoro-phenyl)-5-[3-(4-pyridin- 4-ylethynyl-phenyl)-isoxazol-5- ylmethyl]-5H-imidazo[4,5- d]pyridazine |
| 147 | 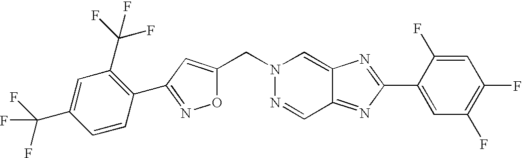 | 5-[3-(2,4-Bis- trifluoromethyl-phenyl)- isoxazol-5-ylmethyl]- 2-(2,4,5-trifluoro- phenyl)-5H-imidazo[4,5- d]pyridazine |
| 148 | 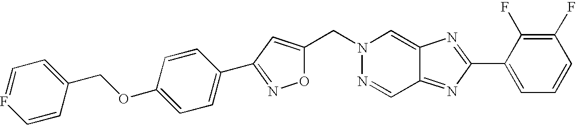 | 2-(2,3-Difluoro-phenyl)-5-{3-[4- (pyridin-4-ylmethoxy)-phenyl]- isoxazol-5-ylmethyl}- 5H-imidazo-4,5- d]pyridazine |
| 149 | 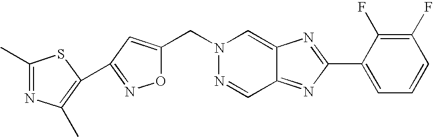 | 2-(2,3-Difluoro-phenyl)-5-[3-(2,4- dimethyl-thiazol-5-yl)-isoxazol-5- ylmethyl]-5H- imidazo[4,5-d]pyridazine |
| 150 | 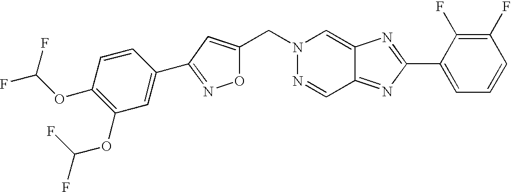 | 5[3-(3,4-Bis- difluoromethoxy-phenyl)- isoxazol-5-ylmethyl]-2-(2,3-difluoro- phenyl)-5H-imidazo [4,5-d]pyridazine |
| 151 | 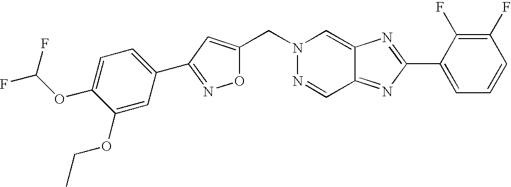 | 5-[3-(4-Difluoromethoxy-3-ethoxy- phenyl)-isoxazol-5-ylmethyl]-2-(2,3- difluoro-phenyl)-5H-imidazo[4,5- d]pyridazine |
| 152 | 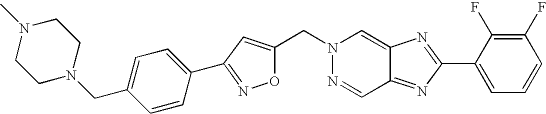 | 2-(2,3-Difluoro-phenyl)-5-{3-[4-(4- methyl-piperazin-1- ylmethyl)-phenyl]- isoxazol-5-ylmethyl}-5H- imidazo[4,5- d]pyridazine |
| 153 | 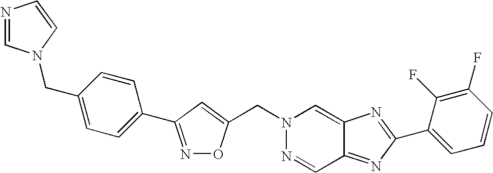 | 2-(2,3-Difluoro-phenyl)-5-[3-(4- imidazol-1-ylmethyl- phenyl)-isoxazol 5-ylmethyl]-5H-imidazo[4,5- d]pyridazine |
| 154 | 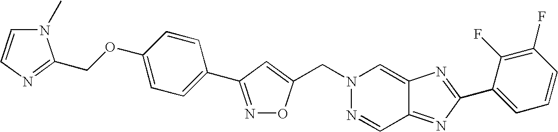 | 2-(2,3-Difluoro-phenyl)-5-{3-[4-(1- methyl-1H-imidazol-2-ylmethoxy)- phenyl]-isoxazol-5-ylmethyl}-5H- imidazo[4,5- d]pyridazine |
| 155 | 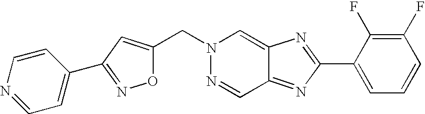 | 2-(2,3-Difluoro-phenyl)- 5-(3-pyridin-4- yl-isoxazol-5-ylmethyl)-5H- imidazo[4,5- d]pyridazine |
| 156 | 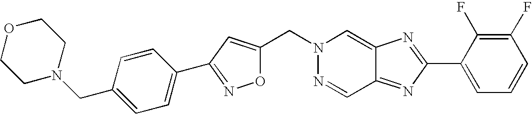 | 2-(2,3-Difluoro-phenyl)-5-[3-(4- morpholin-4-ylmethyl-phenyl)- isoxazol-5-ylmethyl]- 5H-imidazo[4,5- d]pyridazine |
| 157 | 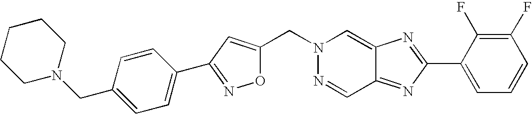 | 2-(2,3-Difluoro-phenyl)-5-[3-(4- piperidin-1-ylmethyl- phenyl)-isoxazol- 5-ylmethyl]-5H-imidazo[4,5- d]pyridazine |
| 158 | 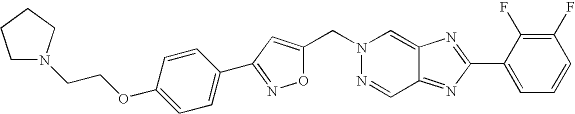 | 2-(2,3-Difluoro-phenyl)-5-{3-[4-(2- pyrrolidin-1-yl-ethoxy)-phenyl]- isoxazol-5-ylmethyl}- 5H-imidazo[4,5- d]pyridazine |
| 159 | 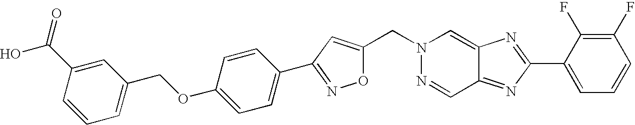 | 3-(4-{5-[2-(2,3-Difluoro-phenyl)- imidazo[4,5- d]pyridazin-5-ylmethyl]- isoxazol-3-yl}-phenoxymethyl)- benzoic acid |
| 160 | 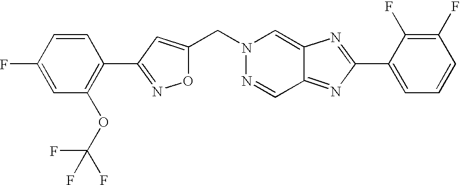 | 2-(2,3-Difluoro-phenyl)-5-[3-(4-fluoro- 2-trifluoromethoxy- phenyl)-isoxazol-5- ylmethyl]-5H-imidazo[4,5- d]pyridazine |
| 161 | 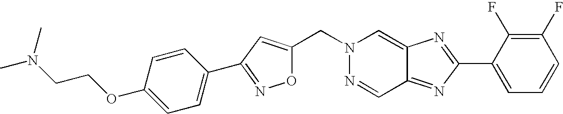 | [2-(4-{5-[2-(2,3-Difluoro-phenyl)- imidazo[4,5- d]pyridazin-5-yl methyl]- isoxazol-3-yl}-phenoxy)-ethyl]- dimethyl-amine |
| 162 | 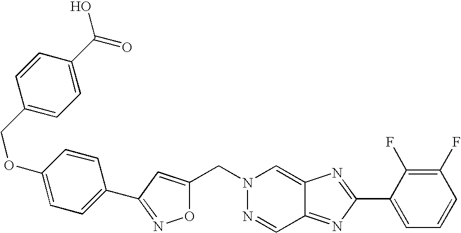 | 4-(4-{5-[2-(2,3-Difluoro-phenyl)- imidazo[4,5- d]pyridazin-5-ylmethyl]- isoxazol-3-yl}-phenoxymethyl)- benzoic acid |
| 163 | 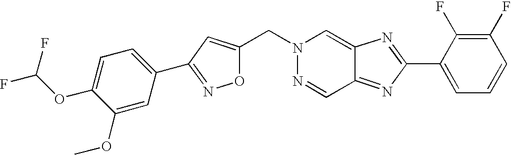 | 5-[3-(4-Difluoromethoxy- 3-methoxy- phenyl)-isoxazol-5-ylmethyl]-2-(2,3- difluoro-phenyl)-5H-imidazo[4,5- d]pyridazine |
| 164 | 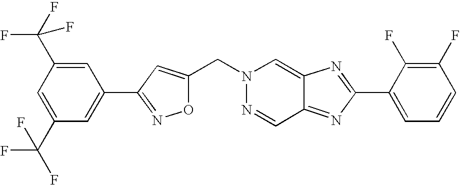 | 5-[3-(3,5-Bis- trifluoromethyl-phenyl)- isoxazol-5-ylmethyl]-2-(2,3-difluoro- phenyl)-5H-imidazo[4,5- d]pyridazine |
| 165 | 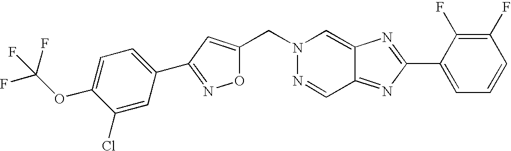 | 5-[3-(3-Chloro-4-trifluoromethoxy- phenyl)-isoxazol-5-ylmethyl]-2-(2,3- difluoro-phenyl)-5H-imidazo[4,5- d]pyridazine |
| 166 | 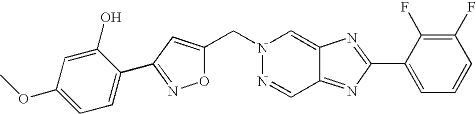 | 2-{5-[2-(2,3-Difluoro-phenyl)- imidazo[4,5- d]pyridazin-5-ylmethyl]- isoxazol-3-yl}-5-methoxy-phenol |
| 167 | 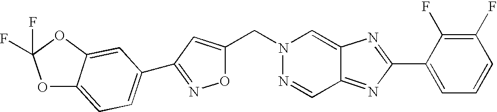 | 5-[3-(2,2-Difluoro-benzo [1,3]dioxol-5- yl)-isoxazol-5-ylmethyl]-2- (2,3-difluoro- phenyl)-5H-imidazo[4,5- d]pyridazine |
| 168 | 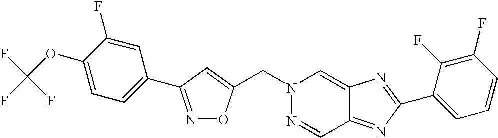 | 2-(2,3-Difluoro-phenyl)-5- [3-(3-fluoro- 4-trifluoromethoxy- phenyl)-isoxazol-5- ylmethyl]-5H-imidazo[4,5- d]pyridazine |
| 169 | 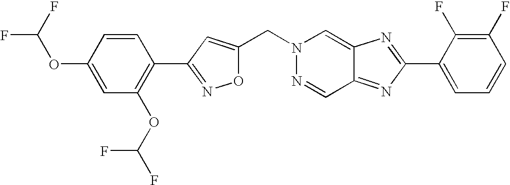 | 5-[3-(2,4-Bis-difluoromethoxy- phenyl)- isoxazol-5-ylmethyl]-2-(2,3-difluoro- phenyl)-5H-imidazo[4,5- d]pyridazine |
| 170 | 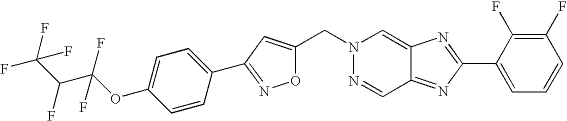 | 2-(2,3-Difluoro-phenyl)-5-{3-[4- (1,1,2,3,3,3-hexafluoro-propoxy)- phenyl]-isoxazol-5-ylmethyl}-5H- imidazo[4,5- d]pyridazine |
| 171 | 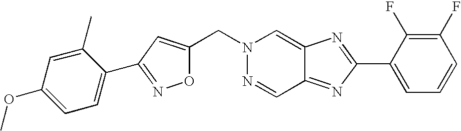 | 2-(2,3-Difluoro-phenyl)-5-[3-(4- methoxy-2-methyl-phenyl)- isoxazol-5- ylmethyl]-5H-imidazo[4,5- d]pyridazine |
| 172 | 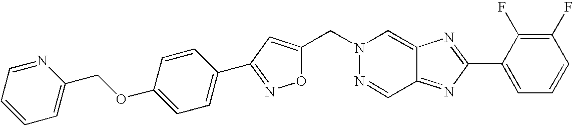 | 2-(2,3-Difluoro-phenyl)-5-{3-[4- (pyridin-2-ylmethoxy)-phenyl]- isoxazol-5-ylmethyl}- 5H-imidazo[4,5- d]pyridazine |
| 173 | 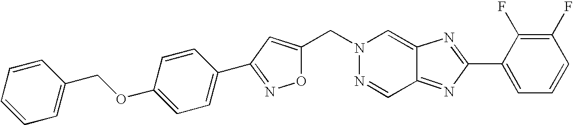 | 5-[3-(4-Benzyloxy-phenyl)- isoxazol-5- ylmethyl]-2-(2,3-difluoro- phenyl)-5H- imidazo[4,5- d]pyridazine |
| 174 | 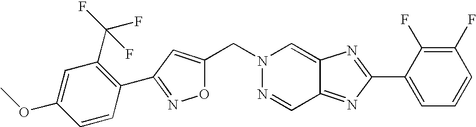 | 2-(2,3-Difluoro-phenyl)-5-[3-(4- methoxy-2-trifluoromethyl-phenyl)- isoxazol-5-ylmethyl]- 5H-imidazo[4,5- d]pyridazine |
| 175 | 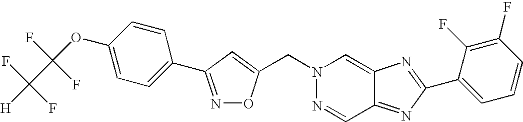 | 2-(2,3-Difluoro-phenyl)-5-{3-[4- (1,1,2,2-tetrafluoro-ethoxy)-phenyl]- isoxazol-5-ylmethyl}- 5H-imidazo[4,5- d]pyridazine |
| 176 | 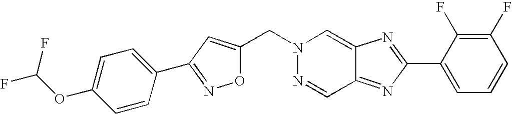 | 5-[3-(4-Difluoromethoxy-phenyl)- isoxazol-5-ylmethyl]-2-(2,3-difluoro- phenyl)-5H-imidazo-4,5- d]pyridazine |
| 177 | 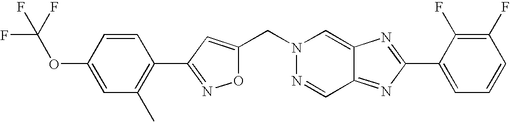 | 2-(2,3-Difluoro-phenyl)-5- [3-(2-methyl- 4-trifluoromethoxy- phenyl)-isoxazol-5- ylmethyl]-5H-imidazo[4,5- d]pyridazine |
| 178 | 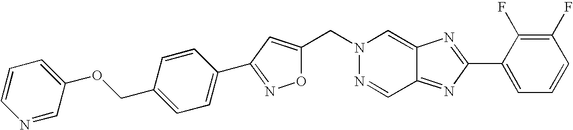 | 2-(2,3-Difluoro-phenyl)-5-{3-[4- (pyridin-3-yloxymethyl)-phenyl]- isoxazol-5-ylmethyl}- 5H-imidazo[4,5- d]pyridazine |
| 179 | 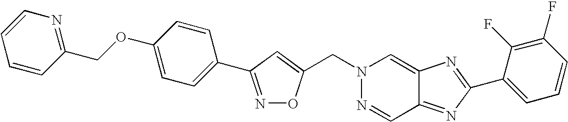 | 2-(2,3-Difluoro-phenyl)-5-{3-[4- (pyridin-3-ylmethoxy)-phenyl]- isoxazol-5-ylmethyl}- 5H-imidazo[4,5- d]pyridazine |
| 180 | 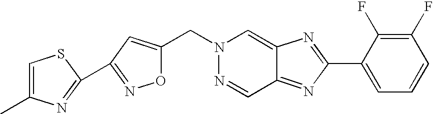 | 2-(2,3-Difluoro-phenyl)- 5-[3-(4-methyl- thiazol-2-yl)-isoxazol- 5-ylmethyl]-5H- imidazo[4,5- d]pyridazine |
| 181 | 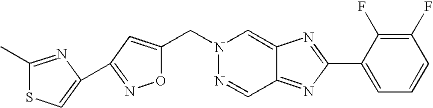 | 2-(2,3-Difluoro-phenyl)- 5-[3-(2-methyl- thiazol-4-yl)-isoxazol- 5-ylmethyl]-5H- imidazo[4,5- d]pyridazine |
| 182 | 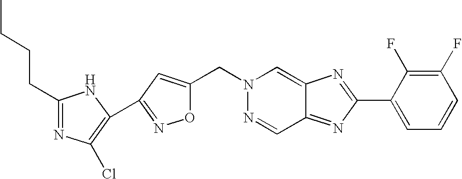 | 5-[3-(2-Butyl-5-chloro- 3H-imidazol-4- yl)-isoxazol-5-ylmethyl]- 2-(2,3-difluoro- phenyl)-5H-imidazo[4,5- d]pyridazine |
| 183 | 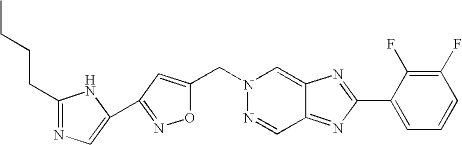 | 5-[3-(2-Butyl-3H-iniidazol-4-yl)- isoxazol-5-ylmethyl]-2-(2,3-difluoro- phenyl)-5H-imidazo[4,5- d]pyridazine |
| 184 | 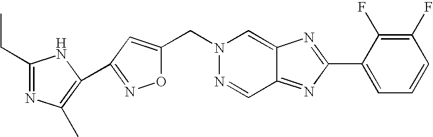 | 2-(2,3-Difluoro-phenyl)- 5-[3-(2-ethyl-5- methyl-3H-imidazol- 4-yl)-isoxazol-5- ylmethyl]-5H-imidazo[4,5- d]pyridazine |
| 185 | 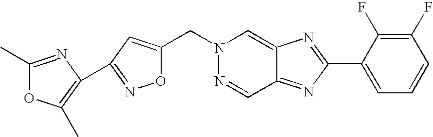 | 2-(2,3-Difluoro-phenyl)-5-[3-(2,5- dimethyl-oxazol-4-yl)-isoxazol-5- ylmethyl]-5H-imidazo[4,5- d]pyridazine |
| 186 | 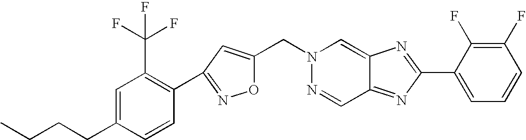 | 5-[3-(4-Butyl-2-trifluoromethyl- phenyl)- isoxazol-5-ylmethyl]-2-(2,3-difluoro- phenyl)-5H-imidazo[4,5- d]pyridazine |
| 187 | 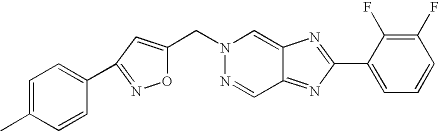 | 2-(2,3-Difluoro-phenyl)-5-(3-p-tolyl- isoxazol-5-ylmethyl)-5H-imidazo[4,5- d]pyridazine |
| 188 | 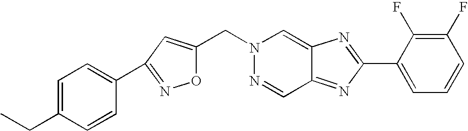 | 2-(2,3-Difluoro-phenyl)-5-[3-(4-ethyl- phenyl)-isoxazol-5-ylmethyl]-5H- imidazo[4,5- d]pyridazine |
| 189 | 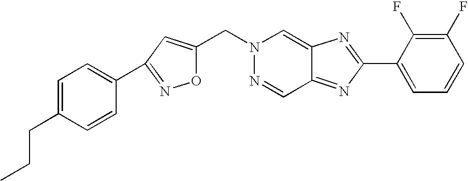 | 2-(2,3-Difluoro-phenyl)-5-[3-(4-propyl- phenyl)-isoxazol-5-ylmethyl]-5H- imidazo[4,5- d]pyridazine |
| 190 | 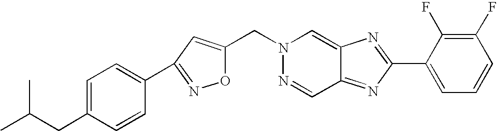 | 2-(2,3-Difluoro-phenyl)-5-[3-(4- isobutyl-phenyl)-isoxazol-5-ylmethyl]- 5H-imidazo[4,5- d]pyridazine |
| 191 | 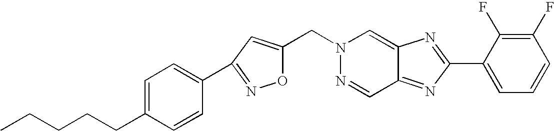 | 2-(2,3-Difluoro-phenyl)-5-[3-(4-pentyl- phenyl)-isoxazol-5-ylmethyl]-5H- imidazo[4,5- d]pyridazine |
| 192 | 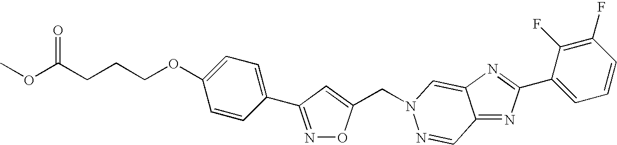 | 4-(4-{5-[2-(2,3-Difluoro-phenyl)- imidazo[4,5- d]pyridazin-5-ylmethyl]- isoxazol-3-yl}-phenoxy)-butyric acid methyl ester |
| 193 | 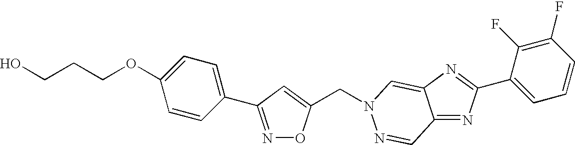 | 3-(4-{5-[2-(2,3-Difluoro-phenyl)- imidazo[4,5- d]pyridazin-5-ylmethyl]- isoxazol-3-yl}-phenoxy)-propan-1-ol |
| 194 | 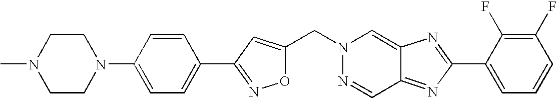 | 2-(2,3-Difluoro-phenyl)-5-{3-[4-(4- methyl-piperazin-1-yl)-phenyl]- isoxazol-5-ylmethyl]-5H-imidazo[4,5- d]pyridazine |
| 195 | 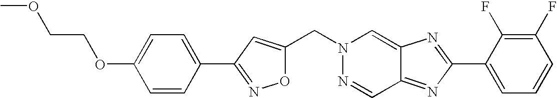 | 2-(2,3-Difluoro-phenyl)-5-{3-[4-(2- methoxy-ethoxy)-phenyl]-isoxazol-5- ylmethyl}-5H-imidazo[4,5- d]pyridazine |
| 196 | 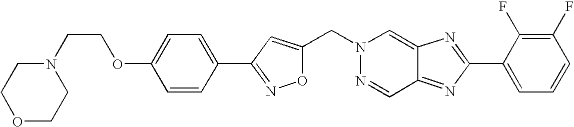 | 2-(2,3-Difluoro-phenyl)-5-{3-[4-(2- morpholin-4-yl-ethoxy)-phenyl]- isoxazol-5-ylmethyl}-5H-imidazo[4,5- d]pyridazine |
| 197 | 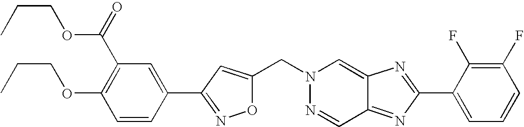 | 5-{5-[2-(2,3-Difluoro-phenyl)- imidazol[4,5- d]pyridazin-5-ylmethyl]- isoxazol-3-yl}-2-propoxy-benzoic acid propyl ester |
| 198 | 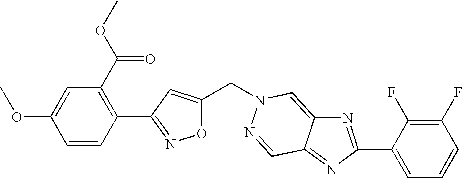 | 2-{5-[2-(2,3-Difluoro-phenyl)- imidazo[4,5- d]pyridazin-5-ylmethyl]- isoxazol-3-yl}-5-methoxy-benzoic acid methylester |
| 199 | 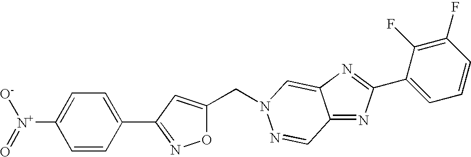 | 2-(2,3-Difluoro-phenyl)-5-[3-(4-nitro- phenyl)-isoxazol-5-ylmethyl]-5H- imidazo[4,5- d]pyridazine |
| 200 | 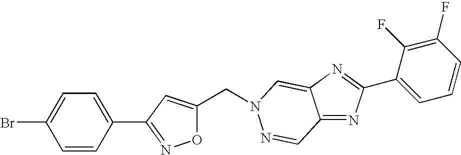 | 5-[3-(4-Bromo-phenyl)-isoxazol-5- ylmethyl]-2-(2,3-difluoro-phenyl)-5H- imidazo[4,5- d]pyridazine |
| 201 | 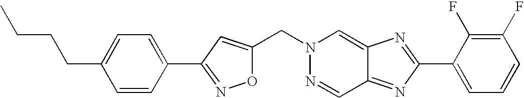 | 5-[3-(4-Butyl-phenyl)-isoxazol-5- ylmethyl]-2-(2,3-difluoro-phenyl)-5H- imidazo[4,5- d]pyridazine |
| 202 | 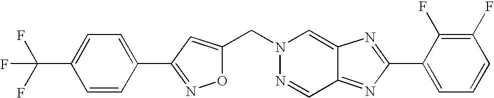 | 2-(2,3-Difluoro-phenyl)-5-[3-(4- trifluoromethyl-phenyl)-isoxazol-5- ylmethyl]-5H-imidazo[4,5- d]pyridazine |
| 203 | 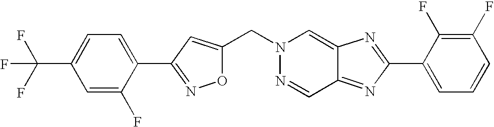 | 2-(2,3-Difluoro-phenyl)-5-[3-(2-fluoro- 4-trifluoromethyl-phenyl)-isoxazol-5- ylmethyl]-5H-imidazo[4,5- d]pyridazine |
| 204 | 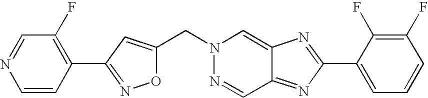 | 2-(2,3-Difluoro-phenyl)-5-[3-(3-fluoro- pyridin-4-yl)-isoxazol-5-ylmethyl]-5H- imidazo[4,5- d]pyridazine |
| 205 | 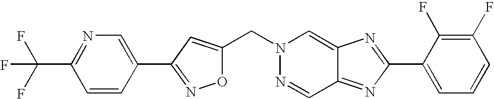 | 2-(2,3-Difluoro-phenyl)-5-[3-(6- trifluoromethyl-pyridin-3-yl)-isoxazol-5- ylmethyl]-5H-imidazo[45-d]pyridazine |
| 206 | 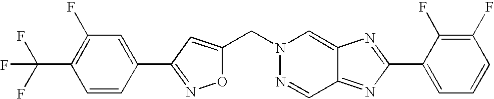 | 2-(2,3-Difluoro-phenyl)-5-[3-(3-fluoro- 4-trifluoromethyl-phenyl)-isoxazol-5- ylmethyl]-5H-imidazo[4,5- d]pyridazine |
| 207 | 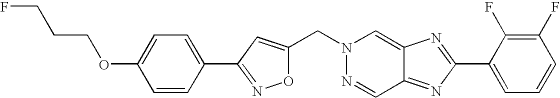 | 2-(2,3-Difluoro-phenyl)-5-{3-[4-(3- fluoro-propoxy)-phenyl]-isoxazol-5- ylmethyl}-5H-imidazo[4,5- d]pyridazine |
| 208 | 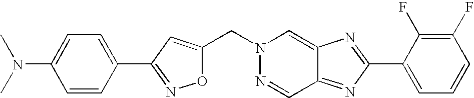 | (4-{5-[2-(2,3-Difluoro-phenyl)- imidazo[4,5- d]pyridazin-5-ylmethyl]- isoxazol-3-yl}-phenyl)-dimethyl-amine |
| 209 | 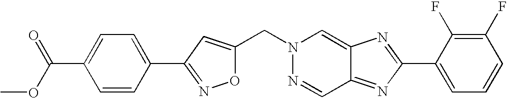 | 4-{5-[2-(2,3-Difluoro-phenyl)- imidazo[4,5- d]pyridazin-5-ylmethyl]- isoxazol-3-yl}-benzoic acid methyl ester |
| 210 | 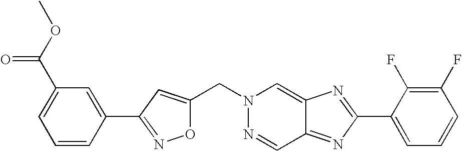 | 3-{5-[2-(2,3-Difluoro-phenyl)- imidazo[4,5- d]pyridazin-5-yl methyl]- isoxazol-3-yl}-benzoic acid methyl ester |
| 211 | 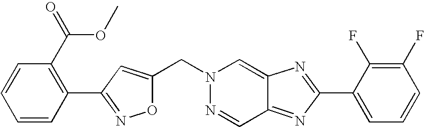 | 2-{5-[2-(2,3-Difluoro-phenyl)- imidazo[4,5- d]pyridazin-5-ylmethyl]- isoxazol-3-yl}-benzoic acid methyl ester |
| 212 | 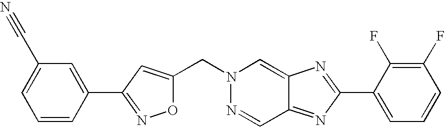 | 3-{542-(2,3-Difluoro-phenyl)- imidazo[4,5- d]pyridazin-5-ylmethyl]- isoxazol-3-yl}-benzonitrile |
| 213 | 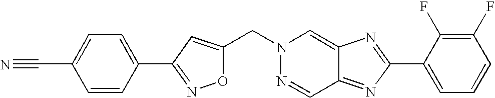 | 4-{5-[2-(2,3-Difluoro-phenyl)- imidazo[4,5- d]pyridazin-5-ylmethyl]- isoxazol-3-yl}-benzonitrile |
| 214 | 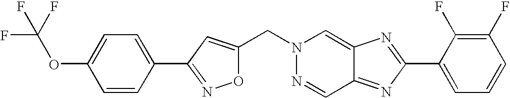 | 2-(2,3-Difluoro-phenyl)-5-[3-(4- trifluoromethoxy-phenyl)-isoxazol-5- ylmethyl]-5H-imidazo[4,5- d]pyridazine |
| 215 | 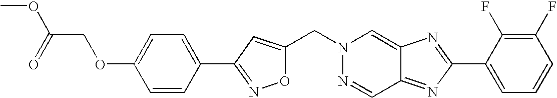 | (4-{5-[2-(2,3-Difluoro-phenyl)- imidazo[4,5- d]pyridazin-5-ylmethyl]- isoxazol-3-yl}-phenoxy)-acetic acid methyl ester |
| 216 | 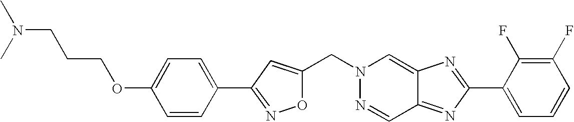 | [3-(4-{5-[2-(2,3-Difluoro-phenyl)- imidazo[4,5- d]pyridazin-5-ylmethyl]- isoxazol-3-yl}-phenoxy)-propyl]- dimethyl-amine |
| 217 | 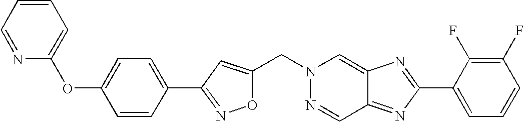 | 2-(2,3-Difluoro-phenyl)-5-{3-[4- (pyridin-2-ylpxy)-phenyl]-isoxazol-5- ylmethyl}-5H-imidazo[4,5- d]pyridazine |
| 218 | 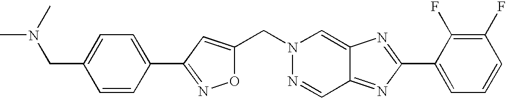 | (4-{5-{2-(2,3-Difluoro-phenyl)- imidazo[4,5- d]pyridazin-5-ylmethyl]- isoxazol-3-yl}-benzyl)-dimethyl-amine |
| 219 | 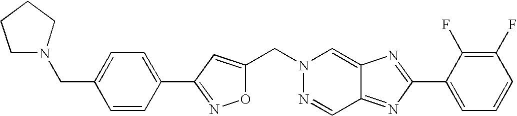 | 2-(2,3-Difluoro-phenyl)-5-[3-(4- pyrrolidin-1-ylmethyl-phenyl)-isoxazol- 5-ylmethyl]-5H-imidazo[4,5- d]pyridazine |
| 220 | 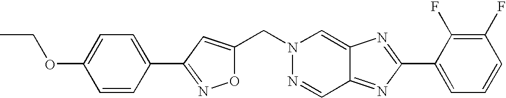 | 2-(2,3-Difluoro-phenyl)-5-[3-(4-ethoxy- phenyl)-isoxazol-5-ylmethyl]-5H- imidazo[4,5- d]pyridazine |
| 221 | 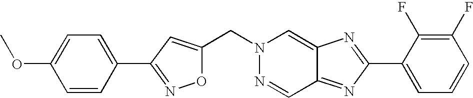 | 2-(2,3-Difluoro-phenyl)-5-[3-(4- methoxy-phenyl)-isoxazol-5-ylmethyl]- 5H-imidazo[4,5- d]pyridazine |
| 222 | 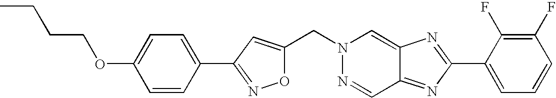 | 5-[3-(4-Butoxy-phenyl)-isoxazol-5- ylmethyl-2-(2,3-difluoro-phenyl)-5H- imidazo[4,5- d]pyridazine |
| 223 | 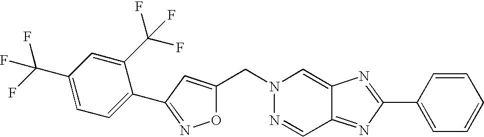 | 5-[3-(2,4-Bis- trifluoromethyl-phenyl)- isoxazol-5-ylmethyl]-2-phenyl-5H- imidazo[4,5- d]pyridazine |
| 224 | 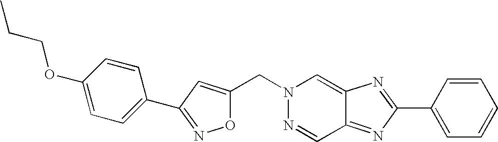 | 2-Phenyl-5-[3-(4-propoxy-phenyl)- isoxazol-5-ylmethyl]-5H-imidazo[4,5- d]pyridazine |
| 225 | 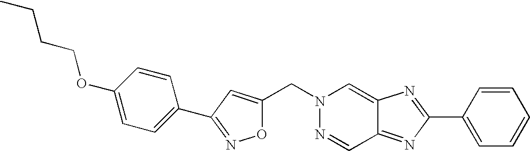 | 4-Butoxy-phenyl)-isoxazol-5- ylmethyl]-2-phenyl-5H-imidazo[4,5- d]pyridazine |
| 226 | 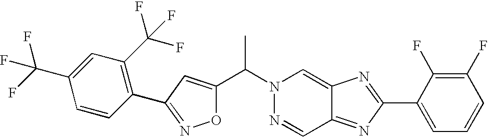 | 5-{1-[3-(2,4-Bis- trifluoromethyl- phenyl)-isoxazol-5-yl]-ethyl}-2-(2,3- difluoro-phenyl)-5H-imidazo[4,5- d]pyridazine |
| 227 | 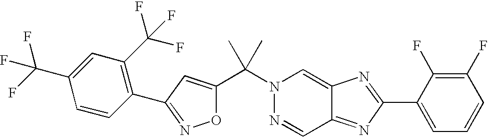 | 5-{1-[3-(2,4-Bis- trifluoromethyl- phenyl)-isoxazol-5-yl]-1-methyl-ethyl}- 2-(2,3-difluoro-phenyl)-5H- imidazo[4,5- d]pyridazine |
| 228 | 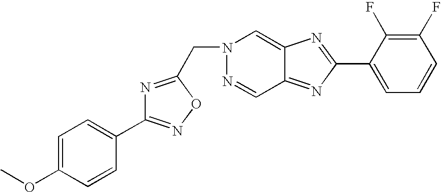 | 2-(2,3-Difluoro-phenyl)-5-[3-(4- methoxy-phenyl)-[1,2,4]oxadiazol-5- ylmethyl]-5H-imidazo[4,5- d]pyridazine |
| 229 | 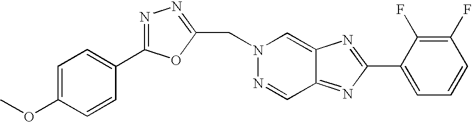 | 2-(2,3-Difluoro-phenyl)-5-[5-(4- methoxy-phenyl)-[1,3,4]oxadiazol-2- ylmethyl]-5H-imidazo[4,5- d]pyridazine |
| 230 | 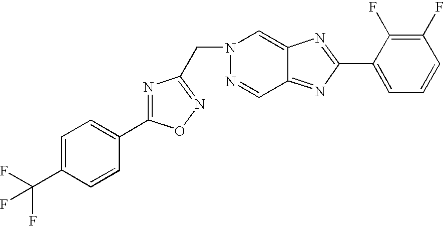 | 2-(2,3-Difluoro-phenyl)-5-[5-(4- trifluoromethyl-phenyl) [1,2,4]oxadiazoi-3-ylmethyl]-5H- imidazo[4,5- d]pyridazine |
| 231 | 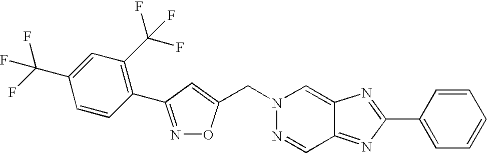 | 5-[3-(2,4-Bis- trifluoromethyl-phenyl)- isoxazol-5-ylmethyl]-2-pyridin-2-yl-5H- imidazo[4,5- d]pyridazine |
| 232 | 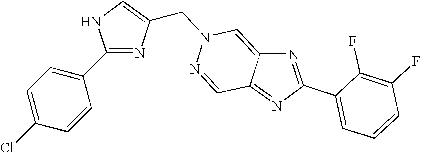 | 5-[2-(4-Chloro-phenyl)-1H-imidazol-4- ylmethyl]-2-(2,3-difluoro-phenyl)-5H- imidazo[4,5- d]pyridazine |
| 233 | 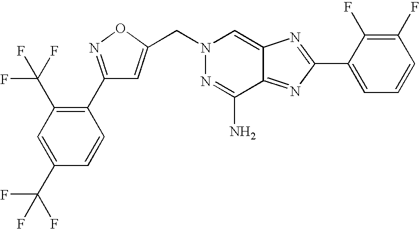 | 6-{3-(2,4-Bis- trifluoromethyl-phenyl)- isoxazol-5-ylmethyl]-2-(2,3-difluoro- phenyl)-6H-imidazo[4,5- d]pyridazin-4- ylamine |
| 234 | 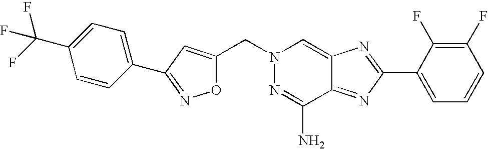 | 2-(2,3-Difluoro-phenyl)-6-[3-(4- trifluoromethyl-phenyl)-isoxazol-5- ylmethyl]-6H-imidazo[4,5- d]pyridazin- 4-ylamine |
| 235 | 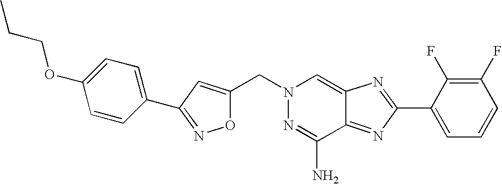 | 3-Difluoro-phenyl)-6-[3-(4- propoxy-phenyl)-isoxazol-5-ylmethyl]- 6H-imidazo[4,5- d]pyridazin-4-ylamine |
| 236 | 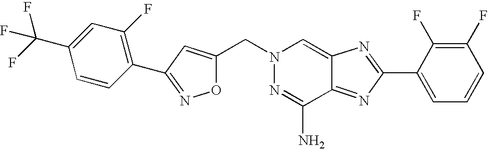 | 2-(2,3-Difluoro-phenyl)-6-[3-(2-fluoro- 4-trifluoromethyl-phenyl)-isoxazol-5- ylmethyl]-6H-imidazo[4,5- d]pyridazin- 4-ylamine |
| 237 | 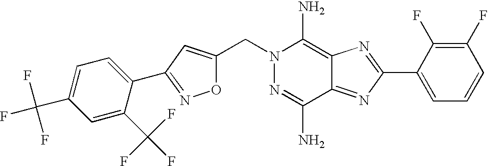 | 5-[3-(2,4-Bis- trifluoromethyl-phenyl)- isoxazol-5-ylmethyl]-2-(2,3-difluoro- phenyl)-5H-imidazo[4,5- d]pyridazine- 4,7-diamine |
| 238 | 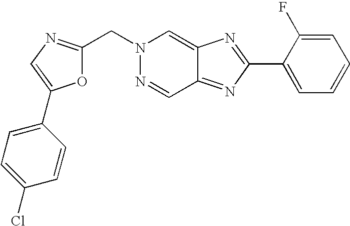 | 5-[5-(4-Chloro-phenyl)-oxazol-2- ylmethyl]-2-(2-fluoro-phenyl)-5H- imidazo[4,5- d]pyridazine |
| 239 | 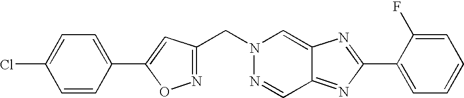 | 6-[5-(4-Chloro-phenyl)-isoxazol-3- ylmethyl]-2-(2-fluoro-phenyl)-5H- imidazo[4,5- d]pyridazine |
| 240 | 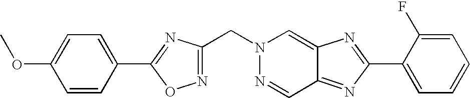 | 2-(2-Fluoro-phenyl)-5-[5-(4-methoxy- phenyl)-[1,2,4]oxadiazol-3-ylmethyl]- 5H-imidazo[4,5- d]pyridazine |
| 241 | 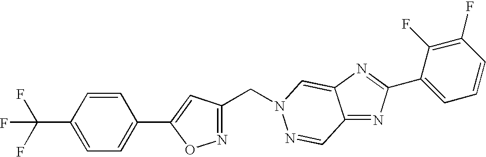 | 2-(2,3-Difluoro-phenyl)-5-[5-(4- trifluoromethyl-phenyl)-isoxazol-3- ylmethyl]-5H-imidazo[4,5- d]pyridazine |
| 242 | 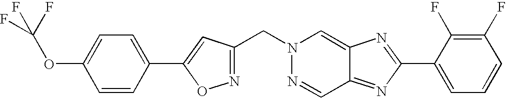 | 2-(2,3-Difluoro-phenyl)-5-[5-(4- trifluoromethoxy-phenyl)-isoxazol-3- ylmethyl]-5H-imidazo[4,5- d]pyridazine |
| 243 | 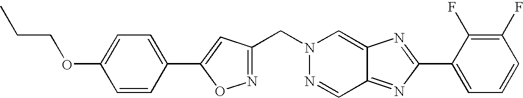 | 2-(2,3-Difluoro-phenyl)-5-[5-(4- propoxy-phenyl)-isoxazol- 3-ylmethyl]- 5H-imidazo[4,5- d]pyridazine |
| 244 | 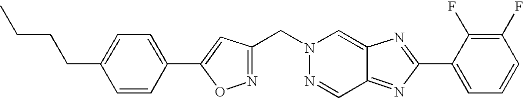 | 5-[5-(4-Butyl-phenyl)-isoxazol-3- ylmethyl]-2-(2,3-difluoro- phenyl)-5H- imidazo[4,5- d]pyridazine |
| 245 | 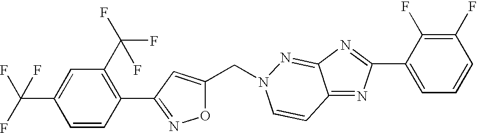 | 2-[3-(2,4-Bis- trifluoromethyl-phenyl)- isoxazol-5-ylmethyl]-6-(2,3-difluoro- phenyl)-2H-imidazo[4,5- c]pyridazine |
| 246 | 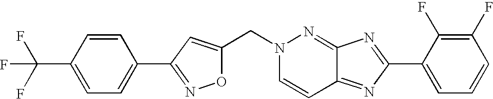 | 6-(2,3-Difluoro-phenyl)-2-[3-(4- trifluoromethyl-phenyl)-isoxazol-5- ylmethyl]-2H-imidazo[4,5- c]pyridazine |
| 247 | 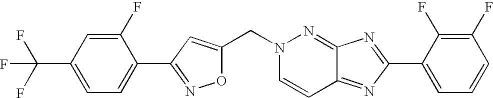 | 6-(2,3-Difluoro-phenyl)- 2-[3-(2-fluoro- 4-trifluoromethyl-phenyl)- isoxazol-5- ylmethyl]-2H-imidazo[4,5- c]pyridazine |
| 248 | 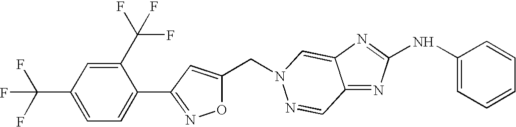 | {5-[3-(2,4-Bis- trifluoromethyl-phenyl)- isoxazol-5-ylmethyl]-5H- imidazo[4,5- d]pyridazin-2-yl}-phenyl-amine |
| 249 | 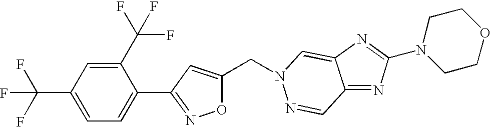 | 5-[3-(2,4-Bis- trifluoromethyl-phenyl)- isoxazol-5-ylmethyl]- 2-morpholin-4-yl- 5H-imidazo[4,5- d]pyridazine |
| 250 | 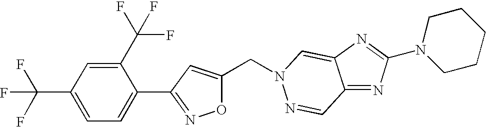 | 5-[3-(2,4-Bis- trifluoromethyl-phenyl)- isoxazol-5-ylmethyl]- 2-piperidin-1-yl- 5H-imidazo[4,5- d]pyridazine |
| 251 | 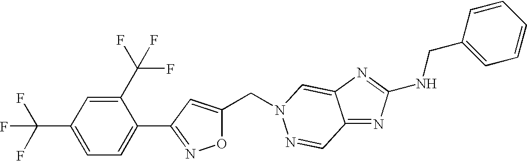 | Benzyl-{5-[3-(2,4-bis- trifluoromethyl- phenyl)-isoxazol-5-ylmethyl]-5H- imidazo[4,5- d]pyridazin-2-yl}-amine |
| 252 | 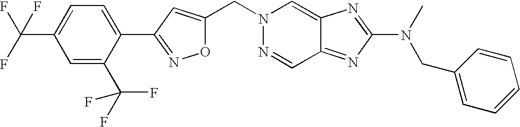 | Benzyl-{5-[3-(2,4-bis- trifluoromethyl- phenyl)-isoxazol-5-ylmethyl]-5H- imidazo[4,5- d]pyridazin-2-yl}-methyl- amine |
| 253 | 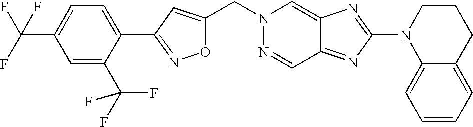 | 1-{5-[3-(2,4-Bis- trifluoromethyl- phenyl)-isoxazol-5-ylmethyl]-5H- imidazo[4,5- d]pyridazin-2-yl}-1,2,3,4- tetrahydro-quinoline |
| 254 | 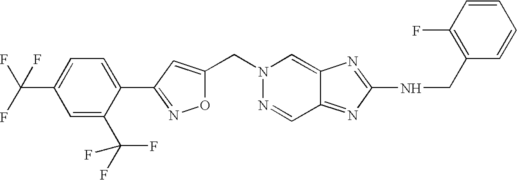 | {5-[3-(2,4-Bis- trifluoromethylphenyl)- isoxazol-5-ylmethyl]- 5H-imidazo[4,5- d]pyridazin-2-yl}-(2-fluoro-benzyl)- amine |
| 255 | 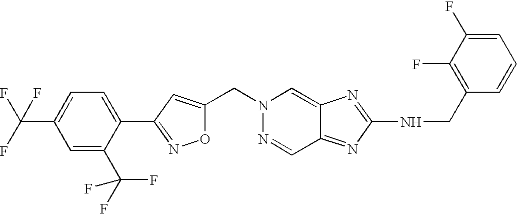 | {5-[3-(2,4-Bis- trifluoromethyl-phenyl)- isoxazol-5-ylmethyl]- 5H-imidazo[4,5- d]pyridazin-2-yl}- (2,3-difluoro-benzyl)- amine |
| 256 | 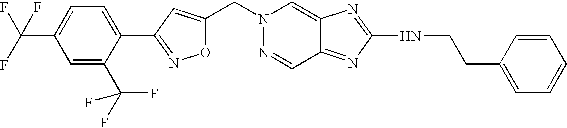 | {5-[3-(2,4-Bis- trifluoromethyl-phenyl)- isoxazol-5-ylmethyl]- 5H-imidazo[4,5- d]pyridazin-2-yl}-phenethyl-amine |
| 257 | 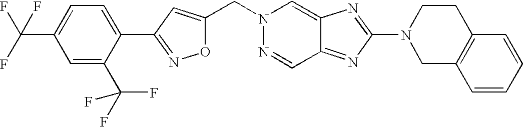 | 2-{5-[3-(2,4-Bis- trifluoromethyl- phenyl)-isoxazol-5-ylmethyl]-5H- imidazo[4,5- d]pyridazin-2-yl}-1 2,3,4- tetrahydro-isoquinoline |
| 258 | 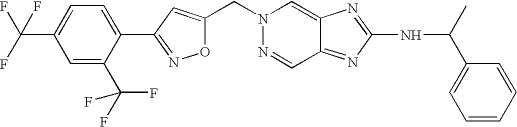 | {5-[3-(2,4-Bis- trifluoromethyl-phenyl)- isoxazol-5-ylmethyl]- 5H-imidazo[4,5- d]pyridazin-2-yl}-(1-phenyl-ethyl)- amine |
| 259 | 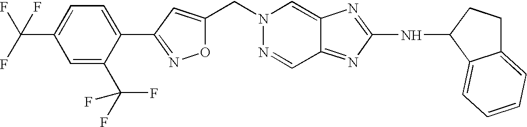 | {5-[3-(2,4-Bis- trifluoromethyl-phenyl)- isoxazol-5-ylmethyl]- 5H-imidazo[4,5- d]pyridazin-2-yl}-indan-1-yl-amine |
| 260 | 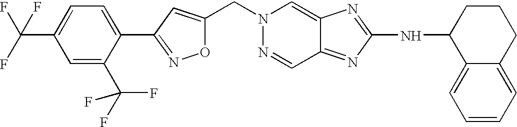 | {5-[3-(2,4-Bis- trifluoromethyl-phenyl)- isoxazol-5-ylmethyl]- 5H-imidazo[4,5- d]pyridazin-2-yl}-(1,2,3,4- tetrahydro- naphthalen-1-yl)-amine |
| 261 | 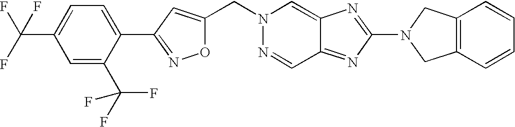 | 5-[3-(2,4-Bis- trifluoromethyl-phenyl)- isoxazol-5-ylmethyl]-2-(1,3-dihydro- isoindol-2-yl)-5H-imidazo[4,5- d]pyridazine |
| 262 | 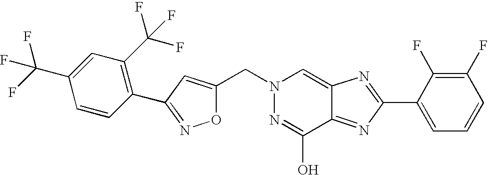 | 6-[3-(2,4-Bis- trifluoromethyl-phenyl)- isoxazol-5-ylmethyl]-2-(2,3-difluoro- phenyl)-6H- imidazo[4,5-d]pyridazin-4-ol |
| 263 | 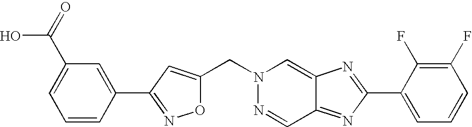 | 3-{5-[2-(2,3-Difluoro-phenyl)- imidazo[4,5- d]pyridazin-5-ylmethyl]- isoxazol-3-yl}-benzoic acid |
| 264 | 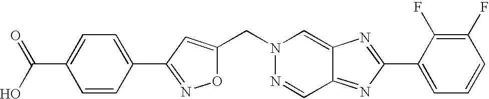 | 4-{5-[2-(2,3-Difluoro-phenyl)- imidazo[4,5- d]pyridazin-5-ylmethyl]- isoxazol-3-yl}-benzoic acid |
| 265 | 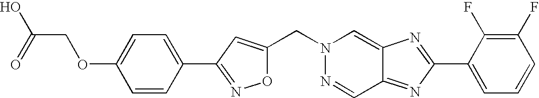 | (4-{5-[2-(2,3-Difluoro-phenyl)- imidazo[4,5- d]pyridazin-5-ylmethyl]- isoxazol-3-yl}-phenoxy)-acetic acid |
| 266 | 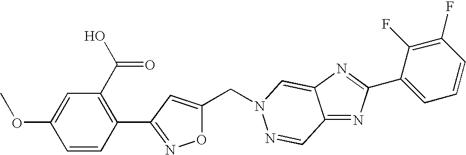 | 2-{5-[2-(2,3-Difluoro-phenyl)- imidazo[4,6-d]pyridazin- 5-ylmethyl]- isoxazol-3-yl}-5-methoxy-benzoic acid |
| 267 | 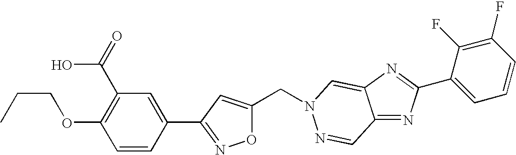 | 5-{5-[2-(2,3-Difluoro-phenyl)- imidazo[4,5- d]pyridazin-5-yl methyl]- isoxazol-3-yl}-2-propoxy-benzoic acid |
| 268 | 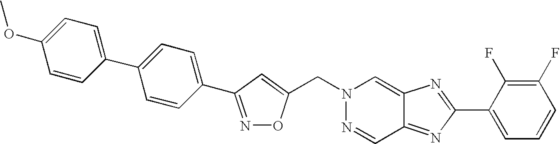 | 2-(2,3-Difluoro-phenyl)-5-[3-(4′- methoxy-biphenyl-4-yl)-isoxazol-5- ylmethyl]-5H-imidazo[4,5- d]pyridazine |
| 269 | 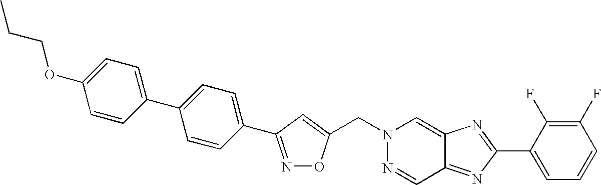 | 2-(2,3-Difluoro-phenyl)-5-[3-(4′- propoxy-biphenyl-4-yl)-isoxazol-5- ylmethyl]-5H-imidazo[4,5- d]pyridazine |
| 270 | 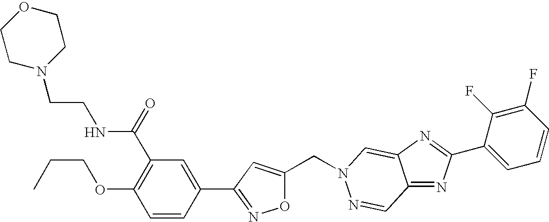 | 5-{5-[2-(2,3-Difluoro-phenyl)- imidazo[4,5- d]pyridazin-5-ylmethyl]- isoxazol-3-yl}-N-(2-morpholin-4-yl- ethyl)-2-propoxy-benzamide |
| 271 | 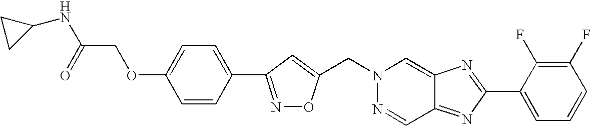 | N-Cyclopropyl-2-(4-{5- [2-(2,3-difluoro- phenyl)-imidazo[4,5- d]pyridazin-5- ylmethyl]-isoxazol-3-yl}-phenoxy)- acetamide |
| 272 | 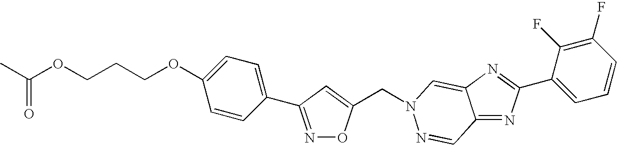 | Acetic acid 3-(4-{5-[2-(2,3-difluoro- phenyl)-imidazo[4,5- d]pyridazin-5- ylmethyl]-isoxazol-3-yl}-phenoxy)- propyl ester |
| 273 | 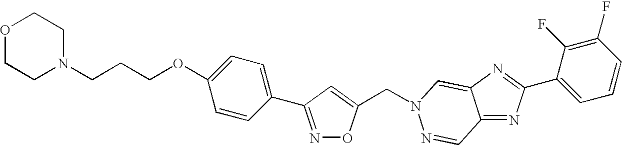 | 2-(2,3-Difluoro-phenyl)-5-{3-[4-(3- morpholin-4-yl-propoxy)-phenyl]- isoxazol-5-ylmethyl}- 5H-imidazo[4,5- d]pyridazine |
| 274 | 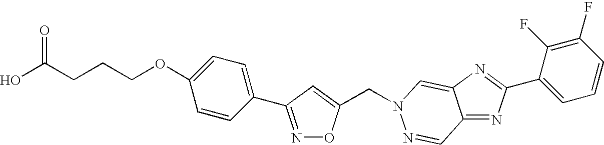 | 4-(4-{5-[2-(2,3-Difluoro-phenyl)- imidazo[4,5- d]pyridazin-5-ylmethyl]- isoxazol-3-yl}-phenoxy)-butyric acid |
| 275 | 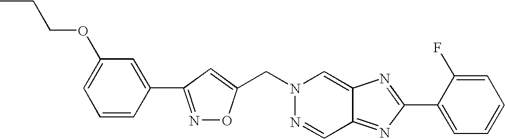 | 2-(2-Fluoro-phenyl)-5- [3-(3-propoxy- phenyl)-isoxazol-5-ylmethyl]-5H- imidazo[4,5- d]pyridazine |
| 276 | 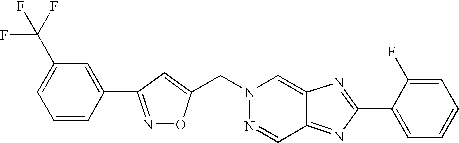 | 2-(2-Fluoro-phenyl)-5-[3-(3- trifluoromethyl-phenyl)-isoxazol-5- ylmethyl]-5H-imidazo[4,5- d]pyridazine |
| 277 | 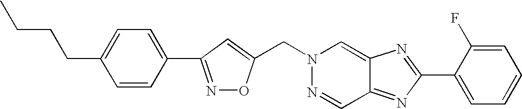 | 5-[3-(4-Butyl-phenyl)-isoxazol-5- ylmethyl]-2-(2-fluoro-phenyl)-5H- imidazo[4,5- d]pyridazine |
| 278 | 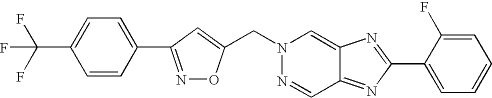 | 2-(2-Fluoro-phenyl)-5-[3-(4- trifluoromethyl-phenyl)-isoxazol-5- ylmethyl]-5H-imidazo[4,5- d]pyridazine |
| 279 | 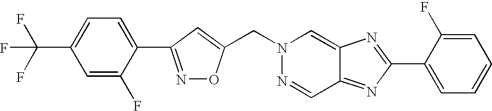 | 2-(2-Fluoro-phenyl)- 5-[3-(2-fluoro-4- trifluoromethyl-phenyl)-isoxazol-5- ylmethyl]-5H-imidazo[4,5- d]pyridazine |
| 280 | 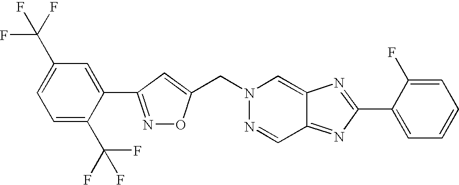 | 5-[3-(2,5-Bis- trifluoromethyl-phenyl)- isoxazol-5-ylmethyl]-2-(2-fluoro- phenyl)-5H-imidazo[4,5- d]pyridazine |
| 281 | 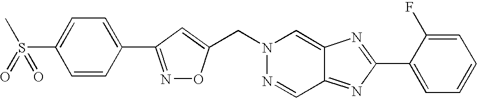 | 2-(2-Fluoro-phenyl)-5-[3-(4- methanesulfonyl-phenyl)-isoxazol-5- ylmethyl]-5H-imidazo[4,5- d]pyridazine |
| 282 | 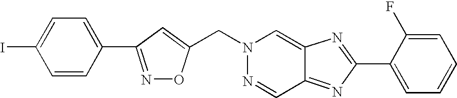 | 2-(2-Fluoro-phenyl)-5-[3-(4-iodo- phenyl)-isoxazol-5-ylmethyl]-5H- imidazo[4,5- d]pyridazine |
| 283 | 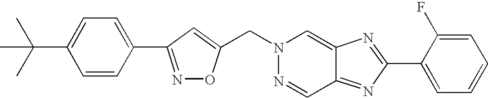 | 5-[3-(4-tert-Butyl-phenyl)- isoxazol-5- ylmethyl]-2-(2-fluoro-phenyl)-5H- imidazo[4,5- d]pyridazine |
| 284 | 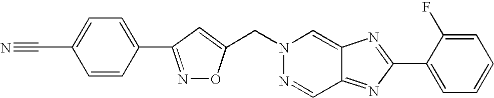 | 4-{5-[2-(2-Fluoro-phenyl)- imidazo[4,5- d]pyridazin-5-ylmethyl]- isoxazol-3-yl}- benzonitrile |
| 285 | 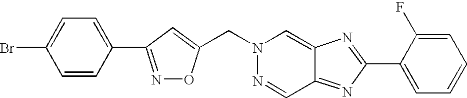 | 5-[3-(4-Bromo-phenyl)-isoxazol-5- ylmethyl]-2-(2-fluoro-phenyl)-5H- imidazo[4,5- d]pyridazine |
| 286 | 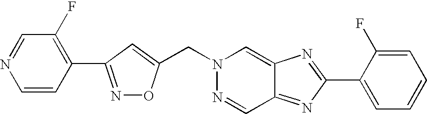 | 2-(2-Fluoro-phenyl)-5-[3-(3-fluoro- pyridin-4-yl)-isoxazol- 5-ylmethyl]-5H- imidazo[4,5- d]pyridazine |
| 287 | 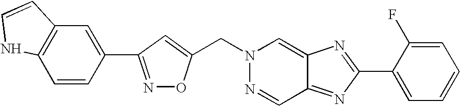 | 2-(2-Fluoro-phenyl)-5-[3- (1H-indol-5- yl)-isoxazol-5-ylmethyl]-5H- imidazo[4,5- d]pyridazine |
| 288 | 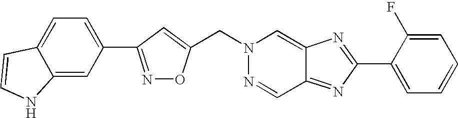 | 2-(2-Fluoro-phenyl)-5-[3- (1H-indol-6- yl)-isoxazol-5-ylmethyl]-5H- imidazo[4,5- d]pyridazine |
| 289 | 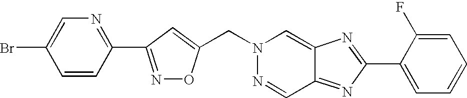 | 5-[3-(5-Bromo-pyridin- 2-yl)-isoxazol-5- ylmethyl]-2-(2-fluoro-phenyl)-5H- imidazo[4,5- d]pyridazine |
| 290 | 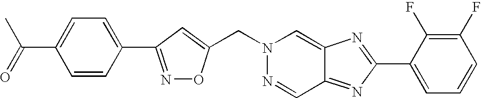 | 1-(4-{5-[2-(2,3-Difluoro-phenyl)- imidazo[4,5- d]pyridazin-5-yl methyl]- isoxazol-3-yl}-phenyl)-ethanone |
| 291 | 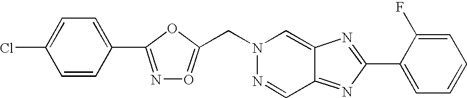 | 5-[5-(4-Chloro-phenyl)- [1,3,4]oxadiazol-2-ylmethyl]-2-(2- fluoro-phenyl)-5H-imidazo[4,5- d]pyridazine |
| 292 | 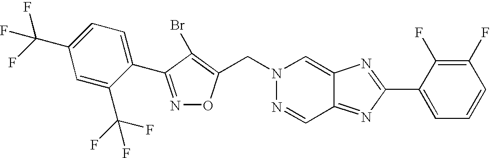 | 5-[3-(2,4-Bis- trifluoromethyl-phenyl)-4- bromo-isoxazol-5-ylmethyl]-2-(2,3- difluoro-phenyl)-5H-imidazo[4,5- d]pyridazine |

In some embodiments, the processes described herein are used to prepare the compounds included in Table 2 or pharmaceutically acceptable salts or solvates thereof.

TABLE 2

| Compound # | Structure | Name |
|---|---|---|
| 6101 | 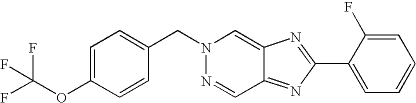 | 2-(2-Fluoro-phenyl)-5-(4- trifluoromethoxy-benzyl)-5H- imidazo[4,5-d]pyridazine |
| 6102 | 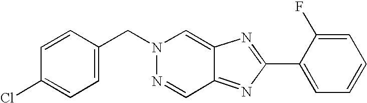 | 5-(4-Chloro-benzyl)-2-(2- fluoro-phenyl)-5H- imidazo[4,5-d]pyridazine |
| 6103 | 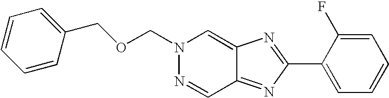 | 5-Benzyloxymethyl-2-(2- fluoro-phenyl)-5H- imidazo[4,5-d]-pyridazine |
| 6104 | 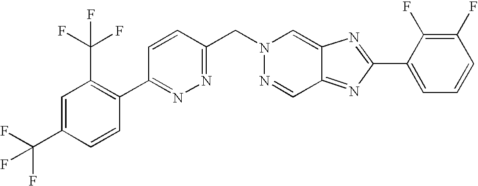 | 5-[6-(2,4-Bis-trifluoromethyl- phenyl)-pyridazin-3- ylmethyl]-2-(2,3-difluoro- phenyl)-5H-imidazo[4,5- d]pyridazine |
| 6105 | 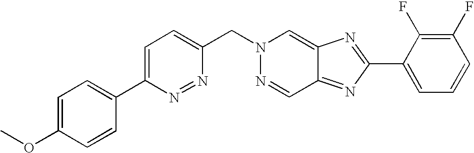 | 2-(2,3-Difluoro-phenyl)-5-[6- (4-methoxy-phenyl)- pyridazin-3-ylmethyl]-5H- imidazo[4,5-d]pyridazine |
| 6106 | 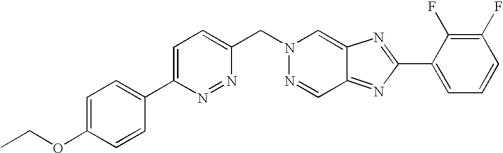 | 2-(2,3-Difluoro-phenyl)-5-[6- (4-ethoxy-phenyl)-pyridazin- 3-ylmethyl]-5H-imidazo[4,5- d]pyridazine |
| 6107 | 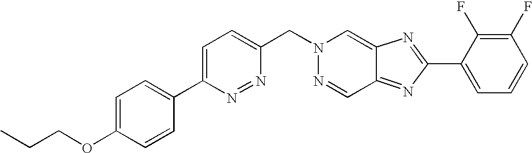 | 2-(2,3-Difluoro-phenyl)-5-[6- (4-propoxy-phenyl)-pyridazin- 3-ylmethyl]-5H-imidazo[4,5- d]pyridazine |
| 6108 | 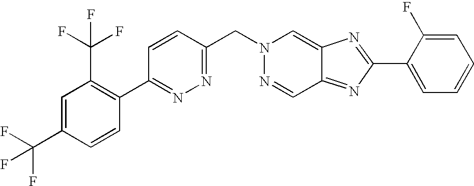 | 5-[6-(2,4-Bis-trifluoromethyl- phenyl)-pyridazin-3- ylmethyl]-2-(2-fluoro-phenyl)- 5H-imidazo[4,5-d]pyridazine |
| 6109 | 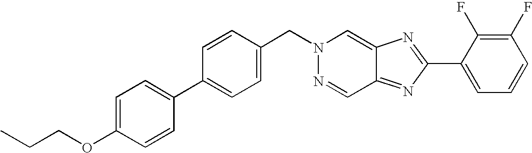 | 2-(2,3-Difluoro-phenyl)-5-(4′- propoxy-biphenyl-4- ylmethyl)-5H-imidazo[4,5- d]pyridazine |
| 6110 | 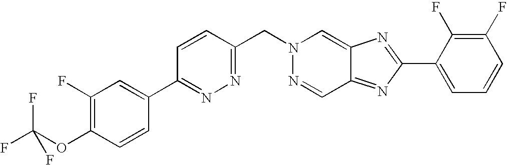 | 2-(2,3-Difluoro-phenyl)-5-[6- (3-fluoro-4-trifluoromethoxy- phenyl)-5H-imidazo[4,5- d]pyridazine |
| 6111 | 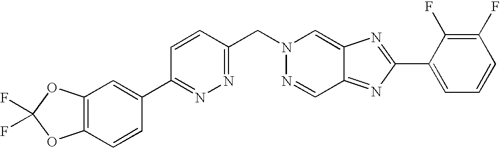 | 5-[6-(2,2-Difluoro- benzo[1,3]dioxol-5-yl)- pyridazin-3-ylmethyl]-2-(2,3- difluoro-phenyl)-5H- imidazo[4,5-d]pyridazine |
| 6112 | 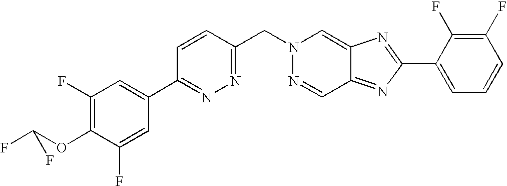 | 5-[6-(4-Difluoromethoxy-3,5- difluoro-phenyl)-pyridazin-3- ylmethyl]-2-(2,3-difluoro- phenyl)-5H-imidazo[4,5- d]pyridazine |
| 6113 | 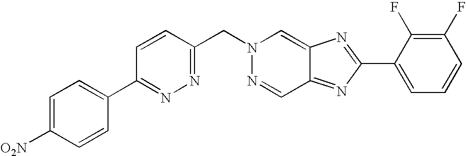 | 2-(2,3-Difluoro-phenyl)-5-[6- (4-nitro-phenyl)-pyridazin-3- ylmethyl]-5H-imidazo[4,5- d]pyridazine |
| 6114 | 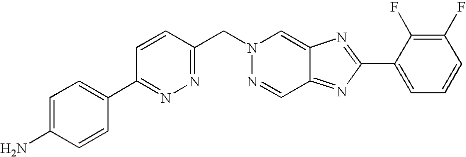 | 4-{6-[2-(2,3-Difluoro- phenyl)-imidazo[4,5- d]pyridazin-5-ylmethyl]- pyridazin-3-yl}-phenylamine |
| 6115 | 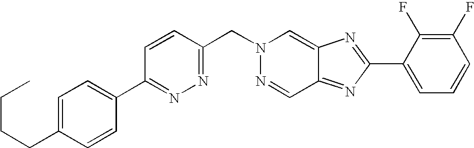 | 5-[6-(4-Butyl-phenyl)- pyridazin-3-ylmethyl]-2-(2,3- difluoro-phenyl)-5H- imidazo[4,5-d]pyridazine |
| 6116 | 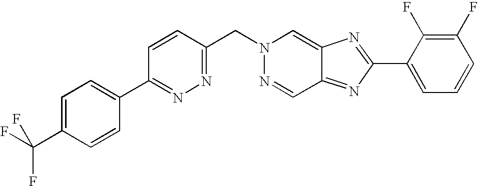 | 2-(2,3-Difluoro-phenyl)-5-[6- (4-trifluoromethyl-phenyl)- pyridazin-3-ylmethyl]-5H- imidazo[4,5-d]pyridazine |
| 6117 | 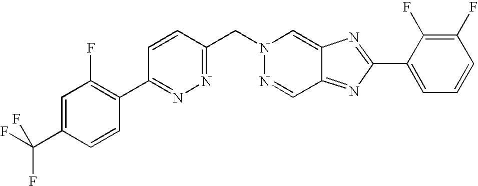 | 2-(2,3-Difluoro-phenyl)-5-[6- (2-fluoro-4-trifluoromethyl- phenyl)-pyridazin-3- ylmethyl]-5H-imidazo[4,5- d]pyridazine |
| 6118 | 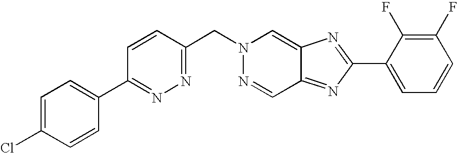 | 5-[6-(4-Chloro-phenyl)- pyridazin-3-ylmethyl]-2-(2,3- difluoro-phenyl)-5H- imidazo[4,5-d]pyridazine |
| 6119 | 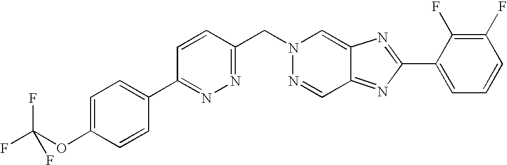 | 2-(2,3-Difluoro-phenyl)-5-[6- (4-trifluoromethoxy-phenyl)- pyridazin-3-ylmethyl]-5H- imidazo[4,5-d]pyridazine |
| 6120 | 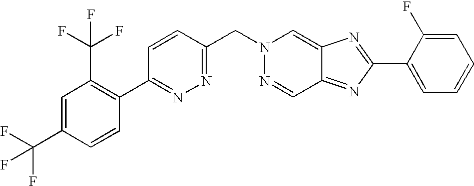 | 5-[6-(2,4-Bis-trifluoromethyl- phenyl)-pyridazin-3- ylmethyl]-2-(2-fluoro-phenyl)- 5H-imidazo[4,5-d]pyridazine |
| 6121 | 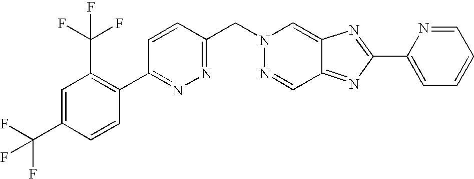 | 5-[6-(2,4-Bis-trifluoromethyl- phenyl)-pyridazin-3- ylmethyl]-2-pyridin-2-yl-5H- imidazo[4,5-d]pyridazine |
| 6122 | 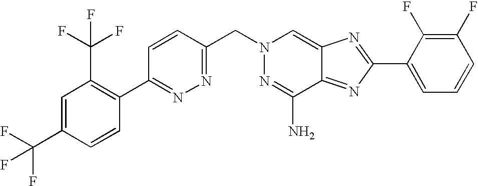 | 6-[6-(2,4-Bis-trifluoromethyl- phenyl)-pyridazin-3- ylmethyl]-2-(2,3-difluoro- phenyl)-6H-imidazo[4,5- d]pyridazin-4-ylamine |
| 6123 | 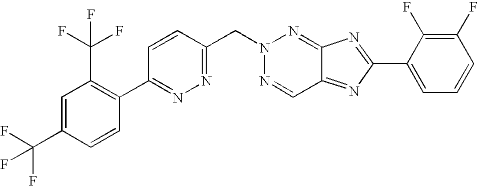 | 2-[6-(2,4-Bis-trifluoromethyl- phenyl)-pyridazin-3- ylmethyl]-6-(2,3-difluoro- phenyl)-2H-imidazo[4,5- d][1,2,3]triazine |
| 6124 | 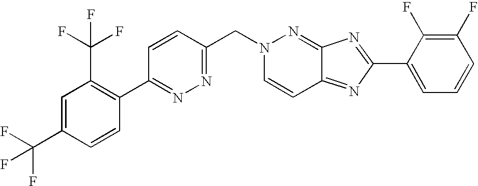 | 2-[6-(2,4-Bis-trifluoromethyl- phenyl)-pyridazin-3- ylmethyl]-6-(2,3-difluoro- phenyl)-2H-imidazo[4,5- c]pyridazine |
| 6125 | 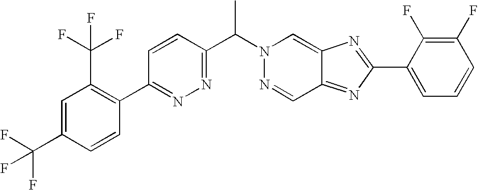 | 5-{1-[6-(2,4-Bis- trifluoromethyl-phenyl)- pyridazin-3-yl]-ethyl}-2-(2,3- difluoro-phenyl)-5H- imidazo[4,5-d]pyridazine |
| 6126 | 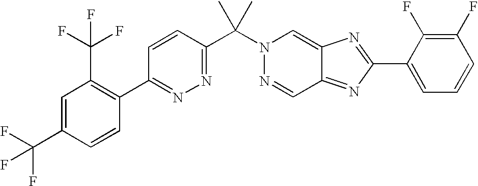 | 5-{1-[6-(2,4-Bis- trifluoromethyl-phenyl)- pyridazin-3-yl]-1-methyl- ethyl}-2-(2,3-difluoro- phenyl)-5H-imidazo[4,5- d]pyridazine |
| 6127 | 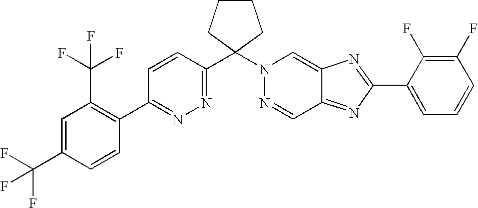 | 5-{1-[6-(2,4-Bis- trifluoromethyl-phenyl)- pyridazin-3-yl]-cyclopentyl}- 2-(2,3-difluoro-phenyl)-5H- imidazo[4,5-d]pyridazine |
| 6128 | 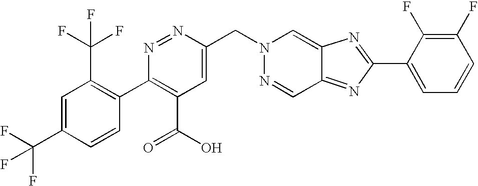 | 3-(2,4-Bis-trifluoromethyl- phenyl)-6-[2-(2,3-difluoro- phenyl)-imidazo[4,5- d]pyridazin-5-ylmethyl]- pyridazine-4-carboxylic acid |
| 6129 | 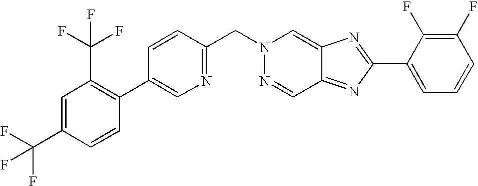 | 5-[5-(2,4-Bis-trifluoromethyl- phenyl)-pyridin-2-ylmethyl]- 2-(2,3-difluoro-phenyl)-5H- imidazo[4,5-d]pyridazine |
| 6130 | 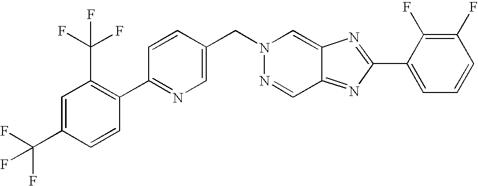 | 5-[6-(2,4-Bis-trifluoromethyl- phenyl)-pyridin-3-ylmethyl]- 2-(2,3-difluoro-phenyl)-5H- imidazo[4,5-d]pyridazine |
| 6131 | 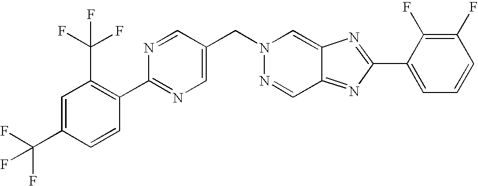 | 5-[2-(2,4-Bis-trifluoromethyl- phenyl)-pyrimidin-5- ylmethyl]-2-(2,3-difluoro- phenyl)-5H-imidazo[4,5- d]pyridazine |
| 6132 | 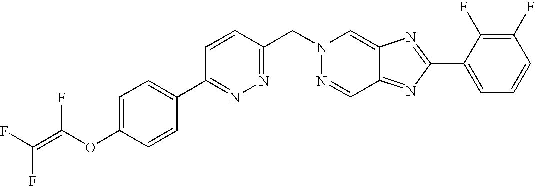 | 2-(2,3-Difluoro-phenyl)-5-[2- (4-trifluorovinyloxy-phenyl)- pyrimidin-5-ylmethyl]-5H- imidazo[4,5-d]pyridazine |
| 6133 | 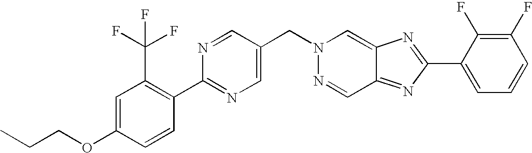 | 2-(2,3-Difluoro-phenyl)-5-[2- (4-propoxy-2-trifluoromethyl- phenyl)-pyrimidin-5- ylmethyl]-5H-imidazo[4,5- d]pyridazine |

### General Synthetic Methods

Representative examples of the processes disclosed herein are set forth below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds, or pharmaceutically acceptable salts or solvates, prepared herein contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

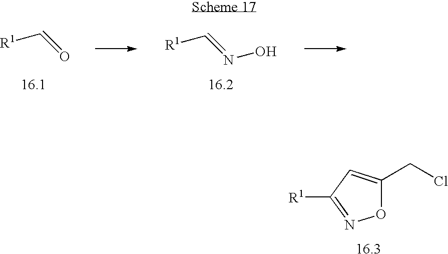

Scheme 17 shows the synthesis of 3-substituted chloromethylisoxazole intermediates wherein R¹ is as defined for formula I. Aldehyde 16.1 is treated with hydroxylamine under oxime forming conditions to give 16.2 that is then cyclized to isoxazole 16.3 through treatment with propargyl chloride and an oxidizing agent such as NaOCl.

The foregoing and other aspects of the present invention may be better understood in connection with the following representative examples.

### Examples

In the examples below and the synthetic schemes above, the following abbreviations have the following meanings. If an abbreviation is not defined, it has its generally accepted meaning.
aq.=aqueous
μL=microliters
μM=micromolar
NMR=nuclear magnetic resonance
br=broad
d=doublet
δ=chemical shift
° C=degrees celsius
dd=doublet of doublets
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
EtOH=ethanol
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H⁺
MeOH=methanol
mg=milligram
ML=milliliter
mM=millimolar
mmol=millimole
MS=mass spectrum
nm=nanomolar
ng=nanogram
ppm=parts per million
s=Singlet
t=triplet
wt %=weight percent

### General Procedure A: Synthesis of 2-Substituted 5H-imidazo[4,5-d]pyridazines

4,5-Diamino-2-benzyloxymethyl-2H-pyridazin-3-one (5.0 g, from J. Het. Chem. 21, 481, 1984) was dissolved in pyridine (25 mL) and an acid chloride (1.1 eq) was added dropwise at room temperature. The mixture was allowed to stir at ambient temperature for 2 hours. The solvent was removed, yielding the amide as a mixture of regioisomers.

The dried amide was dissolved in HOAc (5 mL/gram) and heated to 170° C. for 30 minutes to give 2-substituted 5-benzyloxymethyl-1,5-dihydro-imidazo[4,5-d]pyridazin-4-ones. The products can be purified by trituration with MeOH.

The products and P₂S₅ (1 g/mmol) were then dissolved in pyridine (30 mL/gram) and water (0.75%). The reactions were refluxed overnight. More P₂S₅ was added if the reaction was incomplete. The reaction mixture was cooled and the solution decanted. The solids were washed with hot pyridine and the organic solvent removed. The resulting oil was partitioned between chloroform (100 mL) and NaHCO₃ (sat. aq. 50 mL). The organics were dried (Na₂SO₄) and purified by silica gel chromatography (CH₂C₂/MeOH) giving 2-substituted 5-benzyloxymethyl-1,5-dihydro-imidazo[4,5-d]pyridazine-4-thiones.

The thiones were then dissolved in EtOH (20 mL/gram) and treated with Raney Nickel (unwashed, 1 g/1 g thione) and heated to 70° C. If the reaction was incomplete after 1 hour more Nickel was added. The reactions were then cooled, filtered, the solids were thoroughly washed with hot EtOH and the organics combine and removed yielding the 2-substituted 5-benzyloxymethyl-5H-imidazo[4,5-d]pyridazines.

The products were dissolved in CH₂Cl₂ (35 mL/mmol) and cooled to −78° C. A solution of BCl₃ (1M in CH₂Cl₂, 8 mL mmol) was added and the mixture stirred for 30 minutes. Upon completion, MeOH (5 mL) was added and the mixture warmed to room temperature. The solvents were removed yielding the pure 2-substituted 5H-imidazo[4,5-d]pyridazines. They can be further purified by tritruation with MeOH.

### General Procedure B: Synthesis of Chloromethyl Aryl Isoxazole

The aldehyde (20 mmol) was dissolved in ethanol (15 mL) and hydroxyl amine (50% aq. solution, 3 mL) was added. The mixture was allowed to stir at ambient temperature for 2 hours. The solvent was removed, and no further purification steps were taken.

The oxime (7.65 mmol) was dissolved in dichloromethane (8 mL), and the solution was cooled to 0° C. Propargyl chloride (0.548 mL, 7.65 mmol) was added followed by the dropwise addition of NaOCl (6.5% aq. solution, 13 mL). The reaction was stirred at 0° C. for 15 minutes and then heated to 50° C. for 3 hours. After cooling, the reaction was partitioned between dichloromethane and water, and the aqueous layer was extracted with dichloromethane (3×20 mL). The organic layers were combined, washed with brine (40 mL), dried with anhydrous magnesium sulfate, and filtered. The solvent was removed to give the desired product, and no further purification steps were taken.

### General Procedure C: Synthesis of Compounds 101-105

2-(2,3-Difluoro-phenyl)-5H-1-imidazo[4,5-d]pyridazine (23.8 mg, 0.10 mmol), chloromethyl aryl isoxazole (1 equivalent), and cesium carbonate (66.7 mg, 0.20 mmol) were dissolved in DMF (3 mL) and microwaved at 120° C. for 10 minutes. The reaction was filtered and purified by reverse phase HPLC to give the desired product. The product was converted to the HCl salt by the addition of 1N HCl before concentration.

### Example 1

### 2-(2,3-Difluoro-phenyl)-5-[3-(4-fluoro-2-trifluoromethyl-phenyl)-isoxazol-5-ylmethyl]-5H-imidazo[4,5-d]pyridazine (Compound 101)

From 1 equivalent of 5-chloromethyl-3-(4-fluoro-2-trifluoromethyl-phenyl)-isoxazole. Yield 17.3 mg. MS 476.0 (M+H⁺); ¹H NMR (DMSO-d₆): δ (ppm) 10.17 (d, 1H), 9.55 (d, 1H), 8.12-8.18 (m, 1H), 7.84-7.90 (m, 1H), 7.55-7.77 (m, 3H), 7.34-7.41 (m, 1H), 6.98 (s, 1H), 6.24 (s, 2H).

### Example 2

### 2-(2,3-Difluoro-phenyl)-5-[3-(4-isopropoxy-phenyl)-isoxazol-5-ylmethyl]-5H-imidazo[4,5-d]pyridazine (Compound 102)

From 1 equivalent of 5-chloromethyl-3-(4-isopropoxy-phenyl)-isoxazole. Yield 16.7 mg. MS 448.1 (M+H⁺); ¹H NMR (DMSO-d₆): δ (ppm) 10.18 (d, 1H), 9.55 (d, 1H), 8.12-8.19 (m, 1H), 7.72-7.78 (m, 2H), 7.55-7.65 (m, 1H), 7.34-7.42 (m, 1H), 7.13 (s, 1H), 6.97-7.03 (m, 2H), 6.18 (s, 2H), 4.64-4.73 (m, 1H), 1.27 (d, 6H).

### Example 3

### 5-[3-(2,4-Bis-trifluoromethyl-phenyl)-isoxazol-5-ylmethyl]-2-(2,3-difluoro-phenyl)-5H-imidazo[4,5-d]pyridazine (Compound 103)

From 1 equivalent of 3-(2,4-bis-trifluoromethyl-phenyl)-5-chloromethyl-isoxazole. Yield 12.2 mg. MS 526.0 (M+H⁺); ¹H NMR (DMSO-d₆): δ (ppm) 10.17 (d, 1H), 9.55 (d, 1H), 8.12-8.19 (m, 8.26 (m, 3H), 7.93 (d, 1H), 7.54-7.65 (m, 1H), 7.33-7.41 (m, 1H), 7.06 (s, 1H), 6.26 (s, 2H).

### Example 4

### 5-[3-(4-Chloro-phenyl)-isoxazol-5-ylmethyl]-2-(2,3-difluoro-phenyl)-5H-imidazo[4,5-d]pyridazine (Compound 104)

From 1 equivalent of 5-chloromethyl-3-(4-chloro-phenyl)-isoxazole. Yield 16.9 mg. MS: 424.0 (M+H⁺); ¹H NMR (DMSO-d₆): δ (ppm) 10.21 (d, 1H), 9.57 (d, 1H), 8.12-8.18 (m, 1H), 7.85-7.91 (m, 2H), 7.54-7.67 (m, 3H), 7.35-7.42 (m, 1H), 7.24 (s, 1H), 6.23 (s, 2H).

### Example 5

### 2-(2,3-Difluoro-phenyl)-5-[3-(4-propoxy-phenyl)-isoxazol-5-ylmethyl]-5H-imidazo[4,5-d]pyridazine (Compound 105)

From 1 equivalent of 5-chloromethyl-3-(4-propoxy-phenyl)-isoxazole. Yield 21.9 mg. MS 448.1 (M+H⁺); ¹H NMR (DMSO-d₆): δ (ppm) 10.36 (s, 1H), 9.67 (d, 1H), 8.13-8.18 (m, 1H), 7.63-7.79 (m, 3H), 7.40-7.47 (m, 1H), 7.16 (s, 1H), 7.00-7.05 (m, 2H), 6.26 (s, 2H), 3.95-4.00 (t, 2H), 1.67-1.80 (m, 2H), 0.95-1.02 (t, 3H).

### General Procedure D: Synthesis of Compounds 106-118

2-(2-Fluoro-phenyl)-5H-imidazo[4,5-d]pyridazine (30.0 mg, 0.14 mmol), chloromethyl aryl heterocycle (1 equivalent), and cesium carbonate (91.3 mg, 0.28 mmol) were dissolved in DMF (3 mL) and heated in a microwave reactor at 120° C. for 10 minutes. The reaction was filtered and purified by reverse phase HPLC to give the desired product. The product was converted to the HCl salt by the addition of 1N HCl before concentration.

### Example 6

### 5-[3-(4-Butoxy-phenyl)-isoxazol-5-ylmethyl]-2-(2-fluoro-phenyl)-5H-imidazo[4,5-d]pyridazine (Compound 106)

From 1 equivalent of 3-(4-butoxy-phenyl)-5-chloromethyl-isoxazole. Yield 8.8 mg. MS 444.1 (M+H⁺); ¹H NMR (DMSO-d₆): δ (ppm) 10.45 (s, 1H), 9.74 (s, 1H), 8.32-8.39 (m, 1H), 7.67-7.78 (m, 3H), 7.44-7.56 (m, 2H), 7.17 (s, 1H), 7.03 (d, 2H), 6.31 (s, 2H), 3.98-4.04 (t, 2H), 1.65-1.74 (m, 2H), 1.36-1.49 (m, 2H), 0.90-0.97 (t, 3H).

### Example 7

### 2-(2-Fluoro-phenyl)-5-[3-(4-pentyloxy-phenyl)-isoxazol-5-ylmethyl]-5H-imidazo[4,5-d]pyridazine (Compound 107)

From 1 equivalent of 5-chloromethyl-3-(4-pentyloxy-phenyl)-isoxazole. Yield 10.1 mg. MS 458.1 (M+H⁺); ¹H NMR (DMSO-d₆): δ (ppm) 10.42 (s, 1H), 9.72 (s, 1H), 8.32-8.40 (m, 1H), 7.65-7.79 (m, 3H), 7.43-7.54 (m, 2H), 7.16 (s, 1H), 7.03 (d, 2H), 6.30 (s, 2H), 3.97-4.03 (t, 2H), 1.65-1.78 (m, 2H), 1.27-1.45 (m, 4H), 0.86-0.92 (t, 3H).

### Example 8

### 2-(2-Fluoro-phenyl)-5-[3-(4-trifluoromethoxy-phenyl)-isoxazol-5-ylmethyl]-5H-imidazo[4,5-d]pyridazine (Compound 108)

From 1 equivalent of 5-chloromethyl-3-(4-trifluoromethoxy-phenyl)-isoxazole. Yield 10.4 mg. MS 456.1 (M+H⁺); ¹H NMR (DMSO-d₆): δ (ppm) 10.37 (s, 1H), 9.68 (s, 1H), 8.31-8.38 (m, 1H), 7.95-8.02 (m, 2H), 7.61-7.71 (m, 1H), 7.41-7.54 (m, 4H), 7.28 (s, 1H), 6.31 (s, 2H).

### Example 9

### 2-(2-Fluoro-phenyl)-5-[3-(4-methoxy-phenyl)-isoxazol-5-ylmethyl]-5H-imidazo[4,5-d]pyridazine (Compound 109)

From 1 equivalent of 5-chloromethyl-3-(4-methoxy-phenyl)-isoxazole. Yield 12.1 mg. MS 402.1 (M+H⁺); ¹H NMR (DMSO-d₆): δ (ppm) 10.59 (s, 1H), 9.80 (d, 1H), 8.33-8.40 (m, 1H), 7.70-7.80 (m, 3H), 7.46-7.59 (m, 2H), 7.19 (s, 1H), 7.01-7.07 (m, 2H), 6.37 (s, 2H), 3.80 (s, 3H).

### Example 10

### 5-[3-(4-Ethoxy-phenyl)-isoxazol-5-ylmethyl]-2-(2-fluoro-phenyl)-5H-imidazo[4,5-d]pyridazine (Compound 110)

From 1 equivalent of 5-chloromethyl-3-(4-ethoxy-phenyl)-isoxazole. Yield 10.2 mg. MS 416.1 (M+H⁺); ¹H NMR (DMSO-d₆): δ (ppm) 10.39 (s, 1H), 9.70 (s, 1H), 8.31-8.39 (m, 1H), 7.64-7.79 (m, 3H), 7.42-7.54 (m, 2H), 7.16 (s, 1H), 7.00-7.05 (m, 2H), 6.28 (s, 2H), 4.02-4.12 (q, 2H), 1.30-1.38 (t, 3H).

### Example 11

### 2-(2-Fluoro-phenyl)-5-(3-phenyl-isoxazol-5-ylmethyl)-5H-imidazo[4,5-d]pyridazine (Compound 111)

From 1 equivalent of 5-chloromethyl-3-phenyl-isoxazole. Yield 15.2 mg. MS 372.1 (M+H⁺); ¹H NMR (DMSO-d₆): δ (ppm) 10.59 (s, 1H), 9.80 (d, 1H), 8.33-8.40 (m, 1H), 7.70-7.87 (m, 3H), 7.46-7.59 (m, 5H), 7.26 (s, 1H), 6.39 (s, 2H).

### Example 12

### 2-(2-Fluoro-phenyl)-5-[3-(4-isopropoxy-phenyl)-isoxazol-5-ylmethyl]-5H-imidazo[4,5-d]pyridazine (Compound 112)

From 1 equivalent of 5-chloromethyl-3-(4-isopropoxy-phenyl)-isoxazole. Yield 34.6 mg. MS 430.1 (M+H⁺); ¹H NMR (DMSO-d₆): δ (ppm) 10.51 (s, 1H), 9.77 (d, 1H), 8.32-8.39 (m, 1H), 7.69-7.77 (m, 3H), 7.45-7.59 (m, 2H), 7.16 (s, 1H), 6.96-7.04 (m, 2H), 6.33 (s, 2H), 4.63-4.72 (m, 1H), 1.28 (d, 6H).

### Example 13

### 5-[3-(4-Chloro-phenyl)-[1,2,4]oxadiazol-5-ylmethyl]-2-(2-fluoro-phenyl)-5H-imidazo[4,5-d]pyridazine (Compound 113)

From 5-chloromethyl-3-(4-chloro-phenyl)-[1,2,4]oxadiazole. MS: 407.8 (M+H⁺); ¹H NMR (DMSO-d₆): δ (ppm) 10.3 (s, 1H), 9.6 (s, 1H), 8.3 (m, 1H), 7.9 (m, 2H), 7.6 (m, 3H), 7.4 (m, 2H), 6.5 (s, 2H).

### Example 14

### 5-[3-(2,4-Bis-trifluoromethyl-phenyl)-isoxazol-5-ylmethyl]-2-(2-fluoro-phenyl)-5H-imidazo[4,5-d]pyridazine (Compound 114)

From 3-(2,4-bis-trifluoromethyl-phenyl)-5-chloromethyl-isoxazole. MS: 508.4 (M+H⁺); ¹H NMR (DMSO-d₆): δ (ppm) 10.3 (s, 1H), 9.6 (s, 1H), 8.3 (m, 1H), 8.2 (m, 2H), 7.9 (d, 1H), 7.6 (m, 1H), 7.4 (m, 2H), 7.0 (s, 1H), 6.3 (s, 2H).

### Example 15

### 2-(2-Fluoro-phenyl)-5-[3-(4-fluoro-2-trifluoromethyl-phenyl)-isoxazol-5-ylmethyl]-5H-imidazo[4,5-d]pyridazine (Compound 115)

From 5-chloromethyl-3-(4-fluoro-2-trifluoromethyl-phenyl)-isoxazole. MS: 458.4 (M+H⁺); ¹H NMR (DMSO-d₆): δ (ppm) 10.3 (s, 1H), 9.6 (s, 1H), 8.2 (m, 1H), 7.8 (m, 1H), 7.6 (m, 3H), 7.4 (m, 2H), 6.9 (s, 1H), 6.3 (s, 2H).

### Example 16

### 5-[3-(4-Chloro-phenyl)-isoxazol-5-ylmethyl]-2-(2-fluoro-phenyl)-5H-imidazo[4,5-d]pyridazine (Compound 116)

From 5-chloromethyl-3-(4-chloro-phenyl)-isoxazole. MS: 406 (M+H⁺); ¹H NMR (DMSO-d₆): δ (ppm) 10.4 (s, 1H), 9.7 (s, 1H), 8.3 (m, 1H), 7.8 (m, 2H), 7.7 (m, 1H), 7.6-7.4 (m, 4H), 7.2 (s, 1H), 6.3 (s, 2H).

### Example 17

### 2-(2-Fluoro-phenyl)-5-[3-(4-propoxy-phenyl)-isoxazol-5-ylmethyl]-5H-imidazo[4,5-d]pyridazine (Compound 117)

From 5-chloromethyl-3-(4-propoxy-phenyl)-isoxazole. MS: 430 (M+H⁺); ¹H NMR (DMSO-d₆): δ (ppm) 10.4 (s, 1H), 9.7 (s, 1H), 8.3 (m, 1H), 7.8-7.7 (m, 3H), 7.6-7.4 (m, 2H), 7.1 (m, 1H), 7.0 (m, 2H), 6.3 (s, 2H), 3.9 (t, 2H), 1.7 (m, 2H), 0.9 (t. 3H).

### Example 18

### 5-[5-(4-Chloro-phenyl)-[1,3,4]oxadiazol-2-ylmethyl]-2-(2-fluoro-phenyl)-5H-imidazo[4,5-d]pyridazine (Compound 118)

2-(2-Fluoro-phenyl)-5H-imidazo[4,5-d]pyridazine (30.0 mg, 0.14 mmol), 2-chloromethyl-5-phenyl-[1,3,4]oxadiazole (32.1 mg, 0.14 mmol), and cesium carbonate (91.3 mg, 0.28 mmol) were dissolved in DMF and microwaved at 120° C. for 10 minutes. The reaction was filtered and purified by reverse phase HPLC to give the desired product. Yield 12.7 mg. MS 407.0 (M+H⁺); ¹H NMR (DMSO-d₆): δ (ppm) 10.22 (s, 1H), 9.58 (d, 1H), 8.31-8.38 (m, 1H), 7.96-8.01 (m, 2H), 7.57-7.70 (m, 3H), 7.37-7.47 (m, 2H), 6.40 (s, 2H).

### General Procedure E: Synthesis of Compounds 119-145

### Synthesis of 2-Aryl-5-substituted-imidazo[4,5-d]pyridazines

### 2-Bromo-5H-imidazo[4,5-d]pyridazine

A solution of 2-Bromo-1H-imidazole-4,5-dicarbonitrile (2 g, 10 mmol from Heterocycles 29, 1325, 1989) in THF (100 mL) was cooled to −78° C. and treated with DIBALH (50 mL of 1M solution in THF, 5 eq.) over 10 minutes. The mixture was stirred for 15 minutes then quenched with potassium tartrate (aq. 10% w/vol, 80 mL) stirred for 15 minutes at 15° C. then treated with hydrazine (anhydrous, 5 mL) and stirred at room temperature for 1 hr. The reaction was then cooled to 0° C. overnight, then filtered. The solids were washed with MeOH (2×100 mL) and the organic fraction concentrated. The crude product was then purified on silica gel eluting with 0-60% CH₂Cl₂: MeOH (w/10% NH₄OH). Yield 350 mg, (18%) MS 199/201 (M+H⁺).

### 5-Substituted-2-bromo-imidazo[4,5-d]pyridazine

To a solution of 2-Bromo-5H-imidazo[4,5-d]pyridazine (1 eq) in DMF (5 mL/mmol) was added an excess of K₂CO₃ and 3-aryl-chloromethyl-isoxazole (1 eq) and heated to 40° C. for 1 hr. The mixture was then cooled and poured into H₂O (30 mL/mmol) and the precipitate collected and dried to give the products.

### 2-Aryl-5-substituted-imidazo[4,5-d]pyridazines

A solution of the 5-substituted-2-bromo-imidazo[4,5-d]pyridazine (1 eq.), aryl boronic acid (1.3 eq.) tetrakistriphenylphosphine palladium (5 mol %), K₂CO₃ (3 eq. 1M, aq) in isopropanol (10 mL/mmol) was degassed and heated to 120° C. with microwave irradiation for 20 minutes. The reaction was filtered and purified by reverse phase HPLC to give the desired product. The product was converted to the HCl salt by the addition of 1N HCl before concentration.

### Example 19

### 2-{5-[3-(2,4-Bis-trifluoromethyl-phenyl)-isoxazol-5-ylmethyl]-5H-imidazo[4,5-d]pyridazine-2-yl}-phenylamine (Compound 119)

From 5-[3-(2,4-Bis-trifluoromethyl-phenyl)-isoxazol-5-ylmethyl]-2-bromo-5H-imidazo[4,5-d]pyridazine and 2-amino-phenylboronic acid. MS 505.1 (M+H⁺); H¹ NMR (DMSO-d₆): δ (ppm) 10.22 (s, 1H), 9.57 (s, 1H), 8.18-8.09 (m, 3H), 7.86-7.83 (d, 1H), 7.21 (t, 1H), 7.02 (s, 1H), 6.84-6.82 (d, 1H), 6.63 (t, 1H), 6.30 (s, 2H).

### Example 20

### 2-Benzo[b]thiophen-2-yl-5-[3-(2,4-bis-trifluoromethyl-phenyl)-isoxazol-5-ylmethyl]-5H-imidazo[4,5-d]pyridazine (Compound 120)

From 5-[3-(2,4-Bis-trifluoromethyl-phenyl)-isoxazol-5-ylmethyl]-2-bromo-5H-imidazo[4,5-d]pyridazine and 2-benzo[b]thiophene boronic acid. MS 546.1 (M+H⁺); H¹ NMR (DMSO-d₆): δ (ppm) 10.20 (s, 1H), 9.54 (s, 1H), 8.39 (s, 1H), 8.17-8.13 (m, 2H), 8.02-7.94 (m, 2H), 7.87-7.84 (d, 1H), 7.41-7.38 (m, 2H), 7.00 (s, 1H), 6.26 (s, 2H).

### Example 21

### 5-[3-(2,4-Bis-trifluoromethyl-phenyl)-isoxazol-5-ylmethyl]-2-(4-methyl-thiophen-3-yl)-5H-imidazo[4,5-d]pyridazine (Compound 121)

From 5-[3-(2,4-Bis-trifluoromethyl-phenyl)-isoxazol-5-ylmethyl]-2-bromo-5H-imidazo[4,5-d]pyridazine and 4-methyl-thiophene 3-boronic acid. MS 510.0 (M+H⁺); H¹ NMR (DMSO-d₆): δ (ppm) 10.45 (s, 1H), 9.74 (s, 1H), 8.62-6.61 (d, 1H), 8.24-8.20 (m, 2H), 7.91-7.89 (d, 1H), 7.62-7.45 (m, 1H), 7.09 (s, 1H), 6.37 (s, 2H), 2.65 (s, 3H).

### Example 22

### 5-[3-(2,4-Bis-trifluoromethyl-phenyl)-isoxazol-5-ylmethyl]-2-thiophen-3-yl-5H-imidazo[4,5-d]pyridazine (Compound 122)

From 5-[3-(2,4-Bis-trifluoromethyl-phenyl)-isoxazol-5-ylmethyl]-2-bromo-5H-imidazo[4,5-d]pyridazine and thiophene 3-boronic acid. MS 496.0 (M+H⁺); H¹ NMR (DMSO-d₆): δ (ppm) 10.39 (s, 1H), 9.67 (s, 1H), 8.66 (s, 1H), 8.18-8.14 (m, 2H), 7.91-7.76 (m, 3H), 7.02 (s, 1H), 6.32 (s, 2H).

### Example 23

### 5-[3-(2,4-Bis-trifluoromethyl-phenyl)-isoxazol-5-ylmethyl]-2-(3,5-dimethyl-isoxazol-4-yl)-5H-imidazo[4,5-d]pyridazine (Compound 123)

From 5-[3-(2,4-Bis-trifluoromethyl-phenyl)-isoxazol-5-ylmethyl]-2-bromo-5H-imidazo[4,5-d]pyridazine and 3,5-dimethyl-isoxazole 4-boronic acid. MS 509.1 (M+H⁺); H¹ NMR (DMSO-d₆): δ (ppm) 10.37 (s, 1H), 9.66 (s, 1H), 8.23-8.19 (m, 2H), 7.91-7.89 (d, 1H), 7.08 (s, 1H), 6.36 (s, 2H), 2.84 (s, 3H), 2.56 (s, 3H).

### Example 24

### 5-[3-(2,4-Bis-trifluoromethyl-phenyl)-isoxazol-5-ylmethyl]-2-(2-fluoro-3-methoxy-phenyl)-5H-imidazo[4,5-d]pyridazine (Compound 124)

From 5-[3-(2,4-Bis-trifluoromethyl-phenyl)-isoxazol-5-ylmethyl]-2-bromo-5H-imidazo[4,5-d]pyridazine and 2-Fluoro-3-methoxy-phenylboronic acid. MS 538.1 (M+H⁺); H¹ NMR (DMSO-d₆): δ (ppm) 10.11 (s, 1H), 9.50 (s, 1H), 8.23-8.19 (m, 2H), 7.93-7.81 (m, 2H), 7.31-7.26 (m, 2H), 7.04 (s, 1H), 6.24 (s, 2H), 3.85 (s, 3H).

### Example 25

### 5-[3-(2,4-Bis-trifluoromethyl-phenyl)-isoxazol-5-ylmethyl]-2-(2-methoxy-phenyl)-5H-imidazo[4,5-d]pyridazine (Compound 125)

From 5-[3-(2,4-Bis-trifluoromethyl-phenyl)-isoxazol-5-ylmethyl]-2-bromo-5H-imidazo[4,5-d]pyridazine and 2-methoxyphenyl boronic acid. MS 520.1 (M+H⁺); H¹ NMR (DMSO-d₆): δ (ppm) 9.80 (s, 1H), 8.43-8.40 (d, 1H), 8.25-8.21 (m, 3H), 7.91-7.88 (d, 1H), 7.34-7.68 (t, 1H), 7.40-7.37 (d, 1H), 7.26-7.22 (t, 1H), 7.11 (s, 1H), 6.47 (s, 2H), 4.10 (s, 3H).

### Example 26

### 5-[3-(2,4-Bis-trifluoromethyl-phenyl)-isoxazol-5-ylmethyl]-2-o-tolyl-5H-imidazo[4,5-d]pyridazine (Compound 126)

From 5-[3-(2,4-Bis-trifluoromethyl-phenyl)-isoxazol-5-ylmethyl]-2-bromo-5H-imidazo[4,5-d]pyridazine and 2-methylphenylboronic acid. MS 504.1 (M+H⁺); H¹ NMR (DMSO-d₆): δ (ppm) 10.53 (s, 1H), 9.80 (s, 1H), 8.25-8.21 (m, 2H), 8.01-7.97 (d, 1H), 7.92-7.89 (d, 1H), 7.53-7.44 (m, 3H), 7.10 (s, 1H), 6.40 (s, 2H), 2.70 (s, 3H).

### Example 27

### 2-(3-Fluoro-phenyl)-5-[3-(4-propoxy-phenyl)-isoxazol-5-ylmethyl]-5H-imidazo[4,5-d]pyridazine (Compound 127)

From 2-bromo-5-[3-(4-propoxy-phenyl)-isoxazol-5-ylmethyl]-5H-imidazo[4,5-d]pyridazine and 3-fluorophenyl boronic acid. MS 430.1 (M+H⁺); H¹ NMR (DMSO-d₆): δ (ppm) 10.20 (s, 1H), 9.54 (s, 1H), 8.19-8.17 (d, 1H), 8.08-8.05 (d, 1H), 7.71-7.68 (d, 2H), 7.59-7.57 (m, 1H), 7.37 (t, 1H), 7.08 (s, 1H), 6.97-6.94 (d, 2H), 6.15 (s, 2H), 3.92-3.88 (t, 2H), 1.70-1.63 (m, 2H), 0.93-0.88 (t, 3H).

### Example 28

### 5-[3-(2,4-Bis-trifluoromethyl-phenyl)-isoxazol-5-ylmethyl]-2-(4-fluoro-phenyl)-5H-imidazo[4,5-d]pyridazine (Compound 128)

From 5-[3-(2,4-Bis-trifluoromethyl-phenyl)-isoxazol-5-ylmethyl]-2-bromo-5H-imidazo[4,5-d]pyridazine and 4-fluorophenyl boronic acid. MS 508.1 (M+H⁺); H¹ NMR (DMSO-d₆): δ (ppm) 10.27 (s, 1H), 9.62 (s, 1H), 8.46-8.42 (q, 2H), 8.24-8.20 (m, 2H), 7.92-7.89 (d, 1H), 7.47-7.41 (t, 2H), 6.03 (s, 2H).

### Example 29

### 5-[3-(4-Butoxy-phenyl)-isoxazol-5-ylmethyl]-2-(3-fluoro-phenyl)-5H-imidazo[4,5-d]pyridazine (Compound 129)

From 2-bromo-5-[3-(4-butoxy-phenyl)-isoxazol-5-ylmethyl]-5H-imidazo[4,5-d]pyridazine and 3-fluorophenyl boronic acid. MS 444.2 (M+H⁺); H¹ NMR (DMSO-d₆): δ (ppm) 10.22 (s, 1H), 9.55 (s, 1H), 8.20-8.17 (d, 1H), 8.10-8.09 (d, 1H), 7.70-7.67 (d, 2H), 7.60-7.57 (m, 1H), 7.37 (t, 1H), 7.08 (s, 1H) 6.97-6.94 (d, 2H), 6.16 (s, 2H), 3.96-3.92 (t, 2H), 1.66-1.61 (m, 2H), 1.40-1.33 (m, 2H), 0.89-0.84 (t, 3H).

### Example 30

### 5-[3-(2,4-Bis-trifluoromethyl-phenyl)-isoxazol-5-ylmethyl]-2-(3-fluoro-phenyl)-5H-imidazo[4,5-d]pyridazine(Compound (Compound 130)

From 5-[3-(2,4-Bis-trifluoromethyl-phenyl)-isoxazol-5-ylmethyl]-2-bromo-5H-imidazo[4,5-d]pyridazine and 3-fluorophenyl boronic acid. MS 508.1 (M+H⁺); H¹ NMR (DMSO-d₆): δ (ppm) 10.50 (s, 1H), 9.76 (s, 1H), 8.29-8.20 (m, 4H), 7.91-7.89 (d, 1H), 7.70-7.67 (m, 1H), 7.05 (t, 1H), 7.09 (s, 1H), 6.39 (s, 2H).

### Example 31

### 5-[3-(2,4-Bis-trifluoromethyl-phenyl)-isoxazol-5-ylmethyl]-2-(4-methoxy-phenyl)-5H-imidazo[4,5-d]pyridazine (Compound 131)

From 5-[3-(2,4-Bis-trifluoromethyl-phenyl)-isoxazol-5-ylmethyl]-2-bromo-5H-imidazo[4,5-d]pyridazine and 4-methoxyphenyl boronic acid. MS 520.1 (M+H⁺); H¹ NMR (DMSO-d₆): δ (ppm) 10.45 (s, 1H), 9.73 (s, 1H), 8.39-8.36 (d, 2H), 8.25-8.20 (m, 2H), 7.91-7.89 (d, 1H), 7.22-7.19 (d, 2H), 7.09 (s, 1H), 6.39 (s, 2H), 3.88 (s, 3H).

### Example 32

### 5-[3-(2,4-Bis-trifluoromethyl-phenyl)-isoxazol-5-ylmethyl]-2-(2,4-difluoro-phenyl)-5H-imidazo[4,5-d]pyridazine (Compound 132)

From 5-[3-(2,4-Bis-trifluoromethyl-phenyl)-isoxazol-5-ylmethyl]-2-bromo-5H-imidazo[4,5-d]pyridazine and 2,4 difluorophenyl boronic acid. MS 526.1 (M+H⁺); H¹ NMR (DMSO-d₆): δ (ppm) 10.10 (s, 1H), 9.49 (s, 1H), 8.44-8.39 (m, 1H), 8.23-8.19 (m, 2H), 7.93-7.90 (d, 1H), 7.42 (t, 1H), 7.26 (t, 1H), 7.04 (s, 1H), 6.24 (s, 2H).

### Example 33

### 2-{5-[3-(2,4-Bis-trifluoromethyl-phenyl)-isoxazol-5-ylmethyl]-5H-imidazo[4,5-d]pyridazin-2-yl}-benzamide (Compound 133)

From 5-[3-(2,4-Bis-trifluoromethyl-phenyl)-isoxazol-5-ylmethyl]-2-bromo-5H-imidazo[4,5-d]pyridazine and benzamide 2-boronic acid. MS 533.1 (M+H⁺); H¹ NMR (DMSO-d₆): δ (ppm) 10.64 (s, 1H), 9.86 (s, 1H), 8.24-8.13 (m, 4H), 7.92-7.88 (m, 2H), 7.77-7.68 (m, 3H), 7.13 (s, 1H), 6.43 (s, 2H).

### Example 34

### 2-{5-[3-(2,4-Bis-trifluoromethyl-phenyl)-isoxazol-5-ylmethyl]-5H-imidazo[4,5-d]pyridazin-2-yl}-phenol (Compound 134)

From 5-[3-(2,4-Bis-trifluoromethyl-phenyl)-isoxazol-5-ylmethyl]-2-bromo-5H-imidazo[4,5-d]pyridazine and phenol 2-boronic acid. MS 506.1 (M+H⁺); H¹ NMR (DMSO-d₆): δ (ppm) 10.16 (s, 1H), 9.55 (s, 1H), 8.30-8.27 (dd, 1H), 8.18-8.14 (m, 2H), 7.87-7.84 (d, 1H), 7.38 (t, 1H), 7.03-6.96 (m, 3H), 6.26 (s, 2H).

### Example 35

### 5-[3-(2,4-Bis-trifluoromethyl-phenyl)-isoxazol-5-ylmethyl]-2-(4-trifluoromethyl-phenyl)-5H-imidazo[4,5-d]pyridazine (Compound 135)

From 5-[3-(2,4-Bis-trifluoromethyl-phenyl)-isoxazol-5-ylmethyl]-2-bromo-5H-imidazo[4,5-d]pyridazine and 4-trifluoromethylphenyl boronic acid. MS 558.1 (M+H⁺); H¹ NMR (DMSO-d₆): δ (ppm) 10.45 (s, 1H), 9.73 (s, 1H), 8.63-8.61 (d, 2H), 8.23-8.19 (m, 2H), 8.01-7.89 (m, 3H), 7.08 (s, 1H), 6.37 (s, 2H).

### Example 36

### 5-[3-(2,4-Bis-trifluoromethyl-phenyl)-isoxazol-5-ylmethyl]-2-(1H-indol-4-yl)-5H-imidazo[4,5-d]pyridazine (Compound 136)

From 5-[3-(2,4-Bis-trifluoromethyl-phenyl)-isoxazol-5-ylmethyl]-2-bromo-5H-imidazo[4,5-d]pyridazine and 1H indole 4-boronic acid. MS 529.1 (M+H⁺); H¹ NMR (DMSO-d₆): δ (ppm) 10.68 (s, 1H), 9.97 (s, 1H), 8.75 (s, 1H); 8.47-8.42 (m, 3H), 8.29-8.26 (d. 1H), 8.14-8.11 (d, 1H), 8.01-7.99 (d, 1H), 7.87 (m, 1H), 7.32 (s, 1H), 6.80 (s, 1H), 6.64 (s, 2H).

### Example 37

### 1-(3-{5-[3-(2,4-Bis-trifluoromethyl-phenyl)-isoxazol-5-ylmethyl]-5H-imidazo[4,5-d]pyridazin-2-yl}-4-fluoro-phenyl)-ethanone (Compound 137)

From 5-[3-(2,4-Bis-trifluoromethyl-phenyl)-isoxazol-5-ylmethyl]-2-bromo-5H-imidazo[4,5-d]pyridazine and 2-fluoro 5-acetylphenyl boronic acid. MS 550.1 (M+H⁺); H¹ NMR (DMSO-d₆): δ (ppm) 10.35 (s, 1H), 9.68 (s, 1H), 8.92-8.89 (dd, 1H), 8.24-8.19 (m, 3H), 7.93-7.90 (d, 1H), 7.58 (m, 1H), 7.08 (s, 1H), 6.35 (s, 2H), 2.67 (s, 3H).

### Example 38

### 2-(4-Methoxy-phenyl)-5-[3-(4-propoxy-phenyl)-isoxazol-5-ylmethyl]-5H-imidazo[4,5-d]pyridazine (Compound 138)

From 2-bromo-5-[3-(4-propoxy-phenyl)-isoxazol-5-ylmethyl]-5H-imidazo[4,5-d]pyridazine and 4-methoxyphenyl boronic acid. MS 442.2 (M+H⁺); H¹ NMR (DMSO-d₆): δ (ppm) 10.31 (s, 1H), 9.63 (s, 1H), 8.31-8.28 (d, 2H), 7.71-7.68 (d, 2H), 7.16-7.13 (d, 2H), 7.09 (s, 1H), 6.70-6.95 (d, 2H), 6.22 (s, 2H), 3.93-3.88 (t, 2H), 3.18 (s, 3H), 1.70-1.63 (m, 2H), 0.94-0.89 (t, 3H).

### Example 39

### 5-[3-(2,4-Bis-trifluoromethyl-phenyl)-isoxazol-5-ylmethyl]-2-(1H-indol-5-yl)-5H-imidazo[4,5-d]pyridazine (Compound 139)

From 5-[3-(2,4-Bis-trifluoromethyl-phenyl)-isoxazol-5-ylmethyl]-2-bromo-5H-imidazo[4,5-d]pyridazine and 1H indole 5-boronic acid. MS 529.1 (M+H⁺); H¹ NMR (DMSO-d₆): δ (ppm) 10.81 (s, 1H), 10.11 (s, 1H), 9.07 (s, 1H), 8.61 (m, 3H), 8.28-8.26 (d, 1H), 8.01-7.88 (m, 3H), 7.46 (s, 1H), 7.02 (s, 1H), 6.77 (s, 2H).

### Example 40

### 5-[3-(2,4-Bis-trifluoromethyl-phenyl)-isoxazol-5-ylmethyl]-2-(2,6-difluoro-phenyl)-5H-imidazo[4,5-d]pyridazine (Compound 140)

From 5-[3-(2,4-Bis-trifluoromethyl-phenyl)-isoxazol-5-ylmethyl]-2-bromo-5H-imidazo[4,5-d]pyridazine and 2,6 difluorophenyl boronic acid. MS 526.1 (M+H⁺); H¹ NMR (DMSO-d₆): δ (ppm) 10.42 (s, 1H), 9.72 (s, 1H), 8.25 (m, 3H), 7.92 (d, 1H), 7.38-7.33 (m, 2H), 7.08 (s, 1H), 6.35 (s, 2H).

### Example 41

### 5-[3-(4-Butoxy-phenyl)-isoxazol-5-ylmethyl]-2-(4-methoxy-phenyl)-5H-imidazo[4,5-d]pyridazine (Compound 141)

From 2-bromo-5-[3-(4-butoxy-phenyl)-isoxazol-5-ylmethyl]-5H-imidazo[4,5-d]pyridazine and 4-methoxyphenyl boronic acid. MS 456.2 (M+H⁺); H¹ NMR (DMSO-d₆): δ (ppm) 10.35 (s, 1H), 9.71 (s, 1H), 8.40-8.37 (d, 2H), 7.76-7.73 (d, 2H), 7.22-7.19 (d, 2H), 7.15 (s, 1H), 7.03-7.00 (d, 2H), 6.30 (s, 2H), 4.02-3.98 (t, 2H), 3.88 (s, 3H), 1.69 (m, 2H), 1.43-1.41 (m, 2H), 0.94-0.90 (t, 3H).

### Example 42

### 5-[3-(2,4-Bis-trifluoromethyl-phenyl)-isoxazol-5-ylmethyl-2-furan-2-yl-5H-imidazo[4,5-d]pyridazine (Compound 142)

From 5-[3-(2,4-Bis-trifluoromethyl-phenyl)-isoxazol-5-ylmethyl]-2-bromo-5H-imidazo[4,5-d]pyridazine and furan 2-boronic acid. MS 480.1 (M+H⁺); H¹ NMR (DMSO-d₆): δ (ppm) 10.24 (s, 1H), 9.58 (s, 1H), 8.24-8.20 (m, 2H), 8.06 (s, 1H), 7.92 (d, 1H), 7.51-7.50 (d, 1H), 7.06 (s, 1H), 6.81-6.80 (m, 1H), 6.31 (s, 2H).

### Example 43

### 5-[3-(2,4-Bis-trifluoromethyl-phenyl)-isoxazol-5-ylmethyl]-2-thiophen-2-yl-5H-imidazo[4,5-d]pyridazine (Compound 143)

From 5-[3-(2,4-Bis-trifluoromethyl-phenyl)-isoxazol-5-ylmethyl]-2-bromo-5H-imidazo[4,5-d]pyridazine and thiophene 2-boronic acid. MS 496.0 (M+H⁺); H¹ NMR (DMSO-d₆): δ (ppm) 10.34 (s, 1H), 9.65 (s, 1H), 8.23-8.20 (m, 3H), 7.80-7.89 (m, 2H), 7.34-7.31 (t, 1H), 7.07 (s, 1H), 6.35 (s, 2H).

### Example 44

### 2-Furan-2-yl-5-[3-(4-propoxy-phenyl)-isoxazol-5-ylmethyl]-5H-imidazo[4,5-d]pyridazine (Compound 144)

From 2-bromo-5-[3-(4-propoxy-phenyl)-isoxazol-5-ylmethyl]-5H-imidazo[4,5-d]pyridazine and furan 2-boronic acid. MS 402.2 (M+H⁺); H¹ NMR (DMSO-d₆): δ (ppm) 10.270 (s, 1H), 9.57 (s, 1H), 8.05 (s, 1H), 7.70-7.67 (d, 2H), 7.54-7.53 (d, 1H), 7.09 (s, 1H), 6.97-6.94 (d, 2H), 6.78-6.76 (m, 1H), 6.19 (s, 2H), 3.92-3.88 (t, 2H), 1.68-1.65 (m, 2H), 0.93-0.88 (t, 3H).

### Example 45

### 2-(4-Fluoro-phenyl)-5-[3-(4-propoxy-phenyl)-isoxazol-5-ylmethyl]-5H-imidazo[4,5-d]pyridazine (Compound 145)

From 2-bromo-5-[3-(4-propoxy-phenyl)-isoxazol-5-ylmethyl]-5H-imidazo[4,5-d]pyridazine and 4-fluorophenyl boronic acid. MS 430.2 (M+H⁺); H¹ NMR (DMSO-d₆): δ (ppm) 10.21 (s, 1H), 9.55 (s, 1H), 8.46-8.41 (m, 2H), 7.78-7.74 (m, 2H), 7.45-7.39 (m, 2H), 7.13 (s, 1H), 7.03-7.00 (d, 2H), 6.19 (s, 2H), 3.98-3.94 (m, 2H), 1.74-1.71 (q, 2H), 0.99-0.94 (m, 3H).

### General Procedure F

### Synthesis of 2-Aryl-5H-imidazo[4,5-d]pyridazines

To a solution of diaminomaleonitrile in THF (1 mL/mmol) was added aryl aldehyde (1 eq) and then catalytic H₂SO₄ (1 drop/20 mmol) and stirred at room temperature for 90 minutes. The solvent was evaporated to dryness then the solid washed with 1:1 ethyl ether and hexane giving the pure product: 2-amino-3-aryl-but-2-enedinitrile.

The 2-amino-3-aryl-but-2-enedinitrile is dissolved in DMF (3 mL/mmol) and then treated with NCS (1.5 eq) followed by nicotinamide (1.5 eq). The solution turned to dark brown in 2 minutes. After 1 hour the precipitated nicotinamide HCl salt was filtered off and the solution concentrated to oil. The reaction mixture was then poured into cold water with the product oiling out. Ethyl acetate was added to dissolve the oil and the organics were washed with brine. The organics were dried with MgSO₄ and evaporated to give a black oil. The oil was dissolved in a minimum amount of DCM and filtered through silica gel (3 g/mmol) with DCM:MeOH (4:1). The solvent was evaporated to give the product-2-aryl-1H-imidazole-4,5-dicarbonitrile.

The 2-aryl-1H-imidazole-4,5-dicarbonitrile was dissolved in THF (1.5 mL/mmol), cooled to −78° C. and treated with DIBAL-H (6.5 eq, 1M in THF) dropwise. Water was carefully added to the cold mixture until the excess DIBAL-H was fully quenched. Hydrazine (3 eq. hydrate) was added to the solution and then the reaction was warmed to room temperature. MeOH (1 mL/mmol) was added and the aluminum salts were filtered. The solid was washed with another 50 mL of MeOH. The filtrate was evaporated and purified by silica column with the gradient from 10% to 30% DCM/MeOH (with 10% v/v NH₄OH) to provide 2-aryl-5H-imidazo[4,5-d]pyridazines.

### General Procedure G

### Synthesis of Methanesulfonic Acid 5-Aryl-isoxazol-3-ylmethyl Esters

A mixture of phenyl isocyanate (2.2 eq, 1.1 g), 2-(2-Nitro-ethoxy)-tetrahydro-pyran (1 eq, 875 mg) and aryl alkyne (1 eq, 5 mmol) in benzene (20 mL) was treated with DIEA (20 drops, excess) then heated to 75° C. overnight in a sealed vial. The mixture was cooled, the solution decanted, concentrated and purified on silica gel eluting with EtOAc : hexanes 0-40% to give the 3-(Tetrahydro-pyran-2-yloxymethyl)-aryl-isoxazole.

A solution of the 3-(Tetrahydro-pyran-2-yloxymethyl)-5-aryl-isoxazole (725 mg) in HOAc:H₂O:THF (4:2:1, 10 mL) was heated to 75° C. for 5 hrs. The mixture was cooled to room temperature, concentrated and the product, 5-aryl-isoxazol-3-yl-methanol, which was used directly.

To a solution of the 5-aryl-isoxazol-3-yl-methanol (2.2 mmol) in DCM (20 mL) was added triethylamine (0.5 mL, 2 eq.) and mesyl chloride (1.5 eq, 0.26 mL) and stirred at room temperature for 1 hr. The reaction was then quenched with water (10 mL) and the organics partitioned and concentrated to give the crude product methanesulfonic acid 5-aryl-isoxazol-3-ylmethyl ester.

### General Procedure H

### Synthesis of Compounds 146-244 and 275-289

A solution of 2-aryl-5H-imidazo[4,5-d]pyridazine (0.10 mmol), chloromethyl-, or methanesulfonic acid methyl ester- of aryl isoxazole compound (1 equivalent), and alkali carbonate (0.20 mmol) in DMF (3 mL) was heated under microwave irradiation at 60-120° C. for 10 minutes. The reaction was filtered and purified by reverse phase HPLC to give the desired product. The product was converted to the HCl salt by the addition of 1N HCl before concentration.

### Example 46

### 2-(2,3-Difluoro-phenyl)-5-[3-(4-pyridin-4-ylethynyl-phenyl)-isoxazol-5-ylmethyl]-5H-imidazo[4,5-d]pyridazine (Compound 146)

### (4-Pyridin-4-ylethynyl-phenyl)-methanol

A mixture of 4-ethynylpyridine hydrochloride (210 mg, 1.5 mmol), 4-iodobenzyl alcohol (351 mg, 1.5 mmol), and triethylamine (4 mL) was sparged with Ar for 2 min in a microwave vial. To this mixture was added Cu(I)I (29 mg, 0.15 mmol) and tetrakis(triphenylphosphine)palladium (92 mg, 0.08 mmol). The vial was sealed, and the contents were heated to 130° C. in a microwave for 10 min. The cooled reaction mixture was heterogeneous with a heavy black ppt. The reaction mixture was partitioned between EtOAc and water. The organic layer was washed with brine, dried over sodium sulfate, and adsorbed onto Celite. The product was purified by SiO₂ flash chromatography using EtOAc in hexanes (50-100%) to give the product as a white flaky solid. Yield: 110 mg.

### 4-Pyridin-4-ylethynyl-benzaldehyde

(4-Pyridin-4-ylethynyl-phenyl)-methanol (100 mg) was suspended in DCM (20 mL) and excess MnO₂ (ca. 1 g) was added. The reaction mixture was stirred for 1 h, filtered, and concentrated onto Celite. The product was isolated by SiO₂ flash chromatography using EtOAc in hexanes (30-100%) to give the product as a white crystalline solid. Yield: 57 mg.

### 4-(2-Pyridin-4-yl-ethyl)-benzaldehyde

(4-Pyridin-4-ylethynyl-phenyl)-methanol (180 mg) was dissolved in EtOH (50 mL) and the solution was sparged with Ar. Pd (10% on carbon (50 mg) was added and mixture was stirred for 1 h under a balloon filled with H₂. The reaction mixture was filtered through Celite, and was then concentrated onto Celite. The product was isolated by SiO₂ flash chromatography using 50-100% EtOAc in hexanes to give the product as a flaky solid. This material was dissolved in DCM (25 mL) and a large excess of MnO2 (ca. 1 g) was added. The reaction mixture was stirred for 30 min, and then was filtered through Celite and concentrated onto Celite. The product was purified by SiO₂ flash chromatography using EtOAc in hexanes (50-100%) to give the product as white crystals. Yield: 57 mg.

### 2-(2,3-Difluoro-phenyl)-5-[3-(4-pyridin-4-ylethynyl-phenyl)-isoxazol-5-ylmethyl]-5H-imidazo[4,5-d]pyridazine (Compound 146)

From 2-(2,3-difluoro-phenyl)-5H-imidazo[4,5-d]pyridazine and 4-[4-(5-chloromethyl-isoxazol-3-yl)-phenylethynyl]-pyridine (General Procedure B, from 4-(2-pyridin-4-yl-ethyl)-benzaldehyde). 491.0 (M+H⁺); H¹ NMR (DMSO-d₆): δ (ppm) 10.54 (s, 1H), 9.76 (s, 1H), 8.83-8.81 (dd, 2H), 8.19-8.15 (m, 1H), 7.99-7.91 (m, 4H), 7.81-7.68 (m, 3H), 7.50-7.43 (m, 1H), 7.33 (s, 1H), 6.38 (s, 2H).

### Example 47

### 5-[3-(2,4-Bis-trifluoromethyl-phenyl)-isoxazol-5-ylmethyl]-2-(2,4,5-trifluoro-phenyl)-5H-imidazo[4,5-d]pyridazine (Compound 147)

From 3-(2,4-bis-trifluoromethyl-phenyl)-5-chloromethyl-isoxazole and 2-(2,4,5-trifluoro-phenyl)-5H-imidazo[4,5-d]pyridazine. 544.0 (M+H⁺); H¹ NMR (DMSO-d₆): δ (ppm) 10.45 (s, 1H), 9.71 (s, 1H), 8.42-8.33 (m, 1H), 8.24-8.20 (m, 2H), 7.93-7.82 (m, 2H), 7.08 (s, 1H), 6.37 (s, 2H).

### Example 48

### 2-(2,3-Difluoro-phenyl)-5-{3-[4-(pyridin-4-ylmethoxy)-phenyl]-isoxazol-5-ylmethyl}-H-imidazo[4,5-d]pyridazine (Compound 148)

From 2-(2,3-difluoro-phenyl)-5H-imidazo[4,5-d]pyridazine and 4-[4-(5-chloromethyl-isoxazol-3-yl)-phenoxymethyl]-pyridine. MS 497.1 (M+H⁺); H¹ NMR (DMSO-d₆): δ (ppm) 10.61 (s, 1H), 9.79(s, 1H), 8.92-8.90 (d, 2H), 8.19-8.16 (d, 1H), 8.06-8.04 (d, 2H), 7.84-7.69(m, 3H), 7.51-7.45 (t, 1H), 7.19-7.16 (m, 3H), 5.53 (s, 2H), 6.36 (s, 2H).

### Example 49

### 2-(2,3-Difluoro-phenyl)-5-[3-(2,4-dimethyl-thiazol-5-yl)-isoxazol-5-ylmethyl]-5H-imidazo[4,5-d]pyridazine (Compound 149)

From 2-(2,3-difluoro-phenyl)-5H-imidazo[4,5-d]pyridazine and 5-chloromethyl-3-(2,4-dimethyl-thiazol-5-yl)-isoxazole. 425.0 (M+H⁺); H¹ NMR (DMSO-d₆): δ (ppm) 10.62(s, 1H), 9.82 (s, 1H), 8.16-8.14 (t, 1H), 7.79-7.75 (q, 1H), 7.5(m, 1H), 7.14(s, 1H), 6.37 (s, 2H), 2.62 (s, 3H), 2.49 (s, 3H).

### Example 50

### 5-[3-(3,4-Bis-difluoromethoxy-phenyl)-isoxazol-5-ylmethyl]-2-(2,3-difluoro-phenyl)-5H-imidazo[4,5-d]pyridazine (Compound 150)

From 2-(2,3-difluoro-phenyl)-5H-imidazo[4,5-d]pyridazine and 3-(3,4-Bis-difluoromethoxy-phenyl)-5-chloromethyl-isoxazole. 522.0 (M+H⁺); H¹ NMR (DMSO-d₆): δ (ppm) 10.50 (s, 1H), 9.74 (s, 1H), 8.18-8.13 (t, 1H), 7.15-7.71(m, 3H), 7.54-7.45(m, 2H), 7.30 (s, 2H), 7.05 (s, 1H), 6.35 (s, 2H).

### Example 51

### 5-[3-(4-Difluoromethoxy-3-ethoxy-phenyl)-isoxazol-5-ylmethyl-2-(2,3-difluoro-phenyl)-5H-imidazo[4,5-d]pyridazine (Compound 151)

From 2-(2,3-difluoro-phenyl)-5H-imidazo[4,5-d]pyridazine and 5-chloromethyl-3-(4-difluoromethoxy-3-ethoxy-phenyl)-isoxazole. 500.7 (M+H⁺); H¹ NMR (DMSO-d₆): δ (ppm) 10.12 (s, 1H), 9.50 (s, 1H), 8.16 (t, 1H), 7.56-7.52 (m, 2H), 7.46-7.43 (dd, 1H), 7.38-7.34 (m, 1H), 7.27-7.23(m, 2H), 7.13 (s, 1H), 6.18 (s, 2H), 4.17-4.10 (q, 2H), 1.36-1.32 (t, 3H).

### Example 52

### 2-(2,3-Difluoro-phenyl)-5-{3-[4-(4-methyl-piperazin-1-ylmethyl)-phenyl]-isoxazol-5-ylmethyl}-5H-imidazo[4,5-d]pyridazine (Compound 152)

From 2-(2,3-difluoro-phenyl)-5H-imidazo[4,5-d]pyridazine and 1-[4-(5-chloromethyl-isoxazol-3-yl)-benzyl]-4-methyl-piperazine. 502.1 (M+H⁺), H¹ NMR (DMSO-d₆): δ (ppm) 10.58 (s, 1H), 9.76 (s, 1H), 8.18-8.14 (t, 1H), 7.93-7.90 (d, 2H), 7.78-7.71(m, 3H), 7.50-7.46 (m, 1H), 7.29 (s, 1H), 6.36 (s, 2H), 4.40 (b, 2H), 3.59-3.51 (d, 8H), 2.79 (s, 3H).

### Example 53

### 2-(2,3-Difluoro-phenyl)-5-[3-(4-imidazol-1-ylmethyl-phenyl)-isoxazol-5-ylmethyl]-5H-imidazo[4,5-d]pyridazine (Compound 153)

From 2-(2,3-difluoro-phenyl)-5H-imidazo[4,5-d]pyridazine and 5-chloromethyl-3-(4-imidazol-1-ylmethyl-phenyl)-isoxazole. MS 470.1 (M+H⁺); H¹ NMR (DMSO-d₆): δ (ppm) 10.49 (s, 1H), 9.71 (s, 1H), 9.33 (s, 1H), 8.17-8.15 (d, 1H), 7.89-7.86 (d, 2H), 7.80 (s, 1H), 7.71-7.70 (m, 2H), 7.53-7.44 (m, 3H), 7.25 (d, 1H), 6.33(s, 2H), 5.49 (s, 2H).

### Example 54

### 2-(2,3-Difluoro-phenyl)-5-{3-[4-(1-methyl-1H-imidazol-2-ylmethoxy)-phenyl]-isoxazol-5-ylmethyl}-5H-imidazo[4,5-d]pyridazine (Compound 154)

From 2-(2,3-difluoro-phenyl)-5H-imidazo[4,5-d]pyridazine 5-chloromethyl-3-[4-(1-methyl-1H-imidazol-2-ylmethoxy)-phenyl]-isoxazole. MS 500.1 (M+H⁺); H¹ NMR (DMSO-d₆): δ (ppm) 10.59 (s, 1H), 9.78 (s, 1H), 8.19-8.15 (m, 1H), 7.85-7.70 (m, 5H), 7.50-7.47 (t, 1H), 7.26-7.21 (m, 3H), 6.36 (s, 2H), 5.55 (s, 2H), 3.87 (s, 3H).

### Example 55

### 2-(2,3-Difluoro-phenyl)-5-(3-pyridin-4-yl-isoxazol-5-ylmethyl)-5H-imidazo[4,5-d]pyridazine (Compound 155)

From 2-(2,3-difluoro-phenyl)-5H-imidazo[4,5-d]pyridazine and 4-(5-chloromethyl-isoxazol-3-yl)-pyridine. MS 391.1 (M+H⁺); H¹ NMR (DMSO-d₆): δ (ppm) 10.42 (s, 1H), 9.70 (s, 1H), 8.86-8.84 (d, 2H), 8.17-8.12 (t, 1H), 8.07-8.05 (dd, 2H), 7.72-7.68 (m, 1H), 7.48-7.44 (m, 2H), 6.37 (s, 2H).

### Example 56

### 2-(2,3-Difluoro-phenyl)-5-[3-(4-morpholin-4-ylmethyl-phenyl)-isoxazol-5-ylmethyl]-5H-imidazo[4,5-d]pyridazine (Compound 156)

From 2-(2,3-difluoro-phenyl)-5H-imidazo[4,5-d]pyridazine and 4-[4-(5-chloromethyl-isoxazol-3-yl)-benzyl]-morpholine. MS 489.2 (M+H⁺); H¹ NMR (DMSO-d₆): δ (ppm) 10.43 (s, 1H), 9.70 (s, 1H), 8.17-8.14 (m, 1H), 7.94-7.91 (d, 2H), 7.75-7.72 (m, 3H), 7.48-7.41 (m, 1H), 7.27 (s, 1H), 6.32 (s, 2H), 4.37 (s, 2H), 3.94-3.74 (m, 4H), 3.24-3.10 (m, 4H).

### Example 57

### 2-(2,3-Difluoro-phenyl)-5-[3-(4-piperidin-1-ylmethyl-phenyl)-isoxazol-5-ylmethyl]-5H-imidazo[4,5-d]pyridazine (Compound 157)

From 2-(2,3-difluoro-phenyl)-5H-imidazo[4,5-d]pyridazine and 1-[4-(5-chloromethyl-isoxazol-3-yl)-benzyl]-piperidine. MS 487.2 (M+H⁺); H¹ NMR (DMSO-d₆): δ (ppm) 10.87 (s, 1H), 9.66 (s, 1H), 8.17-8.14 (m, 1H), 7.94-7.91 (d, 2H), 7.72-7.66 (m, 3H), 7.46-7.39 (m, 1H), 7.26 (s, 1H), 6.30 (s, 2H), 4.03-4.29 (d, 2H), 3.29-3.25 (d, 2H), 2.84 (b, 2H), 1.75-1.66 (m, 6H).

### Example 58

### 2-(2,3-Difluoro-phenyl)-5-{3-[4-(2-pyrrolidin-1-yl-ethoxy)-phenyl]-isoxazol-5-ylmethyl}-5H-imidazo[4,5-d]pyridazine (Compound 158)

From 2-(2,3-difluoro-phenyl)-5H-imidazo[4,5-d]pyridazine and 5-chloromethyl-3-[4-(2-pyrrolidin-1-yl-ethoxy)-phenyl]-isoxazole. MS 503.1 (M+H⁺); H¹ NMR (DMSO-d₆): δ (ppm) 10.32 (s, 1H), 9.62 (s, 1H), 8.17-8.13 (m, 1H), 7.83-7.80 (d, 2H), 7.66-7.63 (m, 1H), 7.43-7.37 (m, 1H), 7.16-7.09 (m, 3H), 6.24 (s, 2H), 4.39-4.36 (m, 2H), 3.60-3.56 (m, 4H), 3.12-3.06 (b, 2H), 2.01-1.86 (m, 4H).

### Example 59

### 3-(4-{5-[2-(2,3-Difluoro-phenyl)-imidazo[4,5-d]pyridazin-5-ylmethyl]-isoxazol-3-yl}-phenoxymethyl)-benzoic acid (Compound 159)

From 2-(2,3-difluoro-phenyl)-5H-imidazo[4,5-d]pyridazine and 3-[4-(5-chloromethyl-isoxazol-3-yl)-phenoxymethyl]-benzoic acid. MS 540.2 (M+H⁺); H¹ NMR (DMSO-d₆): (ppm) 10.27 (s, 1H), 9.60 (s, 1H), 8.13-8.09 (m, 1H), 7.80 (s, 1H), 7.86-7.83 (d, 1H), 7.76-7.73 (d, 2H), 7.66-7.60 (m, 2H), 7.49-7.37 (m, 2H), 7.10-7.07 (d, 3H), 6.19 (s, 2H), 5.19 (s, 2H).

### Example 60

### 2-(2,3-Difluoro-phenyl)-5-[3-(4-fluoro-2-trifluoromethoxy-phenyl)-isoxazol-5-ylmethyl]-5H-imidazo[4,5-d]pyridazine (Compound 160)

### 4-Fluoro-2-trifluoromethoxy-benzaldehyde

To a solution of 1-fluoro-3-trifluoromethoxy-benzene (1.73g, 9.6 mmol) in THF (20 mL) at −78° C. was added nBuLi (1.2 eq, 4.6 mL of 2.5M in hexanes). The mixture was stirred for 180 minutes and quenched with DMF (2 mL) and allowed to warm to room temperature. Solvents were removed, the reaction was washed with H₂O (10 mL) and the organics concentrated giving the crude product.

### 2-(2,3-Difluoro-phenyl)-5-[3-(4-fluoro-2-trifluoromethoxy-phenyl)-isoxazol-5-ylmethyl]-5H-imidazo[4,5-d]pyridazine (Compound 160)

From 2-(2,3-difluoro-phenyl)-5H-imidazo[4,5-d]pyridazine and 5-chloromethyl-3-(4-fluoro-2-trifluoromethoxy-phenyl)-isoxazole (General Procedure B, from 4-fluoro-2-trifluoromethoxy-benzaldehyde). MS 492.0 (M+H⁺); H¹ NMR (DMSO-d₆): δ (ppm) 10.67 (s, 1H), 9.67 (s, 1H), 8.16-8.12 (m, 1H), 7.76-7.66 (m, 2H), 7.53-7.39 (m, 3H), 7.07 (s, 1H), 6.34 (s, 2H).

### Example 61

### [2-(4-{5-[2-(2,3-Difluoro-phenyl)-imidazo[4,5-d]pyridazin-5-ylmethyl]-isoxazol-3-yl}-phenoxy)-ethyl]-dimethyl-amine (Compound 161)

From 2-(2,3-difluoro-phenyl)-5H-imidazo[4,5-d]pyridazine and {2-[4-(5-chloromethyl-isoxazol-3-yl)-phenoxy]-ethyl}-dimethyl-amine. MS 477.2 (M+H⁺); H¹ NMR (DMSO-d₆): δ (ppm) 10.47 (s, 1H), 9.72 (s, 1H), 8.18-8.15 (m, 1H), 7.83-7.66 (m, 3H), 7.49-7.42 (m, 1H), 7.18 (s, 1H), 7.12-7.08 (m, 2H), 6.31 (s, 2H), 4.43-4.39 (t, 2H), 3.53-3.45 (q, 2H), 2.84-2.82 (d, 6H).

### Example 62

### 4-(4-{5-[2-(2,3-Difluoro-phenyl)-imidazo[4,5-d]pyridazin-5-ylmethyl]-isoxazol-3-yl}-phenoxymethyl)-benzoic acid (Compound 162)

From 2-(2,3-difluoro-phenyl)-5H-imidazo[4,5-d]pyridazine and 4-[4-(5-chloromethyl-isoxazol-3-yl)-phenoxymethyl]-benzoic acid. MS 540.7 (M+H⁺); H¹ NMR (DMSO-d₆): δ (ppm) 10.41 (s, 1H), 9.69 (s, 1H), 8.18-8.14 (m, 1H), 7.95-7.93 (m, 2H), 7.79-7.76 (m, 2H), 7.70-7.67 (m, 1H), 7.56-7.54 (d, 2H), 7.47-7.40 (m, 1H), 7.157.11 (m, 3H), 6.47 (s, 2H), 5.24 (s, 2H).

### Example 63

### 5-[3-(4-Difluoromethoxy-3-methoxy-phenyl)-isoxazol-5-ylmethyl-2-(2,3-difluoro-phenyl)-5H-imidazo[4,5-d]pyridazine (Compound 163)

From 2-(2,3-difluoro-phenyl)-5H-imidazo[4,5-d]pyridazine and 5-chloromethyl-3-(4-difluoromethoxy-3-methoxy-phenyl)-isoxazole. MS 486.0 (M+H⁺); H¹ NMR (DMSO-d₆): δ (ppm) 10.27 (s, 1H) 9.61 (s, 1H), 8.17-8.13 (m, 1H), 7.45-7.62 (m, 1H), 7.54 (s, 1H), 7.47-7.37 (m, 2H), 7.28-7.28 (m, 2H), 7.14 (s, 1H), 6.24 (s, 2H), 3.87 (s, 3H).

### Example 64

### 5-[3-(3,5-Bis-trifluoromethyl-phenyl)-isoxazol-5-ylmethyl]-2-(2,3-difluoro-phenyl)-5H-imidazo[4,5-d]pyridazine (Compound 164)

From 2-(2,3-difluoro-phenyl)-5H-imidazo[4,5-d]pyridazine and 3-(3,5-Bis-trifluoromethyl-phenyl)-5-chloromethyl-isoxazole. MS 526.0 (M+H⁺); H¹ NMR (DMSO-d₆): δ (ppm) 10.18 (s, 1H), 9.55 (s, 1H), 8.48 (s, 2H), 8.26 (s, 1H), 8.16-8.12 (m, 1H), 7.61-7.57 (m, 1H), 7.45 (s, 1H), 7.40-7.33 (m, 1H), 6.26 (s, 2H).

### Example 65

### 5-[3-(3-Chloro-4-trifluoromethoxy-phenyl)-isoxazol-5-ylmethyl]-2-(2,3-difluoro-phenyl)-5H-imidazo[4,5-d]pyridazine (Compound 165)

From 2-(2,3-difluoro-phenyl)-5H-imidao[4,5-d]pyridazine and 5-chloromethyl-3-(3-chloro-4-trifluoromethoxy-phenyl)-isoxazole. MS 508.0 (M+H⁺); H¹ NMR (DMSO-d₆): δ (ppm) 10.50 (s, 1H), 9.75 (s, 1H), 8.17-8.13 (t, 2H), 7.98-7.94 (dd, 1H), 7.73-7.69 (m, 2H), 7.48-7.45(m, 1H), 7.34 (s, 1H), 6.36 (s, 2H).

### Example 66

### 2-{5-[2-(2,3-Difluoro-phenyl)-imidazo[4,5-d]pyridazin-5-ylmethyl]-isoxazol-3-yl}-5-methoxy-phenol (Compound 166)

From 2-(2,3-difluoro-phenyl)-5H-imidazo[4,5-d]pyridazine and 2-(5-chloromethyl-isoxazol-3-yl)-5-methoxy-phenol. MS 436.1 (M+H⁺); H¹ NMR (DMSO-d₆): δ (ppm) 10.45 (s, 1H), 10.30 (s, 1H), 9.71 (s, 1H), 8.17-8.14 (m, 1H), 7.74-7.61 (m, 2H), 7.47-7.41 (m, 1H), 7.17 (s, 1H), 6.57-6.56 (d, 1H), 6.50-6.46 (dd, 1H), 6.27 (s, 2H), 3.72 (s, 3H).

### Example 67

### 5-[3-(2,2-Difluoro-benzo[1,3]dioxol-5-yl)-isoxazol-5-ylmethyl]-2-(2,3-difluoro-phenyl)-5H-imidazo[4,5-d]pyridazine (Compound 167)

From 2-(2,3-difluoro-phenyl)-5H-imidazo[4,5-d]pyridazine and 5-chloromethyl-3-(2,2-difluoro-benzo[1,3]dioxol-5-yl)-isoxazole. MS 470.1 (M+H⁺); H¹ NMR (DMSO-d₆): δ (ppm) 10.62 (s, 1H), 9.79 (s, 1H), 8.18-8.14 (m, 1H), 7.89 (d, 1H), 7.75-7.70 (m, 2H), 7.55-7.44 (m, 2H), 7.27 (s, 1H), 6.40 (s, 2H).

### Example 68

### 2-(2,3-Difluoro-phenyl)-5-[3-(3-fluoro-4-trifluoromethoxy-phenyl)-isoxazol-5-ylmethyl]-5H-imidazo[4,5-d]pyridazine (Compound 168)

From 2-(2,3-difluoro-phenyl)-5H-imidazo[4,5-d]pyridazine and 5-chloromethyl-3-(3-fluoro-4-trifluoromethoxy-phenyl)-isoxazole. MS 492.1 (M+H⁺); H¹ NMR (DMSO-d₆): δ (ppm) 10.26 (s, 1H), 9.61 (s, 1H), 8.16-8.12 (m, 1H), 8.03-7.99 (dd, 1H), 7.84-7.81 (d, 1H), 7.74-7.62 (m, 2H), 7.43-7.38 (m, 1H), 7.28 (s, 1H), 6.26 (s, 2H).

### Example 69

### 5-[3-(2,4-Bis-difluoromethoxy-phenyl)-isoxazol-5-ylmethyl]-2-(2,3-difluoro-phenyl)-5H-imidazo[4,5-d]pyridazine (Compound 169)

From 2-(2,3-difluoro-phenyl)-5H-imidazo[4,5-d]pyridazine and 3-(2,4-Bis-difluoromethoxy-phenyl)-5-chloromethyl-isoxazole. MS 522.0 (M+H⁺); H¹ NMR (DMSO-d₆): δ ppm) 10.09 (s, 1H), 9.57 (s, 1H), 8.17-8.13 (m, 1H), 7.90-7.87 (d, 1H), 7.62-7.56 (m, 1H), 7.38-7.32 (m, 2H), 7.20-7.10 (m, 1H), 7.05 (s, 1H), 6.19 (s, 2H), 3.32 (s, 2H).

### Example 70

### 2-(2,3-Difluoro-phenyl)-5-{3-[4-(1,1,2,3,3,3-hexafluoro-propoxy)-phenyl]-isoxazol-5-ylmethyl}-5H-imidazo[4,5-d]pyridazine (Compound 170)

From 2-(2,3-difluoro-phenyl)-5H-imidazo[4,5-d]pyridazine and 5-chloromethyl-3-[4-(1,1,2,3,3,3-hexafluoro-propoxy)-phenyl]-isoxazole. MS 556.0 (M+H⁺); H¹ NMR (DMSO-d₆): δ (ppm) 10.48 (s, 1H), 9.23 (s, 1H), 8.17-8.13 (m, 1H), 7.96-7.93 (d, 2H), 7.75-7.66 (m, 1H), 7.48-7.38 (m, 3H), 7.27 (s, 1H), 6.54-6.52 (m, 1H), 6.40-6.34 (m, 2H).

### Example 71

### 2-(2,3-Difluoro-phenyl)-5-[3-(4-methoxy-2-methyl-phenyl)-isoxazol-5-ylmethyl]-5H-imidazo[4,5-d]pyridazine (Compound 171)

From 2-(2,3-difluoro-phenyl)-5H-imidazo[4,5-d]pyridazine and 5-chloromethyl-3-(4-methoxy-2-methyl-phenyl)-isoxazole. MS 434.1 (M+H⁺); H¹ NMR (DMSO-d₆): δ (ppm) 10.43 (s, 1H), 9.71 (s, 1H), 8.18-8.13 (m, 1H), 7.71-7.67 (m, 1H), 7.48-7.41 (m, 2H), 7.03 (s, 1H), 6.91-6.83 (m, 2H), 6.28 (s, 2H), 3.76 (s, 3H), 2.40 (s, 3H).

### Example 72

### 2-(2,3-Difluoro-phenyl)-5-{3-[4-(pyridin-2-ylmethoxy)-phenyl]-isoxazol-5-ylmethyl}-5H-imidazo[4,5-d]pyridazine (Compound 172)

From 2-(2,3-difluoro-phenyl)-5H-imidazo[4,5-d]pyridazine and 2-[4-(5-chloromethyl-isoxazol-3-yl)-phenoxymethyl]-pyridine. MS 497.0 (M+H⁺); H¹ NMR (DMSO-d₆): δ (ppm) 10.57 (s, 1H), 9.79 (s, 1H), 8.72-8.71 (d, 1H), 8.19-8.12 (m, 2H), 7.82-7.74 (m, 4H), 7.64-7.60 (m, 1H), 7.51-7.47 (m, 1H), 7.19-7.16 (d, 3H), 6.34 (s, 2H), 5.38 (s, 2H).

### Example 73

### 5-[3-(4-Benzyloxy-phenyl)-isoxazal-5-ylmethyl]-2-(2,3-difluoro-phenyl)-5H-imidazo[4,5-d]pyridazine (Compound 173)

From 2-(2,3-difluoro-phenyl)-5H-imidazo[4,5-d]pyridazine and 3-(4-benzyloxy-phenyl)-5-chloromethyl-isoxazole. MS 496.0 (M+H⁺); H¹ NMR (DMSO-d₆): δ (ppm) 10.10 (s, 1H), 9.48 (s, 1H), 8.19-8.14 (m, 1H), 7.80-7.56 (m, 2H), 7.60-7.51 (m, 1H), 7.46-7.31 (m, 6H), 7.14-7.07 (m, 3H), 6.14 (s, 2H), 5.15 (s, 2H).

### Example 74

### 2-(2,3-Difluoro-phenyl)-5-[3-(4-methoxy-2-trifluoromethyl-phenyl)-isoxazol-5-ylmethyl]-5H-imidazo[4,5-d]pyridazine (Compound 174)

From 2-(2,3-difluoro-phenyl)-5H-imidazo[4,5-d]pyridazine and 5-chloromethyl-3-(4-methoxy-2-trifluoromethyl-phenyl)-isoxazole. MS 488.2 (M+H⁺); H¹ NMR (DMSO-d₆): δ (ppm) 10.58 (s, 1H), 9.8 (s, 1H), 8.18-8.14 (m, 1H), 7.75-7.73 (q, 1H), 7.58-7.56 (d, 1H), 7.50-7.44 (m, 1H), 7.377.31 (m, 2H), 6.95 (s, 1H), 6.38 (s, 2H), 3.87 (s, 3H).

### Example 75

### 2-(2,3-Difluoro-phenyl)-5-{3-[4-(1,1,2,2-tetrafluoro-ethoxy)-phenyl]-isoxazol-5-ylmethyl}-5H-imidazo[4,5-d]pyridazine (Compound 175)

From 2-(2,3-difluoro-phenyl)-5H-imidazo[4,5-d]pyridazine and 5-chloromethyl-3-[4-(1,1,2,2-tetrafluoro-ethoxy)-phenyl]-isoxazole. MS 506.1 (M+H⁺); H¹ NMR (DMSO-d₆): δ (ppm) 10.55 (s, 1H), 9.76 (s, 1H), 8.17-8.14 (m, 1H), 7.97-7.92 (m, 2H), 7.74-7.71 (m, 1H), 7.49-7.39 (m, 3H), 7.27 (s, 1H), 7.00-6.66 (t, 1H), 6.37 (s, 2H).

### Example 76

### 5-[3-(4-Difluoromethoxy-phenyl)-isoxazol-5-ylmethyl]-2-(2,3-difluoro-phenyl)-5H-imidazo[4,5-d]pyridazine (Compound 176)

From 2-(2,3-difluoro-phenyl)-5H-imidazo[4,5-d]pyridazine and 5-chloromethyl-3-(4-difluoromethoxy-phenyl)-isoxazole. MS 456.0 (M+H⁺); H¹ NMR (DMSO-d₆): δ (ppm) 10.40 (s, 1H), 9.72 (s, 1H), 8.17-8.13 (t, 1H), 7.91-7.88 (m, 2H), 7.75-7.66 (m, 1H), 7.49-7.41 (m, 1H), 7.30-7.23 (t, 3H), 7.58,7.33,7.09,(t, 1H), 6.31 (s, 2H).

### Example 77

### 2-(2,3-Difluoro-phenyl)-5-[3-(2-methyl-4-trifluoromethoxy-phenyl)-isoxazol-5-ylmethyl]-5H-imidazo[4,5-d]pyridazine (Compound 177)

### 2-Methyl-4-trifluoromethoxy-benzaldehyde

To a solution of 1-bromo-2-methyl-4-trifluoromethoxy-benzene (1.25 g, 5 mmol) in THF (20 mL) at −78° C. was added nBuLi (1.2 eq, 2.4 mL of 2.5M in hexanes). The mixture was stirred for 180 minutes and quenched with DMF (2 mL) and allowed to warm to room temperature. Solvents were removed, the reaction was washed with H₂O (10 mL) and the organics concentrated giving the crude product.

### 2-(2,3-Difluoro-phenyl)-5-[3-(2-methyl-4-trifluoromethoxy-phenyl)-isoxazol-5-ylmethyl]-5H-imidazo[4,5-d]pyridazine (Compound 177)

From 2-(2,3-difluoro-phenyl)-5H-imidazo[4,5-d]pyridazine and 5-chloromethyl-3-(2-methyl-4-trifluoromethoxy-phenyl)-isoxazole (General Procedure B, from 2-methyl-4-trifluoromethoxy-benzaldehyde). MS 488.0 (M+H⁺); H¹ NMR (DMSO-d₆): δ (ppm) 10.47 (s, 1H), 9.73 (s, 1H), 8.17-8.13 (m, 1H), 7.76-7.63 (m, 2H), 7.48-7.39 (m, 2H), 7.32-7.28 (d, 1H), 7.11 (s, 1H), 6.34 (s, 2H), 2.45 (s, 3H).

### Example 78

### 2-(2,3-Difluoro-phenyl)-5-{3-[4-(pyridin-3-yloxymethyl)-phenyl]-isoxazol-5-ylmethyl}-5H-imidazo[4,5-d]pyridazine (Compound 178)

From 2-(2,3-difluoro-phenyl)-5H-imidazo[4,5-d]pyridazine and 3-[4-(5-chloromethyl-isoxazol-3-yl)-benzyloxy]-pyridine. MS 497.2 (M+H⁺); H¹ NMR (DMSO-d₆): δ (ppm) 10.52 (s, 1H), 9.75 (s, 1H), 8.71-8.70 (d, 1H), 8.47-8.45 (d, 1H), 8.19-8.10 (m, 2H), 7.91-7.84 (m, 3H), 7.34-7.71 (m, 1H), 7.61-7.59 (d, 2H), 7.49-7.45 (m, 1H), 7.26 (s, 1H), 6.34 (s, 2H), 5.37 (s, 2H).

### Example 79

### 2-(2,3-Difluoro-phenyl)-5-{3-[4-(pyridin-3-ylmethoxy)-phenyl]-isoxazol-5-ylmethyl}-5H-imidazo[4,5-d]pyridazine (Compound 179)

From 2-(2,3-difluoro-phenyl)-5H-imidazo[4,5-d]pyridazine and 3-[4-(5-chloromethyl-isoxazol-3-yl)-phenoxymethyl]-pyridine. MS 497.2 (M+H⁺); H¹ NMR (DMSO-d₆): δ (ppm) 10.62 (s, 1H), 9.78 (s, 1H), 8.90 (s, 1H), 8.87-8.85 (d, 1H), 8.58-8.55 (d, 1H), 8.18-8.16 (d, 1H), 8.04-7.99 (t, 1H), 7.82-7.73 (m, 3H), 7.50-7.47 (m, 1H), 7.20-7.16 (m, 3H), 6.36 (s, 2H), 5.38 (s, 2H).

### Example 80

### 2-(2,3-Difluoro-phenyl)-5-[3-(4-methyl-thiazol-2-yl)-isoxazol-5-ylmethyl]-5H-imidazo[4,5-d]pyridazine (Compound 180)

From 2-(2,3-difluoro-phenyl)-5H-imidazo[4,5-d]pyridazine and 5-chloromethyl-3-(4-methyl-thiazol-2-yl)-isoxazole. MS 411.0 (M+H⁺); H¹ NMR (DMSO-d₆): δ (ppm) 10.41 (s, 1H), 9.71 (s, 1H), 8.17-8.12 (t, 1H), 7.74-7.69 (m, 1H), 7.53 (s, 1H), 7.45-7.41 (m, 1H), 7.22 (s, 1H), 6.33 (s, 2H), 2.42 (s, 3H).

### Example 81

### 2-(2,3-Difluoro-phenyl)-5-[3-(2-methyl-thiazol-4-yl)-isoxazol-5-ylmethyl]-5H-imidazo[4,5-d]pyridazine (Compound 181)

From 2-(2,3-difluoro-phenyl)-5H-imidazo[4,5-d]pyridazine and 5-chloromethyl-3-(2-methyl-thiazol-4-yl)-isoxazole. MS 411.0 (M+H⁺); H¹ NMR (DMSO-d₆): δ (ppm) 10.67 (s, 1H), 9.83 (s, 1H), 8.19-8.13 (m, 2H), 7.79-7.76 (m, 1H), 7.53-7.49 (m, 1H), 7.13 (s, 1H), 6.40 (s, 2H), 2.68 (s, 3H).

### Example 82

### 5-[3-(2-Butyl-5-chloro-1H-imidazol-4-yl)-isoxazol-5-ylmethyl]-2-(2,3-difluoro-phenyl)-5H-imidazo[4,5-d]pyridazine (Compound 182)

From 2-(2,3-difluoro-phenyl)-5H-imidazo[4,5-d]pyridazine and 3-(2-butyl-5-chloro-1H-imidazol-4-yl)-5-chloromethyl-isoxazole. MS 470.2 (M+H⁺); H¹ NMR (DMSO-d₆): δ (ppm) 10.59 (s, 1H), 9.80 (s, 1H), 8.18-8.13 (t, 1H), 7.80-7.71 (m, 1H), 7.52-7.45 (m, 1H), 7.21 (s, 1H), 6.37 (s, 2H), 2.64-2.59 (t, 2H), 1.63-1.55 (m, 2H), 1.30-1.21 (m, 2H), 0.87-0.83 (t, 3H).

### Example 83

### 5-[3-(2-Butyl-1H-imidazol-4-yl)-isoxazol-5-ylmethyl]-2-(2,3-difluoro-phenyl)-5H-imidazo[4,5-d]pyridazine (Compound 183)

From 2-(2,3-difluoro-phenyl)-5H-imidazo[4,5-d]pyridazine and 3-(2-butyl-1H-imidazol-4-yl)-5-chloromethyl-isoxazole. MS 436.2 (M+H⁺); H¹ NMR (DMSO-d₆): δ (ppm) 10.43 (s, 1H), 9.68 (s, 1H), 8.23 (s, 1H), 8.17-8.13 (m, 1H), 7.69-7.66 (m, 1H), 7.46-7.40 (m, 1H), 7.24 (s, 1H), 6.35 (s, 2H), 2.96-2.91 (m, 2H), 1.75-1.67 (m, 2H), 1.30-1.21 (m, 2H), 0.89-0.83 (m, 3H).

### Example 84

### 2-(2,3-Difluoro-phenyl)-5-[3-(2-ethyl-5-methyl-1H-imidazol-4-yl)-isoxazol-5-ylmethyl]-5H-imidazo[4,5-d]pyridazine (Compound 184)

From 2-(2,3-difluoro-phenyl)-5H-imidazo[4,5-d]pyridazine and 5-chloromethyl-3-(2-ethyl-5-methyl-1H-imidazol-4-yl)-isoxazole. MS 422.2 (M+H); H¹ NMR (DMSO-d₆): δ (ppm) 10.50 (s, 1H), 9.71 (s, 1H), 8.18-8.14 (m, 1H), 7.71-7.68 (m, 1H), 7.47-7.42 (m, 1H), 7.36 (s, 1H), 6.38 (s, 2H), 2.96-2.87 (m, 2H), 2.45 (s, 3H), 1.33-1.28 (m, 3H).

### Example 85

### 2-(2,3-Difluoro-phenyl)-5-[3-(2,5-dimethyl-isoxazol-4-yl)-isoxazol-5-ylmethyl]-5H-imidazo[4,5-d]pyridazine (Compound 185)

From 2-(2,3-difluoro-phenyl)-5H-imidazo[4,5-d]pyridazine and 5-chloromethyl-3-(2,5-dimethyl-oxazol-4-yl)-isoxazole. MS 409.1 (M+H⁺); H¹ NMR (DMSO-d₆): δ (ppm) 10.66 (s, 1H), 9.81 (s, 1H), 8.19-8.14 (m, 1H), 7.81-7.72 (m, 1H), 7.52-7.46 (m, 1H), 6.70 (s, 1H), 6.39 (s, 2H), 2.49 (s, 3H), 2.38 (s, 3H).

### Example 86

### 5-[3-(4-Butyl-2-trifluoromethyl-phenyl)-isoxazol-5-ylmethyl]-2-(2,3-difluoro-phenyl)-5H-imidazo[4,5-d]pyridazine (Compound 186)

### 4-Butyl-2-fluoro-benzaldehyde

4-Bromo-2-fluoro-benzaldehyde (Tetrahedron 61, 6590, 2005) (253 mg, 1.0 mmol), butaneboronic acid (165 mg, 1.6 mmol), potassium carbonate (1.0 mL, 2 M, 2.0 mmol), and toluene (2.0 mL) were combined in a vial and sparged with argon. Tetrakis(triphenylphosphine)palladium (58 mg, 0.05 mmol) was added, and the vial was sealed. The reaction was magnetically stirred at 100° C. overnight. The cooled reaction mixture was extracted with ether (3×4 mL), and the combined extract was concentrated onto celite. The product was isolated by silica gel flash chromatography (EtOAc in hexanes, 0-15%). The product was collected as colorless oil. Yield 165 mg, 72%.

### 5-[3-(4-Butyl-2-trifluoromethyl-phenyl)-isoxazol-5-ylmethyl]-2-(2,3-difluoro-phenyl)-5H-imidazo[4,5-d]pyridazine (Compound 186)

From 2-(2,3-difluoro-phenyl)-5H-imidazo[4,5-d]pyridazine and 3-(4-butyl-2-trifluoromethyl-phenyl)-5-chloromethyl-isoxazole (General Procedure B, from 4-butyl-2-fluoro-benzaldehyde). MS 514.3 (M+H⁺); H¹ NMR (DMSO-d₆): δ (ppm) 10.40 (s, 1H), 10.22 (s, 1H), 9.70 (s, 1H), 8.17-8.13 (m, 1H), 7.72-7.40 (m, 4H), 6.32 (s, 2H), 6.95 (s, 1H), 2.74-2.69 (t, 2H), 1.60-1.52 (m, 2H), 1.33-1.25 (m, 2H), 0.90-0.85 (t, 3H).

### Example 87

### 2-(2,3-Difluoro-phenyl)-5-(3-p-tolyl-isoxazol-5-ylmethyl)-5H-imidazo[4,5-d]pyridazine (Compound 187)

From 2-(2,3-difluoro-phenyl)-5H-imidazo[4,5-d]pyridazine and 5-chloromethyl-3-p-tolyl-isoxazole. MS 404.1 (M+H⁺); H¹ NMR (DMSO-d₆): δ (ppm) 10.53 (s, 1H), 9.75 (s, 1H), 8.18-8.14 (m, 1H), 7.28-7.70 (q, 3H), 7.49-7.42 (m, 1H), 7.30-7.28 (d, 2H), 7.19 (s, 1H), 6.33 (s, 2H), 2.32 (s, 3H).

### Example 88

### 2-(2,3-Difluoro-phenyl)-5-[3-(4-ethyl-phenyl)-isoxazol-5-ylmethyl]-5H-imidazo[4,5-d]pyridazine (Compound 188)

From 2-(2,3-difluoro-phenyl)-5H-imidazo[4,5-d]pyridazine and 5-chloromethyl-3-(4-ethyl-phenyl)-isoxazole. MS 418.1 (M+H⁺); H¹ NMR (DMSO-d₆): δ (ppm) 10.53 (s, 1H) 9.75 (s, 1H), 8.18-8.14 (m, 1H), 7.76-7.67 (m, 3H), 7.49-7.42 (m, 1H), 7.33-7.31 (d, 2H), 7.19 (s, 1H), 6.33 (s, 2H), 2.66-2.59 (q, 2H), 1.19-1.14 (t, 3H).

### Example 89

### 2-(2,3-Difluoro-phenyl)-5-[3-(4-propyl-phenyl)-isoxazol-5-ylmethyl]-5H-imidazo[4,5-d]pyridazine (Compound 189)

From 2-(2,3-difluoro-phenyl)-5H-imidazo[4,5-d]pyridazine and 5-chloromethyl-3-(4-propyl-phenyl)-isoxazole. MS 432.1 (M+H⁺); H¹ NMR (DMSO-d₆): δ (ppm) 10.51 (s, 1H), 9.74 (s, 1H), 8.18-8.14 (m, 1H), 7.74-7.67 (m, 3H), 7.46-7.42 (m, 1H), 7.31-7.28 (d, 2H), 7.19 (s, 1H), 6.32 (s, 2H), 2.60-2.46 (t, 2H), 1.61-1.54 (m, 2H), 0.89-0.84 (t, 3H).

### Example 90

### 2-(2,3-Difluoro-phenyl)-5-[3-(4-isobutyl-phenyl)-isoxazol-5-ylmethyl]-5H-imidazo[4,5-d]pyridazine (Compound 190)

From 2-(2,3-difluoro-phenyl)-5H-imidazo[4,5-d]pyridazine and 5-chloromethyl-3-(4-isobutyl-phenyl)-isoxazole. MS 446.1 (M+H⁺); H¹ NMR (DMSO-d₆): δ (ppm) 10.48 (s, 1H), 9.73 (s, 1H), 8.18-8.14 (m, 1H), 7.75-7.66 (m, 3H), 7.48-7.40 (m, 1H), 7.28-7.23 (d, 2H), 7.19 (s, 1H), 6.31 (s, 2H), 2.45 (m, 2H), 1.90-1.73 (m, 1H), 0.85-0.83 (d, 6H).

### Example 91

### 2-(2,3-Difluoro-phenyl)-5-[3-(4-pentyl-phenyl)-isoxazol-5-ylmethyl]-5H-imidazo[4,5-d]pyridazine (Compound 191)

From 2-(2,3-difluoro-phenyl)-5H-imidazo[4,5-d]pyridazine and 5-chloromethyl-3-(4-pentyl-phenyl)-isoxazole. MS 460.1 (M+H⁺); H¹ NMR (DMSO-d₆): δ (ppm) 10.54 (s, 1H), 9.76 (s, 1H), 8.18-8.15 (m, 1H), 7.74-7.67 (m, 3H), 7.48-7.42 (m, 1H), 7.31-7.28 (d, 2H), 7.19 (s, 1H), 6.34 (s, 2H), 2.61-2.50 (t, 2H), 1.58-1.51 (m, 2H), 1.29-1.20 (m, 4H), 0.85-0.80 (t, 3H).

### Example 92

### 4-(4-{5-[2-(2,3-Difluoro-phenyl)-imidazo[4,5-d]pyridazin-5-ylmethyl]-isoxazol-3-yl}-phenoxy)-butyric acid methyl ester (Compound 192)

A flask was charged with 4-hydroxybenzaldehyde (1.22 g, 10 mmol) and DMF (10 mL). The resulting solution was cooled in an ice bath and treated with NaH (380 mg, 60% in mineral oil, 9.5 mmol). After 5 min, methyl 4-bromobutyrate (1.4 mL, 11 mmol) was added dropwise. The reaction was treated with ultrasound for 15 min, and then stirred overnight at room temp. The reaction mixture was partitioned between ethyl acetate (250 mL) and water (100 mL). The organic layer was washed with water and brine. The organic layer was dried over sodium sulfate and concentrated onto celite. The product was isolated via SiO₂ flash chromatography using 0-5% methanol in dichloromethane to give an oil (1.4 g).

From 2-(2,3-difluoro-phenyl)-5H-imidazo[4,5-d]pyridazine and 3-(4-butyl-2-trifluoromethyl-phenyl)-5-chloromethyl-isoxazole (General Procedure B, from 4-(4-Formyl-phenoxy)-butyric acid methyl ester. MS 506.1 (M+H⁺); H¹ NMR (DMSO-d₆): δ (ppm) 10.09 (d, 1H), 9.48 (d, 1H), 8.14-8.19 (m, 1H), 7.77 (d, 2H), 7.52-7.60 (m, 1H), 7.31-7.37 (m, 1H), 7.12 (s, 1H), 7.01 (d, 2H), 6.14 (s, 2H), 4.03 (t, 2H), 3.59 (s, 3H), 2.44-2.50 (m, 2H), 1.97 (quintet, 2H).

### Example 93

### 3-(4-{5-[2-(2,3-Difluoro-phenyl)-imidazo[4,5-d]pyridazin-5-ylmethyl]-isoxazol-3-yl}-phenoxy)-propan-1-ol (Compound 193)

A flask was charged with 4-hydroxybenzaldehyde (0.96 g, 7.9 mmol) and DMF (10 mL). The resulting solution was cooled in an ice bath and treated with NaH (350 mg, 60% in mineral oil, 8.7 mmol). After 5 min, (3-Bromopropoxy)-tert-butyldimethylsilane (2.0 mL, 8.7 mmol) was added dropwise. The reaction was treated with ultrasound for 15 min, and then stirred overnight at room temp. The reaction mixture was partitioned between ethyl acetate (250 mL) and water (100 mL). The organic layer was washed with water and brine. The organic layer was dried over sodium sulfate and concentrated to give the crude TBS-alcohol. The TBS-alcohol was suspended in 120 mL of a 1:1 mixture of acetonitrile and 1 N HCl. The reaction was stirred at room temp for 1.5 h, and then the solvents were removed in vacuo. The residue was adsorbed onto celite and purified via SiO₂ flash chromatography using 1:1 hexanes:ethyl acetate to give the product alcohol (1.0 g) as a colorless oil.

From 2-(2,3-difluoro-phenyl)-5H-imidazo[4,5-d]pyridazine and 3-(4-butyl-2-trifluoromethyl-phenyl)-5-chloromethyl-isoxazole (General Procedure B, from 4-(3-Hydroxy-phenoxy)-benzaldehyde. MS 464.0 (M+H⁺); H¹ NMR (DMSO-d₆): δ (ppm) 10.08 (d, 1H), 9.47 (d, 1H), 8.13-8.18 (m, 1H), 7.76 (d, 2H), 7.51-7.60 (m, 1H), 7.31-7.38 (m, 1H), 7.12 (s, 1H), 7.02 (d, 2H), 6.14 (s, 2H), 4.56 (t, 1H), 4.07 (t, 2H), 3.55 (q, 2H), 1.86 (m, 2H).

### Example 94

### 2-(2,3-Difluoro-phenyl)-5-{3-[4-(4-methyl-piperazin-1-yl)-phenyl]-isoxazol-5-ylmethyl}-5H-imidazo[4,5-d]pyridazine (Compound 194)

From 2-(2,3-difluoro-phenyl)-5H-imidazo[4,5-d]pyridazine and 1-[4-(5-chloromethyl-isoxazol-3-yl)-phenyl]-4-methyl-piperazine. MS: 488.1 (M+H⁺); H¹-NMR (DMSO-d₆): δ (ppm) 11.0 (bs, 1H), 10.5 (s, 1H), 9.7 (s, 1H), 8.1-8.2 (m, 1H), 7.7 (m, 3H), 7.4 (m, 1H), 7.1 (s, 1H), 7.1 (d, 2H), 6.3 (s, 2H), 3.9 (d, 2H), 3.4 (d, 2H), 3.0-3.2 (m, 4H), 2.8 (d, 3H).

### Example 95

### 2-(2,3-Difluoro-phenyl)-5-{3-[4-(4-methyl-piperazin-1-yl)-phenyl]-isoxazol-5-ylmethyl}-5H-imidazo[4,5-d]pyridazine (Compound 195)

From 2-(2,3-difluoro-phenyl)-5H-imidazo[4,5-d]pyridazine and 5-chloromethyl-3-[4-(2-methoxy-ethoxy)-phenyl]-isoxazole. MS: 464.2 (M+H⁺); H¹-NMR (DMSO-d₆): δ (ppm) 10.3 (s, 1H), 9.7 (s, 1H), 8.1-8.2 (m, 1H), 7.6-7.8 (m, 3H), 7.4 (m, 1H), 7.2 (s, 1H), 7.0 (d, 2H), 6.3 (s, 2H), 4.2 (m, 2H), 3.6 (m, 2H), 3.3 (s, 1H).

### Example 96

### 2-(2,3-Difluoro-phenyl)-5-{3-[4-(2-morpholin-4-yl-ethoxy)-phenyl]-isoxazol-5-ylmethyl}-5H-imidazo[4,5-d]pyridazine (Compound 196)

From 2-(2,3-difluoro-phenyl)-5H-imidazo[4,5-d]pyridazine and 4-{2-[4-(5-chloromethyl-isoxazol-3-yl)-phenoxy]-ethyl}-morpholine. MS: 519.2 (M+H⁺); H¹-NMR (DMSO-d₆): δ (ppm) 10.4 (s, 1H), 9.7 (s, 1H), 8.1-8.2 (m, 1H), 7.8 (d, 2H), 7.7 (m, 1H), 7.4 (m, 1H), 7.2 (s, 1H), 7.0 (d, 2H), 6.3 (s, 2H), 4.4 (m, 2H), 3.9 (m, 2H), 3.8 (m, 2H), 3.4-3.6 (m, 4H), 3.2 (m, 2H).

### Example 97

### 5-{5-[2-(2,3-Difluoro-phenyl)-imidazo[4,5-d]pyridazin-5-ylmethyl]-isoxazol-3-yl}-2-propoxy-benzoic acid propyl ester (Compound 197)

From 2-(2,3-difluoro-phenyl)-5H-imidazo[4,5-d]pyridazine and 5-(5-chloromethyl-isoxazol-3-yl)-2-propoxy-benzoic acid propyl ester. MS: 534.2 (M+H⁺); H¹-NMR (DMSO-d₆): δ (ppm) 10.3 (s, 1H), 9.7 (s, 1H), 8.1-8.2 (m, 1H), 8.1 (s, 1H), 7.9-8.0 (m, 1H), 7.7 (m, 1H), 7.4 (m, 1H), 7.2 (m, 2H), 7.0 (d, 2H), 6.3 (s, 2H), 4.2 (tr, 2H), 4.0 (tr, 2H), 1.6-1.8 (m, 4H), 3.4-3.6 (m, 4H), 0.9-1.0 (m, 6H).

### Example 98

### 2-{5-[2-(2,3-Difluoro-phenyl)-imidazo[4,5-d]pyridazin-5-ylmethyl]-isoxazol-3-yl}-5-methoxy-benzoic acid methyl ester (Compound 198)

From 2-(2,3-difluoro-phenyl)-5H-imidazo[4,5-d]pyridazine and

5-(5-chloromethyl-isoxazol-3-yl)-2-methoxy-benzoic acid methyl ester. MS: 478.1 (M+H⁺); H¹-NMR (DMSO-d₆): δ (ppm) 10.3 (s, 1H), 9.6 (s, 1H), 8.2 (m, 1H), 7.6-7.7 (m, 2H), 7.4 (m, 1H), 7.3 (d, 1H), 7.2 (dd,1H), 6.9 (s, 1H), 6.3 (s, 2H), 3.8 (s, 3H), 3.7 (s, 3H).

### Example 99

### 2-(2,3-Difluoro-phenyl)-5-[3-(4-nitro-phenyl)-isoxazol-5-ylmethyl]-5H-imidazo[4,5-d]pyridazine (Compound 199)

From 2-(2,3-difluoro-phenyl)-5H-imidazo[4,5-d]pyridazine and 5-chloromethyl-3-(4-nitro-phenyl)-isoxazole. MS: 435.1 (M+H⁺); H¹-NMR (DMSO-d₆): δ (ppm) 10.2 (s, 1H), 9.6 (s, 1H), 8.8 (m, 1H), 8.3 (d, 2H), 8.1 (d,2H), 7.8 (m, 1H), 7.6 (m, 1H), 7.4 (m, 2H), 6.3 (s, 2H).

### Example 100

### 5-[3-(4-Bromo-phenyl)-isoxazol-5-ylmethyl]-2-(2,3-difluoro-phenyl)-5H-imidazo[4,5-d]pyridazine (Compound 200)

From 2-(2,3-difluoro-phenyl)-5H-imidazo[4,5-d]pyridazine and 3-(4-bromo-phenyl)-5-chloromethyl-isoxazole. MS: 468.0 (M+H⁺); H¹-NMR (DMSO-d₆): δ (ppm) 10.3 (d, 1H), 9.6 (d, 1H), 8.1 (m, 1H), 7.8 (d, 2H), 7.7 (d, 2H), 7.6 (m, 1H), 7.5 (m, 1H), 7.2 (s, 1H), 6.2 (s, 2H).

### Example 101

### 5-[3-(4-Butyl-phenyl)-isoxazol-5-ylmethyl]-2-(2,3-difluoro-phenyl)-5H-imidazo[4,5-d]pyridazine (Compound 201)

From 2-(2,3-difluoro-phenyl)-5H-imidazo[4,5-d]pyridazine and 3-(4-butyl-phenyl)-5-chloromethyl-isoxazole. MS: 446.1 (M+H⁺); H¹ NMR (DMSO-d₆): δ (ppm) 10.26 (s, 1H), 9.6 (d, (s, 1H), 8.11-8.20 (m, 1H), 7.71-7.77 (m, 2H), 7.55-7.69 (m, 1H), 7.27-7.45 (m, 3H), 7.18 (m, 1H), 6.23 (s, 2H), 2.62 (t, 2H), 1.48-1.63 (m, 2H), 1.21-1.36 (m, 2H), 0.90 (t, 3H).

### Example 102

### 2-(2,3-Difluoro-phenyl)-5-[3-(4-trifluoromethyl-phenyl)-isoxazol-5-ylmethyl]-5H-imidazo[4,5-d]pyridazine (Compound 202)

From 2-(2,3-difluoro-phenyl)-5H-imidazo[4,5-d]pyridazine and 5-chloromethyl-3-(4-trifluoromethyl-phenyl)-isoxazole MS: 458.1 (M+H⁺); H¹ NMR (DMSO-d₆): δ (ppm) 10.46 (s, 1H), 9.73 (s, 1H), 8.04-8.20 (m, 3H), 7.84-7.91 (m, 2H), 7.64-7.77 (m, 1H), 7.34-7.50 (m, 2H), 6.35 (s, 1H).

### Example 103

### 2-(2,3-Difluoro-phenyl)-5-[3-(2-fluoro-4-trifluoromethyl-phenyl)-isoxazol-5-ylmethyl-5H-imidazo[4,5-d]pyridazine (Compound 203)

From 2-(2,3-difluoro-phenyl)-5H-imidazo[4,5-d]pyridazine and 5-chloromethyl-3-(2-fluoro-4-trifluoromethyl-phenyl)-isoxazole. MS: 476.1 (M+H⁺); H¹ NMR (DMSO-d₆): δ (ppm) 10.39 (s, 1H), 9.68 (s, 1H), 8.07-8.20 (m, 2H), 7.90-7.98 (m, 1H), 7.62-7.77 (m, 2H), 7.38-7.48 (m, 1H), 7.24-7.29 (m, 1H), 6.34 (s, 1H).

### Example 104

### 2-(2,3-Difluoro-phenyl)-5-[3-(3-fluoro-pyridin-4-yl)-isoxazol-5-ylmethyl]-5H-imidazo[4,5-d]pyridazine (Compound 204)

From 2-(2,3-difluoro-phenyl)-5H-imidazo[4,5-d]pyridazine and 4-(5-chloromethyl-isoxazol-3-yl)-3-fluoro-pyridine. MS: 409.1 (M+H⁺); H¹ NMR (DMSO-d₆): δ (ppm) 10.60 (s, 1H), 9.81 (s, 1H), 8.78-8.83 (m, 1H), 8.55-8.61 (m, 1H), 8.12-8.20 (m, 1H), 7.87-7.95 (m, 1H), 7.73-7.82 (m, 1H), 7.45-7.55 (m, 1H), 7.31-7.36 (m, 1H), 6.44 (s, 2H).

### Example 105

### 2-(2,3-Difluoro-phenyl)-5-[3-(6-trifluoromethyl-pyridin-3-yl)-isoxazol-5-ylmethyl]-5H-imidazo[4,5-d]pyridazine (Compound 205)

From 2-(2,3-difluoro-phenyl)-5H-imidazo[4,5-d]pyridazine and 5-(5-chloromethyl-isoxazol-3-yl)-2-trifluoromethyl-pyridine. MS: 459.1 (M+H⁺); H¹ NMR (DMSO-d₆): δ (ppm) 10.24 (s, 1H), 9.58 (s, 1H), 9324 (s, 1H), 8.50-8.57 (m, 1H), 8.11-8.19 (m, 1H), 8.02-8.09 (m, 1H), 7.56-7.68 (m, 1H), 7.34-7.44 (m, 2H), 6.29 (s, 2H).

### Example 106

### 2-(2,3-Difluoro-phenyl)-5-[3-(3-fluoro-4-trifluoromethyl-phenyl)-isoxazol-5-ylmethyl]-5H-imidazo[4,5-d]pyridazine (Compound 206)

From 2-(2,3-difluoro-phenyl)-5H-imidazo[4,5-d]pyridazine and 5-chloromethyl-3-(3-fluoro-4-trifluoromethyl-phenyl)-isoxazole. MS: 476.1 (M+H⁺); H¹ NMR (DMSO-d₆): δ (ppm) 10.18 (s, 1H), 9.56 (s, 1H), 8.10-8.20 (m, 1H), 7.88-8.07 (m, 3H), 7.55-7.65 (m, 1H), 7.30-7.42 (m, 2H), 6.25 (s, 2H).

### Example 107

### 2-(2,3-Difluoro-phenyl)-5-{3-[4-(3-fluoro-propoxy)-phenyl]-isoxazol-5-ylmethyl}-5H-imidazo[4,5-d]pyridazine (Compound 207)

From 2-(2,3-difluoro-phenyl)-5H-imidazo[4,5-d]pyridazine and 5-chloromethyl-3-[4-(3-fluoro-propoxy)-phenyl]-isoxazole. MS: 466.1 (M+H⁺); H¹ NMR (DMSO-d₆): δ (ppm) 10.17 (s, 1H), 9.54 (s, 1H), 8.11-8.19 (s, 1H), 7.73-7.81 (m, 2H), 7.53-7.66 (m, 1H), 7.32-7.43 (m, 1H), 7.14 (s, 1H), 7.00-7.09 (m, 2H), 6.18 9s, 2H), 4.68 (t, 1H), 4.53 (t, 1H), 4.12 (t, 2H), 2.01-2.22 (m, 2H).

### Example 108

### (4-{5-[2-(2,3-Difluoro-phenyl)-imidazo[4,5-d]pyridazin-5-ylmethyl]-isoxazol-3-yl}-phenyl)-dimethyl-amine (Compound 208)

From 2-(2,3-difluoro-phenyl)-5H-imidazo[4,5-d]pyridazine and [4-(5-chloromethyl-isoxazol-3-yl)-phenyl]-dimethyl-amine. MS: 433.2 (M+H⁺); H¹ NMR (DMSO-d₆): δ (ppm) 10.21 (s, 1H), 9.57 (s, 1H), 8.11-8.19 (m, 1H), 7.60-7.68 (m, 3H), 7.34-7.44 (m, 1H), 7.07 (s, 1H), 6.71-6.79 (m, 2H), 6.17 (s, 2H), 2.96 (s, 6H).

### Example 109

### 4-{5-[2-(2,3-Difluoro-phenyl)-imidazo[4,5-d]pyridazin-5-ylmethyl]-isoxazol-3-yl}-benzoic acid methyl ester (Compound 209)

From 2-(2,3-difluoro-phenyl)-5H-imidazo[4,5-d]pyridazine and 4-(5-chloromethyl-isoxazol-3-yl)-benzoic acid methyl ester. MS: 448.1 (M+H⁺); H¹ NMR (DMSO-d₆): δ (ppm) 10.12 (s, 1H), 9.51 (s, 1H), 8.11-8.20 (m, 1H), 7.77-7.84 (m, 1H), 7.52-7.73 (m, 4H), 7.30-7.40 (m, 1H), 6.94 (s, 1H), 6.18 (s, 2H), 3.68 (s, 3H).

### Example 110

### 3-{5-[2-(2,3-Difluoro-phenyl)-imidazo[4,5-d]pyridazin-5-ylmethyl]-isoxazol-3-yl}-benzoic acid methyl ester (Compound 210)

From 2-(2,3-difluoro-phenyl)-5H-imidazo[4,5-d]pyridazine and 3-(5-chloromethyl-isoxazol-3-yl)-benzoic acid methyl ester. MS: 448.1 (M+H⁺); H¹ NMR (DMSO-d₆): δ (ppm) 10.43 (s, 1H), 9.71 (s, 1H), 8.34-8.39 (m, 1H), 8.02-8.20 (m, 3H), 7.64-7.74 (m, 2H), 7.39-7.50 (m, 1H), 7.36 (s, 1H), 6.33 (s, 2H), 3.88 (s, 3H).

### Example 111

### 2-{5-[2-(2,3-Difluoro-phenyl)-imidazo[4,5-d]pyridazin-5-ylmethyl]-isoxazol-3-yl}-benzoic acid methyl ester (Compound 211)

From 2-(2,3-difluoro-phenyl)-5H-imidazo[4,5-d]pyridazine and 2-(5-chloromethyl-isoxazol-3-yl)-benzoic acid methyl ester. MS: 448.1 (M+H⁺); H¹ NMR (DMSO-d₆): δ (ppm) 10.26 (s, 1H), 9.62 (s, 1H), 8.12-8.18 (m, 1H), 7.77-7.84 (m, 1H), 7.57-7.72 (m, 4H), 7.35-7.44 (m, 1H), 6.95 (s, 1H), 6.24 (s, 2H), 6.69 (s, 3H).

### Example 112

### 3-{5-[2-(2,3-Difluoro-phenyl)-imidazo[4,5-d]pyridazin-5-ylmethyl]-isoxazol-3-yl}-benzonitrile (Compound 212)

From 2-(2,3-difluoro-phenyl)-5H-imidazo[4,5-d]pyridazine and 3-(5-chloromethyl-isoxazol-3-yl)-benzonitrile. MS: 415.1 (M+H⁺); H¹ NMR (DMSO-d₆): δ (ppm) 10.38 (s, 1H), 9.69 (s, 1H), 8.29-8.34 (m, 1H), 8.11-8.23 (m, 2H), 7.94-8.01 (m, 1H), 7.62-7.76 (m, 2H), 7.38-7.66 (m, 1H), 7.32 (s, 1H), 6.33 (s, 2H).

### Example 113

### 4-{5-[2-(2,3-Difluoro-phenyl)-imidazo[4,5-d]pyridazin-5-ylmethyl]-isoxazol-3-yl}-benzonitrile (Compound 213)

From 2-(2,3-difluoro-phenyl)-5H-imidazo[4,5-d]pyridazine and 4-(5-chloromethyl-isoxazol-3-yl)-benzonitrile. MS: 415.1 (M+H⁺); H¹ NMR (DMSO-d₆): δ (ppm) 10.23 (s, 1H), 9.58 (s, 1H), 8.12-8.18 (m, 1H), 7.94-8.09 (m, 4H), 7.56-7.68 (m, 1H), 7.31-7.44 (m, 2H), 6.26 (s, 2H).

### Example 114

### 2-(2,3-Difluoro-phenyl)-5-[3-(4-trifluoromethoxy-phenyl)-isoxazol-5-ylmethyl]-5H-imidazo[4,5-d]pyridazine (Compound 214)

From 2-(2,3-difluoro-phenyl)-5H-imidazo[4,5-d]pyridazine and 5-chloromethyl-3-(4-trifluoromethoxy-phenyl)-isoxazole. MS: 474.1 (M+H⁺); H¹ NMR (DMSO-d₆): δ (ppm) 10.21 (s, 1H), 9.57 (s, 1H), 8.11-8.19 (m, 1H), 7.95-8.02 (m, 2H), 7.34-7.67 (m, 4H), 7.25 (s, 1H), 6.24 (s, 2H).

### Example 115

### (4-{5-[2-(2,3-Difluoro-phenyl)-imidazo[4,5-d]pyridazin-5-ylmethyl]-isoxazol-3-yl}-phenoxy)-acetic acid methyl ester (Compound 215)

From 2-(2,3-difluoro-phenyl)-5H-imidazo[4,5-d]pyridazine and [4-(5-chloromethyl-isoxazol-3-yl)-phenoxy]-acetic acid methyl ester. MS: 478.1 (M+H⁺); H¹ NMR (DMSO-d₆): δ (ppm) 10.09 (s, 1H), 9.47 (s, 1H), 8.11-8.20 (m, 1H), 7.74-7.81 (m, 2H), 7.48-7.61 (m, 1H), 7.29-7.40 (m, 1H), 7.13 (s, 1H), 7.00-7.06 (m, 2H), 6.07 (s, 2H), 4.69 (s, 2H), 3.70 (s, 3H).

### Example 116

### [3-(4-{5-[2-(2,3-Difluoro-phenyl)-imidazo[4,5-d]pyridazin-5-ylmethyl]-isoxazol-3-yl}-phenoxy)-propyl]-dimethyl-amine (Compound 216)

From 2-(2,3-difluoro-phenyl)-5H-imidazo[4,5-d]pyridazine and {3-[4-(5-chloromethyl-isoxazol-3-yl)-phenoxy]-propyl}-dimethyl-amine. MS: 491.2 (M+H⁺); H¹ NMR (DMSO-d₆): δ (ppm) 10.18 (s, 1H), 9.54 (s, 1H), 8.11-8.20 (m, 1H), 7.76-7.83 (m, 2H), 7.52-7.68 (m, 1H), 7.33-7.43 (m, 1H), 7.14 (s, 1H), 7.00-7.07 (m, 2H), 6.19 (s, 2H), 4.10 (t, 2H), 3.15-3.26 (m, 2H), 7.76-2.82 (m, 6H), 2.06-2.18 (m, 2H).

### Example 117

### 2-(2,3-Difluoro-phenyl)-5-[3-(4-pyridin-2-ylmethyl-phenyl)-isoxazol-5-ylmethyl]-5H-imidazo[4,5-d]pyridazine (Compound 217)

From 2-(2,3-difluoro-phenyl)-5H-imidazo[4,5-d]pyridazine and 2-[4-(5-chloromethyl-isoxazol-3-yl)-benzyl]-pyridine. MS: 483.1 (M+H⁺); H¹ NMR (DMSO-d₆): δ (ppm) 10.70 (s, 1H), 9.88 9s, 1H), 8.11-8.20 (m, 2H), 7.73-7.92 (m, 4H), 7.46-7.57 (m, 1H), 7.04-7.50 (m, 5H), 6.42 (s, 2H).

### Example 118

### (4-{5-[2-(2,3-Difluoro-phenyl)-imidazo[4,5-d]pyridazin-5-ylmethyl]-isoxazol-3-yl}-benzyl)-dimethyl-amine (Compound 218)

From 2-(2,3-difluoro-phenyl)-5H-imidazo[4,5-d]pyridazine and [4-(5-chloromethyl-isoxazol-3-yl)-benzyl]-dimethyl-amine. Additional purification by silica gel chromatography gave the desired product. MS: 447.1 (M+H⁺); H¹ NMR (DMSO-d₆): δ (ppm) 10.09 (s, 1H), 9.48 (s, 1H), 8.11-8.20 (m, 1H), 7.77-7.84 (m, 2H), 7.48-7.62 (m, 1H), 7.29-7.44 (m, 3H), 7.18 (s, 1H), 6.17 (s, 2H), 3.50 (s, 2H), 2.20 (s, 6H).

### Example 119

### 2-(2,3-Difluoro-phenyl)-5-[3-(4-pyrolidin-1-ylmethyl-phenyl)-isoxazol-5-ylmethyl]-5H-imidazo[4,5-d]pyridazine (Compound 219)

From 2-(2,3-difluoro-phenyl)-5H-imidazo[4,5-d]pyridazine and 5-chloromethyl-3-(4-pyrrolidin-1-ylmethyl-phenyl)-isoxazole. MS: 473.2 (M+H⁺); H¹ NMR (DMSO-d₆): δ (ppm) 10.44 (s, 1H), 9.70 (s, 1H), 8.12-8.20 (m, 1H), 7.88-7.94 (m, 2H), 7.63-7.76 (m, 3H), 7.39-7.50 (m, 1H), 7.28 (s, 1H), 6.33 (s, 2H), 4.34-4.40 (m, 2H), 3.26-3.40 (m, 1H), 2.96-3.10 (m, 1H), 1.82-2.05 (m, 4H).

### Example 120

### 2-(2,3-Difluoro-phenyl)-5-[3-(4-ethoxy-phenyl)-isoxazol-5-ylmethyl]-5H-imidazo[4,5-d]pyridazine (Compound 220)

From 2-(2,3-difluoro-phenyl)-5H-imidazo[4,5-d]pyridazine and 5-chloromethyl-3-(4-ethoxy-phenyl)-isoxazole. MS: 434.1 (M+H⁺); H¹ NMR (DMSO-d₆): δ (ppm) 10.42 (s, 1H), 9.71 (s, 1H), 8.13-8.22 (m, 1H), 7.64-7.81 (m, 3H), 7.40-7.51 (m, 1H), 7.17 (s, 1H), 6.97-7.06 (m, 2H), 6.28 (s, 2H), 4.07 (q, 2H), 1.33 (t, 3H).

### Example 121

### 2-(2,3-Difluoro-phenyl)-5-[3-(4-methoxy-phenyl)-isoxazol-5-ylmethyl]-5H-imidazo[4,5-d]pyridazine (Compound 221)

From 2-(2,3-difluoro-phenyl)-5H-imidazo[4,5-d]pyridazine and 5-chloromethyl-3-(4-methoxy-phenyl)-isoxazole. MS: 420.2 (M+H⁺); H¹ NMR (DMSO-d₆): δ (ppm) 10.48 (s, 1H), 9.74 (s, 1H), 8.13-8.22 (m, 1H), 7.66-7.84 (m, 3H), 7.41-7.52 (m, 1H), 7.18 (s, 1H), 7.00-7.09 (m, 2H), 6.31 (s, 2H), 3.80 (s, 3H).

### Example 122

### 5-[3-(4-Butoxy-phenyl)-isoxazol-5-ylmethyl]-2-(2,3-difluoro-phenyl)-5H-imidazo[4,5-d]pyridazine (Compound 222)

From 2-(2,3-difluoro-phenyl)-5H-imidazo[4,5-d]pyridazine and 3-(4-butoxy-phenyl)-5-chloromethyl-isoxazole. MS: 462.3 (M+H⁺); H¹ NMR (DMSO-d₆): δ (ppm) 10.43 (s, 1H), 9.72 (s, 1H), 8.13-8.21 (m, 1H), 7.64-7.81 (m, 3H), 7.40-7.53 (m, 1H), 7.17 (s, 1H), 7.00-7.07 (m, 2H), 6.29 (s, 2H), 4.02 (t, 2H), 1.63-1.76 (m, 2H), 1.34-1.51 (m, 2H), 0.93 (t, 3H).

### Example 123

### 5-[3-(2,4-Bis-trifluoromethyl-phenyl)-isoxazol-5-ylmethyl]-2-phenyl-5H-imidazo[4,5-d]pyridazine (Compound 223)

From 3-(2,4-bis-trifluoromethyl-phenyl)-5-chloromethyl-isoxazole and 2-phenyl-5H-imidazo[4,5-d]pyridazine. MS 490 (M+H⁺); H¹ NMR (DMSO-d₆): δ (ppm) 10.45 (s, 1H), 9.74 (s, 1H), 8.4 (m, 2H), 8.2 (m, 2H), 7.91 (m, 1H), 7.65 (s, 3H), 7.03 (s, 1H), 6.38 (s, 2H).

### Example 124

### 2-Phenyl-5-[3-(4-propoxy-phenyl)-isoxazol-5-ylmethyl]-5H-imidazo[4,5-d]pyridazine (Compound 224)

From 35-chloromethyl-3-(4-propoxy-phenyl)-isoxazole and 2-phenyl-5H-imidazo[4,5-d]pyridazine. MS 412.1 (M+H⁺); H¹ NMR (DMSO-d₆): δ (ppm) 10.37 (s, 1H), 9.69 (s, 1H), 8.4 (m, 2H), 7.76 (m, 2H), 7.63 (m, 3H), 7.15 (s, 1H), 7.03 (d, 2H), 6.26 (s, 2H), 3.98 (t, 2H), 1.74 (m, 2H), 0.99 (q, 3H).

### Example 125

### 5-[3-(4-Butoxy-phenyl)-isoxazol-5-ylmethyl]-2-phenyl-5H-imidazo[4,5-d]pyridazine (Compound 225)

From 3-(4-butoxy-phenyl)-5-chloromethyl-isoxazole and 2-phenyl-5H-imidazo[4,5-d]pyridazine. MS 426.1 (M+H⁺); H¹ NMR (DMSO-d₆): δ (ppm) 10.23 (s, 1H), 9.57 (s, 1H), 8.34 (m, 2H), 7.7 (m, 2H), 7.54,(m, 3H), 7.08 (s, 1H), 6.97 (m, 2H), 6.17 (s, 2H), 3.94 (t, 2H), 1.63 (m, 2H), 1.37 (m, 2H), 0.88 (t, 3H).

### Example 126

### 5-{1-[3-(2,4-Bis-trifluoromethyl-phenyl)-isoxazol-5-yl]-ethyl}-2-(2,3-difluoro-phenyl)-5H-imidazo[4,5-d]pyridazine (Compound 226)

From 2-(2,3-Difluoro-phenyl)-5H-imidazo[4,5-d]pyridazine and methanesulfonic acid 1-[3-(2,4-bis-trifluoromethyl-phenyl)-isoxazol-5-yl]-ethyl ester. MS 540.1 (M+H⁺); H¹ NMR (DMSO-d₆): δ (ppm) 10.33 (s, 1H), 9.65 (s, 1H), 8.11-8.26 (m, 3H), 7.90 (m, 1H), 7.6 (m, 1H), 7.40 (m, 1H), 7.01 (s, 1H), 6.64 (q, 1H), 2.1 (d, 3H).

### Example 127

### 5-{1-[3-(2,4-Bis-trifluoromethyl-phenyl)-isoxazol-5-yl]-1-methyl-ethyl}-2-(2,3-difluoro-phenyl)-5H-imidazo[4,5-d]pyridazine (Compound 227)

From 2-(2,3-Difluoro-phenyl)-5H-imidazo[4,5-d]pyridazine and 3-(2,4-Bis-trifluoromethyl-phenyl)-5-(1-chloro-1-methyl-ethyl)-isoxazole. MS 554.1 (M+H⁺); H¹ NMR (DMSO-d₆): δ (ppm) 10.44 (s, 1H), 9.69 (s, 1H), 8.24 (m, 2H), 8.10 (m, 2H), 7.99 (m, 1H), 7.71 (m, 1H), 7.44 (m, 1H), 7.12 (s, 1H), 2.3 (s, 6H).

### Example 128

### 2-(2,3-Difluoro-phenyl)-5-[3-(4-methoxy-phenyl)-[1,2,4]oxadiazol-5-ylmethyl]-5H-imidazo[4,5-d]pyridazine (Compound 228)

From 2-(2,3-difluoro-phenyl)-5H-imidazo[4,5-d]pyridazine and 5-chloromethyl-3-(4-methoxy-phenyl)-[1,2,4]oxadiazole (similar to General Procedure B, using corresponding oxadiazole derivatives in place of isoxazole derivatives). MS 421.1 (M+H⁺); H¹ NMR (DMSO-d₆): δ (ppm) 10.11 (d, 1H), 9.50 (d, 1H), 8.14-8.20 (m, 1H), 7.87 (d, 2H), 7.51-7.60 (m, 1H), 7.31-7.38 (m, 1H), 7.06 (d, 2H), 6.40 (s, 1H), 3.80 (s, 3H).

### Example 129

### 2-(2,3-Difluoro-phenyl)-5-[5-(4-methoxy-phenyl)-[1,3,4]oxadiazol-2-ylmethyl]-5H-imidazo[4,5-d]pyridazine (Compound 229)

From 2-(2,3-difluoro-phenyl)-5H-imidazo[4,5-d]pyridazine and 2-chloromethyl-5-(4-methoxy-phenyl)-[1,3,4]oxadiazole (similar to General Procedure B, using corresponding oxadiazole derivatives in place of isoxazole derivatives). MS 421.1 (M+H⁺); H¹ NMR (DMSO-d₆): δ (ppm) 10.10 (d, 1H), 9.47 (d, 1H), 8.14-8.19 (m, 1H), 7.91 (d, 2H), 7.52-7.60 (m, 1H), 7.31-7.37 (m, 1H), 7.11 (d, 2H), 6.30 (s, 1H), 3.82 (s, 3H).

### Example 130

### 2-(2,3-Difluoro-phenyl)-5-[5-(4-trifluoromethyl-phenyl)-[1,2,4]oxadiazol-3-ylmethyl]-5H-imidazo[4,5-d]pyridazine (Compound 230)

From 2-(2,3-difluoro-phenyl)-5H-imidazo[4,5-d]pyridazine and 3-chloromethyl-5-(4-trifluoromethyl-phenyl)-[1,2,4]oxadiazole (similar to General Procedure B, using corresponding oxadiazole derivatives in place of isoxazole derivatives). MS 458.9 (M+H⁺); H¹ NMR (DMSO-d₆): δ (ppm) 10.13 (d, 1H), 9.48 (d, 1H), 8.28 (d, 2H), 8.14-8.19 (m, 1H), 7.96 (d, 2H), 7.51-7.59 (m, 1H), 7.32-7.37 (m, 1H), 6.24 (s, 2H).

### Example 131

### 5-[3-(2,4-Bis-trifluoromethyl-phenyl)-isoxazol-5-ylmethyl]-2-pyridin-2-yl-5H-imidazo[4,5-d]pyridazine (Compound 231)

### 2-(1H-Imidazol-2-yl)-pyridine

A round bottom flask was charged with 2-pyridinecarboxaldehyde (5.0 g), glyoxal (10.7 mL, 40% in water), and methanol (100 mL). This mixture was stirred at room temp as 26 mL of concentrated aqueous ammonia was added portion-wise. After 1 h, the solvents were removed, and the remaining brown residue was recrystallized in acetonitrile (ca. 40 mL). The product 2-(1H-Imidazol-2-yl)-pyridine was collected as brown crystals.

### 2-Pyridin-2-yl-5H-imidazo[4,5-d]pyridazine

A portion of 2-(1H-Imidazol-2-yl)-pyridine (61 mg, 0.42 mmol) and dimethyl 1,2,4,5-tetrazine-3,6-dicarboxylate (165 mg, 0.84 mmol) were ground together and heated slowly with a heat gun (caution!) in a vial until vigorous evolution of gas was observed. The cooled crude product was combined with DMF (ca. 3 mL) and a few drops of TFA were added. An off-white solid precipitated and was collected. This solid was dissolved in 7 mL of a 2:1 mixture of acetic acid and conc. HCl, and this solution was heated to 95° C. for 3 h. The solvents were removed in vacuo to give 237 mg of the crude 2-pyridin-2-yl-5H-imidazo[4,5-d]pyridazine.

### 5-[3-(2,4-Bis-trifluoromethyl-phenyl)-isoxazol-5-ylmethyl]-2-pyridin-2-yl-5H-imidazo[4,5-d]pyridazine (Compound 231)

2-Pyridin-2-yl-5H-imidazo[4,5-d]pyridazine was coupled to 3-(2,4-bis-trifluoromethyl-phenyl)-5-chloromethyl-isoxazole according General Procedure H.

MS 491.1 (M+H⁺); H¹ NMR (DMSO-d₆): δ (ppm) 10.08 (d, 1H), 9.46 (d, 1H), 8.71-8.73 (m, 1H), 8.43-8.46 (m, 1H), 8.24 (s, 1H), 8.21 (d, 1H), 7.91-7.97 (m, 2H), 7.45-7.49 (m, 1H), 7.05 (s, 1H), 6.24 (s, 2H).

### Example 132

### 5-[2-(4-Chloro-phenyl)-3H-imidazol-4-ylmethyl-2-(2,3-difluoro-phenyl)-5H-imidazo[4,5-d]pyridazine (Compound 232)

4-Chloro-benzonitrile was dissolved in ethanol and HCl was bubbled through the solution for 1 h. The reaction flask was sealed and stored in the freezer overnight. The solvents were removed in vacuo to give 4-chloro-benzimidic acid ethyl ester. The 4-chloro-benzimidic acid ethyl ester was placed in a Parr high-pressure apparatus and 1 equivalent of 1,3-dihydroxyacetone (as the dimer) was added. Liquid NH₃ (ca. 20 mL) was introduced, and the apparatus was sealed and heated to 60° C. overnight. The NH₃ was allowed to evaporate, and the remaining residue was triturated with isopropanol. The isopropanol was concentrated to give [2-(4-chloro-phenyl)-3H-imidazol-4-yl]-methanol. The [2-(4-chloro-phenyl)-3H-imidazol-4-yl]-methanol (45 mg, 0.22 mmol) was suspended in benzene (2 mL) and SOCl₂ (0.05 mL) was added. The reaction was stirred at 78° C. for 3 h, and then the solvents were removed in vacuo.

The crude chloromethyl derivative was coupled to 2-(2,3-difluoro-phenyl)-5H-imidazo[4,5-d]pyridazine according to General Procedure H. MS 423.1 (M+H⁺); H¹ NMR (DMSO-d₆): δ (ppm) 10.60 (s, 1H), 9.76 (s, 1H), 8.12-8.20 (m, 3H), 7.96 (s, 1H), 7.67-7.80 (m, 3H), 7.45-7.52 (m, 1H), 6.21 (s, 1H).

### Example 133

### 6-[3-(2,4-Bis-trifluoromethyl-phenyl)-isoxazol-5-ylmethyl]-2-(2,3-difluoro-phenyl)-6H-imidazo[4,5-d]pyridazin-4-ylamine (Compound 233)

### 2-(2,3-Difluoro-phenyl)-5H-imidazo[4,5-d]pyridazin-4-ylamine

To 2-(2,3-difluoro-phenyl)-1H-imidazole-4,5-dicarbonitrile (1.15 g) in THF (50 mL) at −78° C. was added DIBALH (12.5 mL of 1 M in THF, 2.5 eq.) dropwise and warmed to RT. Hydrazine (5 mL, excess) was added and the mixture stirred for 1 hr. The solvents were removed and the product purified on silica gel 0-20% MeOH CH₂Cl₂. By ¹H NMR the product appeared to be the uncyclized hydrazone. The intermediate was dissolved in MeOH (½ mL) and heated to 145° C. under μ-wave irradiation for 15 min. The MeOH was removed and the product purified on silica gel 0-10% MeOH CH₂Cl₂ yielding the desired product.

### 6-[3-(2,4-Bis-trifluoromethyl-phenyl)-isoxazol-5-ylmethyl]-2-(2,3-difluoro-phenyl)-6H-imidazo[4,5-d]pyridazine-4-ylamine (Compound 233)

Following a procedure similar to General Procedure H, from 3-(2,4-bis-trifluoromethyl-phenyl)-5-chloromethyl-isoxazole and 2-(2,3-difluoro-phenyl)-5H-imidazo[4,5-d]pyridazine-4-ylamine (in place of 2-(2,3-difluoro-phenyl)-5H-imidazo[4,5-d]pyridazine). MS 541.1 (M+H⁺); H¹ NMR (DMSO-d₆): δ (ppm) 9.53 (s, 1H), 8.22 (m, 2H), 8.05 (m, 1H), 7.94 (m, 1H), 7.49 (m, 1H), 7.31 (m, 1H), 7.14 (br s, 2H), 6.98 (s, 1H), 5.94 (s, 2H).

### Example 134

### 2-(2,3-Difluoro-phenyl)-6-[3-(4-trifluoromethyl-phenyl)-isoxazol-5-ylmethyl]-6H-imidazo[4,5-d]pyridazin-4-ylamine (Compound 234)

Following a procedure similar to General Procedure H, from 5-chloromethyl-3-(4-trifluoromethyl-phenyl)-isoxazole and 2-(2,3-difluoro-phenyl)-6H-imidazo[4,5-d]pyridazin-4-ylamine (in place of 2-(2,3-difluoro-phenyl)-5H-imidazo[4,5-d]pyridazine). MS 473.1 (M+H⁺); H¹ NMR (DMSO-d₆): δ (ppm) 9.55 (s, 1H), 8.01-8.1 (m, 3H), 7.86 (d, 2H), 7.41-7.50 (m, 1H), 7.28-7.32 (m, 2H), 7.12 (s, 2H), 5.92 (s, 2H).

### P Example 135

### 2-(2,3-Difluoro-phenyl)-6-[3-(4-propoxy-phenyl)-isoxazol-5-ylmethyl]-6H-imidazo[4,5-d]pyridazin-4-ylamine (Compound 235)

Following a procedure similar to General Procedure H, from 2-(2,3-difluoro-phenyl)-6H-imidazo[4,5-d]pyridazin-4-ylamine (in place of 2-(2,3-difluoro-phenyl)-5H-imidazo[4,5-d]pyridazine) and 5-chloromethyl-3-(4-propoxy-phenyl)-isoxazole. MS 463.1 (M+H⁺); H¹ NMR (DMSO-d₆): δ (ppm) 9.53 (s, 1H), 8.01-8.07 (m, 1H), 7.74-7.79 (m, 2H), 7.40-7.47 (m, 1H), 7.25-7.32 (m, 1H), 7.11 (s, 2H), 7.09 (s, 1H), 6.99-7.04 (m, 2H), 5.86 (s, 2H), 3.97 (t, 2H), 1.73 (sext., 2H), 0.98 (t, 3H).

### Example 136

### 2-(2,3-Difluoro-phenyl)-6-[3-(2-fluoro-4-trifluoromethyl-phenyl)-isoxazol-5-ylmethyl]-6H-imidazo[4,5-d]pyridazin-4-ylamine (Compound 236)

Following a procedure similar to General Procedure H, from 2-(2,3-difluoro-phenyl)-6H-imidazo[4,5-d]pyridazin-4-ylamine (in place of 2-(2,3-difluoro-phenyl)-5H-imidazo[4,5-d]pyridazine) and 5-chloromethyl-3-(2-fluoro-4-trifluoromethyl-phenyl)-isoxazole. MS 491.1 (M+H⁺); H¹ NMR (DMSO-d₆): δ (ppm) 9.55 (s, 1H), 8.12 (t, 1H), 8.00-8.06 (m, 1H), 7.92 (d, 1H), 7.72 (d, 1H), 7.41-7.50 (m, 1H), 7.25-7.32 (m, 1H), 7.18 (d, 1H), 7.12 (s, 2H), 5.94 (s, 2H).

### Example 137

### 5-[3-(2,4-Bis-trifluoromethyl-phenyl)-isoxazol-5-ylmethyl]-2-(2,3-difluoro-phenyl)-5H-imidazo[4,5-d]pyridazine-4,7-diamine (Compound 237)

### 2-(2,3-Difluoro-phenyl)-5H-imidazo[4,5-d]pyridazine-4,7-diamine

To 2-(2,3-Difluoro-phenyl)-1H-imidazole-4,5-dicarbonitrile (100 mg) was added hydrazine (anhydrous, 1 mL) and stirred at room temperature for 16 hrs. The hydrazine was removed and the product purified by HPLC.

### 5-[3-(2,4-Bis-trifluoromethyl-phenyl)-isoxazol-5-ylmethyl]-2-(2,3-difluoro-phenyl)-5H-imidazo[4,5-d]pyridazine-4,7-diamine (Compound 237)

Following a procedure similar to General Procedure H, from 3-(2,4-bis-trifluoromethyl-phenyl)-5-chloromethyl-isoxazole and 2-(2,3-difluoro-phenyl)-5H-imidazo[4,5-d]pyridazine-4,7-diamine (in place of 2-(2,3-difluoro-phenyl)-5H-imidazo[4,5-d]pyridazine). MS 556 (M+H⁺); H¹NMR (DMSO-d₆): δ (ppm) 8.96 (br s, 2H), 8.22 (m, 2H), 8.00 (m, 1H), 7.90 (m, 1H), 7.77 (m, 1H), 7.45 (m, 1H), 7.31 (br s, 2H), 6.95 (s, 1H), 5.71 (s, 1H).

### Example 138

### 5-[5-(4-Chloro-phenyl)-oxazol-2-ylmethyl]-2-(2-fluoro-phenyl)-5H-imidazo[4,5-d]pyridazine (Compound 238)

From 2-(2-Fluoro-phenyl)-5H-imidazo[4,5-d]pyridazine and 2-chloromethyl-5-(4-chloro-phenyl)-oxazole (similar to General Procedure B, using corresponding oxazole derivatives in place of isoxazole derivatives). MS: 406.1 (M+H⁺); H¹ NMR (DMSO-d₆): δ (ppm) 10.56 (s, 1H), 9.77-9.79 (m, 1H), 8.32-8.42 (m, 1H), 7.64-7.83 (m, 4H), 7.44-7.59 (m, 4H), 6.37 (s, 2H).

### Example 139

### 5-[5-(4-Chloro-phenyl)-isoxazol-3-ylmethyl]-2-(2-fluoro-phenyl)-5H-imidazo[4,5-d]pyridazine (Compound 239)

From 2-(2-Fluoro-phenyl)-5H-imidazo[4,5-d]pyridazine and methanesulfonic acid 5-(4-chloro-phenyl)-isoxazol-3-ylmethyl ester. MS: 406.1 (M+H⁺); H¹ NMR (DMSO-d₆): δ (ppm) 10.45 (s, 1H), 9.73 (s, 1H), 8.30-8.40 (m, 1H), 7.81-7.90 (m, 2H), 7.65-7.76 (m, 1H), 7.42-7.64 (m, 4H), 7.19 (s, 1H), 6.23 (s, 2H).

### Example 140

### 2-(2-Fluoro-phenyl)-5-[5-(4-methoxy-phenyl)-[1,2,4]oxadiazol-3-ylmethyl]-5H-imidazo[4,5-d]pyridazine (Compound 240)

From 2-(2-Fluoro-phenyl)-5H-imidazo[4,5-d]pyridazine and 3-chloromethyl-5-(4-methoxy-phenyl)-[1,2,4]oxadiazole. MS: 403.1 (M+H⁺); H¹ NMR (DMSO-d₆): δ (ppm) 10.57 (s, 1H), 9.79 (s, 1H), 8.31-8.40 (m, 1H), 7.97-8.04 (m, 2H), 7.68-7.79 (m, 1H), 7.44-7.58 (m, 2H), 7.09-7.17 (m, 2H), 6.37 (s, 1H), 3.85 (s, 3H).

### Example 141

### 2-(2,3-Difluoro-phenyl)-5-[5-(4-trifluoromethyl-phenyl)-isoxazol-3-ylmethyl]-5H-imidazo[4,5-d]pyridazine (Compound 241)

From methanesulfonic acid 3-(4-trifluoromethyl-phenyl)-isoxazol-5-ylmethyl ester and 2-(2,3-difluoro-phenyl)-5H-imidazo[4,5-d]pyridazine MS 458.1 (M+H⁺); H¹ NMR (DMSO-d₆): δ (ppm) 10.34 (s, 1H), 9.6 (s, 1H), 8.08-8.16 (m, 3H), 7.78 (m, 2H), 7.67 (m, 1H), 7.44 (s, 1H), 7.32 (s, 1H), 6.18 (s, 2H).

### Example 142

### 2-(2,3-Difluoro-phenyl)-5-[5-(4-trifluoromethoxy-phenyl)-isoxazol-3-ylmethyl]-5H-imidazo[4,5-d]pyridazine (Compound 242)

From methanesulfonic acid 5-(4-trifluoromethoxy-phenyl)-isoxazol-3-ylmethyl ester and 2-(2,3-difluoro-phenyl)-5H-imidazo[4,5-d]pyridazine. MS 474.1 (M+H⁺); H¹ NMR (DMSO-d₆): δ (ppm) 10.27 (s, 1H), 9.6 (s, 1H), 8.16 (m, 1H), 7.98 (m, 2H), 7.67 (m, 1H), 7.54 (m, 2H), 7.42 (m, 1H), 7.2 (s, 1H), 6.13 (s, 2H).

### Example 143

### 2-(2,3-Difluoro-phenyl)-5-[5-(4-propoxy-phenyl)-isoxazol-3-ylmethyl]-5H-imidazo[4,5-d]pyridazine (Compound 243)

From methanesulfonic acid 5-(4-propoxy-phenyl)-isoxazol-3-ylmethyl ester and 2-(2,3-difluoro-phenyl)-5H-imidazo[4,5-d]pyridazine. MS 448.1 (M+H⁺); H¹ NMR (DMSO-d₆): δ (ppm) 10.34 (s, 1H), 9.6 (s, 1H), 8.08-8.16 (m, 1H), 7.75 (m, 3H), 7.02 (m, 3H), 6.13 (s, 2H), 3.96 (t, 2H), 1.7 (m, 2H), 0.97 (t, 3H).

### Example 144

### 5-[5-(4-Butyl-phenyl)-isoxazol-3-ylmethyl-2-(2,3-difluoro-phenyl)-5H-imidazo[4,5-d]pyridazine (Compound 244)

From methanesulfonic acid 5-(4-butyl-phenyl)-isoxazol-3-ylmethyl ester and 2-(2,3-difluoro-phenyl)-5H-imidazo[4,5-d]pyridazine. MS 446.1 (M+H⁺); H¹ NMR (DMSO-d₆): δ (ppm) 10.54 (s, 1H), 9.7 (s, 1H), 8.08-8.16 (m, 1H), 7.75 (m, 3H), 7.5 (m, 1H), 7.32 (m, 2H), 7.06 (s, 1H), 6.21 (s, 2H), 2.61 (m, 2H), 1.54 (m, 2H), 1.3 (m. 2H), 0.85 (t, 3H).

### General Procedure J

### Synthesis of Compounds 245-247

### 6-(2,3-difluoro-phenyl)-2H-imidazo[4,5-c]pyridazine

Pyridazine-3,4-diamine was synthesized as described by Kuraishi et al. in J. Het. Chem. 1964, 1, 42-47. MS: 111.1 (M+H⁺); H¹-NMR (DMSO-d₆): δ (ppm) 8.2-8.3 (m, 3H), 7.31 (s, 2H), 6.73 (d, 1H, 6.1 Hz).

2,3-Difluoro-benzoic acid (100 mg), HATU (345.6 mg), and diisopropylethylamine (3 eq.) were added to DMF (900 uL) and stirred for 15 minutes. Pyridazine-3,4-diamine was added and the reaction mixture was stirred at room temperature overnight. The reaction mixture was evaporated, partitioned between water and ethyl acetate. The organic fraction was dried with sodium sulfate and concentrated in vacuo. The residue was then heated in acetic acid at reflux for one day. The mixture was evaporated and purified via reverse-phase HPLC to give 136 mg of 6-(2,3-difluoro-phenyl)-2H-imidazo[4,5-c]pyridazine. MS: 233.1 (M+H⁺) H¹-NMR (DMSO-d₆): δ (ppm) 9.04 (d, 1H, 5.8 Hz), 8.08 (m, 1H), 7.96 (d, 1H, 5.3 Hz) 7.70 (m, 1H) 7.44 (m, 1H)

### Compounds 245-247

A solution of 6-(2,3-difluoro-phenyl)-2H-imidazo[4,5-c]pyridazine and a 5-chloromethyl-2-aryl-isoxazole compound (1 equivalent), and cesium carbonate (66.7 mg, 0.20 mmol) in DMF (3 mL) was heated under microwave irradiation at 120° C. for 10 minutes. The reaction was filtered and purified by reverse phase HPLC to give the desired product. The product was converted to the HCl salt by the addition of 1N HCl before concentration.

### Example 145

### 2-[3-(2,4-Bis-trifluoromethyl-phenyl)-isoxazol-5-ylmethyl]-6-(2,3-difluoro-phenyl)-2H-imidazo[4,5-c]pyridazine (Compound 245)

From 3-(2,4-bis-trifluoromethyl-phenyl)-5-chloromethyl-isoxazole and 6-(2,3-difluoro-phenyl)-2H-imidazo[4,5-c]pyridazine. MS: 526.1 (M+H⁺); H¹-NMR (DMSO-d₆): δ (ppm) 9.4 (d, 1H), 8.4 (d, 1H), 8.2 (m, 3H), 7.9 (d, 2H), 7.7 (m, 1H), 7.4 (m, 1H), 7.1 (s, 1H), 6.4 (s, 2H).

### Example 146

### 6-(2,3-Difluoro-phenyl)-2-[3-(4-trifluoromethyl-phenyl)-isoxazol-5-ylmethyl]-2H-imidazo[4,5-c]pyridazine (Compound 246)

From 5-chloromethyl-3-(4-trifluoromethyl-phenyl)-isoxazole and 6-(2,3-difluoro-phenyl)-2H-imidazo[4,5-c]pyridazine. MS: 458.0 (M+H⁺); H¹-NMR (DMSO-d₆): δ (ppm) 9.4 (d, 1H), 8.4 (d, 1H), 8.2 (m, 1H), 8.1 (d, 2H), 7.9 (d,2H), 7.7 (m, 1H), 7.4 (m, 1), 7.4 (s, 1H), 6.3 (s, 2H).

### Example 147

### 6-(2,3-Difluoro-phenyl)-2-[3-(2-fluoro-4-trifluoromethyl-phenyl)-isoxazol-5-ylmethyl]-2H-imidazo[4,5-c]pyridazine (Compound 247)

From 5-chloromethyl-3-(2-fluoro-4-trifluoro-phenyl)-isoxazole and 6-(2,3-difluoro-phenyl)-2H-imidazo[4,5-c]pyridazine. MS: 476.1 (M+H⁺); H¹-NMR (DMSO-d₆): δ (ppm) 9.4 (d, 1H), 9.4 (d, 1H), 8.1-8.2 (m, 2H), 7.9 (m, 1H), 7.6-7.8 (m, 2H), 7.4-7.5 (m, 1H), 7.4 (d, 1H), 6.4 (s, 1H).

### General Procedure K

### Synthesis of 2-(Substituted amino)-5-substituted-imidazo[4,5-d]pyridazines

### Compounds 248-261

### 5-[3-(2,4-Bis-trifluoromethyl-phenyl)-isoxazol-5-ylmethyl]-2-bromo-5H-imidazo[4,5-d]pyridazine

To a solution of 2-bromo-5H-imidazo[4,5-d]pyridazine (350 mg) in DMF (5 mL) was added an excess of K₂CO₃ (500 mg) and 3-(2,4-Bis-trifluoromethyl-phenyl)-5-chloromethyl-isoxazole (1 eq, 600 mg) and heated to 40 C for 1 hr. The mixture was then cooled and poured into H₂O (30 mL) and the precipitate collected and dried to give the product (590 mg, 70%). MS 492.1, 494.1 (M+H⁺); H¹ NMR (DMSO-d₆): δ (ppm) 10.08 (s, 1H), 9.41 (s, 1H), 8.22 (m, 2H), 7.91 (m, 1H), 7.01 (s, 1H), 6.21 (s, 1H).

### 2-(Substituted amino)-5-substituted-imidazo[4,5-d]pyridazines

5-[3-(2,4-Bis-trifluoromethyl-phenyl)-isoxazol-5-ylmethyl]-2-bromo-5H-imidazo[4,5-d]pyridazine (70 mg) was dissolved in a substituted amino compound (0.5 mL) and heated under microwave irradiation to 160° C. for 10 minutes. The mixture was cooled and the solvent was removed, yielding the amine after HPLC purification.

### Example 148

### {5-[3-(2,4-Bis-trifluoromethyl-phenyl)-isoxazol-5-ylmethyl]-5H-imidazo[4,5-d]pyridazin-2-yl}-phenyl-amine (Compound 248)

From aniline. MS 505.1 (M+H⁺); H¹ NMR (DMSO-d₆): δ (ppm) 11.1 (br s, 1H), 9.8 (s, 1H), 9.31 (s, 1H), 8.2 (m, 2H), 7.91 (m, 1H), 7.77 (m, 2H), 7.4 (m, 2H), 7.11 (m, 1H), 7.05 (s, 1H), 6.27 (s, 2H).

### Example 149

### 5-[3-(2,4-Bis-trifluoromethyl-phenyl)-isoxazol-5-ylmethyl]-2-morpholin-4-yl-5H-imidazo[4,5-d]pyridazine (Compound 249)

From morpholine. MS 499.1 (M+H⁺); H¹ NMR (DMSO-d₆): δ (ppm) 9.86 (s, 1H), 9.26 (s, 1H), 8.23 (m, 2H), 7.91 (m, 1H), 7.03 (s, 1H), 6.28 (s, 2H), 3.77 (m, 8H).

### Example 150

### 5-[3-(2,4-Bis-trifluoromethyl-phenyl)-isoxazol-5-ylmethyl-2-piperidin-1-yl-5H-imidazo[4,5-d]pyridazine (Compound 250)

From piperidine. MS 497.1 (M+H⁺); H¹ NMR (DMSO-d₆): δ (ppm) 9.74 (s, 1H), 9.2 (s, 1H), 8.23 (m, 2H), 7.91 (m, 1H), 7.02 (s, 1H), 6.25 (s, 2H), 3.80 (m, 4H) 1.65 (m, 6H).

### Example 151

### Benzyl-{5-[3-(2,4-bis-trifluoromethyl-phenyl)-isoxazol-5-ylmethyl]-5H-imidazo[4,5-d]pyridazin-2-yl}-amine (Compound 251)

From benzylamine. MS 519 (M+H⁺); H¹ NMR (DMSO-d₆): δ (ppm) 9.7 (s, 1H), 9.37 (br s, 1H), 9.2 (s, 1H), 8.23 (m, 2H), 7.90 (m, 1H), 7.23 (m, 5H), 7.02 (s, 1H), 6.24 (s, 2H), 4.7 (d, 2H).

### Example 152

### Benzyl-{5-[3-(2,4-bis-trifluoromethyl-phenyl)-isoxazol-5-ylmethyl]-5H-imidazo[4,5-d]pyridazin-2-yl}-methyl-amine (Compound 252)

From benzyl-methyl-amine. 533.1 (M+H⁺); H¹ NMR (DMSO-d₆): δ (ppm) 9.71 (s, 1H), 9.18 (s, 1H), 8.18-8.14 (m, 2H), 7.85-7.83 (d, 1H), 7.29-7.25 (m, 5H), 6.98 (s, 1H), 6.20 (s, 2H), 4.88 (s, 2H), 3.17 (s, 3H).

### Example 153

### 1-{5-[3-(2,4-Bis-trifluoromethyl-phenyl)-isoxazol-5-ylmethyl]-5H-imidazo[4,5-d]pyridazin-2-yl}-1,2,3,4-tetrahydro-quinoline (Compound 253)

From 1,2,3,4-tetrahydro-quinoline. 545.1 (M+H⁺); H¹ NMR (DMSO-d₆): δ (ppm) 9.82 (s, 1H), 9.32 (s, 1H), 8.24-8.20 (m, 2H), 7.93-7.89 (t, 2H), 7.30-7.24 (m, 2H), 7.16-7.11 (m, 1H), 7.05 (s, 1H), 6.29 (s, 2H), 4.06 (t, 2H), 2.83-2.79 (t, 2H), 2.05-2.01 (m, 2H).

### Example 154

### {5-[3-(2,4-Bis-trifluoromethyl-phenyl)-isoxazol-5-ylmethyl]-5H-imidazo[4,5-d]pyridazin-2yl}-(2 -fluoro-benzyl)-amine (Compound 254)

From 2-fluoro-benzylamine. 537.1 (M+H⁺); H¹ NMR (DMSO-d₆): δ (ppm) 9.69 (s, 1H), 9.34 (s, 1H), 9.17 (s, 1H), 8.17-8.13 (m, 2H), 7.84-7.82 (d, 1H), 7.40-7.08 (m, 4H), 6.97 (s, 1H), 6.20 (s, 2H), 4.70-4.68 (d, 2H).

### Example 155

### {5-[3-(2,4-Bis-trifluoromethyl-phenyl)-isoxazol-5-ylmethyl]-5H-imidazo[4,5-d]pyridazin-2-yl}-(2,3-difluoro-benzyl)-amine (Compound 255)

From 2,3-difluoro-benzylamine 555.1 (M+H⁺); H¹ NMR (DMSO-d₆): δ (ppm), 9.71 (s, 1H), 9.37 (s, 1H), 9.18 (s, 1H), 8.17-8.13 (m, 2H), 7.84-7.82 (d, 1H), 7.31-7.10 (m, 3H), 6.97 (s, 1H), 6.20 (s, 2H), 4.71-4.72 (d, 2H).

### Example 156

### {5-[3-(2,4-Bis-trifluoromethyl-phenyl)-isoxazol-5-ylmethyl]-5H-imidazo[4,5-d]pyridazin-2-yl}-phenethyl-amine (Compound 256)

From phenethylamine. 533.1 (M+H⁺); H¹ NMR (DMSO-d₆): δ (ppm) 9.69 (s, 1H), 9.20 (s, 1H), 8.96 (b, 1H), 8.23-8.20 (m, 2H), 7.91-7.88 (d, 1H), 7.31-7.16 (m, 5H), 7.02 (s, 1H), 6.24 (s, 2H), 3.71-3.67 (m, 2H), 2.96-2.91 (t, 2H).

### Example 157

### 2-{5-[3-(2,4-Bis-trifluoromethyl-phenyl)-isoxazol-5-ylmethyl]-5H-imidazo[4,5-d]pyridazin-2-yl}-1,2,3,4-tetrahydro-isoquinoline (Compound 257)

From 1,2,3,4-tetrahydro-isoquinoline. 545.1 (M+H⁺); H¹ NMR (DMSO-d₆): δ (ppm) 9.75 (s, 1H), 9.19 (s, 1H), 8.17-8.13 (m, 2H), 7.85-7.82 (d, 1H), 7.19-7.16 (m, 4H), 6.97 (s, 1H), 6.21 (s, 2H), 4.92 (s, 2H), 4.01-3.97 (t, 2H), 2.97-2.93 (t, 2H).

### Example 158

### {5-[3-(2,4-Bis-trifluoromethyl-phenyl)-isoxazol-5-ylmethyl]-5H-imidazo[4,5-d]pyridazin-2-yl}-(1-phenyl-ethyl)-amine (Compound 258)

From 1-phenyl-ethylamine. 533.1 (M+H⁺); H¹ NMR (DMSO-d₆): δ (ppm) 9.70-9.56 (m, 2H), 9.18 (s, 1H), 8.22-8.18 (m, 2H), 7.89-7.87 (d, 1H), 7.47-7.44 (d, 2H), 7.35-7.02 (m, 3H), 7.00 (s, 1H), 6.24 (s, 2H), 5.22-5.17 (q, 1H), 1.57-1.55 (d, 3H).

### Example 159

### 5-[3-(2,4-Bis-trifluoromethyl-phenyl)-isoxazol-5-ylmethyl]-5H-imidazo[4,5-d]pyridazin-2-yl}-indan-1-yl-amine (Compound 259)

From indan-1-ylamine. 545.1 (M+H⁺); H¹ NMR (DMSO-d₆): δ (ppm) 9.75 (s, 1H), 9.40-9.38 (d, 1H), 9.24 (s, 1H), 8.24-8.20 (d, 2H), 7.92-7.89 (d, 1H), 7.31-7.15 (m, 4H), 6.28 (s, 2H), 7.05 (s, 1H), 5.56-5.54 (q, 1H), 3.07-2.84 (m, 2H), 2.62-2.58 (m, 1H), 2.06-1.99 (m, 1H).

### Example 160

### {5-[3-(2,4-Bis-trifluoromethyl-phenyl)-isoxazol-5-ylmethyl]-5H-imidazo[4,5-d]pyridazin-2-yl}-(1,2,3,4-tetrahydro-naphthalen-1-yl)-amine (Compound 260)

From 1,2,3,4-tetrahydro-naphthalen-1-ylamine. 559.2 (M+H⁺); H¹ NMR (DMSO-d₆): δ (ppm) 9.59 (s, 1H), 9.27 (s, 1H), 9.16 (s, 1H), 8.18-8.14 (d, 2H), 7.86-7.83 (d, 1H), 7.21-7.02 (m, 4H), 6.98 (s, 1H), 6.21 (s, 2H), 5.17 (s, 1H), 2.80-2.66 (m, 2H), 2.02-1.68 (m, 5H).

### Example 161

### 5-[3-(2,4-Bis-trifluoromethyl-phenyl)-isoxazol-5-ylmethyl]-2-(1,3-dihydro-isoindol-2-yl)-5H-imidazo[4,5-d]pyridazine (Compound 261)

From 2,3-dihydro-1H-isoindole. 531.1 (M+H⁺); H¹ NMR (DMSO-d₆): δ (ppm) 9.24 (s, 1H), 8.84 (s, 1H), 8.22-8.18 (m, 2H), 7.93-7.91 (d, 1H), 7.42-7.29 (m, 4H), 6.94 (s, 1H), 6.05 (s, 2H), 4.92 (s, 4H).

### Example 162

### 6-[3-(2,4-Bis-trifluoromethyl-phenyl)-isoxazol-5-ylmethyl]-2-(2,3-difluoro-phenyl)-6H-imidazo[4,5-d]pyridazin-4-ol (Compound 262)

To 6-[3-(2,4-Bis-trifluoromethyl-phenyl)-isoxazol-5-ylmethyl]-2-(2,3-difluoro-phenyl)-6H-imidazo[4,5-d]pyridazin-4-ylamine (compound 233, 54 mg) in HOAc (1 mL) was added NOBF₄ (32 mg, 2 eq.) and stirred at RT for 2 hrs. The solvent was removed and the crude product purified by HPLC. MS 542.1 (M+H⁺); H¹ NMR (DMSO-d₆): δ (ppm) 9.87 (s, 1H), 8.23 (m, 2H), 7.93 (m, 2H), 7.77 (m, 1H), 7.43 (m, 1H), 7.06 (s, 1H), 6.08 (s, 2H).

### Example 163

### 3-{5-[2-(2,3-Difluoro-phenyl)-imidazo[4,5-d]pyridazin-5-ylmethyl]-isoxazol-3-yl}-benzoic acid (Compound 263)

To a solution of 3-{5-[2-(2,3-difluoro-phenyl)-imidazo[4,5-d]pyridazin-5-ylmethyl]-isoxazol-3-yl}-benzoic acid methyl ester (compound 210, 138.5 mg, 0.31 mmol) in dichloromethane (3.8 mL), boron tribromide (1.0M in dichloromethane, 2.79 mL) was added. The mixture was heated at 42° C. until completion. The reaction was quenched by the addition of 1N HCl, and the solvent was removed. The resulting residue was purified by reverse phase HPLC to give the desired product. The product was converted to the HCl salt by the addition of 1N HCl before concentration. MS: 434.1 (M+H⁺); H¹ NMR (DMSO-d₆): δ (ppm) 10.26 (s, 1H), 9.61 (s, 1H), 8.33-8.38 (m, 1H), 8.00-8.19 (m, 3H), 7.59-7.68 (m, 2H), 7.35-7.45 (m, 1H), 7.32 (s, 1H), 6.26 (s, 2H).

### Example 164

### 4-{5-[2-(2,3-Difluoro-phenyl)-imidazo[4,5-d]pyridazin-5-ylmethyl]-isoxazol-3-yl}-benzoic acid (Compound 264)

To a solution of 4-{5-[2-(2,3-difluoro-phenyl)-imidazo[4,5-d]pyridazin-5-ylmethyl]-isoxazol-3-yl}-benzoic acid methyl ester (compound 209, 50.0 mg, 0.11 mmol) in dichloromethane (1.4 mL), boron tribromide (1.0M in dichloromethane, 1.00 mL) was added. The mixture was heated at 42° C. until completion. The reaction was quenched by the addition of 1N HCl, and the solvent was removed. The resulting residue was purified by reverse phase HPLC to give the desired product. The product was converted to the HCl salt by the addition of 1N HCl before concentration. MS: 434.1 (M+H⁺); H¹ NMR (DMSO-d₆): δ (ppm) 10.23 (s, 1H), 9.58 (s, 1H), 8.11-8.20 (m, 1H), 7.94-8.07 (m, 4H), 7.56-7.68 (m, 1H), 7.34-7.44 (m, 1H), 7.29 (s, 1H), 6.25 (s, 2H).

### Example 165

### (4-{5-[2-(2,3-Difluoro-phenyl)-imidazo[4,5-d]pyridazin-5-ylmethyl]-isoxazol-3-yl}-phenoxy)-acetic acid (Compound 265)

To a solution of (4-{5-[2-(2,3-difluoro-phenyl)-imidazo[4,5-d]pyridazin-5-ylmethyl]-isoxazol-3-yl}-phenoxy)-acetic acid methyl ester (compound 215, 150 mg, 0.31 mmol) in acetonitrile (3 mL), 2M HCl (3 mL) was added. The mixture was heated allowed to stir at 50° C. overnight. The acetonitrile was removed, and the resulting residue was purified by reverse phase HPLC to give the desired product. The product was converted to the HCl salt by the addition of 1N HCl before concentration. MS: 434.1 (M+H⁺); H¹ NMR (DMSO-d₆): δ (ppm) 10.39 (s, 1H), 9.67 (s, 1H), 8.12-8.20 (m, 1H), 7.60-7.80 (m, 3H), 7.38-7.48 (m, 1H), 7.16 (s, 1H), 6.97-7.07 (m, 2H), 6.27 (s, 2H), 4.74 (s, 2H).

### Example 166

### 2-{5-[2-(2,3-Difluoro-phenyl)-imidazo[4,5-d]pyridazin-5-ylmethyl]-isoxazol-3-yl}-5-methoxy-benzoic acid (Compound 266)

2-{5-[2-(2,3-Difluoro-phenyl)-imidazo[4,5-d]pyridazin-5-ylmethyl]-isoxazol-3-yl}-5-methoxy-benzoic acid methyl ester (compound 198, 100 mg) are heated in 6 mL of 1:1 6N HCl/4MHCl in dioxane for three hours at 95° C. The reaction is cooled, evaporated and purified via reverse phase HPLC to give 2-{5-[2-(2,3-difluoro-phenyl)-imidazo[4,5-d]pyridazin-5-ylmethyl]-isoxazol-3-yl}-5-methoxy-benzoic acid. The product was converted to the HCl salt by the addition of 1N HCl before concentration.

MS: 464.1 (M+H⁺); H¹-NMR (DMSO-d₆): δ (ppm) 10.4 (s, 1H), 9.7 (s, 1H), 8.2 (m, 1H), 8.0 (s, 1H), 7.7 (m, 1H), 7.4-7.5 (m, 2H), 7.3 (d, 1H), 7.2 (m,1H), 6.9 (s, 1H), 6.3 (s, 2H), 3.8 (s, 3H).

### Example 167

### 5-{5-[2-(2,3-Difluoro-phenyl)-imidazo[4,5-d]pyridazin-5-ylmethyl]-isoxazol-3-yl}-2-propoxy-benzoic acid (Compound 267)

5-{5-[2-(2,3-Difluoro-phenyl)-imidazo[4,5-d]pyridazin-5-ylmethyl]-isoxazol-3-yl}-2-propoxy-benzoic acid propyl ester (compound 197, 110 mg) are heated in 6 mL of 1:1 6N HCl aq./4MHCl in dioxane for three hours at 95° C. The reaction is cooled, evaporated and purified via reverse phase HPLC to give 43 mg of 5-{5-[2-(2,3-Difluoro-phenyl)-imidazo[4,5-d]pyridazin-5-ylmethyl]-isoxazol-3-yl}-2-propoxy-benzoic acid. The product was converted to the HCl salt by the addition of 1N HCl before concentration. MS: 492.1 (M+H⁺); H¹-NMR (DMSO-d₆): δ (ppm) 10.2 (s, 1H), 9.6 (s, 1H), 8.2 (m, 1H), 8.0 (s, 1H), 7.9 (dd, 1H), 7.6 (m, 1H), 7.4 (m, 1H), 7.2 (m, 2H), 6.2 (s, 2H), 4.0 (t, 2H), 1.7 (m, 2H), 1.0 (t, 3H).

### Example 168

### 2-(2,3-Difluoro-phenyl)-5-[3-(4′-methoxy-biphenyl-4-yl)-isoxazol-5-ylmethyl]-5H-imidazo[4,5-d]pyridazine (Compound 268)

A reaction vessel is charged with 5-[3-(4-bromo-phenyl)-isoxazol-5-ylmethyl]-2-(2,3-difluoro-phenyl)-5H-imidazo[4,5-d]pyridazine (compound 200, 50 mg, 0.1 mmol), 4-methoxy-phenyl-boronic acid (24.3 mg, 1.5 eq.), tetrakis(triphenylphosphine)-palladium(0) (6 mg, 0.05 eq.), evacuated in vacuo and filled with argon three times. A 2N sodium carbonate solution (107 μL, 2 eq.) and toluene (427 μL) are added and the solution is degassed for 5 minutes. The sealed reaction vessel is then heated to 80° C. for 3 hr. After cooling the reaction mixture is concentrated and purified via reverse phase HPLC to give 17 mg of 2-(2,3-difluoro-phenyl)-5-[3-(4′-methoxy-biphenyl-4-yl)-isoxazol-5-ylmethyl]-5H-imidazo[4,5-d]pyridazine. The product was converted to the HCl salt by the addition of 1N HCl before concentration. MS: 496.2 (M+H⁺); H¹-NMR (DMSO-d₆): δ (ppm) 10.4 (s, 1H), 9.9 (s, 1H), 8.2 (m, 1H), 7.9 (d, 2H), 7.7 (d, 2H), 7.4-7.7 (m, 6H), 7.3 (s, 1H), 7.0 (d, 2H), 6.3 (s, 2H), 3.8 (s, 3H).

### Example 169

### 2-(2,3-Difluoro-phenyl)-5-[3-(4′-propoxy-biphenyl-4-yl)-isoxazol-5-ylmethyl]-5H-imidazo[4,5-d]pyridazine (Compound 269)

A reaction vessel is charged with 5-[3-(4-bromo-phenyl)-isoxazol-5-ylmethyl]-2-(2,3-difluoro-phenyl)-5H-imidazo[4,5-d]pyridazine (compound 200, 50 mg, 0.1 mmol), 4-propoxy-phenyl-boronic acid (28.8 mg, 1.5 eq.), tetrakis(triphenylphosphine)-palladium(0) (6 mg, 0.05 eq.), evacuated in vacuo and filled with argon three times. A 2N sodium carbonate solution (107 μL, 2 eq.) and toluene (427 μL) are added and the solution is degassed for 5 minutes. The sealed reaction vessel is then heated to 80° C. for 3 hr. After cooling the reaction mixture is concentrated and purified via reverse phase HPLC to give 18 of 2-(2,3-Difluoro-phenyl)-5-[3-(4′-propoxy-biphenyl-4-yl)-isoxazol-5-ylmethyl]-5H-imidazo[4,5-d]pyridazine. The product was converted to the HCl salt by the addition of 1N HCl before concentration. MS: 524.2 (M+W+); H¹-NMR (DMSO-d₆): δ (ppm) 10.3 (d, 1H), 9.6 (d, 1H), 8.1-8.2 (m, 1H), 7.9 (m, 1H), 7.7-7.8 (m, 2H), 7.6-7.7 (m, 3H), 7.4 (m, 1H), 7.2 (s, 1H), 7.0 (m, 2H), 6.2 (s, 2H), 4.0 (t, 2H), 1.7-1.8 (m, 2H), 1.0 (t, 3H).

### Example 170

### 5-{5-[2-(2,3-Difluoro-phenyl)-imidazo[4,5-d]pyridazin-5-ylmethyl]-isoxazol-3-yl}-N-(2-morpholin-4-yl-ethyl)-2-propoxy-benzamide (Compound 270)

5-{5-[2-(2,3-Difluoro-phenyl)-imidazo[4,5-d]pyridazin-5-ylmethyl]-isoxazol-2-propoxy-benzoic acid (compound 267, 30 mg), HATU (23.4 mg), and diisopropylethylamine (21.8 uL) are dissolved in 0.5 mL DMF and stirred for 5 minutes. 2-Aminoethyl morpholine (6 uL) is added and the reaction stirred for 2 hours at room temperature. The reaction is then evaporated and purified via reverse phase-HPLC to give 21 mg of 5-{5-[2-(2,3-Difluoro-phenyl)-imidazo[4,5-d]pyridazine-5-ylmethyl]-isoxazol-3-yl}-N-(2-morpholin-4-yl-ethyl)-2-propoxy-benzamide. The product was converted to the HCl salt by the addition of 1 N HCl before concentration. MS: 604.1 (M+H⁺); H¹-NMR (DMSO-d₆): δ (ppm) 10.9 (bs, 1H), 10.4.(s, 1H), 9.7 (s, 1H), 8.5 (tr, 1H), 8.1-8.2 (m, 2H), 7.9 (m, 1H), 7.7 (m, 1H), 7.4 (m, 1H), 7.2-7.3 (m, 2H), 6.3 (s, 2H), 4.1 (tr, 2H), 3.70-4.0 (m, 6H), 3.5 (m, 2H), 3.2 (m, 2H), 3.1 (m, 2H),1.8 (m, 2H), 1.0 (t, 3H).

### Example 171

### N-Cyclopropyl-2-(4-{5-[2-(2,3-difluoro-phenyl)-imidazo[4,5-d]pyridazin-5-ylmethyl]-isoxazol-3-yl}-phenoxy)-acetamide (Compound 271)

The (4-{5-[2-(2,3-difluoro-phenyl)-imidazo[4,5-d]pyridazin-5-ylmethyl]-isoxazol-3-y}-phenoxy)-acetic acid (compound 265, 39 mg, 0.084 mmol), cyclopropyamine (7 μL, 0.10 mmol), diisopropylethylamine (30 μL) and HATU (35 mg, 0.92 mmol) were combined under Ar in a vial and stirred at room temp for 1 h. The reaction mixture was portioned between ethyl acetate and 1 N HCl. The organic layer was washed sequentially with saturated aqueous NaHCO₃, water, and brine. After drying over sodium sulfate, the organics were concentrated onto celite. The product was purified via SiO₂ flash chromatography using 0-20% methanol in ethyl aceate. MS 503.1 (M+H⁺); H¹ NMR (DMF-d₇): δ (ppm) 10.96 (s, 1H), 10.02 (s, 1H), 8.51-8.56 (m, 1H), 8.42 (s, 1H), 7.88-7.97 (m, 1H), 7.65-7.72 (m, 1H), 7.50 (s, 1H), 7.27 (s, 2H), 6.72 (s, 2H), 4.76 (s, 2H), 4.14 (t, 1H), 0.81-0.88 (m, 2H), 0.71-0.76 (m, 2H).

### Example 172

### Acetic acid 3-(4-{5-[2-(2,3-difluoro-phenyl)-imidazo[4,5-d]pyridazin-5-ylmethyl]-isoxazol-3-yl}-phenoxy)-propyl ester (Compound 272)

The 3-(4-{5-[2-(2,3-difluoro-phenyl)-imidazo[4,5-d]pyridazin-5-ylmethyl]-isoxazol-3-yl}-phenoxy)-propan-1-ol (compound 193, 20 mg) was dissolved in 1 mL of acetic anhydride, and excess triethylamine (ca. 0.1 mL) was added. The reaction was warmed to 85° C. for 1 h, and then the volatile components were removed. The residue was portioned between ethyl acetate and water. The organic layer was concentrated to give the pure product. MS 506.0 (M+H⁺); H¹ NMR (CDCl₃): δ (ppm) 9.35 (d, 1H), 9.27 (d, 1H), 8.14-8.19 (m, 1H), 7.68 (d, 2H), 7.19-7.29 (m, 2H), 6.95 (d, 2H), 6.69 (d, 1H), 5.90 (s, 2H), 4.26 (t, 2H), 4.08 (t, 2H), 2.13 (quintet, 2H), 2.06 (s, 3H).

### Example 173

### 2-(2,3-Difluoro-phenyl)-5-{3-[4-(3-morpholin-4-yl-propoxy)-phenyl]-isoxazol-5-ylmethyl}-5H-imidazo[4,5-d]pyridazine (Compound 273)

A solution of 3-(4-{5-[2-(2,3-difluoro-phenyl)-imidazo[4,5-d]pyridazin-5-ylmethyl]-isoxazol-3-yl}-phenoxy)-propan-1-ol (compound 193, 40 mg) in DMF (1 mL) was treated with triethylamine (0.1 mL) then methanesulfonyl chloride (0.1 mL). After 10 min, 0.20 mL of morpholine was added and the mixture was heated to 90° C. for 1 h. The reaction mixture was purified by reverse-phase HPLC to give the product, which was converted to the HCl salt and collected as a white powder. MS 533.0 (M+H⁺); H¹ NMR (DMSO-d₆): β(ppm) 10.49 (s, 1H), 9.75 (s, 1H), 8.18-8.23 (m, 1H), 7.82 (d, 2H), 7.70-7.79 (m, 1H), 7.45-7.52 (m, 1H), 7.20 (s, 1H), 7.08 (d, 2H), 6.33 (s, 2H), 4.15 (t, 2H), 3.98 (dd, 2H), 3.84 (t, 2H), 3.46 (d, 2H), 3.23-3.31 (m, 2H), 3.03-3.14 (m, 2H), 2.21-2.29 (m, 2H).

### Example 174

### 4-(4-{5-[2-(2,3-Difluoro-phenyl)-imidazo[4,5-d]pyridazin-5-ylmethyl]-isoxazol-3-yl}-phenoxy)-butyric acid (Compound 274)

The 4-(4-{5-[2-(2,3-difluoro-phenyl)-imidazo[4,5-d]pyridazin-5-ylmethyl]-isoxazol-3-yl}-phenoxy)-butyric acid methyl ester (compound 192, 60 mg) was suspended in ethanol and magnetically stirred in an ice bath as 5 mL of KOH (20%, aq.) was added. The reaction was stirred at room temp overnight, and then most of the ethanol was removed under vacuum. The remaining liquid was diluted with 50 mL of water, and the pH was adjusted to 3 using concentrated HCl. The product precipitated and was isolated by filtration. MS 492.1 (M+H⁺); H¹ NMR (DMSO-d₆): δ (ppm) 10.37 (s, 1H), 9.66 (s, 1H), 8.14-8.17 (m, 1H), 7.76 (d, 2H), 7.67 (quartet, 1H), 7.40-7.47 (m, 1H), 7.15 (s, 1H), 7.02 (d, 2H), 6.25 (s, 2H), 4.02 (t, 2H), 2.37 (t, 2H), 1.93 (quintet, 2H).

### Example 175

### 2-(2-Fluoro-phenyl)-5-[3-(3-propoxy-phenyl)-isoxazol-5-ylmethyl]-5H-imidazo[4,5-d]pyridazine (Compound 275)

Following General Procedure H, from 2-(2-fluoro-phenyl)-5H-imidazo[4,5-d]pyridazine and 5-chloromethyl-3-(3-propoxy-phenyl)-isoxazole. MS: 430.1 (M+H⁺); H¹ NMR (DMSO-d₆): δ (ppm) 10.59 (s, 1H), 9.82 (s, 1H), 8.32-8.42 (m, 1H), 7.69-7.80 (m, 1H), 7.45-7.60 (m, 2H), 7.32-7.43 (m, 3H), 7.27 (s, 1H), 7.01-7.09 (m, 1H), 6.39 (s, 2H), 3.97 (t, 3H), 1.65-1.80 (m, 2H), 0.98 (t, 3H).

### Example 176

### 2-(2-Fluoro-phenyl)-5-[3-(3-trifluoromethyl-phenyl)-isoxazol-5-ylmethyl-5H-imidazo[4,5-d]pyridazine (Compound 276)

Following General Procedure H, from 2-(2-fluoro-phenyl)-5H-imidazo[4,5-d]pyridazine and 5-chloromethyl-3-(3-trifluoromethyl-phenyl)-isoxazole. MS: 440.1 (M+H⁺); H¹ NMR (DMSO-d₆): δ (ppm) 10.15 (s, 1H), 9.77 (s, 1H), 8.31-8.40 (m, 1H), 8.12-8.21 (m, 2H), 7.85-7.92 (m, 1H), 7.65-7.80 (m, 2H), 7.38-7.57 (m, 3H), 6.39 (s, 2H).

### Example 177

### 5-[3-(4-Butyl-phenyl)-isoxazol-5-ylmethyl]-2-(2-fluoro-phenyl)-5H-imidazo[4,5-d]pyridazine (Compound 277)

Following General Procedure H, from 2-(2-fluoro-phenyl)-5H-imidazo[4,5-d]pyridazine and 3-(4-butyl-phenyl)-5-chloromethyl-isoxazole. MS: 428.1 (M+H⁺); H¹ NMR (DMSO-d₆): δ (ppm) 10.40 (s, 1H), 9.71 (s, 1H), 8.30-8.40 (m, 1H), 7.63-7.77 (m, 3H), 7.41-7.55 (m, 2H), 7.28-7.34 (m, 2H), 7.20 (s, 1H), 6.30 (s, 2H), 2.62 (t, 2H), 1.49-1.63 (m, 2H), 1.22-1.38 (m, 2H), 0.90 (t, 3H).

### Example 178

### 2-(2-Fluoro-phenyl)-5-[3-(4-trifluoromethyl-phenyl)-isoxazol-5-ylmethyl]-5H-imidazo[4,5-d]pyridazine (Compound 278)

Following General Procedure H, from 2-(2-fluoro-phenyl)-5H-imidazo[4,5-d]pyridazine and 5-chloromethyl-3-(4-trifluoromethyl-phenyl)-isoxazole. MS: 440.1 (M+H⁺); H¹ NMR (DMSO-d₆): δ (ppm) 10.43 (s, 1H), 9.72 (s, 1H), 8.30-8.39 (m, 1H), 8.04-8.11 (m, 2H), 7.84-7.91 (m, 2H), 7.60-7.74 (m, 1H), 7.41-7.55 (m, 2H), 7.36 (s, 1H), 6.35 (s, 2H).

### Example 179

### 2-(2-Fluoro-phenyl)-5-[3-(2-fluoro-4-trifluoromethyl-phenyl)-isoxazol-5-ylmethyl]-5H-imidazo[4,5-d]pyridazine (Compound 279)

Following General Procedure H, from 2-(2-fluoro-phenyl)-5H-imidazo[4,5-d]pyridazine and 5-chloromethyl-3-(2-fluoro-4-trifluoromethyl-phenyl)-isoxazole. MS: 458.0 (M+H⁺); H¹ NMR (DMSO-d₆): δ (ppm) 10.37 (s, 1H), 9.68 (s, 1H), 8.30-8.39 (m, 1H), 8.06-8.16 (m, 1H), 7.91-7.98 (m, 1H), 7.58-7.77 (m, 2H), 7.40-7.53 (m, 2H), 7.24-7.29 (m, 1H), 6.35 (s, 2H).

### Example 180

### 5-[3-(2,5-Bis-trifluoromethyl-phenyl)-isoxazol-5-ylmethyl]-2-(2-fluoro-phenyl)-5H-imidazo[4,5-d]pyridazine (Compound 280)

Following General Procedure H, from 2-(2-fluoro-phenyl)-5H-imidazo[4,5-d]pyridazine and 3-(2,5-bis-trifluoromethyl-phenyl)-5-chloromethyl-isoxazole. MS: 508.1 (M+H⁺); H¹ NMR (DMSO-d₆): δ (ppm) 10.34 (s, 1H), 9.68 (s, 1H), 8.29-8.39 (m, 1H), 8.11-8.22 (m, 2H), 8.04 (s, 1H), 7.60-7.70 (m, 1H), 7.39-7.52 (m, 2H), 7.10 (s, 1H), 6.35 (s, 2H).

### Example 181

### 2-(2-Fluoro-phenyl)-5-[3-(4-methanesulfonyl-phenyl)-isoxazol-5-ylmethyl]-5H-imidazo[4,5-d]pyridazine (Compound 281)

Following General Procedure H, from 2-(2-fluoro-phenyl)-5H-imidazo[4,5-d]pyridazine and 5-chloromethyl-3-(4-methanesulfonyl-phenyl)-isoxazole. MS: 45.0.1 (M+H⁺); H¹ NMR (DMSO-d₆): δ (ppm) 10.47 (s, 1H), 9.74 (s, 1H), 8.31-8.40 (m, 1H), 8.01-8.15 (m, 4H), 7.64-7.76 (m, 1H), 7.35-7.56 (m, 3H), 6.38 (s, 1H), 3.28 (s, 3H).

### Example 182

### 2-(2-Fluoro-phenyl)-5-[3-(4-iodo-phenyl)-isoxazol-5-ylmethyl]-5H-imidazo[4,5-d]pyridazine (Compound 282)

Following General Procedure H, from 2-(2-fluoro-phenyl)-5H-imidazo[4,5-d]pyridazine and 5-chloromethyl-3-(4-iodo-phenyl)-isoxazole. MS: 498.0 (M+H⁺); H¹ NMR (DMSO-d₆): δ (ppm) 10.46 (s, 1H), 9.74 (s, 1H), 8.30-8.45 (m, 1H), 7.84-7.91 (m, 2H), 7.43-7.72 (m, 5H), 7.25 (s, 1H), 6.34 (s, 2H).

### Example 183

### 5-[3-(4-tert-Butyl-phenyl)-isoxazol-5-ylmethyl]-2-(2-fluoro-phenyl)-5H-imidazo[4,5-d]pyridazine (Compound 283)

Following General Procedure H, from m 2-(2-fluoro-phenyl)-5H-imidazo[4,5-d]pyridazine and 3-(4-tert-butyl-phenyl)-5-chloromethyl-isoxazole. MS: 428.2 (M+H⁺); H¹ NMR (DMSO-d₆): δ (ppm) 10.41-10.45 (m, 1H), 9.71-9.76 (m, 1H), 8.29-8.43 (m, 1H), 7.62-7.79 (m, 3H), 7.42-7.56 (m, 4H), 7.21 (s, 1H), 6.32 (s, 2H), 1.30 (s, 9H).

### Example 184

### 4-{5-[2-(2-Fluoro-phenyl)-imidazo[4,5-d]pyridazin-5-ylmethyl]-isoxazol-3-yl}-benzonitrile (Compound 284)

Following General Procedure H, from 2-(2-fluoro-phenyl)-5H-imidazo[4,5-d]pyridazine and 4-(5-chloromethyl-isoxazol-3-yl)-benzonitrile. MS: 397.2 (M+H⁺); H¹ NMR (DMSO-d₆): δ (ppm) 10.56 (s, 1H), 9.79 (s, 1H), 8.31-8.40 (m, 1H), 7.94-8.08 (m, 4H), 7.68-7.78 (m, 1H), 7.44-7.58 (m, 1H), 7.38 (s, 1H), 6.41 (s, 2H).

### Example 185

### 5-[3-(4-Bromo-phenyl)-isoxazol-5-ylmethyl]-2-(2-fluoro-phenyl)-5H-imidazo[4,5-d]pyridazine (Compound 285)

Following General Procedure H, from 2-(2-fluoro-phenyl)-5H-imidazo[4,5-d]pyridazine and 3-(4-bromo-phenyl)-5-chloromethyl-isoxazole. MS: 451.0 (M+H⁺); H¹ NMR (DMSO-d₆): δ (ppm) 10.35 (s, 1H), 9.67 (s, 1H), 8.30-8.40 (m, 1H), 7.64-7.84 (m, 5H), 7.39-7.53 m, 2H), 7.26 (s, 1H), 6.30 (s, 2H).

### Example 186

### 2-(2-Fluoro-phenyl)-5-[3-(3-fluoro-pyridin-4-yl)-isoxazol-5-ylmethyl]-5H-imidazo[4,5-d]pyridazine (Compound 286)

Following General Procedure H, from 2-(2-fluoro-phenyl)-5H-imidazo[4,5-d]pyridazine and 4-(5-chloromethyl-isoxazol-3-yl)-3-fluoro-pyridine. MS: 391.1 (M+H⁺); H¹ NMR (DMSO-d₆): δ (ppm) 10.77 (s, 1H), 9.88 (s, 1H), 8.83 (s, 1H), 8.59 (d, 1H), 8.38 (t, 1H), 7.88-7.96 (m, 1H), 7.71-7.84 (m, 1H), 7.47-7.63 (m, 2H), 7.37 (s, 1H), 6.53 (s, 2H).

### Example 187

### 2-(2-Fluoro-phenyl)-5-[3-(1H-indol-5-yl)-isoxazol-5-ylmethyl]-5H-imidazo[4,5-d]pyridazine (Compound 287)

Following General Procedure H, from 2-(2-fluoro-phenyl)-5H-imidazo[4,5-d]pyridazine and 5-(5-chloromethyl-isoxazol-3-yl)-1H-indole. MS: 411.1 (M+H⁺); H¹ NMR (DMSO-d₆): δ (ppm) 11.34 (s, 1H), 10.31 (s, 1H), 9.65 (s, 1H), 8.30-8.40 (m, 1H), 8.03 (s, 1H), 7.38-7.70 (m, 6H), 7.19 (s, 1H), 6.46-6.52 (s, 1H), 6.26 (s, 2H).

### Example 188

### 2-(2-Fluoro-phenyl)-5-[3-(1H-indol-6-yl)-isoxazol-5-ylmethyl]-5H-imidazo[4,5-d]pyridazin (Compound 288)

Following General Procedure H, from 2-(2-fluoro-phenyl)-5H-imidazo[4,5-d]pyridazine and 5-(5-chloromethyl-isoxazol-3-yl)-1H-indole. MS: 411.2 (M+H⁺); H¹ NMR (DMSO-d₆): δ (ppm) 11.43 (s, 1H), 10.63 (s, 1H), 9.82 (s, 1H), 8.32-8.41 (m, 1H), 7.86 (s, 1H), 7.42-7.79 (m, 6H), 7.25 (s, 1H), 6.47 (s, 1H), 6.39 (s, 2H).

### Example 189

### 5-[3-(5-Bromo-pyridin-2-yl)-isoxazol-5-ylmethyl]-2-(2-fluoro-phenyl)-5H-imidazo[4,5-d]pyridazine (Compound 289)

Following General Procedure H, from 2-(2-fluoro-phenyl)-5H-imidazo[4,5-d]pyridazine and 5-bromo-2-(5-chloromethyl-isoxazol-3-yl)-pyridine. MS: 451.0 (M+H⁺); H¹ NMR (DMSO-d₆): δ (ppm) 10.56 (s, 1H), 9.78 (s, 1H), 8.80-8.86 (m, 1H), 8.31-8.40 (m, 1H), 8.18-8.26 (m, 1H), 7.93-8.00 (m, 1H), 7.68-7.80 (m, 1H), 7.43-7.59 (m, 2H), 7.28 (s, 1H), 6.42 (s, 2H).

### Example 190

### 1-(4-{5-[2-(2,3-Difluoro-phenyl)-imidazo[4,5-d]pyridazin-5-ylmethyl]-isoxazol-3-yl}-phenyl)-ethanone (Compound 290)

### 1-(4-{5-[2-(2,3-Difluoro-phenyl)-imidazo[4,5-d]pyridazin-5-ylmethyl]-isoxazol-3-yl}-phenyl)-ethanone oxime

Following General Procedure H, from 2-(2,3-difluoro-phenyl)-5H-imidazo[4,5-d]pyridazine and 1-[4-(5-chloromethyl-isoxazol-3-yl)-phenyl]-ethanone oxime.

### 1-(4-{5-[2-(2,3-Difluoro-phenyl)-imidazo[4,5-d]pyridazin-5-ylmethyl]-isoxazol-3-yl}-phenyl)-ethanone (Compound 290)

1-(4-{5-[2-(2,3-Difluoro-phenyl)-imidazo[4,5-d]pyridazin-5-ylmethyl]-isoxazol-3-yl}-phenyl)-ethanone oxime (171.5 mg, 0.38 mmol) was dissolved in glyoxylic acid (50% aq. solution, 3 mL) and stirred at ambient temperature for two hours. After removal of the solvent, purification by reverse phase HPLC gave the desired product. The product was converted to the HCl salt by the addition of 1N HCl before concentration. MS: 432.1 (M+H⁺); H¹ NMR (DMSO-d₆): δ (ppm) 10.57 (s, 1H), 9.78 (s, 1H), 8.12-8.21 (m, 1H), 8.02 (q, 4H), 7.66-7.80 (m, 1H), 7.41-7.52 (m, 1H), 7.36 (s, 1H), 6.40 (s, 2H), 2.61 (s, 3H).

### Example 191

### 5-[5-(4-Chloro-phenyl)-[1,3,4]oxadiazol-2-ylmethyl]-2-(2-fluoro-phenyl)-5H-imidazo[4,5-d]pyridazine (Compound 291)

2-(2-Fluoro-phenyl)-5H-imidazo[4,5-d]pyridazine (30.0 mg, 0.14 mmol), 2-chloromethyl-5-phenyl-[1,3,4]oxadiazole (similar to General Procedure B, using the oxadiazole derivative in place of the isoxazole derivative, 32.1 mg, 0.14 mmol), and cesium carbonate (91.3 mg, 0.28 mmol) were dissolved in DMF and microwaved at 120° C. for 10 minutes. The reaction was filtered and purified by reverse phase HPLC to give the desired product. Yield 12.7 mg. MS 407.0 (M+H⁺); H¹ NMR (DMSO-d₆): δ (ppm) 10.22 (s, 1H), 9.58 (d, 1H), 8.31-8.38 (m, 1H), 7.96-8.01 (m, 2H), 7.57-7.70 (m, 3H), 7.37-7.47 (m, 2H), 6.40 (s, 2H).

### Example 192

### 5-[3-(2,4-Bis-trifluoromethyl-phenyl)-4-bromo-isoxazol-5-ylmethyl]-2-(2,3-difluoro-phenyl)-5H-imidazo[4,5-d]pyridazine (Compound 292)

To a solution of 5-[3-(2,4-Bis-trifluoromethyl-phenyl)-isoxazol-5-ylmethyl]-2-(2,3-difluoro-phenyl)-5H-imidazo[4,5-d]pyridazine (compound 103, 535 mg, 1 mmol) in HOAc (10 mL) was added H₂SO₄ (5 drops) and NBS (725 mg, 4 eq.) and the mixture heated in a sealed vial to 115° C. for 18 hrs. The solvent was removed and the crude product purified by HPLC. MS 604.1/606.1 (M+H⁺); H¹NMR (DMSO-d₆): δ (ppm) 10.1 (s, 1H), 9.51 (s, 1H), 8.3 (m, 2H), 8.16 (m, 2H), 7.8 (m, 1H), 7.56 (m, 1H), 7.36 (m, 1H), 6.28 (s, 2H).

### Example 601

### 2-(2-Fluoro-phenyl)-5-(4-trifluoromethoxy-benzyl)-5H-imidazo[4,5-d]pyridazine (Compound 6101)

The title compound was obtained following step 4 of Example 604, using appropriate starting materials.

MS: 389.0 (M+H⁺); H¹ NMR (DMSO-d₆): δ (ppm) 10.50 (s, 1H), 9.67 (s, 1H), 8.3 (m, 1H), 7.7 (m, 3H), 7.5 (m, 4H), 6.0 (s, 2H).

### Example 602

### 5-(4-Chloro-benzyl)-2-(2-fluoro-phenyl)-5H-imidazo[4,5-d]pyridazine (Compound 6102)

The title compound was obtained following step 4 of Example 604, using appropriate starting materials.

MS: 339.0 (M+H⁺); H¹ NMR (DMSO-d₆): δ (ppm) 10.34 (s, 1H), 9.60 (s, 1H), 8.3 (m, 1H), 7.6 (m, 1H), 7.4 (m, 7H), 5.94 (s, 2H).

### Example 603

### 5-Benzyloxymethyl-2-(2-fluoro-phenyl)-5H-imidazo[4,5-d]pyridazine (Compound 6103)

The title compound was obtained following step 4 of Example 604, using appropriate starting materials.

MS: 335.0 (M+H⁺); H¹ NMR (DMSO-d₆): δ (ppm) 10.23 (s, 1H), 9.67 (s, 1H), 8.3 (m, 1H), 7.6 (m, 1H), 7.4 (m, 2H), 7.3 (m, 5H), 6.1 (s, 2H), 4.7 (s, 2H).

### Example 604

### 5-[6-(2,4-Bis-trifluoromethyl-phenyl)-pyridazin-3-ylmethyl]-2-(2,3-difluoro-phenyl)-5H-imidazo[4,5-d]pyridazine (Compound 6104)

### Step 1. 2-(2,3-Difluoro-phenyl)-1H-imidazo[4,5-d]pyridazine

Following the general procedure for the synthesis of 2-substituted 5H-imidazo[4,5-d]pyridazines described above, 2,3-difluorobenzoic acid chloride was used to yield 2-(2,3-difluoro-phenyl)-1H-imidazo[4,5-d]pyridazine.

### Step 2. 3-(2,4-Bis-trifluoromethyl-phenyl)-6-methyl-pyridazine

A solution of 3-chloro-6-methyl-pyridazine (2.56 g, 20 mmol), 2,4-bis-trifluoromethyl-phenyl-boronic acid (7.7 g, 30 mmol), tetrakistriphenylphosphine palladium(0) (5 mol %, 1.1 g) in toluene: 2N Na₂CO₃ (4:1, 100 nIL total) was sparged with argon for 3 minutes then heated to 100° C. for 20 hours. The reaction was partitioned, the aqueous phase washed with EtOAc (2×50 mL) and the organics combined, dried (brine, Na₂SO₄) and purified on silica gel eluting with 10-60% hexanes:EtOAc, yielding the product (1.9 g) as a brown solid.

### Step 3. 3-(2,4-Bis-trifluoromethyl-phenyl)-6-chloromethyl-pyridazine

To a solution of 3-(2,4-bis-trifluoromethyl-phenyl)-6-methyl-pyridazine (1.9 g, 6.21 mmol) in dichloroethane (100 mL) was added trichloroisocyanuric acid (580 mg, 0.4 equivalent) and heated to 70° C. After 40 minutes the reaction was cooled, the solids decanted off and the solution washed with NaOH aq (0.5M, 10 mL). The aqueous phase was extracted with dichloromethane (10 mL) and the organics dried (brine, Na₂SO₄) yielding the product as a yellow oil in sufficient purity for the next reaction (1.7 g).

### Step 4. 5-[6-(2,4-Bis-trifluoromethyl-phenyl)-pyridazin-3-ylmethyl]-2-(2,3-difluoro-phenyl)-5H-imidazo[4,5-d]pyridazine (compound 6104)

A solution of 3-(2,4-bis-trifluoromethyl-phenyl)-6-chloromethyl-pyridazine (374 mg, 1.1 mmol), 2-(2,3-difluoro-phenyl)-1H-imidazo[4,5-d]pyridazine (1 equivalent, 250 mg) K₂CO₃ (2 equivalents, 677 mg) in DMF (10 mL) was heated to 80° C. for 30 minutes. The reaction was then cooled, the solids decanted off, washed with DMF (2 mL) and the organics combined and poured into water (40 mL). The resulting precipitate was collected, triturated with MeOH, then converted to the hydrochloride salt with 1M HCl/EtOH (excess) to yield the product as a beige solid-yield 480 mg. MS: 537.0 (M+H⁺); H¹ NMR (DMSO-d₆): δ (ppm) 10.46 (s, 1H), 9.69 (s, 1H), 8.3-7.8 (m, 6H), 7.7 (m, 1H), 7.4 (m, 1H), 6.44 (s, 2H).

### Example 605

### 2-(2,3-Difluoro-phenyl)-5-[6-(4-methoxy-phenyl)-pyridazin-3-ylmethyl]-5H-imidazo[4,5-d]pyridazine (Compound 6105)

### Step 1. 3-Chloro-6-chloromethyl-pyridazine

To a solution of 3-chloro-6-methyl-pyridazine (25 g, 0.2 mol) in chloroform (850 mL) at 60° C. was added trichloroisocyanuric acid (0.4 eqivalent, 18.1 mol) and stirred for 15 hours. An additional charge of trichloroisocyanuric acid (3 g) was added and the mixture heated for an additional hour. The mixture was then cooled in an ice bath and filtered over celite. The organic solution was concentrated to a yellow oil which darkened and solidified upon standing in the freezer (yield 30 g, 95%).

### Step 2. 5-(6-Chloro-pyridazin-3-ylmethyl)-2-(2,3-difluoro-phenyl)-5H-imidazo[4,5-d]pyridazine

To a solution of 3-chloro-6-chloromethyl-pyridazine (1.2 eqivalents, 0.6 mmol, 98 mg) in DMF (1 mL) was added potassium carbonate (2 eqivalents, 140 mg) and 2-(2,3-difluoro-phenyl)-1H-imidazo[4,5-d]pyridazine (1 equivalent, 166 mg) and the mixture heated to 80° C. for 5 minutes. The mixture was cooled to room temperature and partitioned between EtOAc (20 mL) and water (20 mL). The aqueous layer was then washed with EtOAc (2x 20 mL) and the organics combined, dried (brine, Na₂SO₄) to give the product in sufficient purity for the next step (yield 64 mg, 40%).

### Step 3. 2-(2,3-Difluoro-phenyl)-5-[6-(4-methoxy-phenyl)-pyridazin-3-ylmethyl]-5H-imidazo[4,5-d]pyridazine (compound 6105)

A solution of 4-methoxyphenylboronic acid (51.2 mg, 0.34 mmol), 5-(6-chloro-pyridazin-3-ylmethyl)-2-(2,3-difluoro-phenyl)-5H-imidazo[4,5-d]pyridazine (80.5 mg, 0.22 mmol), and Pd[P(Ph)₃]₄ (13 mg, 5 mol %) in Na₂CO₃ (2N, 225 μL) and toluene (900 μL) was degassed and heated to 80° C. for 30 minutes. The reaction was cooled, taken up in distilled water (10 mL) and ethyl acetate (10 mL), and filtered. The aqueous layer was extracted with ethyl acetate (3×10 mL). The organic layers were combined, dried with anhydrous magnesium sulfate, filtered, and concentrated. The crude was purified by reverse phase HPLC, and 2M HCl was added to the appropriate fractions to convert the desired product to the HCl salt. Yield 28.0 mg. MS: 431.1 (M+H⁺); H¹ NMR (DMSO-d₆): δ (ppm)10.64 (s, 1H), 9.79 (s, 1H), 8.25-8.32 (m, 1H), 8.06-8.20 (m, 3H), 7.92-8.00 (m, 1H), 7.71-7.84 (m, 1H), 7.45-7.55 (m, 1H), 7.05-7.12 (m, 2H), 6.43 (s, 1H), 3.83 (s, 3H).

### Example 606

### 2-(2,3-Difluoro-phenyl)-5-[6-(4-ethoxy-phenyl)-pyridazin-3-ylmethyl]-5H-imidazo[4,5-d]pyridazine (Compound 6106)

The title compound was obtained following step 3 of Example 605, using appropriate starting materials.

MS: 445.1 (M+H⁺); H¹ NMR (DMSO-d₆): δ (ppm) 10.63 (s, 1H), 9.78 (s, 1H), 8.24-8.31 (m, 1H), 8.05-8.20 (m, 3H), 7.91-7.98 (m, 1H), 7.71-7.83 (m, 1H), 7.45-7.55 (m, 1H), 7.03-7.10 (m, 2H), 6.42 (s, 1H), 4.04-4.15 (q, 2H), 1.31-1.39 (t, 3H).

### Example 607

### 2-(2,3-Difluoro-phenyl)-5-[6-(4-propoxy-phenyl)-pyridazin-3-ylmethyl]-5H-imidazo[4,5-d]pyridazine (Compound 6107)

The title compound was obtained following step 3 of Example 605, using appropriate starting materials.

MS: 459.2 (M+H⁺); H¹ NMR (DMSO-d₆): δ (ppm)10.45 (s, 1H), 9.66 (s, 1H), 8.23-8.29 (m, 1H), 8.04-8.27 (m, 2H), 7.88-7.95 (m, 1H), 7.39-7.77 (m, 3H), 7.04-7.11 (m, 2H), 6.35 (s, 2H), 3.95-4.04 (t, 2H), 1.67-1.80 (m, 2H), 0.94-1.03 (t, 3H).

### Example 608

### 2-(2,3-Difluoro-phenyl)-5-[2-(4-propoxy-2-trifluoromethyl-phenyl)-pyrimidin-5-ylmethyl]-5H-imidazo[4,5-d]pyridazine (Compound 6133)

### Step 1. 4-(Propyloxy)-2-(trifluoromethyl)benzonitrile

Solid 4-fluoro-2-(trifluoromethyl)benzonitrile (80 g, 423 mmol) was dissolved in a mixture of n-Propanol (500 mL) and sodium hydride (22.4 g, 560 mmol)—washed with hexanes (3×50 mL) and then heated to 85 C for 2 hrs. The mixture was cooled by addition of ice (100 g) and then citric acid (10% w/v aq., 200 mL) was added. The phases were seperated, the aqueous layer washed with EtOAc (150 mL), the organics washed with NaHCO3 (aq. Sat) then brine and finally dried with Na2SO4. The solvents were removed and the product crystallized. This was □riturated with hexanes (50 mL), filtered and put on the hi-vac for 30 minutes yielding the product as a white solid (85 g, 88% yield).

### Step 2. Methyl-2-[bis(methyloxy)methyl]-3-hydroxy-2-propenoate sodium salt

To neat methyl 3,3-bis(methyloxy)propanoate (10.44 g, 70.5 mmol) in a 500 mL RB flask under nitrogen was added 1,2-Dimethoxyethane (DME) (50 mL) then methyl formate (10 g, 70.5 mmol) and then NaH (3.38 g, 85 mmol). To initiate the reaction, the mixture was briefly heated to 50 C and then once H2 evolution commenced, was cooled in an ice bath and slowly brought to room temperature. The mixture was stirred at RT overnight. The slurry was then treated with Et2O (40 mL) filtered under nitrogen and the filtrated washed with Et2O (2×20 mL) and dried under a stream of nitrogen. Yield 11.4 g white solid.

### Step 3. 4-(Propyloxy)-2-(trifluoromethyl)benzenecarboximidamide hydrochloride

Solid 4-(propyloxy)-2-(trifluoromethyl)benzonitrile (10 g, 43.6 mmol) was dissolved in Tetrahydrofuran (THF) (20 mL) and treated with potassium hexamethyldisilazide (13.06 g, 65.4 mmol) and then stirred at RT for 5 minutes. It was then heated to 50 degrees under nitrogen. After 1 hr, still ˜10% starting material, added another 2 g KHDMS. After another hour at 50 C, then the reaction was poured onto ice (ca 100 g) and partitioned. The aqueous layer was washed with Et2O and the organics were combined, washed with more water (2×50 mL) and then brine and dried over Na2SO4. HCl (4M in dioxane, 15 mL 1.4 eq) was added with cooling and the resulting precipitated filtered, washed with Et2O and dried. Yield 14.1 g (114%—contaminated with NH4Cl).

### Step 4. Methyl 2-[4-(propyloxy)-2-(trifluoromethyl)phenyl]-5-pyrimidinecarboxylate

A solution of sodium salt of methyl-2-[bis(methyloxy)methyl]-3-hydroxy-2-propenoate (1141 mg, 5.76 mmol) and 4-(propyloxy)-2-(trifluoromethyl)benzenecarboximidamide hydrochloride (1182 mg, 4.8 mmol) in N,N-Dimethylformamide (DMF) (20 mL) under nitrogen was heated to 100° C. for 1 hour. After 90 minutes another portion of the sodium salt (250 mg) was added and the reaction reheated for 30 minutes. Then the mixture was poured into water (50 mL) and cooled to 0 C. Filtered and dried to yield a white powder (965 mg).

### Step 5. {2-[4-(Propyloxy)-2-(trifluoromethyl)phenyl]-5-pyrimidinyl}methanol

A solution of methyl 2-[4-(propyloxy)-2-(trifluoromethyl)phenyl]-5-pyrimidinecarboxylate (690 mg, 2.028 mmol) in Tetrahydrofuran (THF) (15 mL) was treated with LAH (2.028 mL, 2.028 mmol) at −78 C. The mixture was stirred for 5 minutes and LC-MS showed complete, clean conversion to the product. After 10 more minutes, the reaction was poured into citric acid (40 mL, 10% aq). The mixture was treated with EtOAc (30 mL) and partitioned. The aqueous phase was washed with more EtOAc (30 mL) and the organics combined, dried (brine, Na2SO4), concentrated and purified on silica (30-100% EtOAc:Hexanes). Yield 165 mg.

### Step 6. 5-(Chloromethyl)-2-[4-(propyloxy)-2-(trifluoromethyl)phenyl]pyrimidine

A solution of {2-[4-(propyloxy)-2-(trifluoromethyl)phenyl]-5-pyrimidinyl}methanol (160 mg, 0.512 mmol) in Chloroform (5 mL) was treated with thionyl chloride (0.112 mL, 1.537 mmol) at 0 C. The mixture was stirred for 5 minutes then warmed to RT and stirred for 120 minutes. The solvents were then removed and the product used directly in the following reaction.

### 2-(2,3-Difluoro-phenyl)-5-[2-(4-propoxy-2-trifluoromethyl-phenyl)-pyrimidin-5-ylmethyl]-5H-imidazo[4,5-d]pyridazine.

A solution of 2-(2,3-difluoro-phenyl)-5H-imidazo[4,5-d]pyridazine (119 mg, 0.512 mmol), 5-(chloromethyl)-2-[4-(propyloxy)-2-(trifluoromethyl)phenyl]pyrimidine (169 mg, 0.512 mmol) and potassium carbonate (142 mg, 1.024 mmol) in DMF (5 mL), was heated to 60° C. and stirred for 20 minutes. The reaction was then cooled and poured into 3x water and the resulting percipitate collected, washed with water and dried giving the desired product. MS 527 (M+H⁺); H¹ NMR (DMSO-d₆): δ (ppm) 10.11 (s, 1H), 9.45 (s, 1H), 9.06 (s, 2H), 8.15 (m, 1H), 7.71 (m, 1H), 7.55 (m, 2H), 7.32 (m, 3H), 5.97 (s, 2H), 4.06 (t, 2H), 1.75 (m, 2H), 0.96 (t, 3H).

What is claimed is:

1. A process of preparing a compound of formula I 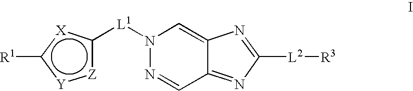
comprising converting a compound of formula 1.1 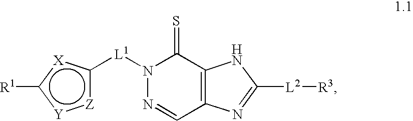
a compound of formula 2.1 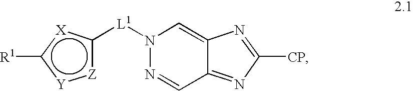
a compound of formula 3.1 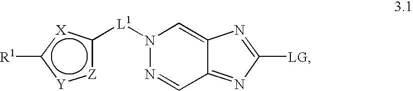
or a compound of formula II 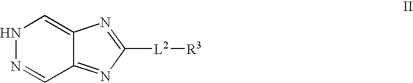
to the compound of formula I,
wherein
a) when X is CR² or N, one of Y or Z is O and the other of Y or Z is N; or one of Y or Z is N and the other of Y or Z is NR^{a};
b) when X is O, NR^{a}, or S(O)ₚ wherein p is 0 or 1, one of Y or Z is N and the other of Y or Z is N or CR²;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;
R^{a} and R^{b} are independently H, alkyl, or substituted alkyl;
R¹ and R³ are independently selected from aryl, substituted aryl, heteroaryl, substituted heteroaryl, heterocyclyl, substituted heterocyclyl, cycloalkyl, and substituted cycloalkyl;
R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano;
CP is a substituent that can undergo a coupling reaction; and
LG is a leaving group

2. The process of claim 1, wherein the compound of formula I 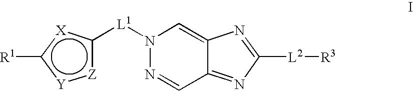
is prepared by a process comprising
reacting the compound of formula 1.1 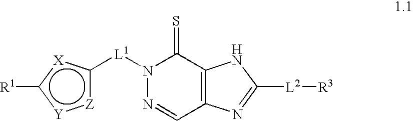
with a desulfurizing reagent.

3. The process of claim 2, wherein the compound of formula 1.1 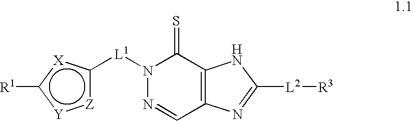
is prepared by a process comprising
reacting a compound of formula 1.2 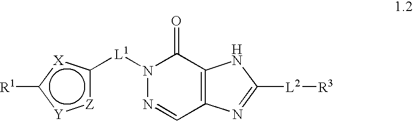
with a sulfurizing reagent.

4. The process of claim 3, wherein the compound of formula 1.2 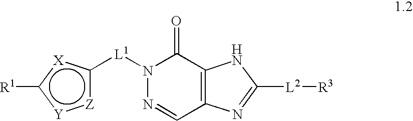
is prepared by cyclizing a compound of formula 1.3 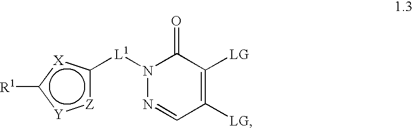
wherein
each LG is independently chosen and each is a leaving group.

5. The process of claim 4, wherein the cyclization of the compound of formula 1.3 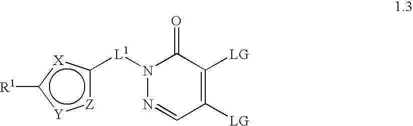
occurs with a compound of formula 1.4

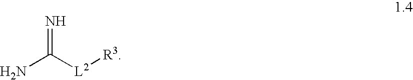

6. The process of claim 5, wherein at least one LG of formula 1.3 is a halogen.

7. The process of claim 5, wherein the compound of formula 1.3 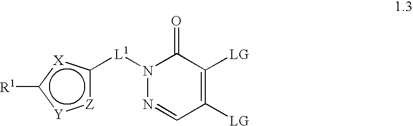
is prepared by a process comprising
cyclizing a compound of formula 1.5

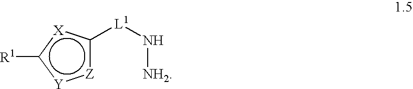

8. The process of claim 7, wherein the compound of formula 1.5 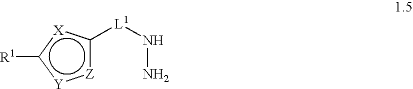
is prepared by a process comprising
reacting a compound of formula 1.6 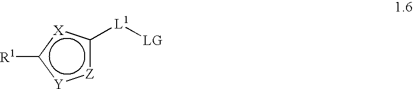
with hydrazine,
wherein
LG is a leaving group.

9. The process of claim 8, wherein LG of formula 1.6 is a halogen, hydroxy, alkoxy, substituted alkoxy, or sulfonyloxy.

10. The process of claim 1, wherein the compound of formula I 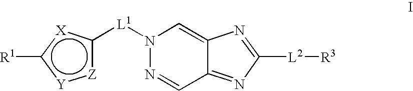
is prepared by a process comprising
coupling a compound of formula 2.1 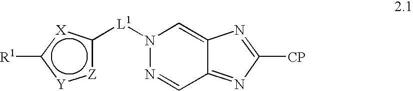
under conditions appropriate to form a new carbon-carbon bond.

11. The process of claim 10, wherein the compound of formula 2.1 is coupled with a compound of formula 2.2
M-L²-R³ 2.2
wherein
M is a substituent that can undergo a coupling reaction.

12. The process of claim 11, wherein the compound of formula 2.2
M-L²-R³ 2.2
is selected from an organotin, organozinc, organomagnesium, organosilyl, organoboron, and organotrifluoroborate compound.

13. The process of claim 12, wherein the organoboron is a boronic acid or boronic ester of formula 2.3 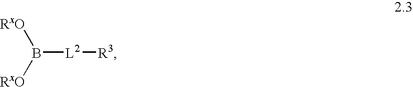
wherein
each R^{x} is independently selected from hydrogen, alkyl, or substituted alkyl and,
wherein
the R^{x} groups, if alkyl or substituted alkyl, can optionally be connected.

14. The process of claim 10, wherein CP is a halogen or sulfonlyoxy.

15. The process of claim 10, wherein the compound of formula 2.1 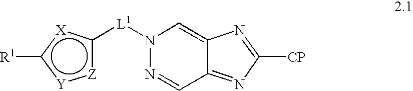
is prepared by a process comprising
reacting a compound of formula 2.5 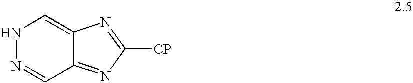
with the compound of formula 1.6 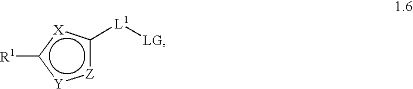
wherein LG is a leaving group.

16. The process of claim 15, wherein LG of formula 1.6 is a halogen, hydroxy, substituted alkoxy, or sulfonyloxy.

17. The process of claim 15, wherein the compound of formula 2.5 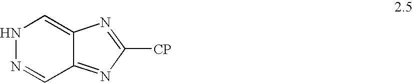
is prepared by a process comprising
a) reacting a compound of formula 2.6 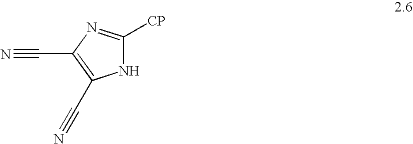
with a reducing reagent
and
b) cyclizing the resulting product to form the compound of formula 2.5.

18. The process of claim 1, wherein the compound of Formula I is prepared by converting the compound of formula 3.1 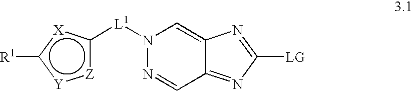
to the compound of formula I 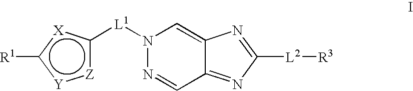
by a process that comprises reacting the compound of formula 3.1 with a compound comprising nitrogen, oxygen or sulfur.

19. The process of claim 18, wherein LG is a halogen, hydroxy, alkoxy, substituted alkoxy, or sulfonyloxy.

20. The process of claim 18, wherein the compound comprising nitrogen is chosen from aniline, morpholine, piperidine, phenylmethanamine, N-methyl(phenyl)methanamine, 1,2,3,4-tetrahydroquinoline, (2-fluorophenyl)methanamine, (2,3-diflurophenyl)methanamine, 2-phenylethanamine, 1-phenylethanamine, 1,2,3,4-tetrahydroisoquinoline, 2,3-dihydro-1H-inden-1-amine, 1,2,3,4-tetrahydronaphthalen-1-amine, and isoindoline.

21. The process of claim 18, wherein the compound of formula 3.1 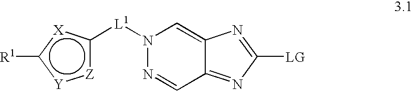
is prepared by a process comprising
reacting a compound of formula 3.2 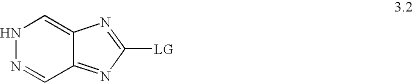
with the compound of formula 1.6 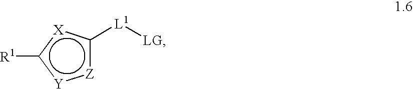
wherein
each LG of formula 3.1 and 1.6 is independently chosen and each is a leaving group.

22. The process of claim 21, wherein at least one LG of formula 3.1 and 1.6 is halogen, hydroxy, alkoxy, substituted alkoxy, or sulfonyloxy.

23. The process of claim 21, wherein the compound of formula 3.2 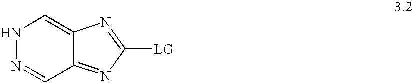
is prepared by a process comprising
a) reacting a compound of formula 3.3 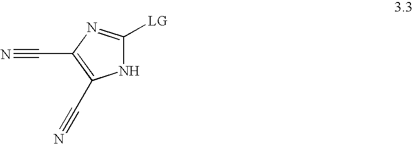
with a reducing reagent
and
b) cyclizing the resulting product to form the compound of formula 3.2.

24. The process of claim 1, wherein the compound of formula I is prepared by converting the compound of formula II 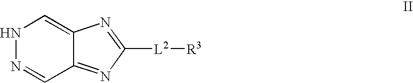
to the compound of formula I 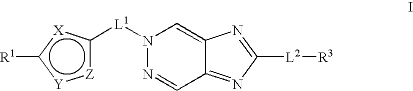
by a process that comprises reacting the compound of formula II with a compound of formula 1.6 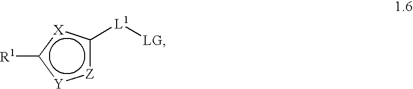
wherein
LG of formula 1.6 is a leaving group.

25. The process of claim 24, wherein LG of formula 1.6 is a halogen, hydroxy, alkoxy, substituted alkoxy, or sulfonyloxy.

26. The process of claim 24, wherein the compound of formula II 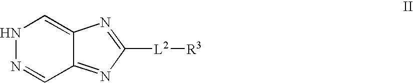
is prepared by a process comprising
a) reacting a compound of formula 4.1 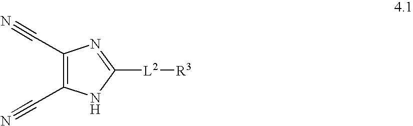
with a reducing reagent
and
b) cyclizing the resulting product to form the compound of formula II.

27. The process of claim 26, wherein the compound of formula 4.1 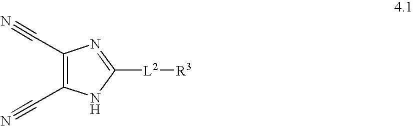
is prepared by a process comprising
cyclizing a compound of formula 4.2

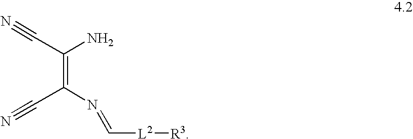

28. The process of claim 27, wherein the compound of formula 4.2 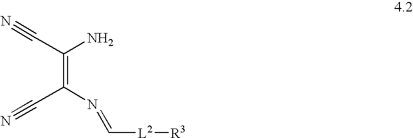
is prepared by a process comprising
reacting a compound of formula 4.3 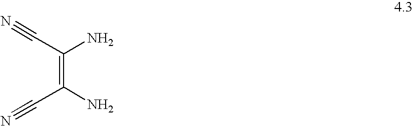
with a compound of formula 4.4

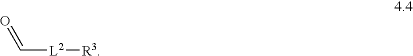

29. The process of claim 24, wherein the compound of formula II 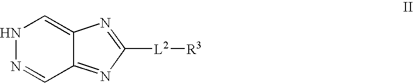
is prepared by a process comprising
a) saponifying a compound of formula 5.1 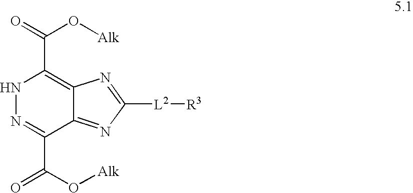
to form a compound of formula 5.2 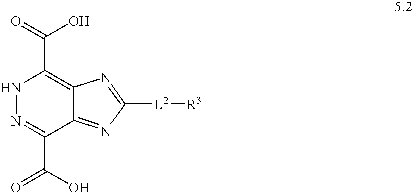
and
b) decarboxylating the compound of formula 5.2 to form the compound of formula II,
wherein
each Alk is independently chosen and each is an alkyl or substituted alkyl.

30. The process of claim 29, wherein Alk is CH₃.

31. The process of claim 29, wherein the compound of formula 5.1 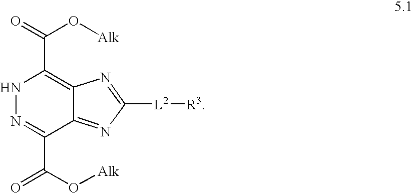
is prepared by a process comprising
reacting a compound of formula 5.3 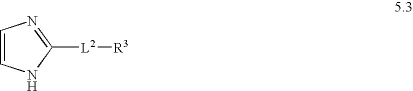
with a compound of formula 5.4 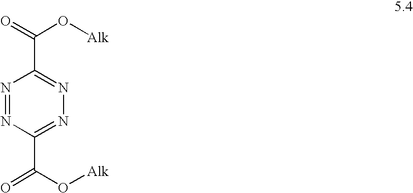
to form the compound of formula 5.1.

32. The process of claim 31, wherein the compound of formula 5.3 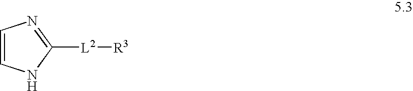
is prepared by a process comprising
reacting a compound of formula 4.4 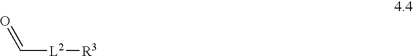
with glyoxal and ammonia.

33. The process of claim 1, wherein the compound of formula I is prepared by converting the compound of formula II 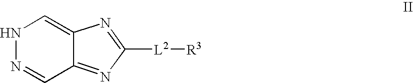
to the compound of formula I 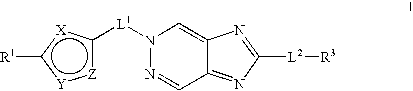
by a process that comprises
a) reacting the compound of formula II with a compound of formula 6.1 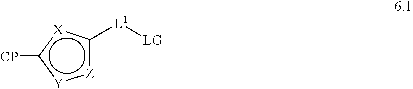
to form a compound of formula 6.2 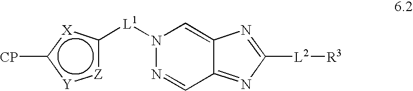
and
b) coupling the compound of formula 6.2 under conditions appropriate to form a carbon-carbon bond,
wherein
LG is a leaving group
and
CP is a substituent that can undergo a coupling reaction.

34. The process of claim 33, wherein LG of formula 6.1 is a halogen, hydroxy, alkoxy, substituted alkoxy, or sulfonyloxy.

35. The process of claim 33, wherein the compound of formula 6.2 is coupled with a compound of formula 6.3
M-R¹ 6.3
wherein
M is a substituent that can undergo a coupling reaction.

36. The process of claim 35, wherein the compound of formula 6.3
M-R¹ 6.3
is selected from an organotin, organozinc, organomagnesium, organosilyl, organoboron, and organotrifluoroborate compound.

37. The process of claim 36, wherein the organoboron is a boronic acid or boronic ester of formula 6.4 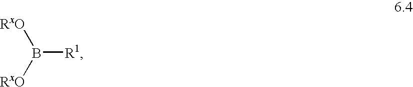
wherein
each R^{x} is independently selected from hydrogen, alkyl, or substituted alkyl
and,
wherein
the R^{x} groups, if alkyl or substituted alkyl, can optionally be connected.

38. The process of claim 33, wherein CP of formula 6.2 is a halogen or sulfonlyoxy.

39. The process of claim 1, wherein the compound of formula I is prepared by converting the compound of formula II 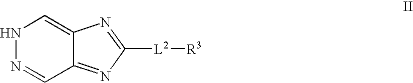
to the compound of formula I 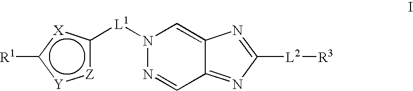
by a process that comprises
a) reacting the compound of formula II with a compound of formula 7.1 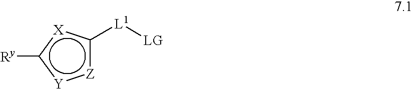
to form a compound of formula 7.2 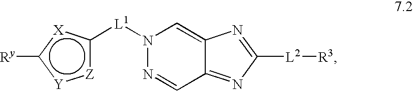
b) reacting the compound of formula 7.2 with a suitable reagent to form a compound of formula 7.3 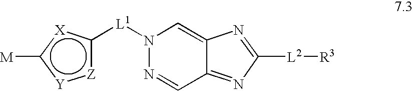
and
c) coupling the compound of formula 7.3 with a compound of formula 7.4
CP—R¹ 7.4
under conditions appropriate to form a carbon-carbon bond,
wherein
LG is a leaving group,
R^{y} is a halogen or sulfonyloxy,
and
M and CP are each substituents that can undergo a coupling reaction.

40. The process of claim 39, wherein LG of formula 7.1 is a halogen, hydroxy, alkoxy, substituted alkoxy, or sulfonyloxy.

41. The process of claim 39, wherein R^{y} is a halogen.

42. The process of claim 39, wherein M comprises tin, zinc, magnesium, silicon, or boron.

43. The process of claim 39, wherein CP of formula 7.4 is a halogen or sulfonlyoxy.

44. A process of preparing a compound of formula III 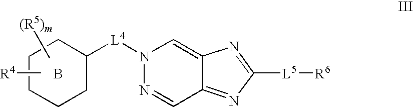
comprising converting a compound of formula 8.1 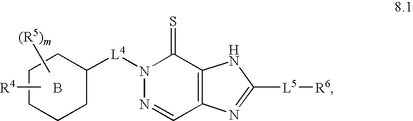
a compound of formula 9.1 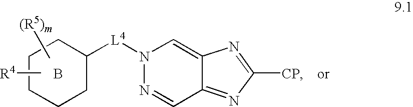
a compound of formula 10.1 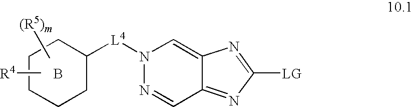
to the compound of formula III,
wherein
ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L⁴is L⁶;
L⁵ is a bond or L⁶;
L⁶ is independently C₃₋₆ cycloalkylene or is C₁₋₅ alkyene where one or two —CH₂— groups of said C₁₋₅ alkylene are optionally replaced with —NR⁷—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵;
R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;
R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁷ is independently H, alkyl, or substituted alkyl;
m is 0, 1, 2, 3, or 4;
CP is a group that can undergo a coupling reaction; and
LG is a leaving group;
provided that the compound of Formula I is not 4′-(2-butyl-imidazo[4,5-d]-pyridazin-5-ylmethyl)-biphenyl-2-carboxylic acid.

45. The process of claim 44, wherein the compound of formula III is prepared by converting the compound of formula 8.3 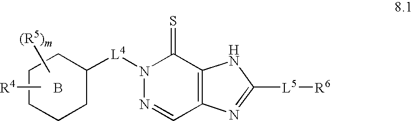
to the compound of formula III 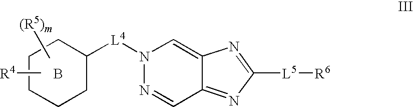
by a process that comprises desulfurization of the compound of formula 8.1.

46. The process of claim 45, wherein the compound of formula 8.1 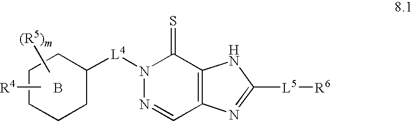
is prepared by a process comprising
reacting a compound of formula 8.2 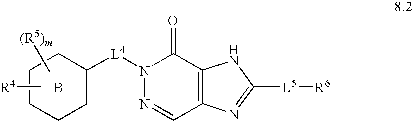
with a sulfurizing reagent.

47. The process of claim 46, wherein the compound of formula 8.2 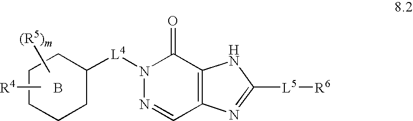
is prepared by a process comprising
cyclizing a compound of formula 8.3 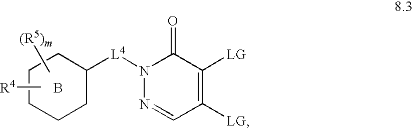
wherein
each LG of formula 8.3 is independently chosen and each is a leaving group.

48. The process of claim 47, wherein the cyclization of the compound of formula 8.3 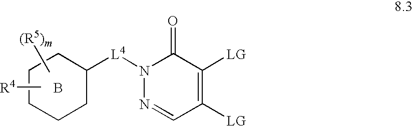
occurs with a compound of formula 8.4

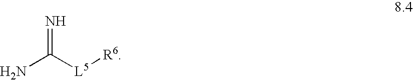

49. The process of claim 48, wherein at least one LG of formula 8.3 is a halogen.

50. The process of claim 48, wherein the compound of formula 8.3 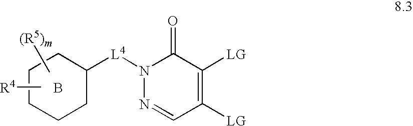
is prepared by a process comprising
cyclizing a compound of formula 8.5

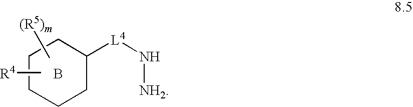

51. The process of claim 50, wherein the compound of formula 8.5 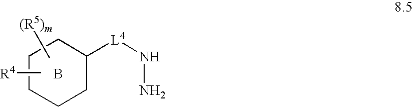
is prepared by a process comprising
reacting a compound of formula 8.6 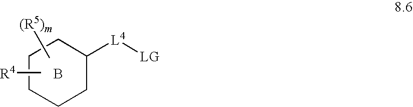
with hydrazine.
wherein
LG is a leaving group.

52. The process of claim 51, wherein LG of formula 8.6 is a halogen, hydroxy, alkoxy, substituted alkoxy, or sulfonyloxy.

53. The process of claim 44, wherein the compound of formula III 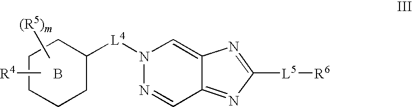
is prepared by a process comprising
coupling a compound of formula 9.1 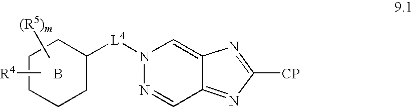
under conditions appropriate to form a new carbon-carbon bond.

54. The process of claim 53, wherein the compound of formula 9.1 is coupled with a compound of formula 9.2
M-L⁵-R⁶ 9.2
wherein
M is a substituent that can undergo a coupling reaction.

55. The process of claim 54, wherein the compound of formula 9.2
M-L⁵-R⁶ 9.2
comprises tin, zinc, magnesium, silicon, or boron.

56. The process of claim 55, wherein the compound of formula 9.2
M-L⁵-R⁶ 9.2
is selected from an organotin, organozinc, organomagnesium, organosilyl, organoboron, or organotrifluoroborate compound.

57. The process of claim 56, wherein the organoboron is a boronic acid or boronic ester of formula 9.3 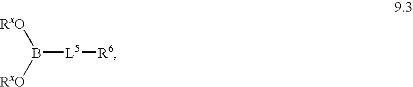
wherein
each R^{x} is independently selected from hydrogen, alkyl, or substituted alkyl
and,
wherein
the R^{x} groups, if alkyl or substituted alkyl, can optionally be connected.

58. The process of claim 53, wherein CP of formula 9.1 is a halogen or sulfonlyoxy.

59. The process of claim 53, wherein the coupling reaction occurs in the presence of at least one compound comprising palladium, nickel, iron, or copper.

60. The process of claim 53, wherein the compound of formula 9.1 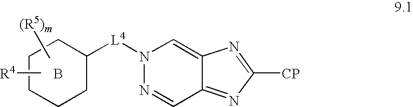
is prepared by a process comprising
reacting a compound of formula 2.5 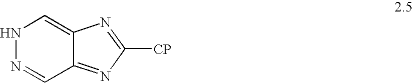
with a compound of formula 9.5 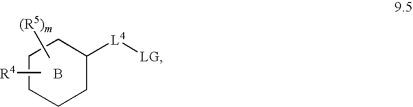
wherein LG is a leaving group.

61. The process of claim 60, wherein LG of formula 9.5 is a halogen, hydroxy, alkoxy, substituted alkoxy, or sulfonyloxy.

62. The process of claim 44, wherein the compound of formula III is prepared by converting the compound of formula 10.1 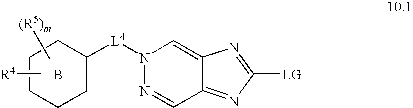
to the compound of formula III 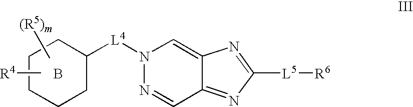
by a process that comprises reacting the compound of formula 10.1 with a compound comprising nitrogen, oxygen or sulfur.

63. The process of claim 62, wherein LG is a hydrogen, hydroxyl, alkoxy, substituted alkoxy, or sulfonyloxy.

64. The process of claim 62, wherein the compound comprising nitrogen is chosen from aniline, morpholine, piperidine, phenylmethanamine, N-methyl(phenyl)methanamine, 1,2,3,4-tetrahydroquinoline, (2-fluorophenyl)methanamine, (2,3-diflurophenyl)methanamine, 2-phenylethanamine, 1-phenylethanamine, 1,2,3,4-tetrahydroisoquinoline, 2,3-dihydro-1H-inden-1-amine, 1,2,3,4-tetrahydronaphthalen-1-amine, and isoindoline.

65. The process of claim 62, wherein the compound of formula 10.1 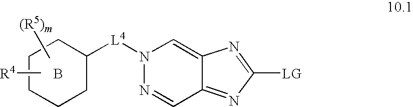
is prepared by a process comprising
reacting the compound of formula 3.2 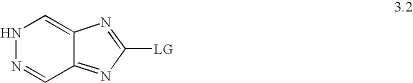
with the compound of formula 9.5 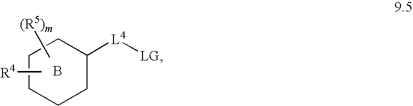
wherein
each LG of formula 3.2 and 9.5 is independently chosen and each is a leaving group.

66. The process of claim 65, wherein at least one LG formula 3.2 and 9.5 is halogen, hydroxy, alkoxy, substituted alkoxy, or sulfonyloxy.

67. A process of preparing a compound of formula III 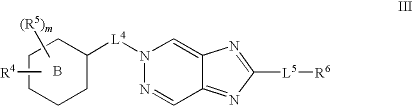
comprising converting a compound of formula IV 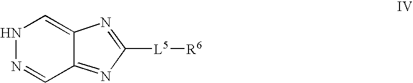
to the compound of formula III,
wherein
ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L⁴ is L⁶;
L⁵ is a bond or L⁶;
L⁶ is independently C₃₋₆ cycloalkylene or is C₁₋₅ alkylene where one or two —CH₂— groups of said C₁₋₅ alkylene are optionally replaced with —NR⁷—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵,
R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;
R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁷ is independently H, alkyl, or substituted alkyl;
m is 0, 1, 2, 3, or 4; and
provided that the compound of Formula I is not 4′-(2-butyl-imidazo[4,5-d]-pyridazin-5-ylmethyl)-biphenyl-2-carboxylic acid.

68. The process of claim 67, wherein converting the compound of formula IV 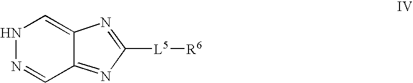
to the compound of formula III 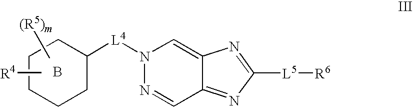
comprises reacting the compound of formula IV with a compound of formula 9.5 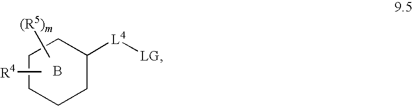
wherein
LG is a leaving group.

69. The process of claim 68, wherein LG is a halogen, hydroxy, alkoxy, substituted alkoxy, or sulfonyloxy.

70. The process of claim 68, wherein the compound of formula IV 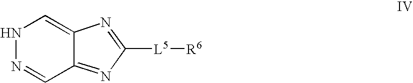
is prepared by a process comprising
a) reacting a compound of formula 10.1 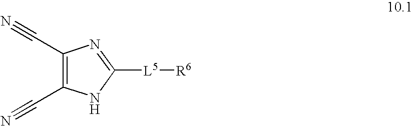
with a reducing reagent
and
b) cyclizing the resulting product to form the compound of formula IV.

71. The process of claim 70, wherein the reducing reagent is diisobutylaluminum hydride.

72. The process of claim 70, wherein the cyclization occurs with hydrazine.

73. The process of claim 70, wherein the compound of formula 10.1 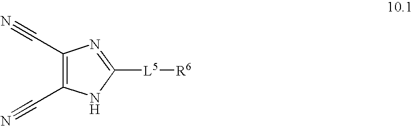
is prepared by a process comprising
cyclizing a compound of formula 10.2

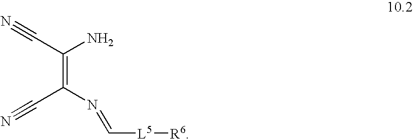

74. The process of claim 73, wherein the cyclization occurs with N-chlorosuccinimide and nicotinamide.

75. The process of claim 73, wherein the compound of formula 10.2 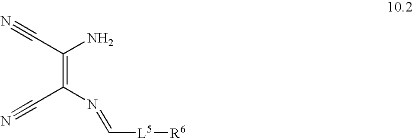
is prepared by a process comprising
reacting a compound of formula 4.3 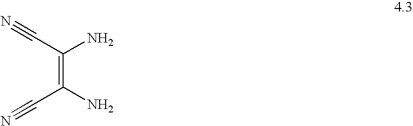
with a compound of formula 10.3

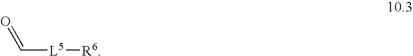

76. The process of claim 67, wherein the compound of formula IV 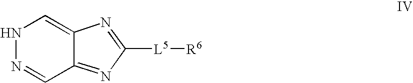
is prepared by a process comprising
a) saponifying a compound of formula 11.1 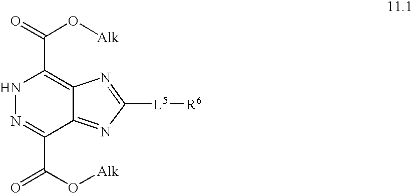
to form a compound of formula 11.2 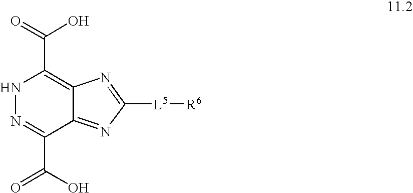
and
b) decarboxylating the compound of formula 11.2 to form the compound of formula IV,
wherein
each Alk is independently chosen and each is an alkyl or substituted alkyl.

77. The process of claim 76, wherein the saponification and decarboxylation occur in the presence of hydrochloric acid and water.

78. The process of claim 76, wherein Alk is CH₃.

79. The process of claim 76, wherein the compound of formula 11.1 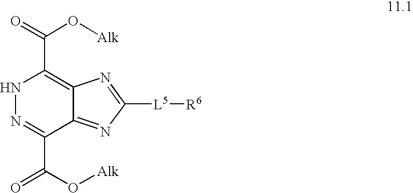
is prepared by a process comprising
reacting a compound of formula 11.3 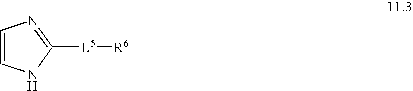
with a compound of formula 11.4 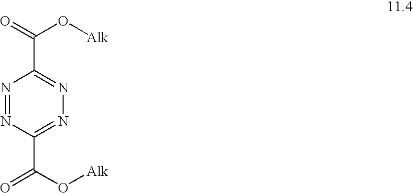
to form the compound of formula 11.1.

80. The process of claim 79, wherein the reaction is heated.

81. The process of claim 79, wherein the compound of formula 11.3 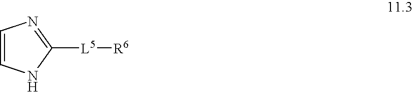
is prepared by a process comprising
reacting a compound of formula 10.3 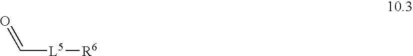
with glyoxal and ammonia.

82. The process of claim 81, wherein converting the compound of formula IV 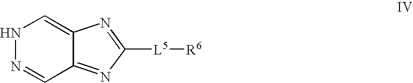
to the compound of formula III 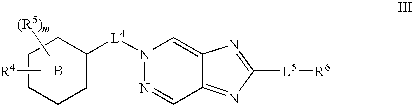
comprises
a) reacting the compound of formula IV with a compound of formula 12.1 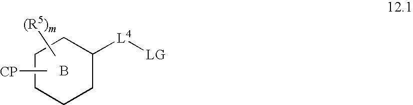
to form a compound of formula 12.2 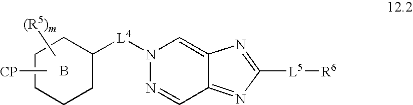
and
b) coupling the compound of formula 12.2 under conditions appropriate to form a new carbon-carbon bond,
wherein
LG is a leaving group
and
CP is a substituent that can undergo a coupling reaction.

83. The process of claim 82, wherein LG is a halogen, hydroxy, alkoxy, substituted alkoxy, or sulfonyloxy.

84. The process of claim 82, wherein the compound of formula 12.2 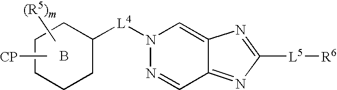
is coupled with a compound of formula 12.3
M-R⁴ 12.3
wherein
M is a substituent that can undergo a coupling reaction.

85. The process of claim 84, wherein the compound of formula 12.3
M-R⁴ 12.3
comprises tin, zinc, magnesium, silicon, or boron.

86. The process of claim 84, wherein the compound of formula 12.3
M-R⁴ 12.3
is selected from an organotin, organozinc, organomagnesium, organosilyl, organoboron, and organotrifluoroborate compound.

87. The process of claim 86, wherein the organoboron is a boronic acid or boronic ester of formula 12.4 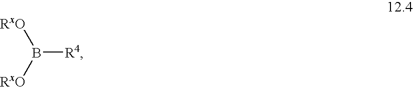
wherein
each R^{x} is independently selected from hydrogen, alkyl, or substituted alkyl
and,
wherein
the R^{x} groups, if alkyl or substituted alky, can optionally be connected.

88. The process of claim 87, wherein the boronic acid is a compound of formula 12.5

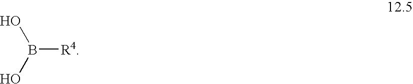

89. The process of claim 82, wherein CP is a halogen or sulfonlyoxy.

90. The process of claim 82, wherein the coupling reaction occurs in the presence of at least one compound comprising palladium, nickel, iron, or copper.

91. The process of claim 90, wherein the coupling reaction occurs in the presence of tetrakistriphenylphosphine palladium.

92. The process of claim 67, wherein converting the compound of formula IV 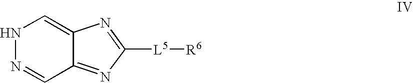
to the compound of formula III 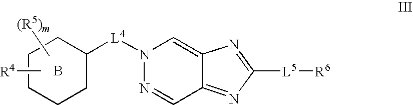
comprises
a) reacting the compound of formula IV with a compound of formula 13.1 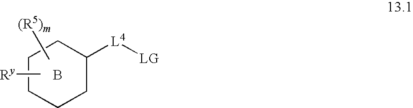
to form a compound of formula 13.2 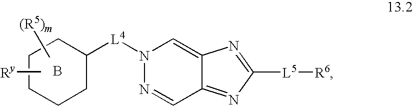
b) reacting the compound of formula 13.2 with a suitable reagent to form a compound of formula 13.3 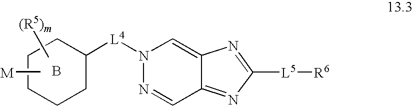
and
c) coupling the compound of formula 13.3 with a compound of formula 13.4
CP—R⁴ 13.4
under conditions appropriate to form a new carbon-carbon bond,
wherein
LG is a leaving group,
R^{y} is a halogen or sulfonyloxy,
and
M and CP are each substituents that can undergo a coupling reaction,

93. The process of claim 92, wherein LG is a halogen, hydroxy, alkoxy, substituted alkoxy, or sulfonyloxy.

94. The process of claim 92, wherein R^{y} is a halogen or sulfonyloxy.

95. The process of claim 92, wherein M comprises tin, zinc, magnesium, silicon, or boron.

96. The process of claim 92, wherein CP is a halogen or sulfonlyoxy.

97. The process of claim 92, wherein the coupling reaction of step (c) occurs in the presence of at least one compound comprising palladium, nickel, iron, or copper.

98. The process of claim 97, wherein the coupling reaction of step (c) occurs in the presence of tetrakistriphenylphosphine palladium.

99. The process of claim 68, wherein the compound of formula 9.5 is 5-(Chloromethyl)-2-[4-propyloxy)-2-(trifluoromethyl)phenyl]pyrimidine.

100. The process of claim 68, wherein the compound of formula 9.5 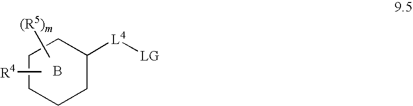
is prepared by a process comprising reacting a compound of formula 16.1 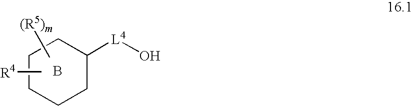
with a compound containing LG to form the compound of formula 9.5.

101. The process of claim 100, wherein the compound containing LG is a chlorinated compound.

102. The process of claim 100, wherein the chlorinated compound is thionyl chloride.

103. The process of claim 100, wherein the compound of formula 16.1 is {2-[4-(Propyloxy)-2-(trifluoromethyl)phenyl]-5-pyrimidinyl}methanol.

104. The process of claim 100, wherein the compound of formula 16.1 is prepared by a process comprising reacting a compound of formula 16.2 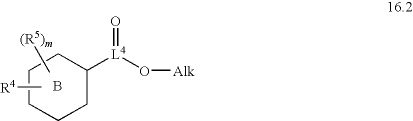
with a reducing reagent to form the compound of formula 16.1, wherein Alk is alkyl or substituted alkyl.

105. The process of claim 104, wherein Alk in the compound of formula 16.2 is CH₃.

106. The process of claim 104, wherein the reducing agent is selected from the group consisting of lithium aluminum hydride, sodium borohydride and diisobutylaluminum hydride.

107. The process of claim 104, wherein the compound of formula 16.2 is Methyl 2-[4-(Propyloxy)-2-(trifluoromethyl)phenyl]-5-pyrimidinecarboxylate.

108. The process of claim 104, wherein the compound of formula 16.2 is prepared by a process comprising reacting a compound of formula 16.3 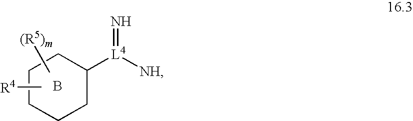
or a salt thereof, with an alkoxy ester to form the compound of formula 16.2.

109. The process of claim 108, wherein reaction of the compound of formula 16.3 or salt thereof with the alkoxy ester occurs in the presence of nitrogen and optionally heat.

110. The process of claim 108, wherein the alkoxy ester is methyl-2-[bis(methyloxy)methyl]-3-hydroxy-2-propenoate or a salt thereof.

111. The process of claim 108, wherein the compound of formula 16.3 or salt thereof is 4-(Propyloxy)-2-(trifluoromethyl)benzenecarboximidamide hydrochloride.

112. The process of claim 108, wherein the compound of formula 16.3 or salt thereof is prepared by a process comprising reacting a compound of formula 16.4 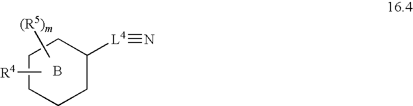
with a nucleophilic base to form the compound of formula 16.3 or a salt thereof.

113. The process of claim 112, wherein reaction of the compound of formula 16.4 occurs in the presence of nitrogen and optionally heat.

114. The process of claim 112, wherein the nucleophilic base is a salt of hexamethyldisilazide.

115. The process of claim 112, wherein the compound of formula 16.4 is 4-(Propyloxy)-2-(trifluoromethyl)benzonitrile.

116. A composition comprising
(1) a compound of formula I or a salt or solvate thereof 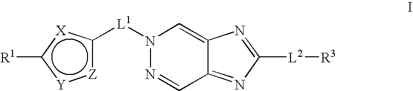
wherein
a) when X is CR² or N, one of Y or Z is O and the other of Y or Z is N; or one of Y or Z is N and the other of Y or Z is NR^{a};
b) when X is O, NR^{a}, or S(O)ₚ wherein p is 0 or 1, one of Y or Z is N and the other of Y or Z is Nor CR²;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;
R^{a} and R^{b} are independently H, alkyl, or substituted alkyl;
R¹ and R³ are independently selected from aryl, substituted aryl, heteroaryl, substituted heteroaryl, heterocyclyl, substituted heterocyclyl, cycloalkyl, and substituted cycloalkyl;
and
R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano; and
(2) a detectable amount of one or more compounds selected from:
a compound of formula 1.1 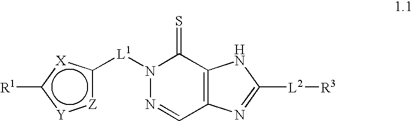
or a salt thereof;
P₂S₅;
a compound of formula 2.1 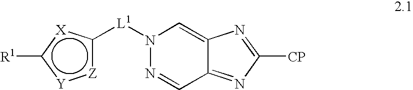
or a salt thereof, wherein CP is a group that can undergo a coupling reaction;
a compound of formula 14.1 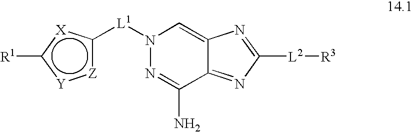
or a salt thereof, wherein L¹, L², R¹, R³, X, Y, and Z are as defined above;
a compound comprising tin, zinc, magnesium, silicon, or boron;
a compound comprising palladium, nickel, iron, or copper;
hydrazine;
and
Cs₂CO₃.

117. A composition comprising
(1) a compound of formula III or a salt or solvate thereof 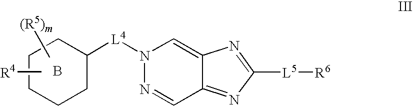
wherein
ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L⁴ is L⁶;
L⁵ is a bond or L⁶;
L⁶ is independently C₃₋₆ cycloalkylene or is C₁₋₅ alkylene where one or two —CH₂— groups of said C₁₋₅ alkylene are optionally replaced with —NR⁷—, —S—, —(C═O)—, or —O— and optionally two —CH₂— groups together form a double bond or triple bond provided that L⁶ does not contain an —O—O—, —S—O—, or —S—S— group, and wherein said C₁ to C₅ alkylene is optionally substituted with one to two groups independently selected from spirocycloalkyl and R⁵;
R⁴ is independently selected from R⁵, aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁵ is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;
R⁶ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁷ is independently H, alkyl, or substituted alkyl;
m is 0, 1, 2, 3, or 4; and
provided that the compound of Formula I is not 4′-(2-butyl-imidazo[4,5-d]-pyridazin-5-ylmethyl)-biphenyl-2-carboxylic acid.
and
(2) a detectable amount of one or more compounds selected from:
a compound of formula 6.1 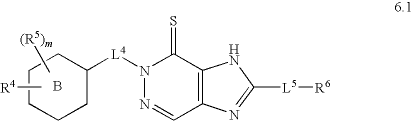
or a salt thereof;
P₂S₅;
a compound of formula 7.1 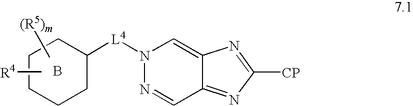
or a salt thereof, wherein CP is a group that can undergo a coupling reaction;
a compound of formula 15.1 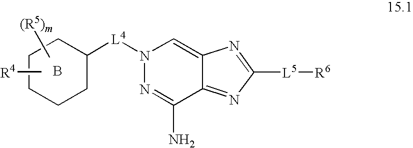
or a salt thereof, wherein L⁴, L⁵, R⁴, R⁵, R⁶, and m are as defined above;
a compound comprising tin, zinc, magnesium, silicon, or boron;
a compound comprising palladium, nickel, iron, or copper;
hydrazine;
and
Cs₂CO₃.
